(19) United States
(12) Patent Application Publication
(10) Pub. No.: US 2026/0242341 A1
(43) Pub. Date: Aug. 20, 2026

(54) REGIO-CONTROLLABLE [2+2] BENZANNULATION WITH TWO ADJACENT C(SP3)-H BONDS

(71) Applicant: THE SCRIPPS RESEARCH INSTITUTE, La Jolla, CA (US)

(72) Inventors: Jin-Quan YU, San Diego, CA (US); Ji-Min YANG, San Diego, CA (US); Yu-Kun LIN, San Diego, CA (US)

(73) Assignee: THE SCRIPPS RESEARCH INSTITUTE, La Jolla, CA (US)

(21) Appl. No.: 19/159,354

(22) PCT Filed: Feb. 27, 2024
(86) PCT No.: PCT/US2024/017390

§ 371 Date: Aug. 25, 2025

Related U.S. Application Data
(60) Provisional application No. 63/487,381, filed on Feb. 28, 2023.

(51) Int. Cl. C07D 221/04; B01J 31/18; B01J 31/22; C07C 51/363; C07C 57/62; C07C 209/46; C07D 207/06; C07D 209/56; C07D 231/14; C07D 241/38; C07D 265/30; C07D 307/52; C07D 319/14; C07D 333/20; C07F 9/40; C07H 19/01; C07J 7/00

(52) U.S. Cl. CPC C07D 221/04; B01J 31/1805; B01J 31/183; B01J 31/2208; B01J 31/2213; B01J 31/2291; C07C 51/363; C07C 57/62; C07C 209/46; C07D 207/06; C07D 209/56; C07D 231/14; C07D 241/38; C07D 265/30; C07D 307/52; C07D 319/14; C07D 333/20; C07F 9/4003; C07H 19/01; C07J 7/002; B01J 2231/46; B01J 2531/004; B01J 2531/824; C07C 2602/06; C07C 2603/12; C07C 2603/40; C07C 2603/90

(57) ABSTRACT

This application discloses methods of twofold β,γ-methylene C(sp³)-H activation/C—C bond formation using a Pd(II) catalyst bound to amide-pyridone ligands, resulting in regio-controllable [2+2] annulation between aliphatic acids and dihaloarenes.

### CROSS-REFERENCE TO RELATED APPLICATION

This application claims the benefit of U.S. Provisional Application No. 63/487,381, filed on Feb. 28, 2023, which is incorporated herein by reference in its entirety.

### GOVERNMENT SUPPORT

This invention was made with government support under GM084019 awarded by the National Institutes of Health. The government has certain rights in the invention.

### FIELD OF THE INVENTION

This application relates to methods of twofold β,γ-methylene C(sp³)-H activation/C—C bond formation using a Pd(II) catalyst bound to amide-pyridone ligands, resulting in regio-controllable [2+2] annulation between aliphatic acids and dihaloarenes.

### BACKGROUND OF THE INVENTION

Pd(II)-catalyzed C—H activation reactions of a single methylene C—H bond in free aliphatic acids have recently been realized through ligand development (1, 2). Double C—H activation of two adjacent inert methylene C—H bonds will open avenues for developing even more efficient disconnections for ideal synthesis, especially for providing solutions to unsolved problems in cycloaddition reactions (FIG. 1A). For example, benzocyclobutenes (BCBs) are an important class of rigid four-membered carbocycles found in natural products (3, 4) that has demonstrated great potential as therapeutical molecular scaffolds (5, 6), versatile synthons (7), and functional motifs in material science and mechanochemistry (8, 9) (FIG. 1). BCB-based rigid and three-dimensional pharmacophores have led to the discovery of Ivabradine, the FDA-approved drug for the treatment of heart failure and heart-related chest pain (5). The BCB-analogue of the psychoactive phenethylamine was found to be a superior affinity ligand for the human 5-HT_{2A} receptor compared with the conformationally flexible parent phenethylamine and the benzocyclopentane analogue (FIG. 1B) (10). Currently, the [2+2]cycloaddition of alkenes and benzynes is one of the most common synthetic approaches towards BCBs (11). However, controlling the regioselectivity of this cycloaddition reaction is an unsolved problem. As illustrated in FIG. 1C, the [2+2]cycloadditions between a substituted alkene and a benzyne typically lead to head-to-head and head-to-tail regioisomers with a mixture of diastereoisomers. A number of alternative intramolecular processes, such as cyclization of o-quinodimethane (12), cyclative C—H arylations (13-16), visible-light induced radical processes (17) and others (18) have been developed. A modular two-component intermolecular synthesis of BCBs has recently been developed using an elegant Pd-catalyzed [2+2] annulation of alkenes bearing an amide directing group and arylboronic acids (19), however, this method doesn't allow regiocontrol. In addition, existing methods for BCB synthesis suffer from scope and efficiency limitations due to the requirement of preinstallation of reactive functional groups such as double or triple bonds. Notably, the synthesis of medicinally important heterocyclic BCB using these reactions is challenging (20, 21). The recently demonstrated Pd-catalyzed activation of β- or γ-C—H bonds of aliphatic acids (1, 2, 22) inspired us to propose a formal [2+2] annulation of aliphatic acids with dihaloarenes through an unprecedented dual sequential methylene C—H activation at the β- and γ-positions (FIG. 1D). The state of the art of dual functionalization of two adjacent methylene C—H bonds is limited to allylic and benzylic positions, or sites α- to a heteroatom (23-26). Thus, there exists a need in the field of synthetic organic chemistry for methods of diverse dual methylene C—H activation at the β- and γ-positions, especially the double functionalization of two adjacent inert methylene C—H bonds.

### BRIEF DESCRIPTION OF THE FIGURES

FIG. 1. [2+2] Annulation reaction via dual methylene C—H bond functionalization.

FIG. 2. Cyclic aliphatic acids scope for the [2+2] annulation reaction.

FIG. 3. Acyclic aliphatic acids scope for the [2+2] annulation reaction and transformations of the products.

FIG. 4. Dihaloarenes scope for the [2+2] annulation reaction.

### SUMMARY OF THE INVENTION

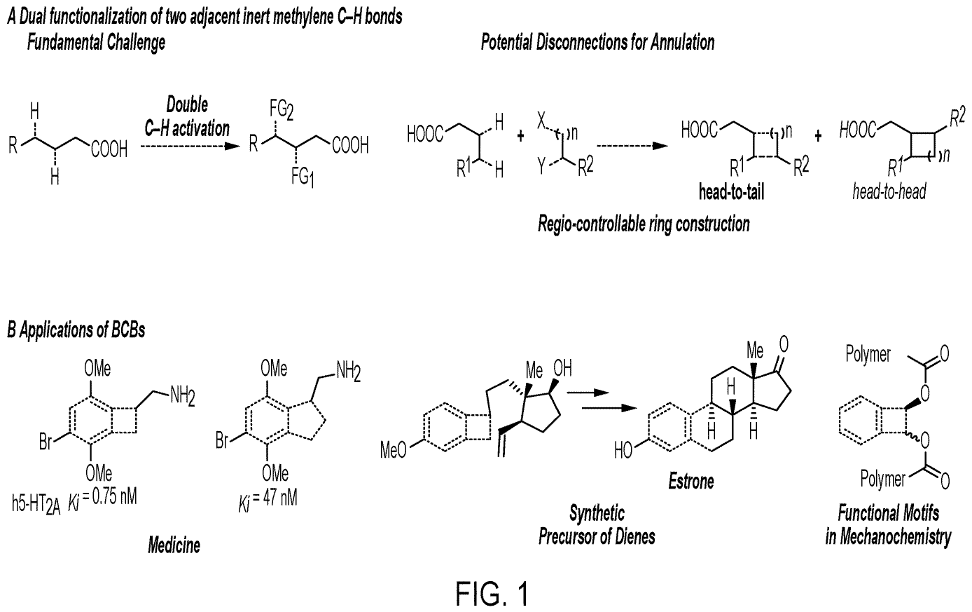

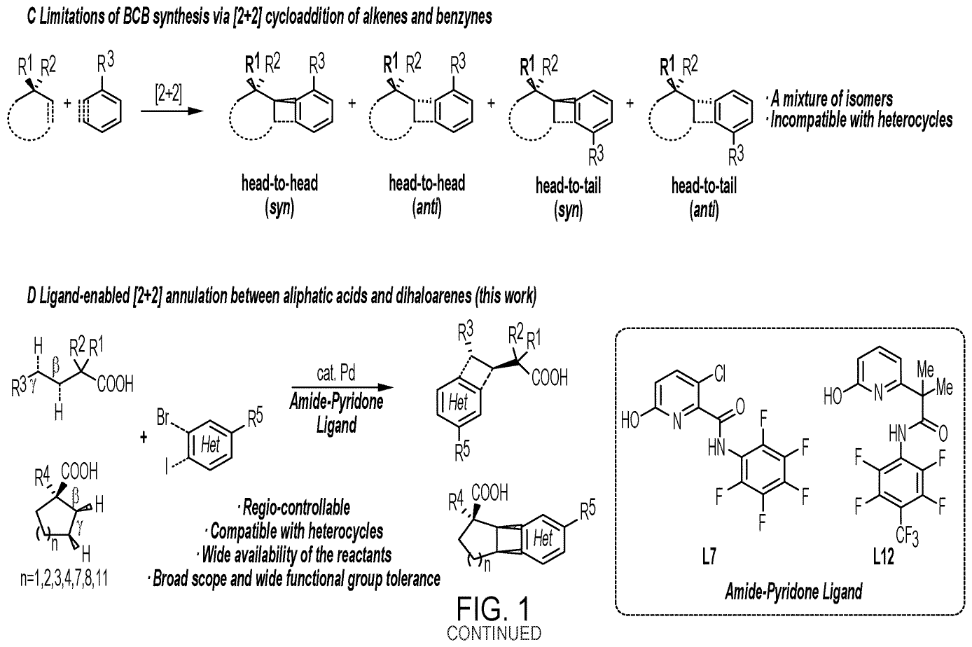

Herein reported is a palladium-catalyzed regio-controllable BCB synthesis through the annulation of aliphatic acids with dihaloarenes enabled by amide-pyridone ligands, in which the regiocontrol is achieved through the differentiation between the aryl iodide and bromide sites. The direct use of abundant and structurally varied acyclic and cyclic acids as substrates without pre-functionalization significantly expands access to BCBs with unprecedented diversity, including heterocyclic BCB scaffolds (FIG. 1D).

The application provides a method of twofold β,γ-methylene C(sp³)-H activation/C—C bond formation, comprising regio-controllable [2+2] annulation between an aliphatic acid and a dihaloarene in the presence of a Pd(II) catalyst bound to an amide-pyridone ligand.

The application provides the above method, wherein the regio-controllable [2+2] annulation between an aliphatic acid and a dihaloarene occurs according to the following reaction scheme:

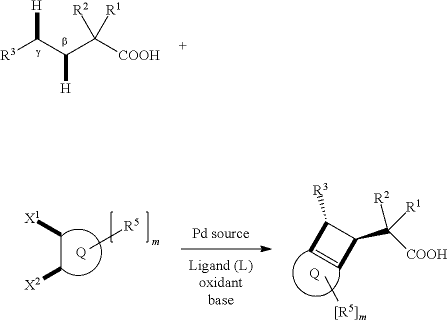

wherein:
Q is Ar or Het;
   Ar is (C₆-C₁₀)aryl, optionally substituted with one or more R^{a};
   Het is (C₅-C₈)heteroaryl, optionally substituted with one or more R^{a};
X¹ and X² are independently Br or I;
R¹ and R² are independently H or (C₁-C₆)alkyl;
R³ is (C₁-C₆)alkyl, (C₁-C₆)heteroalkyl, halo (C₁-C₆)alkyl, (C₁-C₆)alkyl-OTBS, —C(═O)OH, —C(═O)O(C₁-C₆)alkyl, (C₃-C₇)cycloalkyl, (C₃-C₇)heterocycloalkyl, (C₆-C₁₀)aryl, (C₅-C₆)heteroaryl, (C₁-C₆)alkyl (C₃-C₇)cycloalkyl, (C₁-C₆)alkyl (C₃-C₇)heterocycloalkyl, (C₁-C₆)alkyl (C₆-C₁₀)aryl, —O(C₆-C₁₀)aryl, and (C₁-C₆)alkyl (C₅-C₆)heteroaryl, wherein each is optionally and independently substituted with one or more R^{a};
   each R^{a} is independently selected from halo, —OH, —C(═O)O(C₁-C₆)alkyl, CN, NO₂, —P(═O)(O(C₁-C₆)alkyl)₂-C(═O)H, NH₂, (C₁-C₆)alkyl, halo (C₁-C₆)alkyl, (C₁-C₆)heteroalkyl, —C(═O)(C₁-C₆)alkyl, oxo, Ts, —S(═O)₂F, —OS(═O)₂F, or —S(═O)₂(C₁-C₆)alkyl, and —C(═O)NH(C₁-C₆)alkyl;
each R⁵ is independently halo, OH, CN, NO₂, —OTf, (C₁-C₆)alkyl, —C(═O)OH, —C(═O)O(C₁-C₆)alkyl, —C(═O)H, —C(═O)(C₁-C₆)alkyl, (C₁-C₆)alkyl, (C₁-C₆)heteroalkyl, halo (C₁-C₆)alkyl, —C(═O)(C₁-C₆)alkyl, —C(═O)(C₃-C₇)heterocycloalkyl, (C₅-C₆)heteroaryl, or —C(═O)NH(C₁-C₆)alkyl(C₅-C₆)heteroaryl;
   or two R⁵ may together form (C₃-C₇)cycloalkyl, (C₃-C₇)heterocycloalkyl, (C₆-C₁₀)aryl, or (C₅-C₆)heteroaryl, wherein each is optionally and independently substituted with one or more R^{a}; and
m is 0, 1, or 2;
including enantiomers, scalemic or racemic mixtures, and pharmaceutically acceptable salts thereof.

The application alternatively provides the above method, wherein the regio-controllable [2+2] annulation between an aliphatic acid and a dihaloarene occurs according to the following reaction scheme:

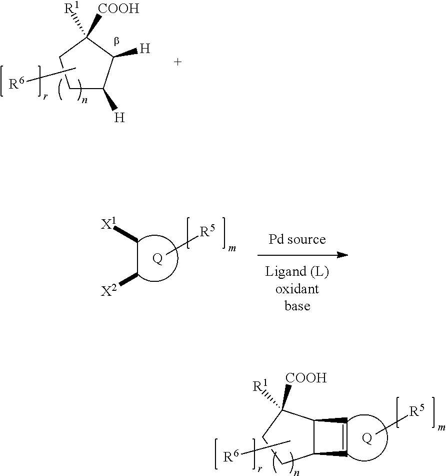

wherein:
Q is Ar or Het;
   Ar is (C₆-C₁₀)aryl, optionally substituted with one or more R^{a};
Het is (C₅-C₈)heteroaryl, optionally substituted with one or more R^{a};
X¹ and X² are independently Br or I;
R¹ is R^{1a} or R^{1b};
   R^{1a} is H, CN, or halo;
   R^{1b} is (C₁-C₆)alkyl, (C₁-C₆)heteroalkyl, halo (C₁-C₆)alkyl, (C₁-C₆)alkyl-OTBS, (C₃-C₇)cycloalkyl, (C₃-C₇)heterocycloalkyl, (C₆-C₁₀)aryl, (C₅-C₆)heteroaryl, (C₁-C₆)alkyl (C₃-C₇)cycloalkyl, (C₁-C₆)alkyl (C₃-C₇)heterocycloalkyl, (C₁-C₆)alkyl (C₆-C₁₀)aryl, and (C₁ -C₆)alkyl (C₅-C₆)heteroaryl, wherein each is optionally and independently substituted with one or more R^{a};
      each R^{a} is independently selected from halo, —OH, (C₁-C₆)alkyl, —C(═O)O(C₁-C₆)alkyl, CN, NO₂, —P(═O)(O(C₁-C₆)alkyl)₂-C(═O)H, —C(═O)(C₁-C₆)alkyl, —C(═O)OH, —C(═O)O(C₁-C₆)alkyl, NH₂, halo (C₁-C₆)alkyl, (C₁-C₆)heteroalkyl, —C(═O)(C₁-C₆)alkyl, oxo, Ts, —S(═O)₂F, —OS(═O)₂F, or —S(═O)₂(C₁-C₆)alkyl, and —C(═O)NH(C₁-C₆)alkyl;
each R⁵ is independently halo, OH, CN, NO₂, —OTf, (C₁-C₆)alkyl, —C(═O)OH, —C(═O)O(C₁-C₆)alkyl, —C(═O)H, —C(═O)(C₁-C₆)alkyl, (C₁-C₆)alkyl, (C₁-C₆)heteroalkyl, halo (C₁-C₆)alkyl, —C(═O)(C₁-C₆)alkyl, —C(═O)(C₃-C₇)heterocycloalkyl, (C₅-C₆)heteroaryl, —C(═O)NH(C₁-C₆)alkyl(C₅-C₆)heteroaryl;
   or two R⁵ may together form (C₃-C₇)cycloalkyl, (C₃-C₇)heterocycloalkyl, (C₆-C₁₀)aryl, or (C₅-C₆)heteroaryl, wherein each is optionally and independently substituted with one or more R^{a}; and
each R⁶ is independently halo, OH, CN, (C₁-C₆)alkyl, (C₁-C₆)heteroalkyl, or halo (C₁-C₆)alkyl;
m is 0, 1, or 2;
n is 1, 2, 3, 4, 7, 8, or 11; and
r is 0, 1, or 2;
including enantiomers, scalemic or racemic mixtures, and pharmaceutically acceptable salts thereof.

The application further provides a compound having the structure selected from the group consisting of:

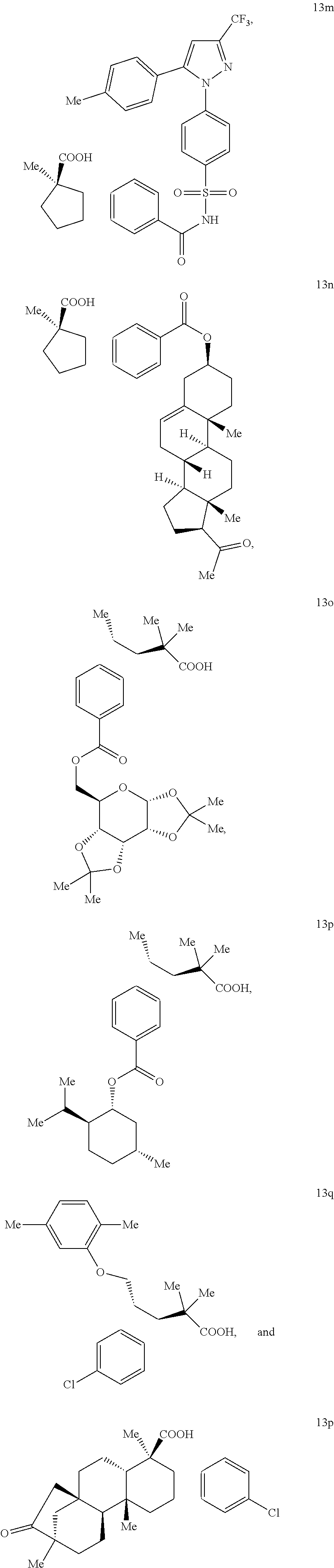

including enantiomers, scalemic or racemic mixtures, and pharmaceutically acceptable salts thereof.

The application further provides a method of preparing the Compound 6, comprising the following steps:

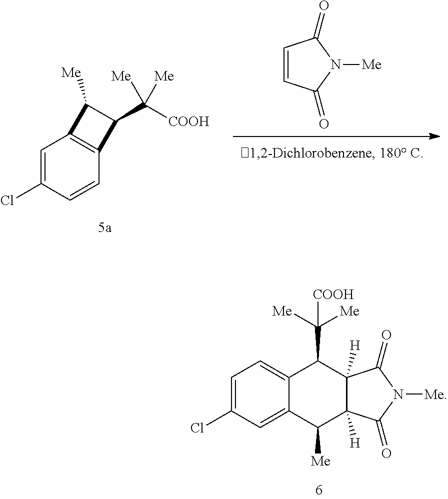

The application further provides a method of preparing the Compound 7, comprising the following steps:

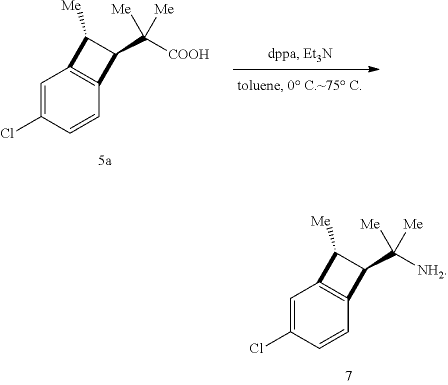

The application further provides a method of preparing the Compound 10, comprising the following steps:

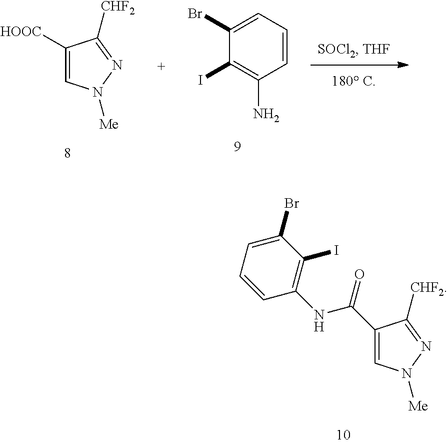

The application further provides a method of preparing the bioreactive agrochemical 11, comprising the following steps:

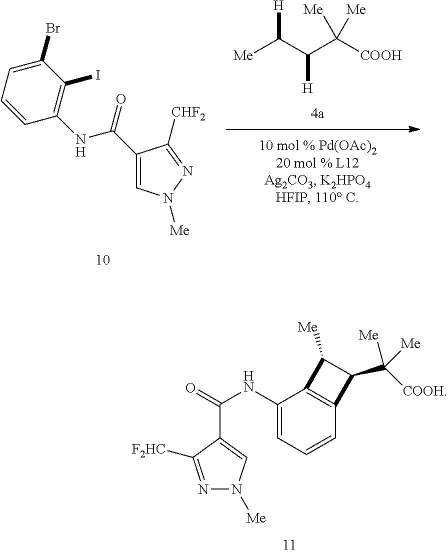

The application further provides a compound having the Formula I

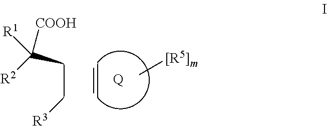

wherein:
Q is Ar or Het;
   Ar is (C₆-C₁₀)aryl, optionally substituted with one or more R^{a};
   Het is (C₅-C₈)heteroaryl, optionally substituted with one or more R^{a};
R¹ and R² are independently H or (C₁-C₆)alkyl, wherein both R¹ and R² are not H;
R³ is (C₁-C₆)alkyl, (C₁-C₆)heteroalkyl, halo (C₁-C₆)alkyl, (C₁-C₆)alkyl-OTBS, —C(═O)OH, —C(═O)O(C₁-C₆)alkyl, (C₃-C₇)cycloalkyl, (C₃-C₇)heterocycloalkyl, (C₆-C₁₀)aryl, (C₅-C₆)heteroaryl, (C₁-C₆)alkyl (C₃-C₇)cycloalkyl, (C₁-C₆)alkyl (C₃-C₇)heterocycloalkyl, (C₁ -C₆)alkyl (C₆-C₁₀)aryl, —O(C₆-C₁₀)aryl, and (C₁-C₆)alkyl (C₅-C₆)heteroaryl, wherein each is optionally and independently substituted with one or more R^{a};
   each R^{a} is independently selected from halo, —OH, —C(═O)O(C₁-C₆)alkyl, CN, NO₂, —P(═O)(O(C₁-C₆)alkyl)₂-C(═O)H, NH₂, (C₁-C₆)alkyl, halo (C₁-C₆)alkyl, (C₁-C₆)heteroalkyl, —C(═O)(C₁-C₆)alkyl, oxo, Ts, —S(═O)₂F, —OS(═O)₂F, or —S(═O)₂(C₁-C₆)alkyl, and —C(═O)NH(C₁-C₆)alkyl;
each R⁵ is independently halo, OH, CN, NO₂, —OTf, (C₁-C₆)alkyl, —C(═O)OH, —C(═O)O(C₁-C₆)alkyl, —C(═O)H, —C(═O)(C₁-C₆)alkyl, (C₁-C₆)alkyl, (C₁-C₆)heteroalkyl, halo (C₁-C₆)alkyl, —C(═O)(C₁-C₆)alkyl, —C(═O)(C₃-C₇)heterocycloalkyl, (C₅-C₆)heteroaryl, or —C(═O)NH(C₁-C₆)alkyl(C₅-C₆)heteroaryl;
   or two R⁵ may together form (C₃-C₇)cycloalkyl, (C₃-C₇)heterocycloalkyl, (C₆-C₁₀)aryl, or (C₅-C₆)heteroaryl, wherein each is optionally and independently substituted with one or more R^{a}; and
m is 0, 1, or 2;
including enantiomers, scalemic or racemic mixtures, and pharmaceutically acceptable salts thereof.

The application further provides a compound having the Formula II

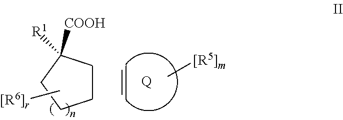

wherein:
Q is Ar or Het;
   Ar is (C₆-C₁₀)aryl, optionally substituted with one or more R^{a};
Het is (C₅-C₈)heteroaryl, optionally substituted with one or more R^{a};
R¹ is (C₁-C₆)alkyl, (C₁-C₆)heteroalkyl, halo (C₁-C₆)alkyl, (C₁-C₆)alkyl-OTBS, (C₃-C₇)cycloalkyl, (C₃-C₇)heterocycloalkyl, (C₆-C₁₀)aryl, (C₅-C₆)heteroaryl, (C₁-C₆)alkyl (C₃-C₇)cycloalkyl, (C₁-C₆)alkyl (C₃-C₇)heterocycloalkyl, (C₁-C₆)alkyl (C₆-C₁₀)aryl, and (C₁-C₆)alkyl (C₅-C₆)heteroaryl, wherein each is optionally and independently substituted with one or more R^{a};
   each R^{a} is independently selected from halo, —OH, (C₁-C₆)alkyl, —C(═O)O(C₁-C₆)alkyl, CN, NO₂, —P(═O)(O(C₁-C₆)alkyl)₂-C(═O)H, —C(═O)(C₁-C₆)alkyl, —C(═O)OH, —C(═O)O(C₁-C₆)alkyl, NH₂, halo (C₁-C₆)alkyl, (C₁-C₆)heteroalkyl, —C(═O)(C₁-C₆)alkyl, oxo, Ts, —S(═O)₂F, —OS(═O)₂F, or —S(═O)₂(C₁-C₆)alkyl, and —C(═O)NH(C₁-C₆)alkyl;
each R⁵ is independently halo, OH, CN, NO₂, —OTf, (C₁-C₆)alkyl, —C(═O)OH, —C(═O)O(C₁-C₆)alkyl, —C(═O)H, —C(═O)(C₁-C₆)alkyl, (C₁-C₆)alkyl, (C₁-C₆)heteroalkyl, halo (C₁-C₆)alkyl, —C(═O)(C₁-C₆)alkyl, —C(═O)(C₃-C₇)heterocycloalkyl, (C₅-C₆)heteroaryl, —C(═O)NH(C₁-C₆)alkyl(C₅-C₆)heteroaryl;
   or two R⁵ may together form (C₃-C₇)cycloalkyl, (C₃-C₇)heterocycloalkyl, (C₆-C₁₀)aryl, or (C₅-C₆)heteroaryl, wherein each is optionally and independently substituted with one or more R^{a}; and
each R⁶ is independently halo, OH, CN, (C₁-C₆)alkyl, (C₁-C₆)heteroalkyl, or halo (C₁-C₆)alkyl;
n is 1, 2, 3, 4, 7, 8, or 11; and
r is 0, 1, or 2;
including enantiomers, scalemic or racemic mixtures, and pharmaceutically acceptable salts thereof.

### DETAILED DESCRIPTION OF THE INVENTION

The double functionalization of two adjacent inert methylene C—H bonds represents a new avenue for developing unprecedented disconnections as it unlocks simple aliphatic fragments as surrogates for double bonds which could overcome persistent limitations of traditional cycloaddition reactions. For example, regiocontrol in traditional cycloaddition is often not possible. In particular, controlling the regioselectivity in the synthesis of benzocyclobutenes (BCBs) through cycloaddition remains an unsolved challenge. The imperative need in modern medicinal chemistry for new scaffolds, such as bicyclo[1.1.1]pentanes (BCPs), speaks to the importance of rapid access to diverse rigid carbocycles rich in sp³ character. Herein reported is a Pd-catalyzed double C—H activation of two adjacent methylene units as enabled by newly developed bidentate amide-pyridone ligands, achieving a regio-controllable synthesis of BCBs through a formal [2+2] cycloaddition involving σ-bonds only (two C—H bonds and two aryl-halogen bonds). A wide range of cyclic and acyclic aliphatic acids, as well as dihaloheteroarenes are compatible, generating diversely functionalized BCBs and hetero-BCBs present in drug molecules and bioactive natural products.

During the mechanistic investigations of the methylene C—H lactonization of dicarboxylic acids, the β,γ-C—H deuteration of 7-ethoxy-7-oxoheptanoic acid was observed (1). This observation prompted the question of whether a sequential twofold C—H coupling process with ortho-dihaloarenes could be developed to construct BCB scaffolds. Using 1-propyl-1-cyclopentanecarboxylic acid 1a and dihaloarene coupling partner 1-bromo-4-chloro-2-iodobenzene 2a, the feasibility of this transformation using a series of pyridine-pyridone ligands recently developed as described herein were tested (Table S1). Encouragingly, the desired BCB 3a was indeed formed with a single isomer. Extensive control experiments using various possible intermediates indicate that a highly complex Pd(II)/Pd(0)/Pd(II)/Pd(IV) catalytic cycle involving sequential dehydrogenation, intermolecular oxidative addition, carbopalladation, intramolecular oxidative addition and reductive elimination could be operative (for preliminary mechanistic investigation, see Examples for details). However, attempts to improve the reaction yield using pyridine-pyridone ligands and other known ligand scaffolds (L28 to L37) under various conditions proved unsuccessful.

These initial results with pyridine-pyridone ligands, while promising, pointed to the need for more effective ligands. Since the 2-pyridone serves a critical role as the internal base for C—H cleavage (27), developing alternatives for the pyridine arm of the scaffold became the focus. Inspired by the superior reactivity of the electron-deficient amide motif as a directing group for Pd(II) in C(sp³)-H activation (28), a class of bidentate ligands bearing both a pyridone and an electron-deficient amide derived from perfluorinated anilines were synthesized. Amide-pyridone ligand L1, posited to coordinate to Pd(II) as five-membered chelate, afforded significantly improved yield in the annulation reaction (38%). Through extensive structural tuning, ligand L7 was identified as optimal, providing BCB 3a in 90% isolated yield.

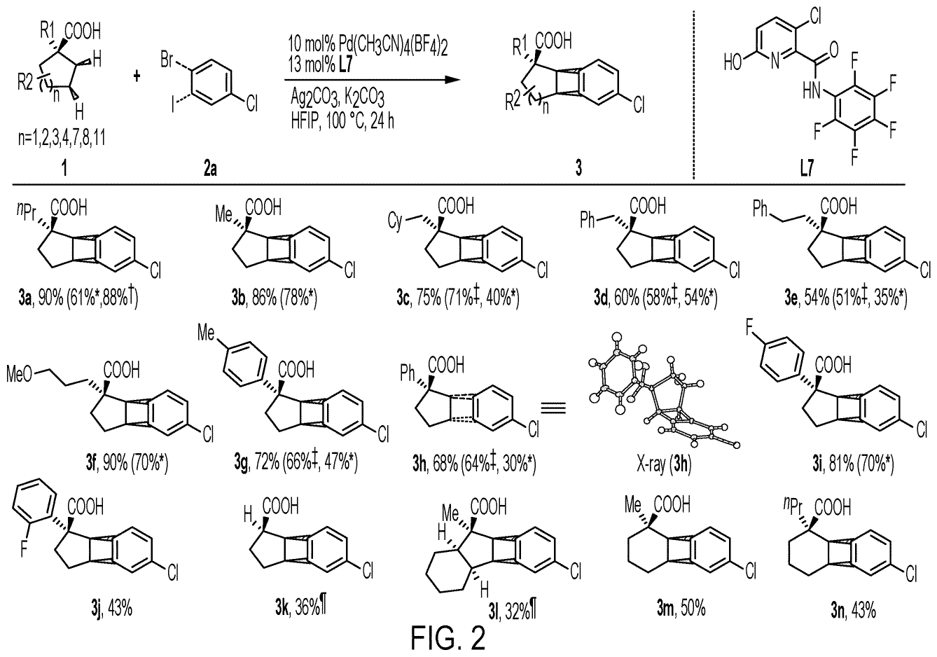

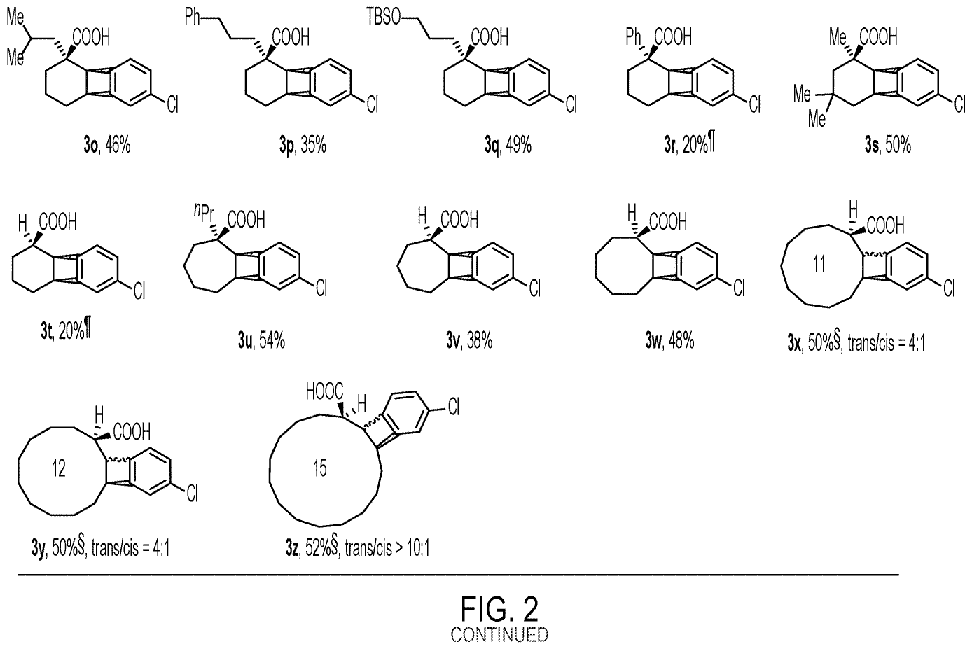

Under these optimized conditions, the substrate scope of the [2+2] annulation reaction (FIG. 2) were subsequently evaluated. A wide range of cyclic aliphatic acids including five- (1a-1), six- (1m-t), seven- (1u-v), and eight-membered (1w) rings were compatible to afford cis-BCB products as single regioisomers (3a-3w). Larger cyclic aliphatic acids including 11- (1x), 12- (1y) and 15-membered (1z) rings produced trans-BCB products as major isomers. Remaining unreacted aliphatic acids starting material were recovered when the yields are relatively low. Notably, ligand L7 allowed reduction of Pd loading from 10% to 5% or 1% while maintaining synthetically useful yields for the [2+2] annulation between acids (1a-1i) as selected examples and dihaloarene 2a. An array of diverse substitutions at the α-position of carboxylic acid were tested in the reaction. Alkyl groups (3a-3f, 3l-q, 3s, 3u) and aryl groups (3g-3j, 3r) with electronically diverse substituents were compatible with this protocol despite the presence of reactive methyl and aryl C—H bonds. Functionalities such as methoxy (3f), OTBS (3q), phenyl (3d-e, 3p) were well-tolerated. Bicyclic aliphatic acid (1l) was converted to the corresponding fused 6-5-4-6 ring (3l) in 32% yield. Cyclohexanecarboxylic acid bearing substitutions at the 3-position (1s) consistently provided the corresponding product (3s) in moderate yield. In addition to the aliphatic acids containing an α-quaternary center, aliphatic acids containing an α-hydrogen (1k, 1t, 1v-1z) afforded the products (3k, 3t, 3v-3z) in 20%-52% yield. The structure of 3h was confirmed by single-crystal X-ray diffraction analysis. Notably, quinoline-pyridone ligand L28 was more suitable for 8-15-membered rings (1w-1z). The scalability of this [2+2] annulation reaction, in which 1.05 gram of the desired product 3a could be obtained in 88% isolated yield were also investigated (see Examples).

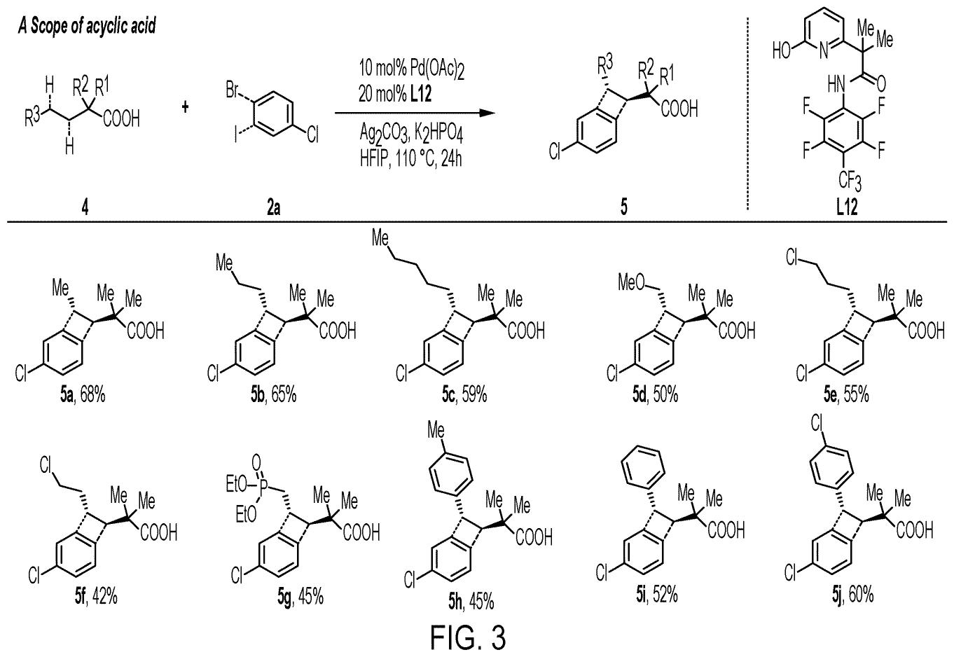

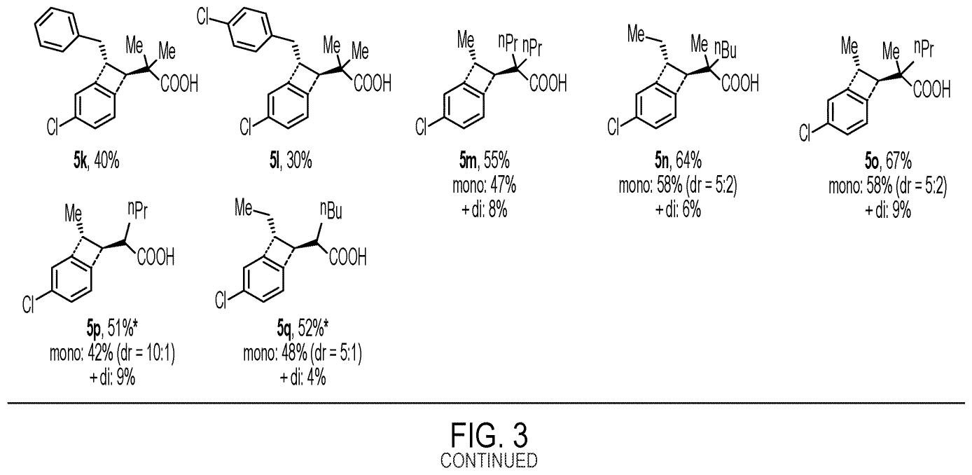

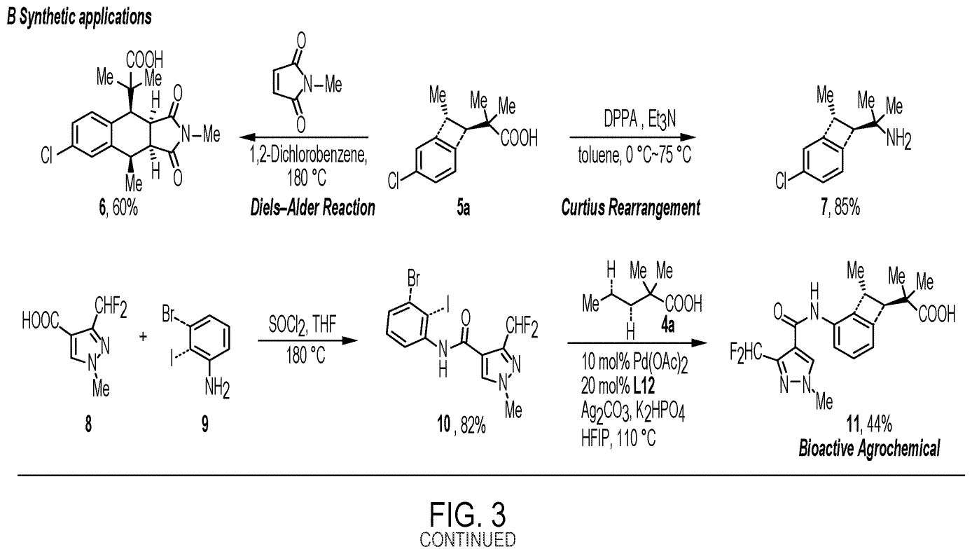

Acyclic aliphatic acid 4a only resulted in a low yield with L7 (2%) (Table S6). It was found that six-membered chelating amide-pyridone ligand L12 emerged as the most effective ligand for these linear substrates, affording diverse trans-BCBs (FIG. 3). A wide range of functional groups, such as methoxy (5d), chloro (5e-5f), phosphonate (5g), and substituted aryl groups (5h-l) were tolerated. Aliphatic acids bearing α-gem-dimethyl groups (4a-1) or a single methyl group (4n-o) are compatible with this protocol despite the presence of the typically more accessible α-methyl C—H bonds. In addition to the substrates containing an α-quaternary center (4a-o), aliphatic acids containing an α-hydrogen (4p-q) were also found to be viable for this protocol, as exemplified with products 5p and 5q, generating the products in moderate yields. A number of synthetic applications of benzocyclobutyl acid products are also demonstrated. Heating a solution of 5a as an o-quinodimethane precursor and N-methylmaleimide in o-dichlorobenzene at 180° C. provided Diels-Alder adduct 6 in 60% yield. 5a could be readily transformed to medicinally important amine (7). The benzocyclobutyl amide 11, a patented bioactive molecule for crop protection (29), could be rapidly synthesized by [2+2] annulation of aliphatic acid 4a and N-dihaloaryl amide 10 in a two-step sequence.

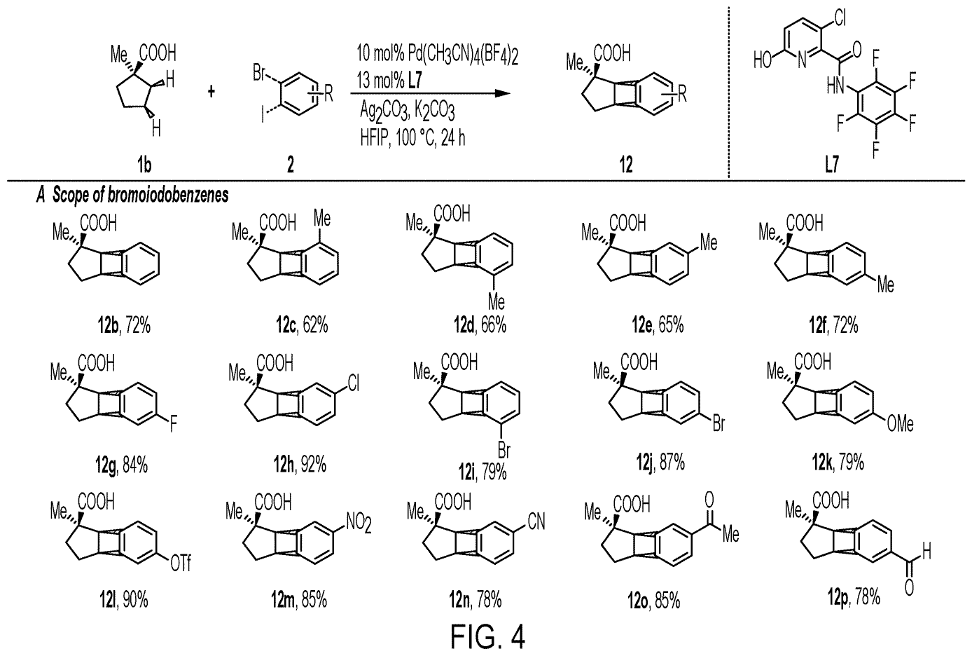

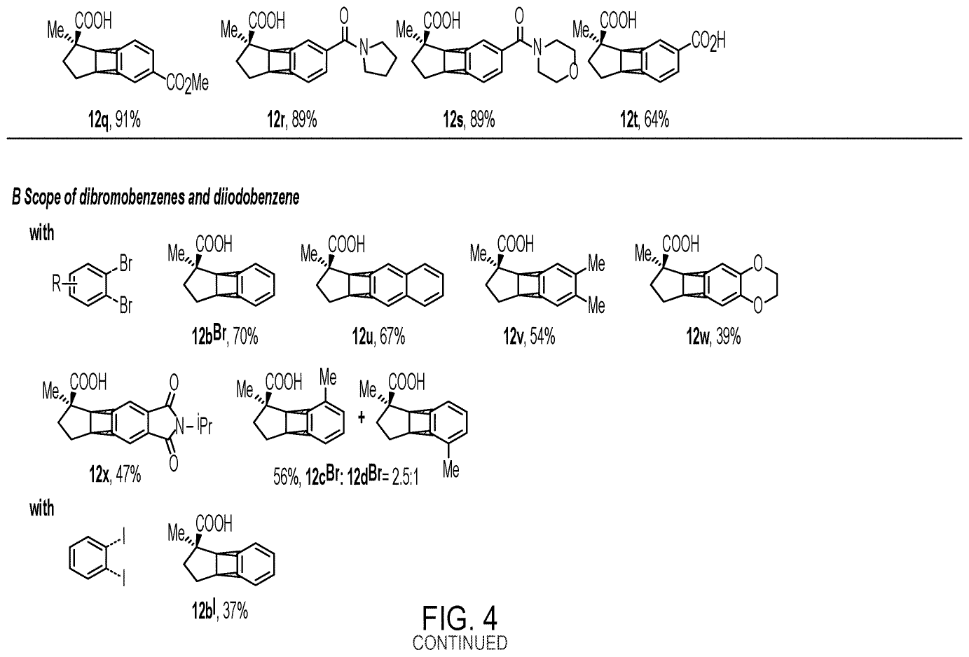

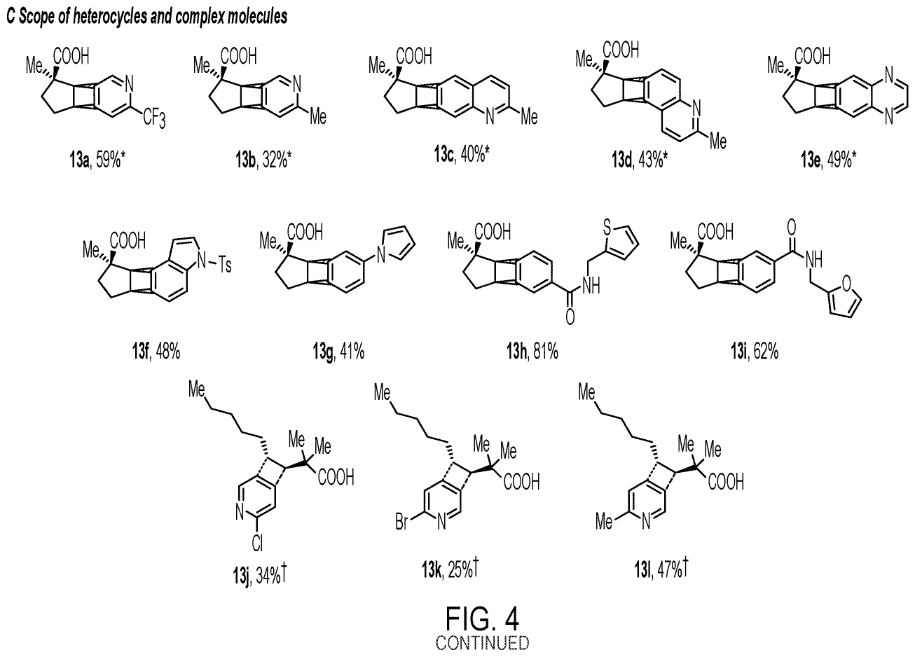

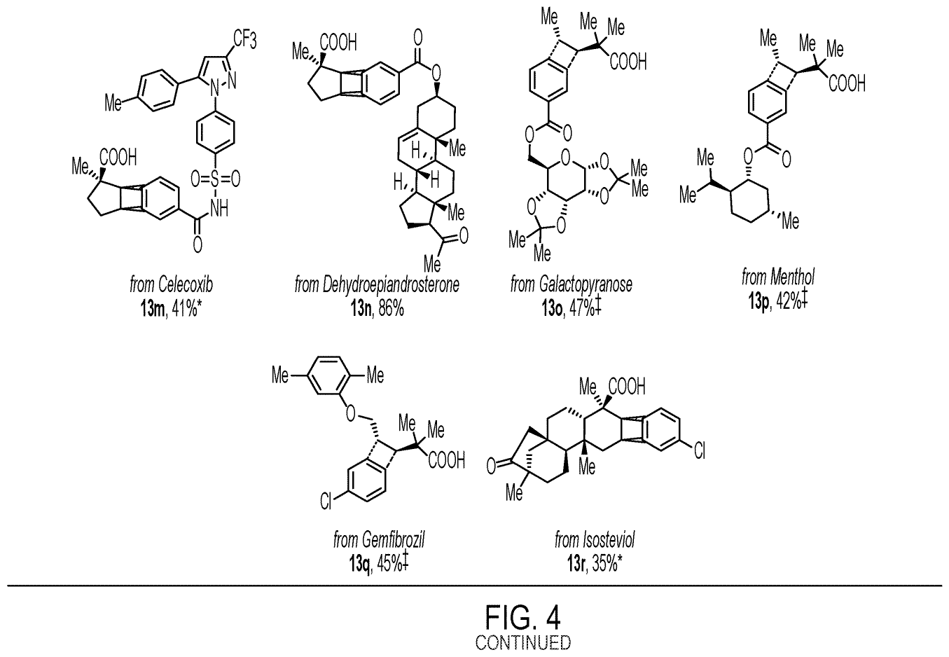

The broad scope of bromoiodoarenes further expand the diversity of BCBs (12b-12t) (FIG. 4A). Substituents at various positions were tolerated in this catalytic system (12c-12f). Halogens such as fluorine (12g), chlorine (12h), and bromine (12i-j) were compatible, providing the desired BCBs in good yields. Arenes bearing electron-donating (12k) and electron-withdrawing (12l-s) groups were also tested, affording the products in good to excellent yields. The amide (12r-s) and carboxylic acid (12t) groups, widely used in C—H activations as directing groups, were also viable for this protocol. An aryl triflate, a highly reactive site in Pd cross-coupling chemistry, was also tolerated (12l).

Moreover, dibromoarenes (2b^{Br}, 2u-x) and diiodoarene (2b^{I}) were amenable to the standard conditions, generating the desired BCBs in moderate to good yields (FIG. 4B). As expected, reaction of 1a with 2,3-dibromotoluene (2c^{Br}) afforded a mixture of two regioisomers (12c^{Br} and 12d^{Br}, rr=2.5:1), indicating that bromoiodoarenes with two different active sites are vital for controlling regioselectivity.

Modular synthesis of hetero-BCBs has been a longstanding challenge using various methods (22, 23). Gratifyingly, pyridine (13a-b, 13j-1), quinoline (13c-d), quinoxaline (13e), and indole (13f) derivatives were also competent in this process, affording the desired hetero-BCBs in 25-59% yield (FIG. 4C). Heterocycles such as pyrrole (13g), thiophene (13h) and furan (13i) were well tolerated, providing the desired products in good to excellent yields. Dihaloarenes bearing bioactive structures such as Celecoxib (13m), dehydroepiandrosterone (13n), galactose (13o), and menthol (13p) were also competent in this process. This protocol could also be applied in late-stage modification of bioactive molecules, like Gemfibrozil and Isosteviol, leading to products 13q and 13r.

In summary, disclosed herein is the achievement of Pd-catalyzed twofold C—H activation and C—C coupling of adjacent methylene groups through ligand development. The potential of this uncovered reactivity is showcased in regio-controllable [2+2] annulation between aliphatic acids and dihaloarenes, providing a solution to longstanding problems in BCB synthesis. The modular synthesis of diverse BCBs using this method is applicable to pharmaceuticals, material science and mechanochemistry, as well as natural product synthesis.

### REFERENCES

1. H. S. S. Chan, J.-M. Yang, J.-Q. Yu, Catalyst-controlled site-selective methylene C—H lactonization of dicarboxylic acids. Science 376, 1481-1487 (2022).
2. L. Hu, G. Meng, J.-Q. Yu, Ligand-enabled Pd(II)-catalyzed β-methylene C(sp³)-H arylation of free aliphatic acids. J. Am. Chem. Soc. 144, 20550-20553 (2022).
3. A. K. Sadana, R. K. Saini, W. E. Billups, Cyclobutarenes and related compounds. Chem. Rev. 103, 1539-1602 (2003).
4. M. S. Elnaggar, W. Ebrahim, A. Mandi, T. Kurtin, W. E. G. Müller, R. Kalscheuer, A. Singab, W. Lin, Z. Liu, P. Proksch, Hydroquinone derivatives from the marine-derived fungus Gliomastix sp. RSC Advances 7, 30640-30649 (2017).
5. M. A. Psotka, J. R. Teerlink, Ivabradine: Role in the chronic heart failure armamentarium. Circulation 133, 2066-2075 (2016).
6. A. Tsotinis, P. A. Afroudakis, P. J. Garratt, A. Bocianowska-Zbrog, D. Sugden, Benzocyclobutane, Benzocycloheptane and HepteneDerivatives as Melatonin Agonists and Antagonists. ChemMedChem 9, 2238-2243 (2014).
7. J. L. Segura, N. Martin, o-Quinodimethanes: Efficient intermediates in organic synthesis. Chem. Rev. 99, 3199-3246 (1999).
8. J. Wang, T. B. Kouznetsova, Z. Niu, M. T. Ong, H. M. Klukovich, A. L. Rheingold, T. J. Martinez, S. L. Craig, Inducing and quantifying forbidden reactivity with single-molecule polymer mechanochemistry. Nat. Chem. 7, 323-327 (2015).
9. C. R. Hickenboth, J. S. Moore, S. R. White, N. R. Sottos, J. Baudry, S. R. Wilson, Biasing reaction pathways with mechanical force. Nature 446, 423-427 (2007).
10. T. H. McLean, J. C. Parrish, M. R. Braden, D. Marona-Lewicka, A. Gallardo-Godoy, D. E. Nichols, 1-Aminomethylbenzocycloalkanes: Conformationally restricted hallucinogenic phenethylamine analogues as functionally selective 5-HT2A receptor agonists. J. Med. Chem. 49, 5794-5803 (2006).
11. S. Kotha, K. Lahiri, Y. Tangella, Recent advances in benzocyclobutene chemistry. Asian J. Org. Chem. 10, 3166-3185 (2021).
12. P. Schiess, M. Heitzmann, S. Rutschmann, R. Staheli, Preparation of benzocyclobutenes by flash vacuum pyrolysis. Tetrahedron Letters, 19, 4569-4572 (1978).
13. M. Chaumontet, R. Piccardi, N. Audic, J. Hitce, J.-L. Peglion, E. Clot, O. Baudoin, Synthesis of benzocyclobutenes by palladium-catalyzed C—H activation of methyl groups: method and mechanistic study. J. Am. Chem. Soc. 130, 15157-15166 (2008).
14. J. Ye, Z. Shi, T. Sperger, Y. Yasukawa, C. Kingston, F. Schoenebeck, M. Lautens, Remote C—H alkylation and C—C bond cleavage enabled by an in situ generated palladacycle. Nat. Chem. 9, 361-368 (2017).
15. P. A. Provencher, J. F. Hoskin, J. J. Wong, X. Chen, J.-Q. Yu, K. N. Houk, E. J. Sorensen, Pd(II)-catalyzed synthesis of benzocyclobutenes by β-methylene-selective C(sp³)-H arylation with a transient directing group. J. Am. Chem. Soc. 143, 20035-20041 (2021).
16. W.-X. Wei, Y. Li, Y.-T. Wen, M. Li, X-S. Li, C.-T. Wang, H.-C. Liu, Y. Xia, B.-S. Zhang, R.-Q. Jiao, Y.-M. Liang, Experimental and Computational Studies of Palladium-catalyzed spirocyclization via a Narasaka-Heck/C(sp³ or sp²)-H activation cascade reaction. J. Am. Chem. Soc. 143, 7868-7875 (2021).
17. J. Liu, T. Hao, L. Qian, M. Shi, Y. Wei, Construction of benzocyclobutenes enabled by visible-light-induced triplet biradical atom transfer of olefins. Angew. Chem. Int. Ed. 61, e202204515 (2022).
18. F. J. T. Talbot, S. Zhang, B. Satpathi, G. P. Howell, G. J. P. Perry, G. E. M. Crisenza, D. J. Procter, Modular synthesis of stereodefined benzocyclobutene derivatives via sequential Cu- and Pd-catalysis. ACS Catalysis 11, 14448-14455 (2021).
19. T. Fujii, S. Gallarati, C. Corminboeuf, Q. Wang, J. Zhu, Modular synthesis of benzocyclobutenes via Pd(II)-catalyzed oxidative [2+2] annulation of arylboronic acids with alkenes. J. Am. Chem. Soc. 144, 8920-8926 (2022).
20. J. M. Rieman, W. S. Trahanovsky, Formation of cyclobuta[b]pyridine and cyclobuta[c]pyridine by the pyrolysis of propargyl 4-pyridyl ether. Tetrahedron Letters, 18, 1867-1870 (1977).
21. A. Naiman, K. P. C. Vollhardt, 1, 2, 4, 5-Tetrahydrodicyclobuta[b,e]pyridine. Angew. Chem. 91, 440-441 (1979).
22. Z. Wang, L. Hu, N. Chekshin, Z. Zhuang, S. Qian, J. X. Qiao, J.-Q. Yu, Ligand-controlled divergent dehydrogenative reactions of carboxylic acids via C—H activation. Science 374, 1281-1285 (2021).
23. E. M. Stang, M. C. White, Molecular complexity via C—H activation: a dehydrogenative Diels-Alder reaction. J. Am. Chem. Soc. 133, 14892-14895 (2011).
24. A. F. Prusinowski, R. K. Twumasi, E. A. Wappes, D. A. Nagib, Vicinal, Double C—H Functionalization of Alcohols via an Imidate Radical-Polar Crossover Cascade. J. Am. Chem. Soc. 142, 5429-5438 (2020).
25. T. Shen, T. H. Lamber, Electrophotocatalytic diamination of vicinal C—H bonds. Science 371, 620-626 (2021).
26. T. Shen, Y.-L. Li, K.-Y. Ye, T. H. Lamber, Electrophotocatalytic Oxygenation of Multiple Adjacent C—H Bonds. Nature (2022). https://doi.org/10.1038/s41586-022-05608-x.
27. Z. Li, Z. Wang, N. Chekshin, S. Qian, J. X. Qiao, P. T. Cheng, K.-S. Yeung, W. R. Ewing, Jin-Quan Yu, A tautomeric ligand enables directed C—H hydroxylation with molecular oxygen. Science 372, 1452-1457 (2021).
28. M. Wasa, K. S. L. Chan, X.-G. Zhang, J. He, M. Miura, J.-Q. Yu, Ligand-enabled methylene C(sp³)-H bond activation with a Pd(II) catalyst. J. Am. Chem. Soc. 134, 18570-18572 (2012).
29. C. Dubost, U. Wachendorff-Neumann, P. Winter, S. Brunet, J.-P. Vors, C. Montagne, “Benzocyclobutane(Thio) Carboxamides,” U.S. Pat. No. 9,878,985 B2 (2018).

### Embodiments

Embodiment 1. A method of twofold β,γ-methylene C(sp³)-H activation/C—C bond formation, comprising regio-controllable [2+2] annulation between an aliphatic acid and a dihaloarene in the presence of a Pd(II) catalyst bound to an amide-pyridone ligand.

Embodiment 2. The method of Embodiment 1, wherein the regio-controllable [2+2] annulation between an aliphatic acid and a dihaloarene occurs according to the following reaction scheme:

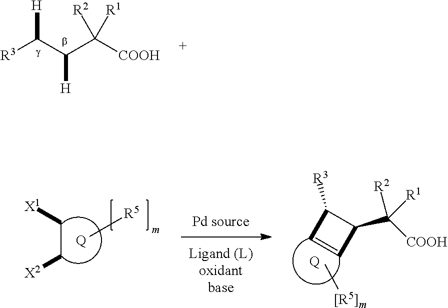

wherein:
Q is Ar or Het;
   Ar is (C₆-C₁₀)aryl, optionally substituted with one or more R^{a};
   Het is (C₅-C₈)heteroaryl, optionally substituted with one or more R^{a};
X¹ and X² are independently Br or I;
R¹ and R² are independently H or (C₁-C₆)alkyl;
R³ is (C₁-C₆)alkyl, (C₁-C₆)heteroalkyl, halo (C₁-C₆)alkyl, (C₁-C₆)alkyl-OTBS, —C(═O)OH, —C(═O)O(C₁-C₆)alkyl, (C₃-C₇)cycloalkyl, (C₃-C₇)heterocycloalkyl, (C₆-C₁₀)aryl, (C₅-C₆)heteroaryl, (C₁-C₆)alkyl (C₃-C₇)cycloalkyl, (C₁-C₆)alkyl (C₃-C₇)heterocycloalkyl, (C₁-C₆)alkyl (C₆-C₁₀)aryl, —O(C₆-C₁₀)aryl, and (C₁-C₆)alkyl (C₅-C₆)heteroaryl, wherein each is optionally and independently substituted with one or more R^{a};
   each R^{a} is independently selected from halo, —OH, —C(═O)O(C₁-C₆)alkyl, CN, NO₂, —P(═O)(O(C₁-C₆)alkyl)₂-C(═O)H, NH₂, (C₁-C₆)alkyl, halo (C₁-C₆)alkyl, (C₁-C₆)heteroalkyl, —C(═O)(C₁-C₆)alkyl, oxo, Ts, —S(═O)₂F, —OS(═O)₂F, or —S(═O)₂(C₁-C₆)alkyl, and —C(═O)NH(C₁-C₆)alkyl;
each R⁵ is independently halo, OH, CN, NO₂, —OTf, (C₁-C₆)alkyl, —C(═O)OH, —C(═O)O(C₁-C₆)alkyl, —C(═O)H, —C(═O)(C₁-C₆)alkyl, (C₁-C₆)alkyl, (C₁-C₆)heteroalkyl, halo (C₁-C₆)alkyl, —C(═O)(C₁-C₆)alkyl, —C(═O)(C₃-C₇)heterocycloalkyl, (C₅-C₆)heteroaryl, or —C(═O)NH(C₁-C₆)alkyl(C₅-C₆)heteroaryl;
   or two R⁵ may together form (C₃-C₇)cycloalkyl, (C₃-C₇)heterocycloalkyl, (C₆-C₁₀)aryl, or (C₅-C₆)heteroaryl, wherein each is optionally and independently substituted with one or more R^{a}; and
m is 0, 1, or 2;
including enantiomers, scalemic or racemic mixtures, and pharmaceutically acceptable salts thereof.

Embodiment 3. The method of Embodiment 2, wherein R¹ is H.

Embodiment 4. The method of Embodiment 2, wherein R¹ is (C₁-C₆)alkyl.

Embodiment 5. The method of Embodiment 4, wherein R¹ is Me.

Embodiment 6. The method of Embodiment 4, wherein R¹ is Et.

Embodiment 7. The method of Embodiment 4, wherein R¹ is nPr.

Embodiment 8. The method of Embodiment 4, wherein R¹ is nBu.

Embodiment 9. The method of any one of Embodiments 2-8, wherein R² is H.

Embodiment 10. The method of any one of Embodiments 2-8, wherein R² is (C₁-C₆)alkyl.

Embodiment 11. The method of Embodiment 10, wherein R² is Me.

Embodiment 12. The method of Embodiment 10, wherein R² is Et.

Embodiment 13. The method of Embodiment 10, wherein R² is nPr.

Embodiment 14. The method of Embodiment 10, wherein R² is nBu.

Embodiment 15. The method of any one of Embodiments 2-14, wherein R³ is (C₁-C₆)alkyl.

Embodiment 16. The method of Embodiment 15, wherein R³ is Me.

Embodiment 17. The method of Embodiment 15, wherein R³ is Et.

Embodiment 18. The method of Embodiment 15, wherein R³ is nPr.

Embodiment 19. The method of Embodiment 15, wherein R³ is nBu.

Embodiment 20. The method of Embodiment 15, wherein R³ is pentane.

Embodiment 21. The method of any one of Embodiments 2-14, wherein R³ is (C₁-C₆)heteroalkyl.

Embodiment 22. The method of Embodiment 21, wherein R³ is —O(C₁-C₆)alkyl.

Embodiment 23. The method of any one of Embodiments 2-14, wherein R³ is halo (C₁-C₆)alkyl.

Embodiment 24. The method of any one of Embodiments 2-8, wherein R³ is —(C₁-C₆)alkyl chloride.

Embodiment 25. The method of any one of Embodiments 2-8, wherein R³ is —CH₂P(═O)(EtO)₂.

Embodiment 26. The method of any one of Embodiments 2-8, wherein R³ is Ph, optionally substituted with one or more R^{a}.

Embodiment 27. The method of any one of Embodiments 2-8, wherein R³ is Bz, optionally substituted with one or more R^{a}.

Embodiment 28. The method of either Embodiment 26 or 27, wherein R^{a} is C₁.

Embodiment 29. The method of any one of Embodiments 2-28, wherein n is 1.

Embodiment 30. The method of Embodiment 29, wherein R⁵ is C₁.

Embodiment 31. The method of any one of Embodiments 2-30, wherein X¹ is Br and X² is I.

Embodiment 32. The method of any one of Embodiments 2-30, wherein X¹ is Br and X² is Br.

Embodiment 33. The method of any one of Embodiments 2-30, wherein X¹ is I and X² is I.

Embodiment 34. The method of Embodiment 1, wherein the regio-controllable [2+2] annulation between an aliphatic acid and a dihaloarene occurs according to the following reaction scheme:

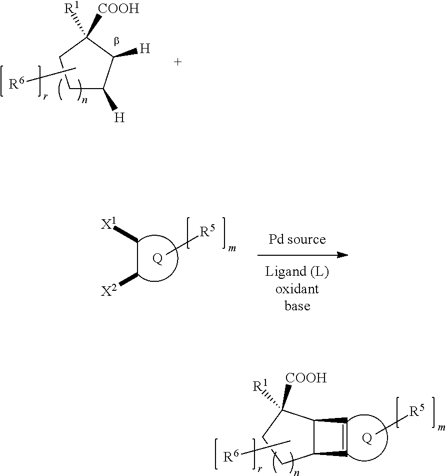

wherein:
Q is Ar or Het;
   Ar is (C₆-C₁₀)aryl, optionally substituted with one or more R^{a};
Het is (C₅-C₈)heteroaryl, optionally substituted with one or more R^{a};
X¹ and X² are independently Br or I;
R¹ is R^{1a} or R^{1b};
   R^{1a} is H, CN, or halo;
   R^{1b} is (C₁-C₆)alkyl, (C₁-C₆)heteroalkyl, halo (C₁-C₆)alkyl, (C₁-C₆)alkyl-OTBS, (C₃-C₇)cycloalkyl, (C₃-C₇)heterocycloalkyl, (C₆-C₁₀)aryl, (C₅-C₆)heteroaryl, (C₁-C₆)alkyl (C₃-C₇)cycloalkyl, (C₁-C₆)alkyl (C₃-C₇)heterocycloalkyl, (C₁-C₆)alkyl (C₆-C₁₀)aryl, and (C₁-C₆)alkyl (C₅-C₆)heteroaryl, wherein each is optionally and independently substituted with one or more R^{a};
      each R^{a} is independently selected from halo, —OH, (C₁-C₆)alkyl, —C(═O)O(C₁-C₆)alkyl, CN, NO₂, —P(═O)(O(C₁-C₆)alkyl)₂-C(═O)H, —C(═O)(C₁-C₆)alkyl, —C(═O)OH, —C(═O)O(C₁-C₆)alkyl, NH₂, halo (C₁-C₆)alkyl, (C₁-C₆)heteroalkyl, —C(═O)(C₁-C₆)alkyl, oxo, Ts, —S(═O)₂F, —OS(═O)₂F, or —S(═O)₂(C₁-C₆)alkyl, and —C(═O)NH(C₁-C₆)alkyl;
each R⁵ is independently halo, OH, CN, NO₂,-OTf, (C₁-C₆)alkyl, —C(═O)OH, —C(═O)O(C₁-C₆)alkyl, —C(═O)H, —C(═O)(C₁-C₆)alkyl, (C₁-C₆)alkyl, (C₁-C₆)heteroalkyl, halo (C₁-C₆)alkyl, —C(═O)(C₁-C₆)alkyl, —C(═O)(C₃-C₇)heterocycloalkyl, (C₅-C₆)heteroaryl, —C(═O)NH(C₁-C₆)alkyl(C₅-C₆)heteroaryl;
   or two R⁵ may together form (C₃-C₇)cycloalkyl, (C₃-C₇)heterocycloalkyl, (C₆-C₁₀)aryl, or (C₅-C₆)heteroaryl, wherein each is optionally and independently substituted with one or more R^{a}; and
each R⁶ is independently halo, OH, CN, (C₁-C₆)alkyl, (C₁-C₆)heteroalkyl, or halo (C₁-C₆)alkyl;
m is 0, 1, or 2;
n is 1, 2, 3, 4, 7, 8, or 11; and
r is 0, 1, or 2;
including enantiomers, scalemic or racemic mixtures, and pharmaceutically acceptable salts thereof.

Embodiment 35. The method of Embodiment 34, wherein R¹ is H.

Embodiment 36. The method of Embodiment 34, wherein R¹ is (C₁-C₆)alkyl.

Embodiment 37. The method of Embodiment 36, wherein R¹ is Me.

Embodiment 38. The method of Embodiment 36, wherein R¹ is Et.

Embodiment 39. The method of Embodiment 36, wherein R¹ is nPr.

Embodiment 40. The method of Embodiment 36, wherein R¹ is sec-Bu.

Embodiment 41. The method of Embodiment 34, wherein R¹ is Ph, optionally substituted with one or more R^{a}.

Embodiment 42. The method of Embodiment 34, wherein R¹ is (C₁-C₆)alkyl (C₆-C₁₀)aryl, optionally substituted with one or more R^{a}.

Embodiment 43. The method of Embodiment 34, wherein R¹ is (C₁-C₆)heteroalkyl.

Embodiment 44. The method of Embodiment 34, wherein R¹ is (C₁-C₆)alkyl (C₃-C₇)cycloalkyl.

Embodiment 45. The method of any one of Embodiments 34-45, wherein r is 0.

Embodiment 46. The method of any one of Embodiments 34-45, wherein r is 1.

Embodiment 47. The method of any one of Embodiments 34-45, wherein r is 2.

Embodiment 48. The method of either one of Embodiments 46 or 47, wherein one R⁶ is Me.

Embodiment 49. The method of Embodiment 47, wherein both R⁶ are Me.

Embodiment 50. The method of any one of Embodiments 34-49, wherein m is 0.

Embodiment 51. The method of any one of Embodiments 34-49, wherein m is 1.

Embodiment 52. The method of any one of Embodiments 34-49, wherein m is 2.

Embodiment 53. The method of any one of Embodiments 34-52, wherein p is 1.

Embodiment 54. The method of any one of Embodiments 34-52, wherein p is 2.

Embodiment 55. The method of any one of Embodiments 34-52, wherein p is 3.

Embodiment 56. The method of any one of Embodiments 34-52, wherein p is 4.

Embodiment 57. The method of any one of Embodiments 34-52, wherein p is 7.

Embodiment 58. The method of any one of Embodiments 34-52, wherein p is 8.

Embodiment 59. The method of any one of Embodiments 34-52, wherein p is 11.

Embodiment 60. The method of any one of Embodiments 1-59, wherein the Pd source is Pd(OAc)₂.

Embodiment 61. The method of any one of Embodiments 1-59, wherein the Pd source is Pd(CH₃CN)₂Cl₂.

Embodiment 62. The method of any one of Embodiments 1-59, wherein the Pd source is Pd(TFA)₂.

Embodiment 63. The method of any one of Embodiments 1-59, wherein the Pd source is Pd₂(dba)₃.

Embodiment 64. The method of any one of Embodiments 1-59, wherein the Pd source is Pd(OPiv)₂.

Embodiment 65. The method of any one of Embodiments 1-59, wherein the Pd source is Pd(CH₃CN)₄(BF₄)₂.

Embodiment 66. The method of any one of Embodiments 1-59, wherein the Pd source is approximately 10 mol %.

Embodiment 67. The method of any one of Embodiments 1-59, wherein the Pd source is approximately 5 mol %.

Embodiment 68. The method of any one of Embodiments 1-59, wherein the Ligand (L) is selected from the group consisting of:

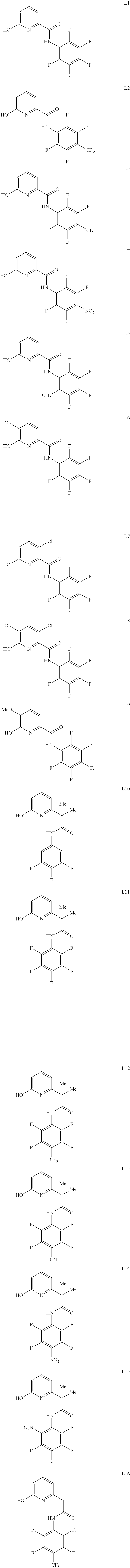

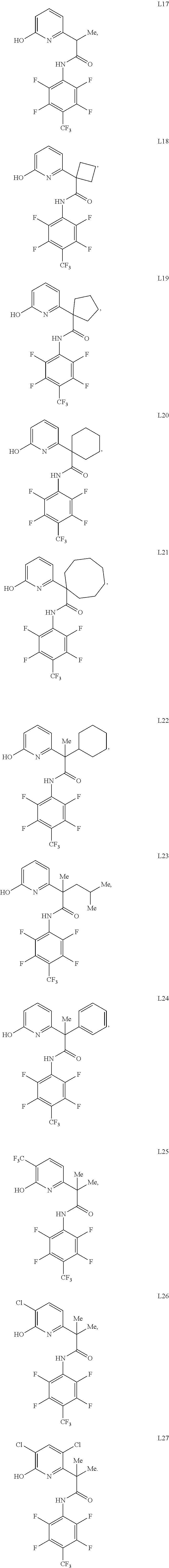

Embodiment 69. The method of any one of Embodiments 1-59, wherein the Ligand (L) is L7.

Embodiment 70. The method of any one of Embodiments 1-59, wherein the Ligand (L) is L12.

Embodiment 71. The method of any one of Embodiments 1-59, wherein the Ligand (L) is selected from the group consisting of:

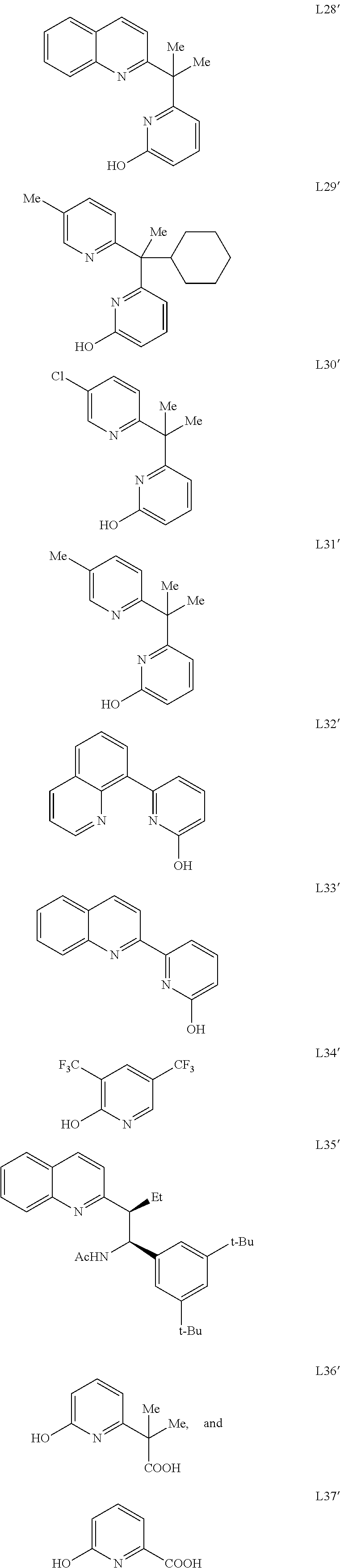

Embodiment 72. The method of any one of Embodiments 2-71, wherein Q is Ph optionally substituted with one or more R^{a}.

Embodiment 73. The method of any one of Embodiments 2-71, wherein Q is Het optionally substituted with one or more R^{a}.

Embodiment 74. The method of Embodiment 73, wherein Het is pyridinyl.

Embodiment 75. The method of Embodiment 73, wherein Het is quinolinyl.

Embodiment 76. The method of Embodiment 73, wherein Het is quinoxalinyl.

Embodiment 77. The method of Embodiment 73, wherein Het is indolyl.

Embodiment 78. The method of any one of Embodiments 2-77, wherein the oxidant is AgOAc.

Embodiment 79. The method of any one of Embodiments 2-77, wherein the oxidant is Ag₂CO₃.

Embodiment 80. The method of any one of Embodiments 2-77, wherein the oxidant is Ag₂O.

Embodiment 81. The method of any one of Embodiments 2-77, wherein the oxidant is AgF.

Embodiment 82. The method of any one of Embodiments 2-77, wherein the oxidant is Ag₃PO₄.

Embodiment 83. The method of any one of Embodiments 2-82, wherein the base is Na₂CO₃.

Embodiment 84. The method of any one of Embodiments 2-82, wherein the base is Li₂CO₃.

Embodiment 85. The method of any one of Embodiments 2-82, wherein the base is K₂CO₃.

Embodiment 86. The method of any one of Embodiments 2-82, wherein the base is Cs₂CO₃.

Embodiment 87. The method of any one of Embodiments 2-82, wherein the base is NaHCO₃.

Embodiment 88. The method of any one of Embodiments 2-82, wherein the base is KHCO₃.

Embodiment 89. The method of any one of Embodiments 2-82, wherein the base is KH₂PO₄.

Embodiment 90. The method of any one of Embodiments 2-82, wherein the base is K₃PO₄.

Embodiment 91. The method of any one of Embodiments 2-82, wherein the base is NaOAc.

Embodiment 92. The method of any one of Embodiments 2-99, wherein the solvent is HFIP.

Embodiment 93. The method of any one of Embodiments 2-92, wherein the reaction temperature is between approximately 60-120° C.

Embodiment 94. The method of any one of Embodiments 2-107, wherein the reaction temperature is between approximately 70-110° C.

Embodiment 95. The method of any one of Embodiments 2-107, wherein the reaction temperature is between approximately 100-110° C.

Embodiment 96. The method of any one of Embodiments 2-95, wherein the mol % of Pd source is approximately between 5-15%.

Embodiment 97. The method of any one of Embodiments 2-95, wherein the mol % of Pd source is approximately 10%.

Embodiment 98. The method of any one of Embodiments 2-97, wherein the mol % of Ligand is approximately between 5-25%.

Embodiment 99. The method of any one of Embodiments 2-97, wherein the mol % of Ligand is approximately between 10-20%.

Embodiment 100. The method of any one of Embodiments 2-97, wherein the mol % of Ligand is approximately 13%.

Embodiment 101. The method of any one of Embodiments 2-100, wherein the equivalents of aliphatic acid compared to dihaloarene is approximately 1:2.

Embodiment 102. The method of any one of Embodiments 2-101, wherein the equivalents of aryl or heteroaryl iodide compared to carboxylic acid substrate is approximately 2.0.

Embodiment 103. The method of any one of Embodiments 2-102, wherein the equivalents of oxidant compared to carboxylic acid substrate is approximately 20:1.

Embodiment 104. The method of any one of Embodiments 2-103, wherein the equivalents of base compared to carboxylic acid substrate is approximately 25:1.

Embodiment 105. A compound having the structure selected from the group consisting of:

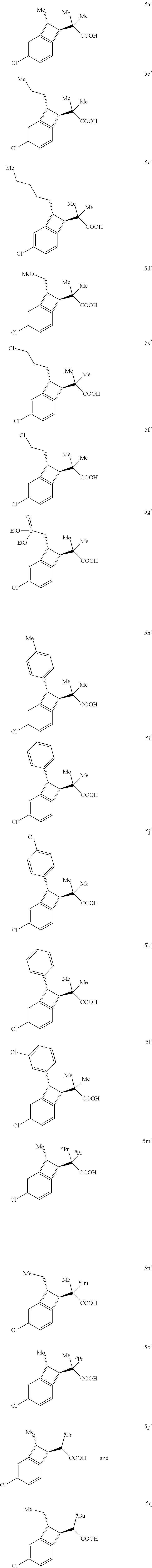

including enantiomers, scalemic or racemic mixtures, and pharmaceutically acceptable salts thereof.

Embodiment 106. A method of preparing the compound of Embodiment 105, comprising the following step:

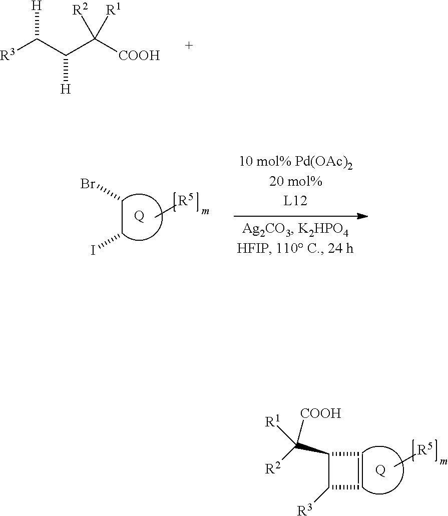

wherein:
Q is Ar or Het;
   Ar is (C₆-C₁₀)aryl, optionally substituted with one or more R^{a};
   Het is (C₅-C₈)heteroaryl, optionally substituted with one or more R^{a};
X¹ and X² are independently Br or I;
R¹ and R² are independently H or (C₁-C₆)alkyl;
R³ is (C₁-C₆)alkyl, (C₁-C₆)heteroalkyl, halo (C₁-C₆)alkyl, (C₁-C₆)alkyl-OTBS, —C(═O)OH, —C(═O)O(C₁-C₆)alkyl, (C₃-C₇)cycloalkyl, (C₃-C₇)heterocycloalkyl, (C₆-C₁₀)aryl, (C₅-C₆)heteroaryl, (C₁-C₆)alkyl (C₃-C₇)cycloalkyl, (C₁-C₆)alkyl (C₃-C₇)heterocycloalkyl, (C₁-C₆)alkyl (C₆-C₁₀)aryl, —O(C₆-C₁₀)aryl, and (C₁-C₆)alkyl (C₅-C₆)heteroaryl, wherein each is optionally and independently substituted with one or more R^{a};
   each R^{a} is independently selected from halo, —OH, —C(═O)O(C₁-C₆)alkyl, CN, NO₂, —P(═O)(O(C₁-C₆)alkyl)₂-C(═O)H, NH₂, (C₁-C₆)alkyl, halo (C₁-C₆)alkyl, (C₁-C₆)heteroalkyl, —C(═O)(C₁-C₆)alkyl, oxo, Ts, —S(═O)₂F, —OS(═O)₂F, or —S(═O)₂(C₁-C₆)alkyl, and —C(═O)NH(C₁-C₆)alkyl;
each R⁵ is independently halo, OH, CN, NO₂,-OTf, (C₁-C₆)alkyl, —C(═O)OH, —C(═O)O(C₁-C₆)alkyl, —C(═O)H, —C(═O)(C₁-C₆)alkyl, (C₁-C₆)alkyl, (C₁-C₆)heteroalkyl, halo (C₁-C₆)alkyl, —C(═O)(C₁-C₆)alkyl, —C(═O)(C₃-C₇)heterocycloalkyl, (C₅-C₆)heteroaryl, or —C(═O)NH(C₁-C₆)alkyl(C₅-C₆)heteroaryl;
   or two R⁵ may together form (C₃-C₇)cycloalkyl, (C₃-C₇)heterocycloalkyl, (C₆-C₁₀)aryl, or (C₅-C₆)heteroaryl, wherein each is optionally and independently substituted with one or more R^{a}; and
m is 0, 1, or 2;
including enantiomers, scalemic or racemic mixtures, and pharmaceutically acceptable salts thereof.

Embodiment 107. A compound having the structure selected from the group consisting of:

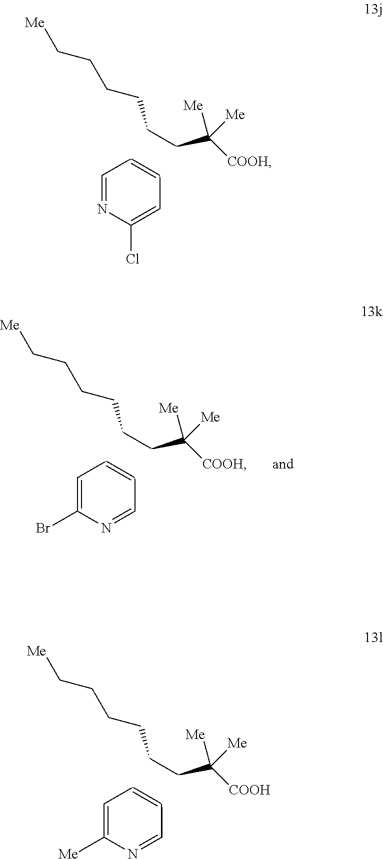

including enantiomers, scalemic or racemic mixtures, and pharmaceutically acceptable salts thereof.

Embodiment 108. A method of preparing the compound of Embodiment 107, comprising the following step:

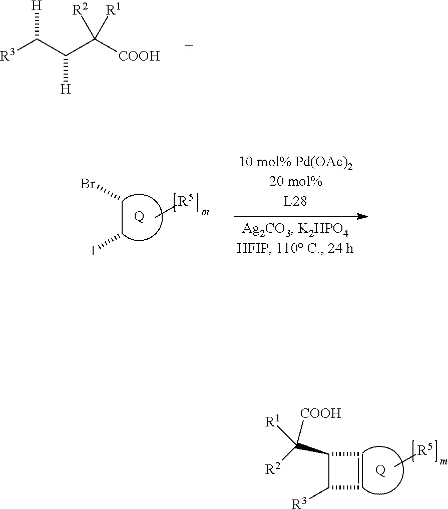

wherein:
X¹ and X² are independently Br or I;
Q is Ar or Het;
   Ar is (C₆-C₁₀)aryl, optionally substituted with one or more R^{a};
   Het is (C₅-C₈)heteroaryl, optionally substituted with one or more R^{a};
R¹ and R² are independently H or (C₁-C₆)alkyl;
R³ is (C₁-C₆)alkyl, (C₁-C₆)heteroalkyl, halo (C₁-C₆)alkyl, (C₁-C₆)alkyl-OTBS, —C(═O)OH, —C(═O)O(C₁-C₆)alkyl, (C₃-C₇)cycloalkyl, (C₃-C₇)heterocycloalkyl, (C₆-C₁₀)aryl, (C₅-C₆)heteroaryl, (C₁-C₆)alkyl (C₃-C₇)cycloalkyl, (C₁-C₆)alkyl (C₃-C₇)heterocycloalkyl, (C₁-C₆)alkyl (C₆-C₁₀)aryl, —O(C₆-C₁₀)aryl, and (C₁-C₆)alkyl (C₅-C₆)heteroaryl, wherein each is optionally and independently substituted with one or more R^{a};
   each R^{a} is independently selected from halo, —OH, —C(═O)O(C₁-C₆)alkyl, CN, NO₂, —P(═O)(O(C₁-C₆)alkyl)₂-C(═O)H, NH₂, (C₁-C₆)alkyl, halo (C₁-C₆)alkyl, (C₁ -C₆)heteroalkyl, —C(═O)(C₁-C₆)alkyl, oxo, Ts, —S(═O)₂F, —OS(═O)₂F, or —S(═O)₂(C₁-C₆)alkyl, and —C(═O)NH(C₁-C₆)alkyl;
each R⁵ is independently halo, OH, CN, NO₂,-OTf, (C₁-C₆)alkyl, —C(═O)OH, —C(═O)O(C₁-C₆)alkyl, —C(═O)H, —C(═O)(C₁-C₆)alkyl, (C₁-C₆)alkyl, (C₁-C₆)heteroalkyl, halo (C₁-C₆)alkyl, —C(═O)(C₁-C₆)alkyl, —C(═O)(C₃-C₇)heterocycloalkyl, (C₅-C₆)heteroaryl, or —C(═O)NH(C₁-C₆)alkyl(C₅-C₆)heteroaryl;
   or two R⁵ may together form (C₃-C₇)cycloalkyl, (C₃-C₇)heterocycloalkyl, (C₆-C₁₀)aryl, or (C₅-C₆)heteroaryl, wherein each is optionally and independently substituted with one or more R^{a}; and
m is 0, 1, or 2;
including enantiomers, scalemic or racemic mixtures, and pharmaceutically acceptable salts thereof.

Embodiment 109. A compound having the structure selected from the group consisting of:

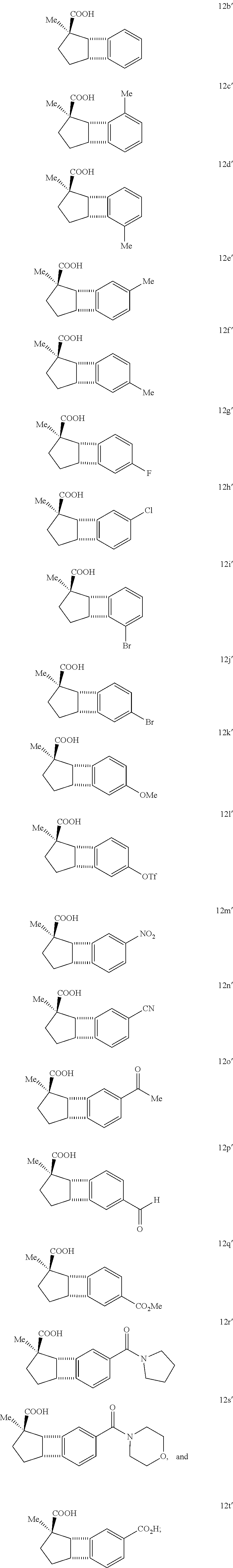

including enantiomers, scalemic or racemic mixtures, and pharmaceutically acceptable salts thereof.

Embodiment 110. A compound having the structure selected from the group consisting of:

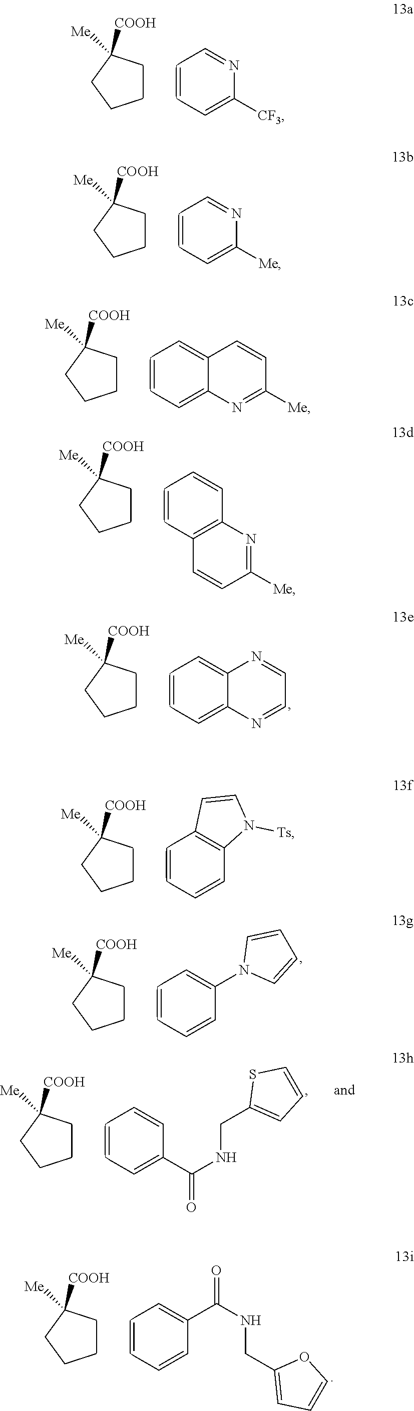

including enantiomers, scalemic or racemic mixtures, and pharmaceutically acceptable salts thereof.

Embodiment 111. A compound having the structure selected from the group consisting of:

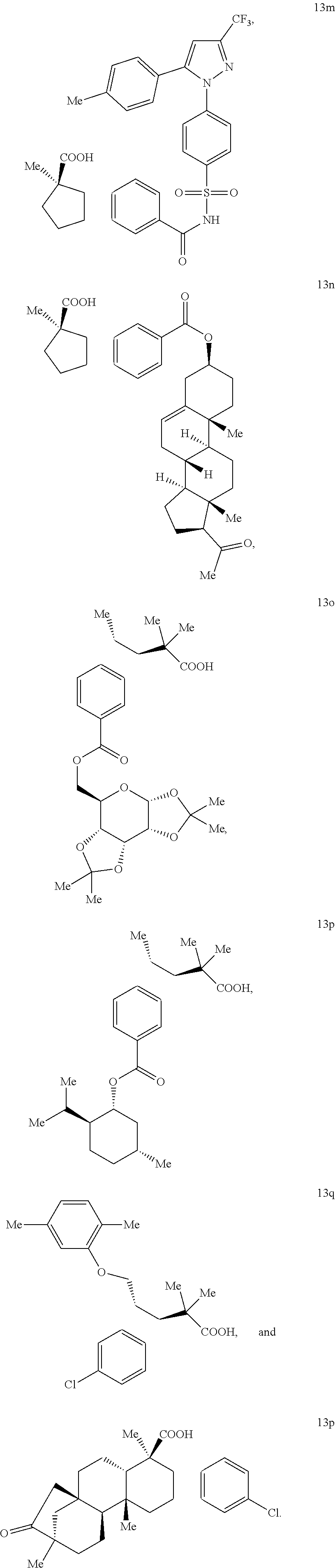

Embodiment 112. A method of preparing the compound of any one of Embodiments 109-111, comprising the following step:

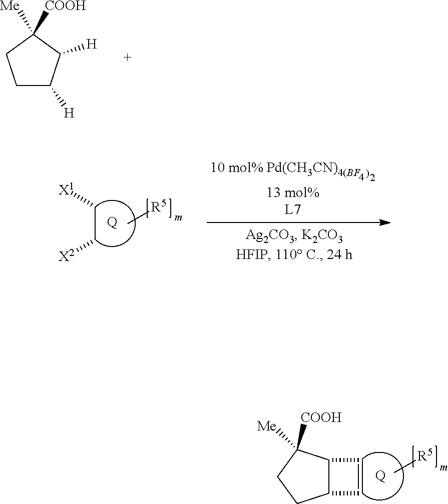

wherein
X¹ and X² are independently Br or I;
Q is Ar or Het;
   Ar is (C₆-C₁₀)aryl, optionally substituted with one or more R^{a};
   Het is (C₅-C₈)heteroaryl, optionally substituted with one or more R^{a};
      each R^{a} is independently selected from halo, —OH, (C₁-C₆)alkyl, —C(═O)O(C₁-C₆)alkyl, CN, NO₂, —P(═O)(O(C₁-C₆)alkyl)₂-C(═O)H, —C(═O)(C₁-C₆)alkyl, —C(═O)OH, —C(═O)O(C₁-C₆)alkyl, NH₂, halo (C₁-C₆)alkyl, (C₁-C₆)heteroalkyl, —C(═O)(C₁-C₆)alkyl, oxo, Ts, —S(═O)₂F, —OS(═O)₂F, or —S(═O)₂(C₁-C₆)alkyl, and —C(═O)NH(C₁-C₆)alkyl;
each R⁵ is independently halo, OH, CN, NO₂,-OTf, (C₁-C₆)alkyl, —C(═O)OH, —C(═O)O(C₁-C₆)alkyl, —C(═O)H, —C(═O)(C₁-C₆)alkyl, (C₁-C₆)alkyl, (C₁-C₆)heteroalkyl, halo (C₁-C₆)alkyl, —C(═O)(C₁-C₆)alkyl, —C(═O)(C₃-C₇)heterocycloalkyl, (C₅-C₆)heteroaryl, —C(═O)NH(C₁-C₆)alkyl(C₅-C₆)heteroaryl;
or two R⁵ may together form (C₃-C₇)cycloalkyl, (C₃-C₇)heterocycloalkyl, (C₆-C₁₀)aryl, or (C₅-C₆)heteroaryl, wherein each is optionally and independently substituted with one or more R^{a}; and
   m is 0, 1, or 2;
   including enantiomers, scalemic or racemic mixtures, and pharmaceutically acceptable salts thereof.

Embodiment 113. A method of preparing the Compound 6, comprising the following steps:

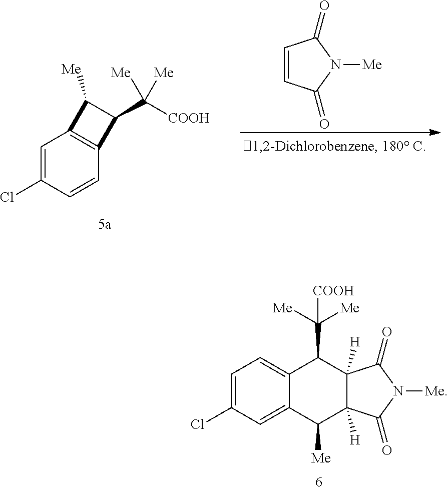

Embodiment 114. A method of preparing the Compound 7, comprising the following steps:

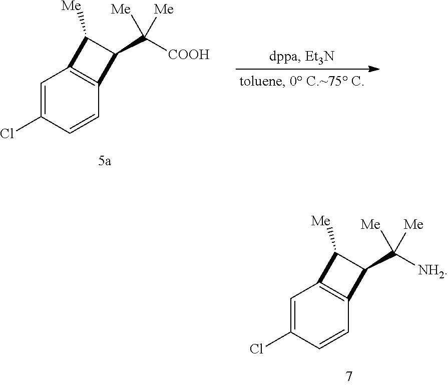

Embodiment 115. A method of preparing the Compound 10, comprising the following steps:

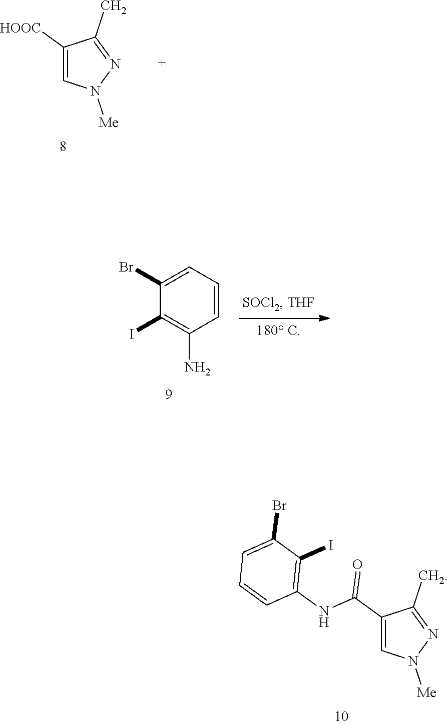

Embodiment 116. A method of preparing the Compound 11, comprising the following steps:

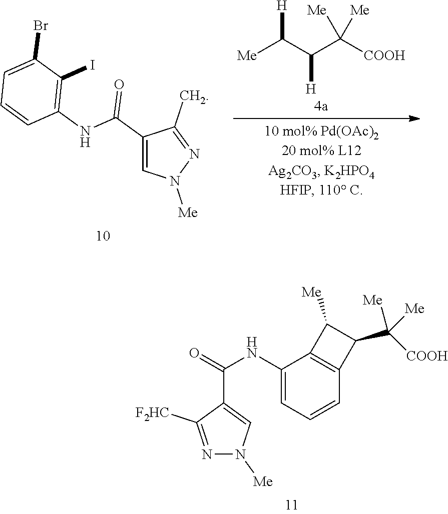

Embodiment 117. A compound having the Formula I

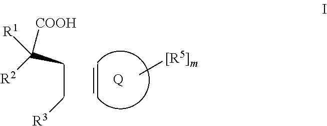

wherein:
Q is Ar or Het;
   Ar is (C₆-C₁₀)aryl, optionally substituted with one or more R^{a};
   Het is (C₅-C₈)heteroaryl, optionally substituted with one or more R^{a};
R¹ and R² are independently H or (C₁-C₆)alkyl, wherein both R¹ and R² are not H;
R³ is (C₁-C₆)alkyl, (C₁-C₆)heteroalkyl, halo (C₁-C₆)alkyl, (C₁-C₆)alkyl-OTBS, —C(═O)OH, —C(═O)O(C₁-C₆)alkyl, (C₃-C₇)cycloalkyl, (C₃-C₇)heterocycloalkyl, (C₆-C₁₀)aryl, (C₅-C₆)heteroaryl, (C₁-C₆)alkyl (C₃-C₇)cycloalkyl, (C₁-C₆)alkyl (C₃-C₇)heterocycloalkyl, (C₁-C₆)alkyl (C₆-C₁₀)aryl, —O(C₆-C₁₀)aryl, and (C₁-C₆)alkyl (C₅-C₆)heteroaryl, wherein each is optionally and independently substituted with one or more R^{a};
   each R^{a} is independently selected from halo, —OH, —C(═O)O(C₁-C₆)alkyl, CN, NO₂, —P(═O)(O(C₁-C₆)alkyl)₂-C(═O)H, NH₂, (C₁-C₆)alkyl, halo (C₁-C₆)alkyl, (C₁-C₆)heteroalkyl, —C(═O)(C₁-C₆)alkyl, oxo, Ts, —S(═O)₂F, —OS(═O)₂F, or —S(═O)₂(C₁-C₆)alkyl, and —C(═O)NH(C₁-C₆)alkyl;
each R⁵ is independently halo, OH, CN, NO₂,-OTf, (C₁-C₆)alkyl, —C(═O)OH, —C(═O)O(C₁-C₆)alkyl, —C(═O)H, —C(═O)(C₁-C₆)alkyl, (C₁-C₆)alkyl, (C₁-C₆)heteroalkyl, halo (C₁-C₆)alkyl, —C(═O)(C₁-C₆)alkyl, —C(═O)(C₃-C₇)heterocycloalkyl, (C₅-C₆)heteroaryl, or —C(═O)NH(C₁-C₆)alkyl(C₅-C₆)heteroaryl;
   or two R⁵ may together form (C₃-C₇)cycloalkyl, (C₃-C₇)heterocycloalkyl, (C₆-C₁₀)aryl, or (C₅-C₆)heteroaryl, wherein each is optionally and independently substituted with one or more R^{a}; and
m is 0, 1, or 2;
including enantiomers, scalemic or racemic mixtures, and pharmaceutically acceptable salts thereof.

Embodiment 118. A compound having the Formula II

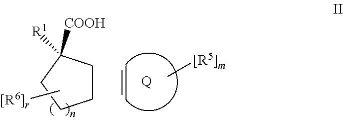

wherein:
Q is Ar or Het;
   Ar is (C₆-C₁₀)aryl, optionally substituted with one or more R^{a};
   Het is (C₅-C₈)heteroaryl, optionally substituted with one or more R^{a};
R¹ is (C₁-C₆)alkyl, (C₁-C₆)heteroalkyl, halo (C₁-C₆)alkyl, (C₁-C₆)alkyl-OTBS, (C₃-C₇)cycloalkyl, (C₃-C₇)heterocycloalkyl, (C₆-C₁₀)aryl, (C₅-C₆)heteroaryl, (C₁-C₆)alkyl (C₃-C₇)cycloalkyl, (C₁-C₆)alkyl (C₃-C₇)heterocycloalkyl, (C₁-C₆)alkyl (C₆-C₁₀)aryl, and (C₁-C₆)alkyl (C₅-C₆)heteroaryl, wherein each is optionally and independently substituted with one or more R^{a};
   each R^{a} is independently selected from halo, —OH, (C₁-C₆)alkyl, —C(═O)O(C₁-C₆)alkyl, CN, NO₂, —P(═O)(O(C₁-C₆)alkyl)₂-C(═O)H, —C(═O)(C₁-C₆)alkyl, —C(═O)OH, —C(═O)O(C₁-C₆)alkyl, NH₂, halo (C₁-C₆)alkyl, (C₁-C₆)heteroalkyl, —C(═O)(C₁-C₆)alkyl, oxo, Ts, —S(═O)₂F, —OS(═O)₂F, or —S(═O)₂(C₁-C₆)alkyl, and —C(═O)NH(C₁-C₆)alkyl;
each R⁵ is independently halo, OH, CN, NO₂,-OTf, (C₁-C₆)alkyl, —C(═O)OH, —C(═O)O(C₁-C₆)alkyl, —C(═O)H, —C(═O)(C₁-C₆)alkyl, (C₁-C₆)alkyl, (C₁-C₆)heteroalkyl, halo (C₁-C₆)alkyl, —C(═O)(C₁-C₆)alkyl, —C(═O)(C₃-C₇)heterocycloalkyl, (C₅-C₆)heteroaryl, —C(═O)NH(C₁-C₆)alkyl(C₅-C₆)heteroaryl;
   or two R⁵ may together form (C₃-C₇)cycloalkyl, (C₃-C₇)heterocycloalkyl, (C₆-C₁₀)aryl, or (C₅-C₆)heteroaryl, wherein each is optionally and independently substituted with one or more R^{a}; and
each R⁶ is independently halo, OH, CN, (C₁-C₆)alkyl, (C₁-C₆)heteroalkyl, or halo (C₁-C₆)alkyl;
n is 1, 2, 3, 4, 7, 8, or 11; and
r is 0, 1, or 2;
including enantiomers, scalemic or racemic mixtures, and pharmaceutically acceptable salts thereof.

Embodiment 119. Any method of twofold β,γ-methylene C(sp³)-H activation/C—C bond formation, comprising regio-controllable [2+2] annulation between an aliphatic acid and a dihaloarene in the presence of a Pd(II) catalyst bound to an amide-pyridone ligand, the product thereof, any amide-pyridone ligand, or preparation thereof, as described herein.

### Definitions

The phrase “a” or “an” entity as used herein refers to one or more of that entity; for example, a compound refers to one or more compounds or at least one compound. As such, the terms “a” (or “an”), “one or more”, and “at least one” can be used interchangeably herein.

The phrase “as defined herein above” refers to the broadest definition for each group as provided in the Summary of the Invention, the Detailed Description of the Invention, the Experimentals, or the broadest claim. In all other embodiments provided below, substituents which can be present in each embodiment and which are not explicitly defined retain the broadest definition provided in the Summary of the Invention.

As used in this specification, whether in a transitional phrase or in the body of the claim, the terms “comprise(s)” and “comprising” are to be interpreted as having an open-ended meaning. That is, the terms are to be interpreted synonymously with the phrases “having at least” or “including at least”. When used in the context of a process, the term “comprising” means that the process includes at least the recited steps, but may include additional steps. When used in the context of a compound or composition, the term “comprising” means that the compound or composition includes at least the recited features or components, but may also include additional features or components.

As used herein, unless specifically indicated otherwise, the word “or” is used in the “inclusive” sense of “and/or” and not the “exclusive” sense of “either/or”.

The term “independently” is used herein to indicate that a variable is applied in any one instance without regard to the presence or absence of a variable having that same or a different definition within the same compound. Thus, in a compound in which “R” appears twice and is defined as “independently selected from” means that each instance of that R group is separately identified as one member of the set which follows in the definition of that R group. For example, “each R¹ and R² is independently selected from carbon and nitrogen” means that both R¹ and R² can be carbon, both R¹ and R² can be nitrogen, or R¹ or R² can be carbon and the other nitrogen or vice versa.

When any variable occurs more than one time in any moiety or formula depicting and describing compounds employed or claimed in the present invention, its definition on each occurrence is independent of its definition at every other occurrence. Also, combinations of substituents and/or variables are permissible only if such compounds result in stable compounds.

The symbols “*” at the end of a bond or a line drawn through a bond or “~~~~” drawn through a bond each refer to the point of attachment of a functional group or other chemical moiety to the rest of the molecule of which it is a part.

A bond drawn into ring system (as opposed to connected at a distinct vertex) indicates that the bond may be attached to any of the suitable ring atoms.

The term “optional” or “optionally” as used herein means that a subsequently described event or circumstance may, but need not, occur, and that the description includes instances where the event or circumstance occurs and instances in which it does not. For example, “optionally substituted” means that the “optionally substituted” moiety may incorporate a hydrogen or a substituent.

The phrase “optional bond” means that the bond may or may not be present, and that the description includes single, double, or triple bonds. If a substituent is designated to be a “bond” or “absent”, the atoms linked to the substituents are then directly connected.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

Certain compounds disclosed herein may exhibit tautomerism. Tautomeric compounds can exist as two or more interconvertable species. Prototropic tautomers result from the migration of a covalently bonded hydrogen atom between two atoms. Tautomers generally exist in equilibrium and attempts to isolate an individual tautomers usually produce a mixture whose chemical and physical properties are consistent with a mixture of compounds. The position of the equilibrium is dependent on chemical features within the molecule. For example, in many aliphatic aldehydes and ketones, such as acetaldehyde, the keto form predominates while; in phenols, the enol form predominates. Common prototropic tautomers include keto/enol (—C(═O)—CH—⇄—C(—OH)═CH—), amide/imidic acid (—C(═O)—NH—⇄—C(—OH)═N—) and amidine (—C(═NR)—NH—⇄—C(—NHR)═N—) tautomers. The latter two are particularly common in heteroaryl and heterocyclic rings and the present invention encompasses all tautomeric forms of the compounds.

Technical and scientific terms used herein have the meaning commonly understood by one of skill in the art to which the present invention pertains, unless otherwise defined. Reference is made herein to various methodologies and materials known to those of skill in the art. Standard reference works setting forth the general principles of pharmacology include Goodman and Gilman's The Pharmacological Basis of Therapeutics, 10^{th} Ed., McGraw Hill Companies Inc., New York (2001). Any suitable materials and/or methods known to those of skill can be utilized in carrying out the present invention. However, preferred materials and methods are described. Materials, reagents and the like to which reference are made in the following description and examples are obtainable from commercial sources, unless otherwise noted.

The definitions described herein may be appended to form chemically-relevant combinations, such as “heteroalkylaryl,” “haloalkylheteroaryl,” “arylalkylheterocyclyl,” “alkylcarbonyl,” “alkoxyalkyl,” and the like. When the term “alkyl” is used as a suffix following another term, as in “phenylalkyl,” or “hydroxyalkyl,” this is intended to refer to an alkyl group, as defined above, being substituted with one to two substituents selected from the other specifically-named group. Thus, for example, “phenylalkyl” refers to an alkyl group having one to two phenyl substituents, and thus includes benzyl, phenylethyl, and biphenyl. An “alkylaminoalkyl” is an alkyl group having one to two alkylamino substituents. “Hydroxyalkyl” includes 2-hydroxyethyl, 2-hydroxypropyl, 1-(hydroxymethyl)-2-methylpropyl, 2-hydroxybutyl, 2,3-dihydroxybutyl, 2-(hydroxymethyl), 3-hydroxypropyl, and so forth. Accordingly, as used herein, the term “hydroxyalkyl” is used to define a subset of heteroalkyl groups defined below. The term -(ar)alkyl refers to either an unsubstituted alkyl or an aralkyl group. The term (hetero)aryl or (het)aryl refers to either an aryl or a heteroaryl group.

The term “acyl” as used herein denotes a group of formula —C(═O)R wherein R is hydrogen or lower alkyl as defined herein. The term or “alkylcarbonyl” as used herein denotes a group of formula C(═O)R wherein R is alkyl as defined herein. The term C₁₋₆ acyl refers to a group —C(═O)R contain 6 carbon atoms. The term “arylcarbonyl” as used herein means a group of formula C(═O)R wherein R is an aryl group; the term “benzoyl” as used herein an “arylcarbonyl” group wherein R is phenyl.

The term “alkyl” as used herein denotes an unbranched or branched chain, saturated, monovalent hydrocarbon residue containing 1 to 12 carbon atoms. The term “lower alkyl” or “C₁-C₆ alkyl” as used herein denotes a straight or branched chain hydrocarbon residue containing 1 to 6 carbon atoms. “C₁₋₁₂ alkyl” as used herein refers to an alkyl composed of 1 to 12 carbons. Examples of alkyl groups include, but are not limited to, lower alkyl groups include methyl, ethyl, propyl, i-propyl, n-butyl, i-butyl, t-butyl or pentyl, isopentyl, neopentyl, hexyl, heptyl, and octyl.

When the term “alkyl” is used as a suffix following another term, as in “phenylalkyl,” or “hydroxyalkyl,” this is intended to refer to an alkyl group, as defined above, being substituted with one to two substituents selected from the other specifically-named group. Thus, for example, “phenylalkyl” denotes the radical R′R″—, wherein R′ is a phenyl radical, and R″ is an alkylene radical as defined herein with the understanding that the attachment point of the phenylalkyl moiety will be on the alkylene radical. Examples of arylalkyl radicals include, but are not limited to, benzyl, phenylethyl, 3-phenylpropyl. The terms “arylalkyl” or “aralkyl” are interpreted similarly except R′ is an aryl radical. The terms “(het)arylalkyl” or “(het)aralkyl” are interpreted similarly except R′ is optionally an aryl or a heteroaryl radical.

When a range of values is listed, it is intended to encompass each value and sub-range within the range. For example, “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

“Alkyl” refers to a radical of a straight-chain or branched saturated hydrocarbon group having from 1 to 20 carbon atoms (“C₁₋₂₀ alkyl”). In some embodiments, an alkyl group has 1 to 15 carbon atoms (“C₁₋₁₅ alkyl”). In some embodiments, an alkyl group has 1 to 14 carbon atoms (“C₁₋₁₄ alkyl”). In some embodiments, an alkyl group has 1 to 13 carbon atoms (“C₁₋₁₃ alkyl”). In some embodiments, an alkyl group has 1 to 12 carbon atoms (“C₁₋₁₂ alkyl”). In some embodiments, an alkyl group has 1 to 11 carbon atoms (“C₁₋₁₁ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like.

“Alkenyl” or “olefin” refers to a radical of a straight-chain or branched hydrocarbon group having from 2 to 10 carbon atoms and 1, 2, 3, or 4 carbon-carbon double bonds (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like.

“Alkynyl” refers to a radical of a straight-chain or branched hydrocarbon group having from 2 to 10 carbon atoms and one or more carbon-carbon triple bonds (e.g., 1, 2, 3, or 4 triple bonds) (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C), and the like. Additional examples of alkynyl include heptynyl (C₇), octynyl (C₈), and the like.

The terms “haloalkyl” or “halo-lower alkyl” or “lower haloalkyl” refers to a straight or branched chain hydrocarbon residue containing 1 to 6 carbon atoms wherein one or more carbon atoms are substituted with one or more halogen atoms.

The term “alkylene” or “alkylenyl” as used herein denotes a divalent saturated linear hydrocarbon radical of 1 to 10 carbon atoms (e.g., (CH₂)ₙ) or a branched saturated divalent hydrocarbon radical of 2 to 10 carbon atoms (e.g., —CHMe- or —CH₂CH(i-Pr)CH₂—), unless otherwise indicated. Except in the case of methylene, the open valences of an alkylene group are not attached to the same atom. Examples of alkylene radicals include, but are not limited to, methylene, ethylene, propylene, 2-methyl-propylene, 1,1-dimethyl-ethylene, butylene, 2-ethylbutylene.

The term “alkoxy” as used herein means an —O-alkyl group, wherein alkyl is as defined above such as methoxy, ethoxy, n-propyloxy, i-propyloxy, n-butyloxy, i-butyloxy, t-butyloxy, pentyloxy, hexyloxy, including their isomers. “Lower alkoxy” as used herein denotes an alkoxy group with a “lower alkyl” group as previously defined. “C₁₋₁₀ alkoxy” as used herein refers to an —O-alkyl wherein alkyl is C₁₋₁₀.

The term “hydroxyalkyl” as used herein denotes an alkyl radical as herein defined wherein one to three hydrogen atoms on different carbon atoms is/are replaced by hydroxyl groups.

The terms “alkylsulfonyl” and “arylsulfonyl” as used herein refers to a group of formula —S(═O)₂R wherein R is alkyl or aryl respectively and alkyl and aryl are as defined herein. The term “heteroalkylsulfonyl” as used herein refers herein denotes a group of formula —S(═O)₂R wherein R is “heteroalkyl” as defined herein.

The terms “alkylsulfonylamino” and “arylsulfonylamino” as used herein refers to a group of formula-NR'S(═O)₂R wherein R is alkyl or aryl respectively, R′ is hydrogen or C₁₋₃ alkyl, and alkyl and aryl are as defined herein.

The term “cycloalkyl” as used herein refers to a saturated carbocyclic ring containing 3 to 8 carbon atoms, i.e. cyclopropyl, cyclobutyl, cyclopentyl, cyclohexyl, cycloheptyl or cyclooctyl. “C₃₇ cycloalkyl” as used herein refers to an cycloalkyl composed of 3 to 7 carbons in the carbocyclic ring.

The term carboxy-alkyl as used herein refers to an alkyl moiety wherein one, hydrogen atom has been replaced with a carboxyl with the understanding that the point of attachment of the heteroalkyl radical is through a carbon atom. The term “carboxy” or “carboxyl” refers to a —CO₂H moiety.

The term “heteroaryl” or “heteroaromatic” as used herein means a monocyclic or bicyclic radical of 5 to 12 ring atoms having at least one aromatic ring containing four to eight atoms per ring, incorporating one or more N, O, or S heteroatoms, the remaining ring atoms being carbon, with the understanding that the attachment point of the heteroaryl radical will be on an aromatic ring. As well known to those skilled in the art, heteroaryl rings have less aromatic character than their all-carbon counter parts. Thus, for the purposes of the invention, a heteroaryl group need only have some degree of aromatic character. Examples of heteroaryl moieties include monocyclic aromatic heterocycles having 5 to 6 ring atoms and 1 to 3 heteroatoms include, but is not limited to, pyridinyl, pyrimidinyl, pyrazinyl, pyrrolyl, pyrazolyl, imidazolyl, oxazol, isoxazole, thiazole, isothiazole, triazoline, thiadiazole and oxadiaxoline which can optionally be substituted with one or more, preferably one or two substituents selected from hydroxy, cyano, alkyl, alkoxy, thio, lower haloalkoxy, alkylthio, halo, lower haloalkyl, alkylsulfinyl, alkylsulfonyl, halogen, amino, alkylamino, dialkylamino, aminoalkyl, alkylaminoalkyl, and dialkylaminoalkyl, nitro, alkoxycarbonyl and carbamoyl, alkylcarbamoyl, dialkylcarbamoyl, arylcarbamoyl, alkylcarbonylamino and arylcarbonylamino. Examples of bicyclic moieties include, but are not limited to, quinolinyl, isoquinolinyl, benzofuryl, benzothiophenyl, benzoxazole, benzisoxazole, benzothiazole and benzisothiazole. Bicyclic moieties can be optionally substituted on either ring; however the point of attachment is on a ring containing a heteroatom.

The term “heterocyclyl”, “heterocycloalkyl” or “heterocycle” as used herein denotes a monovalent saturated cyclic radical, consisting of one or more rings, preferably one to two rings, including spirocyclic ring systems, of three to eight atoms per ring, incorporating one or more ring heteroatoms (chosen from N, O or S(O)₀₋₂), and which can optionally be independently substituted with one or more, preferably one or two substituents selected from hydroxy, oxo, cyano, lower alkyl, lower alkoxy, lower haloalkoxy, alkylthio, halo, lower haloalkyl, hydroxyalkyl, nitro, alkoxycarbonyl, amino, alkylamino, alkylsulfonyl, arylsulfonyl, alkylaminosulfonyl, arylaminosulfonyl, alkylsulfonylamino, arylsulfonylamino, alkylaminocarbonyl, arylaminocarbonyl, alkylcarbonylamino, arylcarbonylamino, unless otherwise indicated. Examples of heterocyclic radicals include, but are not limited to, azetidinyl, pyrrolidinyl, hexahydroazepinyl, oxetanyl, tetrahydrofuranyl, tetrahydrothiophenyl, oxazolidinyl, thiazolidinyl, isoxazolidinyl, morpholinyl, piperazinyl, piperidinyl, tetrahydropyranyl, thiomorpholinyl, quinuclidinyl and imidazolinyl.

“Heterocyclyl” or “heterocyclic” refers to a group or radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include, without limitation, azirdinyl, oxiranyl, and thiiranyl. Exemplary 4-membered heterocyclyl groups containing 1 heteroatom include, without limitation, azetidinyl, oxetanyl and thietanyl. Exemplary 5-membered heterocyclyl groups containing 1 heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include, without limitation, dioxolanyl, oxathiolanyl and dithiolanyl. Exemplary 5-membered heterocyclyl groups containing 3 heteroatoms include, without limitation, triazolinyl, oxadiazolinyl, and thiadiazolinyl. Exemplary 6-membered heterocyclyl groups containing 1 heteroatom include, without limitation, piperidinyl, tetrahydropyranyl, dihydropyridinyl, and thianyl. Exemplary 6-membered heterocyclyl groups containing 2 heteroatoms include, without limitation, piperazinyl, morpholinyl, dithianyl, and dioxanyl. Exemplary 6-membered heterocyclyl groups containing 3 heteroatoms include, without limitation, triazinanyl. Exemplary 7-membered heterocyclyl groups containing 1 heteroatom include, without limitation, azepanyl, oxepanyl and thiepanyl. Exemplary 8-membered heterocyclyl groups containing 1 heteroatom include, without limitation, azocanyl, oxecanyl and thiocanyl. Exemplary bicyclic heterocyclyl groups include, without limitation, indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo-[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

“Aryl” refers to a radical of a monocyclic or polycyclic (e.g., bicyclic or tricyclic) 4n+2 aromatic ring system (e.g., having 6, 10, or 14 pi electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl (α-naphthyl) and 2-naphthyl (β-naphthyl)). In some embodiments, an aryl group has 14 ring carbon atoms (“C₁ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system.

“Heteroaryl” refers to a radical of a 5-14 membered monocyclic or polycyclic (e.g., bicyclic, tricyclic) 4n+2 aromatic ring system (e.g., having 6, 10, or 14 pi electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur.

Exemplary 5-membered heteroaryl groups containing 1 heteroatom include, without limitation, pyrrolyl, furanyl and thiophenyl. Exemplary 5-membered heteroaryl groups containing 2 heteroatoms include, without limitation, imidazolyl, pyrazolyl, oxazolyl, isoxazolyl, thiazolyl, and isothiazolyl. Exemplary 5-membered heteroaryl groups containing 3 heteroatoms include, without limitation, triazolyl, oxadiazolyl, and thiadiazolyl. Exemplary 5-membered heteroaryl groups containing 4 heteroatoms include, without limitation, tetrazolyl. Exemplary 6-membered heteroaryl groups containing 1 heteroatom include, without limitation, pyridinyl. Exemplary 6-membered heteroaryl groups containing 2 heteroatoms include, without limitation, pyridazinyl, pyrimidinyl, and pyrazinyl. Exemplary 6-membered heteroaryl groups containing 3 or 4 heteroatoms include, without limitation, triazinyl and tetrazinyl, respectively. Exemplary 7-membered heteroaryl groups containing 1 heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Exemplary tricyclic heteroaryl groups include, without limitation, phenanthridinyl, dibenzofuranyl, carbazolyl, acridinyl, phenothiazinyl, phenoxazinyl and phenazinyl.

“Saturated” refers to a ring moiety that does not contain a double or triple bond, i.e., the ring contains all single bonds.

Alkyl, cycloalkyl, heterocyclyl, aryl, and heteroaryl groups may be optionally substituted. Optionally substituted refers to a group which may be substituted or unsubstituted. In general, the term “substituted” means that at least one hydrogen present on a group is replaced with a non-hydrogen substituent, and which upon substitution results in a stable compound, e.g., a compound which does not spontaneously undergo transformation such as by rearrangement, cyclization, elimination, or other reaction. Heteroatoms such as nitrogen, oxygen, and sulfur may have hydrogen substituents and/or non-hydrogen substituents which satisfy the valencies of the heteroatoms and results in the formation of a stable compound.

Exemplary non-hydrogen substituents wherein a moiety is “optionally substituted” as used herein means the moiety may be substituted with any additional moiety selected from, but not limited to, the group consisting of halogen, —CN, —NO₂, —N₃, —SO₂H, —SO₃H, —OH, —OR^{aa}, —N(R^{bb})₂, —N(OR^{cc})R^{bb}, —SH, —SR^{aa}, —C(═O)R^{aa}, —CO₂H, —CHO, —CO₂R^{aa}, OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NRC^{bb}(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, NR^{bb}SO₂R^{aa}, —SO₂N(R^{bb})₂, —SO₂R^{aa}, —S(═O)R^{aa}, —OS(═O)R^{aa}, —B(OR^{cc})₂, C₁₋₁₀ alkyl, C₂₋₁₀ alkenyl, C₂₋₁₀ alkynyl, C₃₋₁₄ carbocyclyl, 3- to 14-membered heterocyclyl, C₆₋₁₄ aryl, and 5- to 14-membered heteroaryl, wherein each alkyl, alkenyl, alkynyl, carbocyclyl, heterocyclyl, aryl, and heteroaryl is independently substituted with 0, 1, 2, 3, 4, or 5 R^{dd} groups, or two geminal hydrogens on a carbon atom are replaced with the group ═O; each instance of R^{aa} is, independently, selected from the group consisting of C₁₋₁₀ alkyl, C₁₋₁₀ perhaloalkyl, C₂₋₁₀ alkenyl, C₂₋₁₀ alkynyl, C₃₋₁₄ carbocyclyl, 3- to 14-membered heterocyclyl, C₆₋₁₄ aryl, and 5- to 14-membered heteroaryl, or two R^{aa} groups are joined to form a 3- to 14-membered heterocyclyl or 5- to 14-membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups; each instance of R^{bb} is, independently, selected from the group consisting of hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —SO₂N(R^{cc})₂, —SOR^{aa}, C₁₋₁₀ alkyl, C₁₋₁₀ perhaloalkyl, C₂₋₁₀ alkenyl, C₂₋₁₀ alkynyl, C₃₋₁₄ carbocyclyl, 3- to 14-membered heterocyclyl, C₆₋₁₄ aryl, and 5- to 14-membered heteroaryl, or two R^{bb} groups are joined to form a 3- to 14-membered heterocyclyl or 5- to 14-membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups; each instance of R^{cc} is, independently, selected from the group consisting of hydrogen, C₁₋₁₀ alkyl, C₁₋₁₀ perhaloalkyl, C₂₋₁₀ alkenyl, C₂₋₁₀ alkynyl, C₃₋₁₄ carbocyclyl, 3- to 14-membered heterocyclyl, C₆₋₁₄ aryl, and 5- to 14-membered heteroaryl, or two R^{cc} groups are joined to form a 3- to 14-membered heterocyclyl or 5- to 14-membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups; and each instance of R^{dd} is, independently, selected from the group consisting of halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(OC₁₋₆ alkyl)(C₁₋₆ alkyl), —N(OH)(C₁₋₆ alkyl), —NH(OH), —SH, —SC₁₋₆ alkyl, —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, — OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —B(OH)₂, —B(OC₁₋₆ alkyl)₂, C₁₋₆ alkyl, C₁₋₆ perhaloalkyl, C₂₋₆ alkenyl, C₂₋₆ alkynyl, C₃₋₁₀ carbocyclyl, C₆₋₁₀ aryl, 3- to 10-membered heterocyclyl, and 5- to 10-membered heteroaryl; or two geminal R^{dd} substituents on a carbon atom may be joined to form ═O.

“Halo” or “halogen” refers to fluorine (fluoro, —F), chlorine (chloro, —Cl), bromine (bromo, —Br), or iodine (iodo, —I).

As used herein, the term “composition” is intended to encompass a product comprising the specified ingredients, as well as any product which results, directly or indirectly, from combination of the specified ingredients.

“Salt” includes any and all salts. “Pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describes pharmaceutically acceptable salts in detail in J. Pharmaceutical Sciences (1977) 66:1-19. Pharmaceutically acceptable salts include those derived from inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Pharmaceutically acceptable salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

Unless otherwise indicated, compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC). Compounds described herein can be in the form of individual isomers substantially free of other isomers, and alternatively, as mixtures of various isomers.

Unless otherwise stated, structures depicted herein are also meant to include compounds that differ only in the presence of one or more isotopically enriched atoms. For example, compounds having the present structures except for the replacement of hydrogen by deuterium or tritium, replacement of ¹⁹F with ¹⁸F, replacement of a carbon by a ¹³C- or ⁴C-enriched carbon, and/or replacement of an oxygen atom with ¹⁸O, are within the scope of the disclosure. Other examples of isotopes include ¹⁵N, ¹⁸O, ¹⁷O, ³¹P, ³²P, ³⁵S, ¹⁸F, ³⁶Cl and ¹²³I. Compounds with such isotopically enriched atoms are useful, for example, as analytical tools or probes in biological assays.

Certain isotopically-labelled compounds (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability.

Certain isotopically-labelled compounds of Formula (I) can be useful for medical imaging purposes, for example, those labeled with positron-emitting isotopes like ¹¹C or ¹⁸F can be useful for application in Positron Emission Tomography (PET) and those labeled with gamma ray emitting isotopes like ¹²³I can be useful for application in Single Photon Emission Computed Tomography (SPECT). Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Further, substitution with heavier isotopes such as deuterium (i.e., ²H) may afford certain therapeutic advantages resulting from greater metabolic stability (e.g., increased in vivo half-life or reduced dosage requirements), and hence, may be preferred in some circumstances. Additionally, isotopic substitution at a site where epimerization occurs may slow or reduce the epimerization process and thereby retain the more active or efficacious form of the compound for a longer period of time. Isotopically labeled compounds of Formula (I), in particular those containing isotopes with longer half-lives (t_{1/2}>1 day), can generally be prepared by following procedures analogous to those disclosed in the Schemes and/or in the Examples herein below, by substituting an appropriate isotopically labeled reagent for a non-isotopically labeled reagent.

If there is a discrepancy between a depicted structure and a name given to that structure, then the depicted structure controls. Additionally, if the stereochemistry of a structure or a portion of a structure is not indicated with, for example, bold or dashed lines, the structure or portion of the structure is to be interpreted as encompassing all stereoisomers of it. In some cases, however, where more than one chiral center exists, the structures and names may be represented as single enantiomers to help describe the relative stereochemistry. Those skilled in the art of organic synthesis will know if the compounds are prepared as single enantiomers from the methods used to prepare them.

### DETAILED DESCRIPTION OF THE FIGURES

FIG. 1. [2+2] Annulation Reaction via Dual Methylene C—H Bond Functionalization. (A) Dual functionalization of two adjacent inert methylene C—H bonds. (B) Applications of BCBs. Ki is the inhibitor constant. (C) Limitations of BCB synthesis via [2+2]cycloaddition of alkenes and benzynes. Syn, anti refers to the conformation of R¹ group and the adjacent aryl group. (D) Ligand-enabled [2+2] annulation between aliphatic acids and dihaloarenes.

FIG. 2. Cyclic aliphatic acids scope for the [2+2] annulation reaction. Reaction conditions: aliphatic acid (0.1 mmol), 2a (0.2 mmol), Pd(CH₃CN)₄(BF₄)₂ (10 mol %), L7 (13 mol %), K₂CO₃ (2.5 equiv), Ag₂CO₃ (2.0 equiv), HFIP (1.0 ml), at 100° C. for 20 hours. See supplementary materials for details. *Pd(CH₃CN)₄(BF₄)₂(1 mol %) was used, reaction time 56 hours. \Reaction conducted with of aliphatic acid 1a (4.5 mmol), 1.05 g of 3a was obtained. ‡Pd(CH₃CN)₄(BF₄)₂(5 mol %) was used, reaction time 40 hours. ¶Aliphatic acid (0.2 mmol), 2a (0.1 mmol), K₂CO₃ (3.5 equiv). § L28 instead of L7, KHCO₃ instead of K₂CO₃.

FIG. 3. Acyclic aliphatic acids scope for the [2+2] annulation reaction and transformations of the products. (A) Acyclic aliphatic acid scope. Reaction conditions: aliphatic acid (0.1 mmol), 2a (0.3 mmol), Pd(OAc)₂ (10 mol %), L12 (20 mol %), K₂HPO₄ (3.5 equiv), Ag₂CO₃ (2.0 equiv), HFIP (1.2 ml), at 110° C. for 24 hours. *2a (0.35 mmol), K₂HPO₄ (3.0 equiv), HFIP (1.8 ml) as a replacement. (B) Synthetic applications. DPPA=Diphenylphosphonicazide.

FIG. 4. Dihaloarenes scope for the [2+2] annulation reaction. (A) Scope of bromoiodobenzenes; (B) Scope of dibromobenzenes and diiodobenzene; (C) Scope of heterocycles and complex molecules. Reaction conditions: aliphatic acid (0.1 mmol), dihaloarene (0.2 mmol), Pd(CH₃CN)₄(BF₄)₂(10 mol %), L7 (13 mol %), K₂CO₃ (2.5 equiv), Ag₂CO₃ (2.0 equiv), HFIP (1.0 ml), at 100° C. for 20 hours. *aliphatic acid (0.2 mmol), dihaloarene (0.1 mmol), K₂CO₃ (3.5 equiv). †L28 instead of L7, KHCO₃ instead of K₂CO₃. ‡dihaloarene (0.3 mmol), Pd(OAc)₂ (10 mol %), L12 (20 mol %), K₂HPO₄ (3.5 equiv), Ag₂CO₃ (2.0 equiv), HFIP (1.2 ml), at 110° C. for 24 hours.

### Examples

### Abbreviations

Commonly used abbreviations include: acetyl (Ac), azo-bis-isobutyrylnitrile (AIBN), atmospheres (Atm), 9-borabicyclo[3.3.1]nonane (9-BBN or BBN), tert-butoxycarbonyl (Boc), di-tert-butyl pyrocarbonate or boc anhydride (BOC₂O), benzyl (Bn), butyl (Bu), Chemical Abstracts Registration Number (CASRN), benzyloxycarbonyl (CBZ or Z), carbonyl diimidazole (CDI), 1,4-diazabicyclo[2.2.2]octane (DABCO), diethylaminosulfur trifluoride (DAST), dibenzylideneacetone (dba), 1,5-diazabicyclo[4.3.0]non-5-ene (DBN), 1,8-diazabicyclo[5.4.0]undec-7-ene (DBU), N,N′-dicyclohexylcarbodiimide (DCC), 1,2-dichloroethane (DCE), dichloromethane (DCM), diethyl azodicarboxylate (DEAD), di-iso-propylazodicarboxylate (DIAD), di-iso-butylaluminumhydride (DIBAL or DIBAL-H), 1,3-Diisopropylcarbodiimide (DIC), di-iso-propylethylamine (DIPEA), N,N-dimethyl acetamide (DMA), 4-N,N-dimethylaminopyridine (DMAP), N,N-dimethylformamide (DMF), dimethyl sulfoxide (DMSO), 1,1′-bis-(diphenylphosphino)ethane (dppe), 1,1′-bis-(diphenylphosphino)ferrocene (dppf), 1-(3-dimethylaminopropyl)-3-ethylcarbodiimide hydrochloride (EDCI), ethyl (Et), ethyl acetate (EtOAc), ethanol (EtOH), 2-ethoxy-2H-quinoline-1-carboxylic acid ethyl ester (EEDQ), diethyl ether (Et₂O), O-(7-azabenzotriazole-1-yl)-N, N,N′N′-tetramethyluronium hexafluorophosphate acetic acid (HATU), acetic acid (HOAc), 1-N-hydroxybenzotriazole (HOBt), high pressure liquid chromatography (HPLC), iso-propanol (IPA), lithium hexamethyl disilazane (LiHMDS), methanol (MeOH), melting point (mp), MeSO₂- (mesyl or Ms), methyl (Me), acetonitrile (MeCN), m-chloroperbenzoic acid (MCPBA), mass spectrum (ms), methyl t-butyl ether (MTBE), N-bromosuccinimide (NBS), N-carboxyanhydride (NCA), N-chlorosuccinimide (NCS), N-methylmorpholine (NMM), N-methylpyrrolidone (NMP), pyridinium chlorochromate (PCC), pyridinium dichromate (PDC), phenyl (Ph), propyl (Pr), iso-propyl (i-Pr), pounds per square inch (psi), pyridine (pyr), room temperature (rt or RT), tert-butyldimethylsilyl or t-BuMe₂Si (TBDMS), triethylamine (TEA or Et₃N), 2,2,6,6-tetramethylpiperidine 1-oxyl (TEMPO), triflate or CF₃SO₂-(Tf), trifluoroacetic acid (TFA), 1,1′-bis-2,2,6,6-tetramethylheptane-2,6-dione (TMHD), O-benzotriazol-1-yl-N,N,N′,N′-tetramethyluronium tetrafluoroborate (TBTU), thin layer chromatography (TLC), tetrahydrofuran (THF), trimethylsilyl or Me₃Si (TMS), p-toluenesulfonic acid monohydrate (TsOH or pTsOH), 4-Me-C₆H₄SO₂— or tosyl (Ts), N-urethane-N-carboxyanhydride (UNCA). Conventional nomenclature including the prefixes normal (n), iso (i-), secondary (sec-), tertiary (tert-) and neo have their customary meaning when used with an alkyl moiety. (J. Rigaudy and D. P. Klesney, Nomenclature in Organic Chemistry, IUPAC 1979 Pergamon Press, Oxford.).

### General Information

Pd(OAc)₂, Pd(CH₃CN)₄(BF₄)₂, Ag₂CO₃ were purchased from Strem and Sigma-Aldrich. Solvents were obtained from Sigma-Aldrich, Alfa-Aesar, and Acros, and used directly without further purification. Other reagents were purchased at the highest commercial quality and used without further purification, unless otherwise stated. Analytical thin layer chromatography was performed on 0.25 mm silica gel 60-F254. ¹H NMR spectra were recorded on Bruker AMX-400, Bruker AV-500, or Bruker DRX-600 instruments. The following abbreviations (or combinations thereof) were used to explain multiplicities: s=singlet, d=doublet, t=triplet, q=quartet, m=multiplet, br=broad. Coupling constants, J, were reported in Hertz unit (Hz). ¹³C NMR spectra were recorded on Bruker DRX-600 and were fully decoupled by broad band proton decoupling. Chemical shifts were referenced to the appropriate residual solvent peaks. Column chromatography was performed using E. Merck silica (60, particle size 0.043-0.063 mm), and pTLC was performed on Merck silica plates (60F-254). High-resolution mass spectra (HRMS) were recorded on an Agilent Mass spectrometer using ESI-TOF (electrospray ionization-time of flight). The single crystal X-ray diffraction studies were carried out on a Bruker D8 Venture Ultra diffractometer equipped with Mo K_{α} radiation (λ=0.71073).

### Preparation of Bidentate Amide-Pyridone Ligands

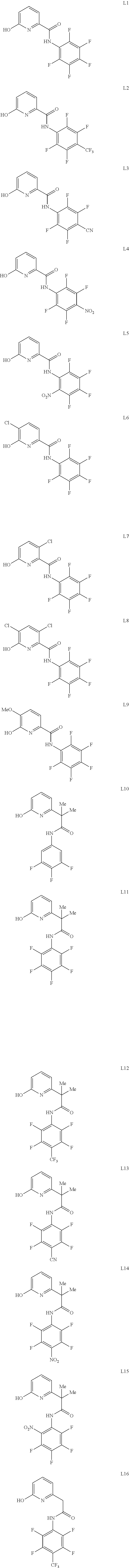

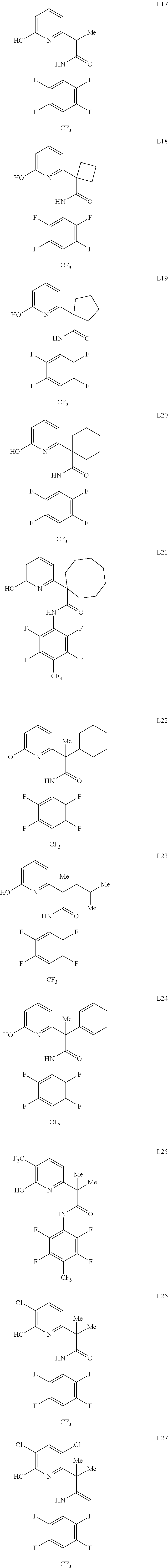

### Preparation of L1

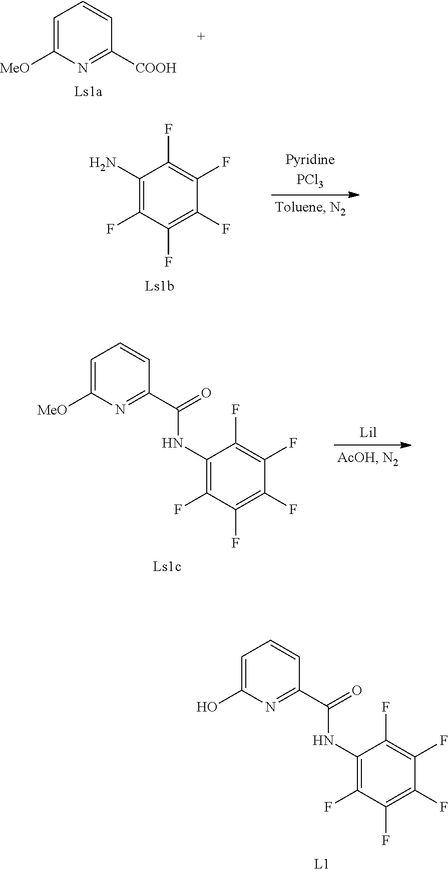 [SMILES: COc1cccc(C(=O)O)n1 ; Nc1c(F)c(F)c(F)c(F)c1F ; COc1cccc(C(=O)Nc2c(F)c(F)c(F)c(F)c2F)n1 ; O=C(Nc1c(F)c(F)c(F)c(F)c1F)c1cccc(O)n1]

To a flame-dried (anhydrous condition is important) 16 mL reaction vial Ls1a (2.2 mmol) and Ls1b (2 mmol) was added. The vial was sealed tightly, evacuated, and refilled with N₂ three times. Then anhydrous toluene (6.6 mL) was added, followed by pyridine (2 drops). Then phosphorus trichloride (1 mmol) was added dropwisely while vigorously stirred at room temperature (Solidification may occur in the solution if stirring is not vigorous enough. And if white smoke appeared above the solution when adding phosphorus trichloride, probably you will not see the product). The vial was put into a 145° C. heating bath with stirring. Solution became clear and red solid formed at the inner surface of the vial in about 1 min. The vial was heated at 145° C. and stirred for 16 h (inner pressure is high!). The vial was removed from the heating bath and cooled down to room temperature. The solution was poured into water (10 mL) and extracted by EtOAc (10 mL×3). The organic phase was washed by conc. brine (10 mL×3), and dried by anhydrous Na₂SO₄. The dried organic phase was passed through a pad of silica gel and washed by EtOAc (30 mL). The solvent was removed by a rotavapor, and Ls1b was removed by blowing with compressed air under room temperature if there is any. Ls1c was obtained as a white solid and used in the next step without further purification.

To a 16 mL vial Ls1c (1 mmol) and LiI (5 mmol) was added, followed by 2 mL AcOH. The vial was sealed, evacuated, and refilled with N₂ three times. The vial was put into a 110° C. heating bath, heated and stirred under this temperature for 2 h. Then the vial was removed from the heating bath and cooled down to room temperature. The reaction mixture was poured into water (10 mL), and solid Na₂CO₃ was added into the solution until pH=2. The solution was extracted by EtOAc (10 mL×3) and washed by conc. brine (10 mL×3). The organic phase was dried by anhydrous Na₂SO₄ and passed through a pad of celite. The solvent was removed, and the residue was purified by flash silica gel column chromatography (2% formic acid with 10% to 50% EtOAc/hexane) to give the L1 (82% over two steps) as a white solid.

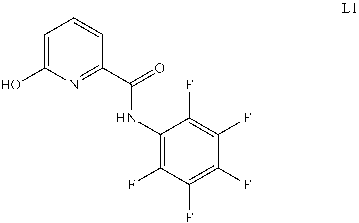 [SMILES: O=C(Nc1c(F)c(F)c(F)c(F)c1F)c1cccc(O)n1]

¹H NMR (600 MHz, Acetone-d₆) δ 7.91 (dd, J=8.4, 7.2 Hz, 1H), 7.68 (dd, J=7.3, 0.9 Hz, 1H), 7.01 (dd, J=8.3, 0.8 Hz, 1H). ¹³C NMR (151 MHz, Acetone-d₆) δ 163.71, 163.28, 146.71, 144.48 (dm, J=247.96 Hz), 141.83, 140.77 (dm, J=252.48 Hz), 138.83 (dm, J=251.73 Hz), 115.92, 115.43, 114.14 (m). ¹⁹F NMR (376 MHz, CDCl₃) δ −146.90, −158.78, −164.59.

HRMS (ESI-TOF) Calculated for C₁₂H₆F₅N₂O₂ [M+H]⁺: 305.0349, Found: 305.0350.

### Preparation of L2 to L5

Preparation of L2 to L5 followed the procedure of the preparation of L1 with corresponding aniline, and the reaction mixture was purified by flash silica gel column chromatography (20 formic acid with 10% to 50% EtOAc in hexane) to give L2, L3 and L5 as a white solid, and L4 as a yellow solid.

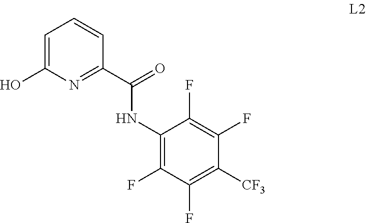 [SMILES: O=C(Nc1c(F)c(F)c(C(F)(F)F)c(F)c1F)c1cccc(O)n1]

¹H NMR (400 MHz, Acetone-d₆) δ 10.07 (s, 1H), 7.94 (dd, J=8.4, 7.2 Hz, 1H), 7.72 (d, J=7.2 Hz, 1H), 7.04 (d, J=8.3 Hz, 1H). ¹³C NMR (151 MHz, Acetone-d₆) δ 163.70, 162.69, 146.57, 145.42 (dm, J=236.0 Hz), 143.73 (dm, J=230.7 Hz), 142.02, 122.92 (m), 122.10 (q, J=272.35 Hz), 115.95, 115.86, 107.16 (m). ¹⁹F NMR (376 MHz, CDCl₃) δ −58.70, −143.38, −145.02.

HRMS (ESI-TOF) Calculated for C₁₃H₆F₇N₂O₂ [M+H]⁺: 355.0317, Found: 355.0319.

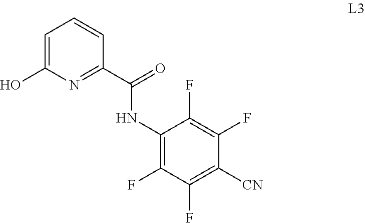 [SMILES: N#Cc1c(F)c(F)c(NC(=O)c2cccc(O)n2)c(F)c1F]

¹H NMR (600 MHz, Acetone-d₆) δ 10.13 (s, 1H), 7.94 (dd, J=8.3, 7.2 Hz, 1H), 7.72 (dd, J=7.3, 0.9 Hz, 1H), 7.04 (dd, J=8.3, 0.8 Hz, 1H). ¹³C NMR (151 MHz, Acetone-d₆) δ 163.72, 162.62, 148.47 (dm, J=257.7 Hz), 146.49, 143.31 (dm, J=247.2 Hz), 142.03, 124.65 (m), 116.00, 115.90, 108.53, 92.06 (m). ¹⁹F NMR (376 MHz, CDCl₃) δ −134.88, −143.60. HRMS (ESI-TOF) Calculated for C₁₃H₆F₄N₃O₂ [M+H]⁺: 312.0396, Found: 312.0393.

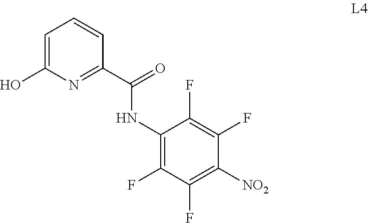 [SMILES: O=C(Nc1c(F)c(F)c([N+](=O)[O-])c(F)c1F)c1cccc(O)n1]

¹H NMR (600 MHz, Acetone-d₆) δ 7.93 (dd, J=8.4, 7.2 Hz, 1H), 7.71 (d, J=7.3 Hz, 1H), 7.03 (d, J=8.3 Hz, 1H). ¹³C NMR (151 MHz, Acetone-d₆) δ 163.73, 162.62, 146.33, 143.70 (ddm, J=200.5, 14.7 Hz), 142.00 (ddm, J=210.77, 16.75 Hz), 141.98, 128.62 (m), 123.42 (m), 116.20, 115.60. ¹⁹F NMR (376 MHz, CDCl₃) δ −143.28, −148.88.

HRMS (ESI-TOF) Calculated for C₁₂H₆F₄N₃O₄ [M+H]⁺: 332.0294, Found: 332.0298.

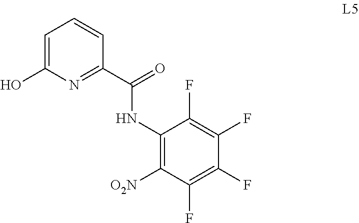 [SMILES: O=C(Nc1c(F)c(F)c(F)c(F)c1[N+](=O)[O-])c1cccc(O)n1]

¹H NMR (600 MHz, Acetone-d₆) δ 10.21 (s, 1H), 7.93 (dd, J=8.3, 7.2 Hz, 1H), 7.69 (dd, J=7.3, 0.9 Hz, 1H), 7.04 (dd, J=8.4, 0.9 Hz, 1H). ¹³C NMR (151 MHz, Acetone-d₆) δ 163.77, 163.11, 146.52, 144.33 (dm, J=258.21 Hz), 143.72 (dm, J=246.13 Hz), 143.07 (dm, J= 273.31 Hz), 140.00 (dm, J=253.68 Hz), 142.05, 133.02 (m), 119.23 (dm, J=15.4 Hz), 116.00, 115.86. ¹⁹F NMR (376 MHz, CDCl₃) δ −141.07, −147.46, −149.56, −156.46.

HRMS (ESI-TOF) Calculated for C₁₂H₆F₄N₃O₄ [M+H]⁺: 332.0294, Found: 332.0292.

### Preparation of L6 to L8

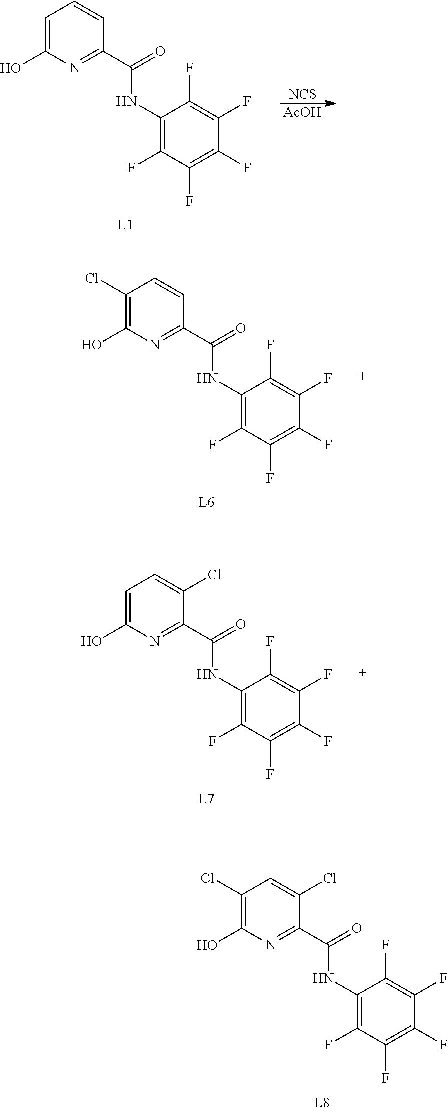 [SMILES: "AcOH" ; O=C(Nc1c(F)c(F)c(F)c(F)c1F)c1cccc(O)n1 ; "NCS" ; O=C(Nc1c(F)c(F)c(F)c(F)c1F)c1ccc(Cl)c(O)n1 ; O=C(Nc1c(F)c(F)c(F)c(F)c1F)c1nc(O)ccc1Cl ; O=C(Nc1c(F)c(F)c(F)c(F)c1F)c1nc(O)c(Cl)cc1Cl]

To a 16 mL vial was added L1 (1 mmol) and followed by 2 mL AcOH. N-chlorosuccinimide (NCS, 1.5 mmol) was added to the solution. The vial was capped and stirred at 120° C. for 16 h. After cooling down, the reaction mixture was poured into water (10 mL), and solid Na₂CO₃ was added into the solution until pH=2. The solution was extracted by EtOAc (10 mL×3) and washed by conc. brine (10 mL×3). The organic phase was dried by anhydrous Na₂SO₄ and passed through a pad of celite. The solvent was removed, and the residue was purified by dedicated silica gel column chromatography (2% formic acid with 10% to 50% EtOAc in hexane) to give the L6 (25%), L7 (33%) and L8 (13%) as white solids.

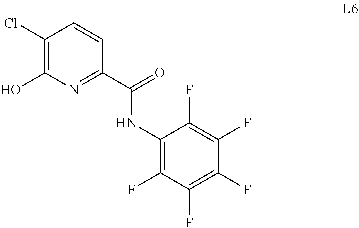 [SMILES: O=C(Nc1c(F)c(F)c(F)c(F)c1F)c1ccc(Cl)c(O)n1]

¹H NMR (600 MHz, Acetone-d₆) δ 7.98 (d, J=7.7 Hz, 1H), 7.57 (d, J=7.7 Hz, 1H). ¹³C NMR (151 MHz, Acetone-d₆) δ 162.17, 159.27, 144.42 (dm, J=249.9 Hz), 142.97, 140.94 (dm, J=251.1 Hz), 140.49, 138.73 (dm, J=248.3 Hz), 124.78, 114.49, 113.64 (tm, J=14.9 Hz). ¹⁹F NMR (376 MHz, CD₃OD) δ −149.67, −162.19, −168.49.

HRMS (ESI-TOF) Calculated for C₁₂H₅ClF₅N₂O₂ [M+H]⁺: 338.9960, Found: 338.9957.

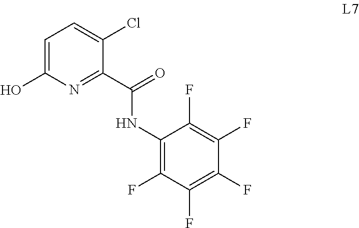 [SMILES: O=C(Nc1c(F)c(F)c(F)c(F)c1F)c1nc(O)ccc1Cl]

¹H NMR (600 MHz, Acetone-d₆) δ 7.87 (d, J=8.8 Hz, 1H), 7.00 (d, J=8.8 Hz, 1H). ¹³C NMR (151 MHz, Acetone-d₆) δ 161.58, 161.17, 143.46 (dm, J=242.6 Hz), 143.12, 143.06, 139.92 (dm, J=250.6 Hz), 137.82 (dm, J=249.0 Hz), 121.91, 114.82, 112.77 (tm, J=14.8 Hz). ¹⁹F NMR (376 MHz, CD₃OD) δ −149.32, −162.46, −168.58.

HRMS (ESI-TOF) Calculated for C₁₂H₅ClF₅N₂O₂ [M+H]⁺: 338.9960, Found: 338.9956.

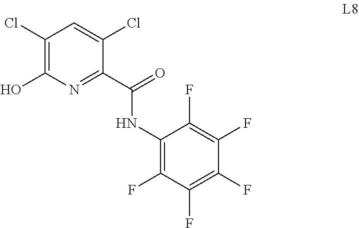 [SMILES: O=C(Nc1c(F)c(F)c(F)c(F)c1F)c1nc(O)c(Cl)cc1Cl]

¹H NMR (600 MHz, Acetone-d₆) δ 8.06 (s, 1H). ¹³C NMR (151 MHz, Acetone-d₆) δ 161.88, 158.05, 144.35 (dm, J=254.0 Hz), 142.46, 142.06, 140.99 (dm, J=250.9 Hz), 138.80 (dm, J=253.2 Hz), 122.23, 121.96, 113.46 (tm, J=13.3 Hz). ¹⁹F NMR (376 MHz, CD₃OD) δ −149.34, −162.26, −168.48.

HRMS (ESI-TOF) Calculated for C₁₂H₄Cl₂F₅N₂O₂[M+H]⁺: 372.9570, Found: 372.9566.

### Preparation of L7 in Large Scale

Preparation of L7 in large scale followed the procedure of the preparation of Li with 3-chloro-6-methoxypicolinic acid (11 mmol) and Ls1b (10 mmol) to give L7 (67% over two steps) as a white solid.

### Preparation of L9

Preparation of L9 followed the procedure of the preparation of L1 with 5,6-dimethoxypicolinic acid and Ls1b and purified by preparative HPLC (water/acetonitrile) to give L9 as a white solid.

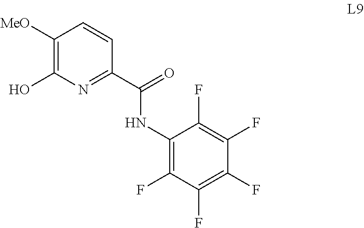 [SMILES: COc1ccc(C(=O)Nc2c(F)c(F)c(F)c(F)c2F)nc1O]

¹H NMR (600 MHz, CD₃OD) δ 7.33 (d, J=7.8 Hz, 1H), 7.11 (d, J=7.8 Hz, 1H), 3.93 (s, 3H)¹³C NMR (151 MHz, CD₃OD) δ 170.12, 162.64, 158.52, 153.78, 144.91 (dm, J=249.7 Hz), 141.49 (dm, J=252.0 Hz), 139.19 (dm, J=248.5 Hz), 114.80, 113.80, 111.99 (m), 56.78. ¹⁹F NMR (376 MHz, Acetone) δ −149.13, −162.58, −168.35.

HRMS (ESI-TOF) Calculated for C₁₃H₈F₅N₂O₃ [M+H]⁺: 335.0455, Found: 335.0453.

### Preparation of L12

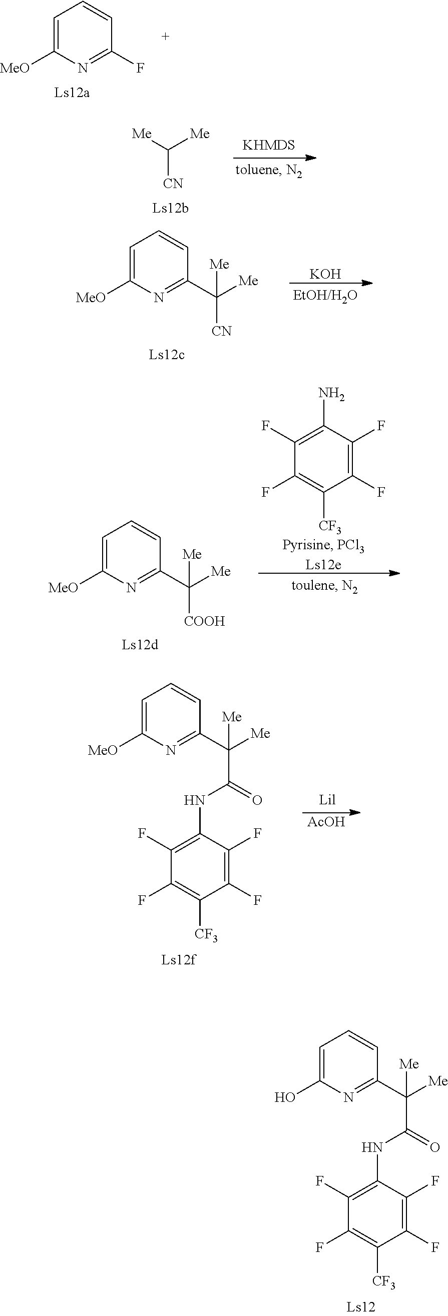

To a dry 100 mL round-bottom flask, Ls12a (10 mmol) was added. The flask was sealed and evacuated and refilled by N₂ three times. Anhydrous toluene (20 mL) and Ls12b (10 mmol) was added to the flask, followed by dropwise addition of potassium bis(trimethylsilyl)amide (KHMDS, 11 mmol, 1.0 M in THF) at room temperature. Then the flask was put into a 50° C. heating bath, heated and stirred under this temperature for 16 h. After the completion of the reaction, the reaction mixture was cooled down and water (20 mL) was added slowly. The mixture was extracted by EtOAc (30 mL×3) and washed by conc. brine (30 mL×3). The organic phase was dried by anhydrous Na₂SO₄ and passed through a pad of silica gel. Solvent was removed from the solution by a rotavapor, and Ls12c was used in the next step without further purification.

Ls12c (10 mmol) was added to a 100 mL round-bottom flask. Potassium hydroxide (300 mmol) was dissolved in 30 mL water, and the solution was poured into the round bottom flask, followed by addition of 30 mL ethanol. The flask was equipped with a condenser, and put into a 115° C. heating bath, and was stirred and refluxed at this temperature for 24 h or longer for substrates with bulky side chain (up to 72 h). After completion of the reaction, the reaction mixture was cooled down, and poured into a 500 mL flask with ice inside. 22.5 mL conc. HCl (aq) was added slowly into the solution while shaking and there was ice inside the flask (until pH=2, diluted HCl (aq) can also be used but will introduce much water). Then the solvent was concentrated to 10 mL by a rotavapor (to remove ethanol). The aqueous solution was extracted by EtOAc (30 mL×3), and the organic phase was washed by conc. brine (30 mL×3) and dried by anhydrous Na₂SO₄. The dried organic phase was passed through a pad of silica gel, washed by 50 mL EtOAc. And solvent was removed by a rotavapor to afford Ls12d, which was used in the next step without purification.

Ls12d (2.2 mmol), with Ls12e (2 mmol), was applied to the procedure of preparation of L1, and the reaction mixture after two steps was purified by flash silica gel column chromatography (2% formic acid with 10% to 50% EtOAc/hexane) to give the L12 (62% over four steps) as a white solid.

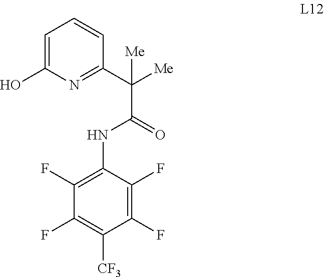 [SMILES: CC(C)(C(=O)Nc1c(F)c(F)c(C(F)(F)F)c(F)c1F)c1cccc(O)n1]

¹H NMR (600 MHz, CDCl₃) δ 9.57 (s, 1H), 7.51 (dd, J=9.0, 7.2 Hz, 1H), 6.49 (dd, J=7.2, 0.9 Hz, 1H), 6.46 (dd, J=9.0, 0.9 Hz, 1H), 1.83 (s, 6H). ¹³C NMR (151 MHz, Acetone-d₆) δ 173.62, 164.67, 155.53, 145.32 (dm, J=224.4 Hz), 143.65 (dm, J=230.3 Hz), 142.03, 123.31 (tm, J=14.3 Hz), 122.17 (q, J=273.3 Hz), 115.26, 108.19, 106.89 (m), 49.00, 25.83.

¹⁹F NMR (376 MHz, CDCl₃) δ −58.76, −143.77, −145.64.

HRMS (ESI-TOF) Calculated for C₁₆H₁₂F₇N₂O₂ [M+H]⁺: 397.0787, Found: 397.0779.

### Preparation of L10, L11, L13 to L15

Preparation of L10, L11, L13 to L15 followed the procedure of preparation of L12 with corresponding aniline and the reaction mixture was purified by flash silica gel column chromatography (2% formic acid with 10% to 50% EtOAc/hexane) to give L10, L11, L13 to L15 as a white solid.

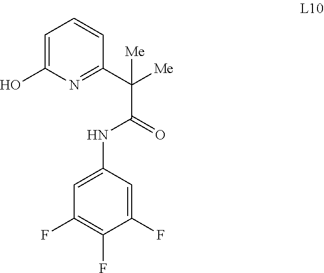 [SMILES: CC(C)(C(=O)Nc1cc(F)c(F)c(F)c1)c1cccc(O)n1]

¹H NMR (600 MHz, CDCl₃) δ 9.32 (s, 1H), 7.57 (dd, J=9.0, 7.2 Hz, 1H), 7.35 (dd, J=9.6, 6.2 Hz, 2H), 6.57 (dd, J=9.0, 0.9 Hz, 1H), 6.49 (dd, J=7.2, 1.0 Hz, 1H), 1.78 (s, 6H). ¹³C NMR (151 MHz, CDCl₃) δ 171.07, 165.37, 151.00 (dm, J=252.1 Hz), 150.83, 142.98, 136.48 (dm, J=241.6 Hz), 134.24 (m), 118.03, 106.00, 104.66 (dd, J=20.9, 5.5 Hz), 47.37, 24.59. ¹⁹F NMR (471 MHz, CDCl₃) δ −134.17, −166.00.

HRMS (ESI-TOF) Calculated for C₁₅H₁₄F₃N₂O₂[M+H]⁺: 311.1007, Found: 311.1007.

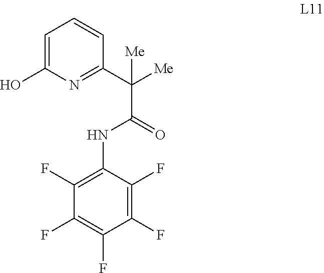 [SMILES: CC(C)(C(=O)Nc1c(F)c(F)c(F)c(F)c1F)c1cccc(O)n1]

¹H NMR (600 MHz, CDCl₃) δ 12.61 (s, 1H), 9.15 (s, 1H), 7.47 (dd, J=9.1, 7.2 Hz, 1H), 6.45 (dd, J=7.2, 1.0 Hz, 1H), 6.42 (dd, J=9.1, 0.9 Hz, 1H), 1.81 (s, 6H). ¹³C NMR (151 MHz, CDCl₃) δ 171.82, 165.34, 150.36, 143.28 (dm, J=245.9 Hz), 142.53, 140.18 (dm, J=257.1 Hz), 137.88 (dm, J=242.1 Hz), 118.26, 112.48 (m), 105.87, 47.03, 24.52. ¹⁹F NMR (471 MHz, CDCl₃) δ −145.30, −156.42, −162.61.

HRMS (ESI-TOF) Calculated for C₁₅H₁₂F₅N₂O₂[M+H]⁺: 347.0819, Found: 347.0817.

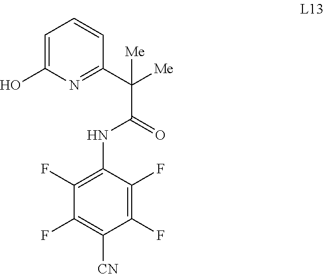 [SMILES: CC(C)(C(=O)Nc1c(F)c(F)c(C#N)c(F)c1F)c1cccc(O)n1]

¹H NMR (600 MHz, CDCl₃) δ 9.70 (s, 1H), 7.53 (dd, J=9.0, 7.2 Hz, 1H), 6.51 (dd, J=7.2, 0.9 Hz, 1H), 6.46 (dd, J=9.0, 0.9 Hz, 1H), 1.83 (s, 6H). ¹³C NMR (151 MHz, CDCl₃) δ 171.16, 165.33, 150.10, 147.42 (dm, J=261.6 Hz), 142.86, 142.16 (dm, J=239.8 Hz), 123.51 (m), 118.13, 107.43, 106.52, 91.54 (m), 47.32, 24.54. ¹⁹F NMR (471 MHz, CDCl₃) δ −133.07, −141.83.

HRMS (ESI-TOF) Calculated for C₁₆H₁₂F₄N₃O₂[M+H]⁺: 354.0866, Found: 354.0864.

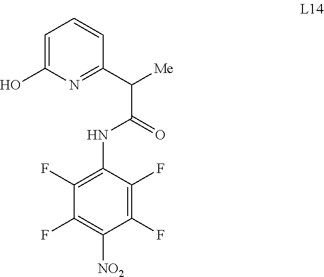 [SMILES: CC(C(=O)Nc1c(F)c(F)c([N+](=O)[O-])c(F)c1F)c1cccc(O)n1]

¹H NMR (600 MHz, CDCl₃) δ 9.68 (s, 1H), 7.54 (dd, J=9.0, 7.2 Hz, 1H), 6.51 (dd, J=7.3, 0.9 Hz, 1H), 6.47 (dd, J=9.0, 0.9 Hz, 1H), 1.83 (s, 6H). ¹³C NMR (151 MHz, CDCl₃) δ 171.21, 165.32, 150.10, 142.35 (dm, J=246.4 Hz), 142.88, 140.68 (dm, J=245.4 Hz), 128.54 (m), 121.53 (m), 118.15, 106.53, 47.29, 24.54. ¹⁹F NMR (376 MHz, CDCl₃) δ −143.96, −149.60.

HRMS (ESI-TOF) Calculated for C₁₅H₁₂F₄N₃O₄[M+H]⁺: 374.0764, Found: 374.0768.

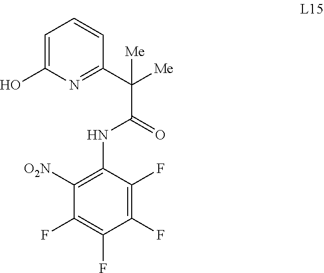 [SMILES: CC(C)(C(=O)Nc1c(F)c(F)c(F)c(F)c1[N+](=O)[O-])c1cccc(O)n1]

¹H NMR (600 MHz, CDCl₃) δ 7.50 (dd, J=9.1, 7.1 Hz, 1H), 6.50 (dd, J=9.0, 0.9 Hz, 1H), 6.44 (dd, J=7.2, 0.9 Hz, 1H), 1.77 (s, 6H). ¹³C NMR (151 MHz, CDCl₃) δ 171.76, 165.11, 149.94, 142.37, 145.00-137.72 (m, four carbons connected to F), 131.76, 118.16, 117.90 (m), 106.28, 47.15, 24.39. ¹⁹F NMR (471 MHz, CDCl₃) δ −140.31, −145.69, −147.93, −154.71.

HRMS (ESI-TOF) Calculated for C₁₅H₁₂F₄N₃O₄[M+H]⁺: 374.0764, Found: 374.0757.

### Preparation of L16

Preparation of L16 followed the procedure of preparation of L12 with 22 mmol (2.2 eq) KHMDS and acetonitrile (10 mmol). and the reaction mixture was purified by flash silica gel column chromatography (2% formic acid with 10% to 50% EtOAc/hexane) to give L16 as a white solid.

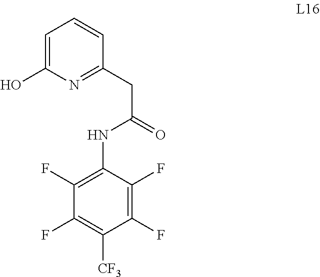 [SMILES: O=C(Cc1cccc(O)n1)Nc1c(F)c(F)c(C(F)(F)F)c(F)c1F]

¹H NMR (600 MHz, Acetone-d₆) δ 7.43 (dd, J=9.2, 6.8 Hz, 1H), 6.32 (d, J=8.9 Hz, 1H), 6.26 (d, J=6.8 Hz, 1H), 3.92 (s, 2H). ¹³C NMR (151 MHz, Acetone-d₆) δ 167.34, 164.12, 145.36 (dm, J=241.3 Hz), 143.75, 143.53 (dm, J=236.8 Hz), 141.82, 122.72 (m), 122.21 (q, J=272.5 Hz), 118.77, 107.24, 79.32, 40.67. ¹⁹F NMR (376 MHz, CDCl₃) δ −58.77, −143.46, −145.45.

HRMS (ESI-TOF) Calculated for C₁₄H₈F₇N₂O₂ [M+H]⁺: 369.0474, Found: 369.0469.

### Preparation of L17

Preparation of L17 followed the procedure of preparation of L12 with 22 mmol (2.2 eq) KHMDS and propiononitrile (10 mmol), and the reaction mixture was purified by flash silica gel column chromatography (2% formic acid with 10% to 50% EtOAc/hexane) to give L17 as a white solid.

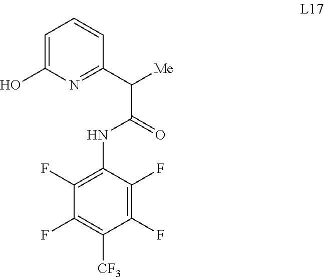 [SMILES: CC(C(=O)Nc1c(F)c(F)c(C(F)(F)F)c(F)c1F)c1cccc(O)n1]

¹H NMR (600 MHz, CDCl₃) δ 9.90 (s, 1H), 7.61 (dd, J=9.0, 7.1 Hz, 1H), 6.91-6.02 (m, 2H), 4.00 (q, J=7.0 Hz, 1H), 1.65 (d, J=7.0 Hz, 3H). ¹³C NMR (126 MHz, CDCl₃) δ 168.69, 165.79, 147.48, 143.86, 117.99, 106.71, 43.97, 14.61. (Signal of seven carbons on the aniline is too low to read due to poor solubility) ¹⁹F NMR (376 MHz, CDCl₃) δ −58.78, −143.63, −145.74.

HRMS (ESI-TOF) Calculated for C₁₅H₁₀F₇N₂O₂ [M+H]⁺: 383.0630, Found: 383.0625.

### Preparation of L18 to L24

Preparation of L18 to L24 followed the procedure of preparation of L12 with corresponding nitriles, and the reaction mixture was purified by flash silica gel column chromatography (2% formic acid with 10% to 50% EtOAc/hexane) to give L18 to L24 as a white solid. Nitriles were commercially available or prepared from commercially available ketones (1).

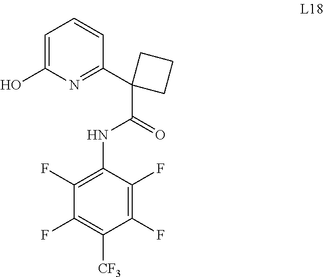 [SMILES: O=C(Nc1c(F)c(F)c(C(F)(F)F)c(F)c1F)C1(c2cccc(O)n2)CCC1]

¹H NMR (600 MHz, CDCl₃) δ 10.11 (s, 1H), 7.55 (dd, J=9.1, 7.1 Hz, 1H), 6.46 (dd, J=9.1, 0.9 Hz, 1H), 6.42 (dd, J=7.1, 1.0 Hz, 1H), 3.34-2.92 (m, 2H), 2.93-2.51 (m, 2H), 2.11 (m, 1H), 1.97 (m, 1H). ¹³C NMR (151 MHz, CDCl₃) δ 170.95, 165.80, 150.01, 143.22, 117.70, 106.97, 52.85, 31.99, 16.63. (Signal of seven carbons on the aniline is too low to read due to poor solubility) ¹⁹F NMR (376 MHz, CDCl₃) δ −58.76, −143.80, −145.92.

HRMS (ESI-TOF) Calculated for C₁₇H₁₂F₇N₂O₂[M+H]⁺: 409.0787, Found: 409.0782.

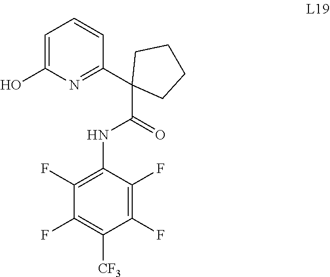 [SMILES: O=C(Nc1c(F)c(F)c(C(F)(F)F)c(F)c1F)C1(c2cccc(O)n2)CCCC1]

¹H NMR (600 MHz, CDCl₃) δ 10.07 (s, 1H), 7.52 (dd, J=9.0, 7.2 Hz, 1H), 6.47 (dd, J=4.4, 0.9 Hz, 1H), 6.46 (dd, J=6.3, 0.9 Hz, 1H), 3.11-2.94 (m, 2H), 2.14-1.99 (m, 2H), 1.90-1.75 (m, 4H). ¹³C NMR (151 MHz, CDCl₃) δ 170.40, 165.53, 149.45, 143.08, 117.97, 107.48, 59.94, 34.81, 23.16. (Signal of seven carbons on the aniline is too low to read due to poor solubility) ¹⁹F NMR (376 MHz, CDCl₃) δ −58.76, −143.90, −145.80.

HRMS (ESI-TOF) Calculated for C₁₈H₁₄F₇N₂O₂[M+H]⁺: 423.0944, Found: 423.0937.

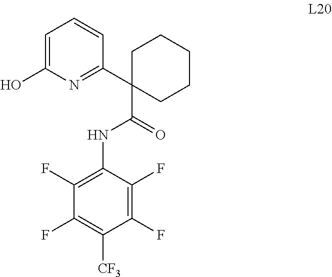 [SMILES: O=C(Nc1c(F)c(F)c(C(F)(F)F)c(F)c1F)C1(c2cccc(O)n2)CCCCC1]

¹H NMR (600 MHz, CDCl₃) δ 9.83 (s, 1H), 7.52 (dd, J=9.0, 7.5 Hz, 1H), 6.48 (d, J=7.2 Hz, 1H), 6.44 (d, J=9.0 Hz, 1H), 2.73 (m, 2H), 1.99 (m, 2H), 1.82-1.70 (m, 2H), 1.70-1.61 (m, 2H), 1.45-1.27 (m, 2H). ¹³C NMR (151 MHz, CDCl₃) δ 170.18, 165.52, 149.43, 142.90, 118.09, 106.90, 51.21, 32.47, 25.31, 23.12. (Signal of seven carbons on the aniline is too low to read due to poor solubility) ¹⁹F NMR (376 MHz, CDCl₃) δ −58.78, −143.85, −145.40.

HRMS (ESI-TOF) Calculated for C₁₉H₁₆F₇N₂O₂[M+H]⁺: 437.1100, Found: 437.1100.

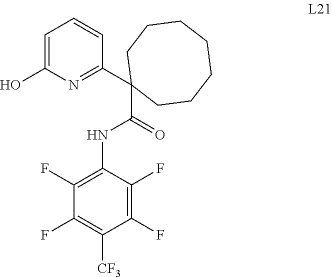 [SMILES: O=C(Nc1c(F)c(F)c(C(F)(F)F)c(F)c1F)C1(c2cccc(O)n2)CCCCCCC1]

¹H NMR (600 MHz, CDCl₃) δ 9.23 (s, 1H), 7.48 (dd, J=9.0, 7.2 Hz, 1H), 6.46 (d, J=6.9 Hz, 1H), 6.43 (d, J=9.1 Hz, 1H), 2.58 (dd, J=15.2, 8.4 Hz, 2H), 2.38 (dd, J=14.9, 8.0 Hz, 2H), 1.61 (d, J=54.1 Hz, 10H). ¹³C NMR (151 MHz, CDCl₃) δ 171.13, 165.06, 149.42, 142.30, 118.18, 107.33, 55.28, 28.95, 28.26, 25.07, 22.65. (Signal of seven carbons on the aniline is too low to read due to poor solubility) ¹⁹F NMR (376 MHz, CDCl₃) δ −58.76, −143.78, −145.46.

HRMS (ESI-TOF) Calculated for C₂₁H₂₀F₇N₂O₂[M+H]⁺: 465.1413, Found: 465.1416.

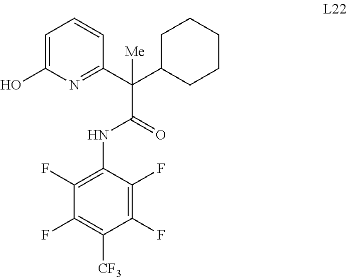 [SMILES: CC(C(=O)Nc1c(F)c(F)c(C(F)(F)F)c(F)c1F)(c1cccc(O)n1)C1CCCCC1]

¹H NMR (600 MHz, Acetone-d₆) δ 7.69 (dd, J=8.4, 7.4 Hz, 1H), 6.89 (d, J=7.5 Hz, 1H), 6.65 (d, J=8.4 Hz, 1H), 2.46 (tt, J=11.5, 3.1 Hz, 1H), 1.87-1.76 (m, 2H), 1.72-1.61 (m, 2H), 1.54 (s, 3H), 1.40-1.20 (m, 2H), 1.20-1.12 (m, 2H), 1.12-0.95 (m, 2H). ¹³C NMR (151 MHz, Acetone-d₆) δ 173.23, 163.69, 157.35, 145.27 (dm, J=244.3 Hz), 143.36 (dm, J=236.7 Hz), 141.70, 123.65 (m), 122.23 (q, J=272.8 Hz), 112.94, 111.37, 106.38 (m), 56.48, 48.69, 29.10, 28.05, 27.75, 27.61, 27.15, 14.61. ¹⁹F NMR (376 MHz, Acetone-d₆) δ −59.37, −146.72, −146.90.

HRMS (ESI-TOF) Calculated for C₂₁H₂₀F₇N₂O₂[M+H]⁺: 465.1413, Found: 465.1413.

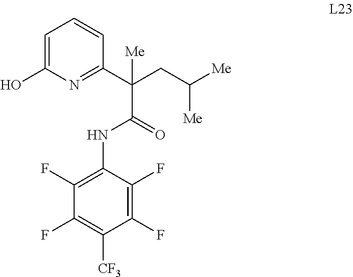 [SMILES: CC(C)CC(C)(C(=O)Nc1c(F)c(F)c(C(F)(F)F)c(F)c1F)c1cccc(O)n1]

¹H NMR (600 MHz, CDCl₃) δ 9.41 (s, 1H), 7.49 (dd, J=9.0, 7.2 Hz, 1H), 6.87-6.27 (m, 2H), 2.33 (dd, J=14.3, 6.1 Hz, 1H), 2.15 (dd, J=14.3, 6.1 Hz, 1H), 1.84-1.72 (m, 4H), 1.02 (d, J=6.7 Hz, 3H), 0.88 (d, J=6.6 Hz, 3H). ¹³C NMR (151 MHz, CDCl₃) δ 171.32, 164.94, 150.40, 142.47, 117.95, 106.62, 50.80, 44.68, 25.39, 24.10, 23.77, 20.46. (Signal of seven carbons on the aniline is too low to read due to poor solubility) ¹⁹F NMR (376 MHz, CDCl₃) δ −58.80, −143.78, −145.43.

HRMS (ESI-TOF) Calculated for C₁₉H₁₈F₇N₂O₂[M+H]⁺: 439.1257, Found: 439.1253.

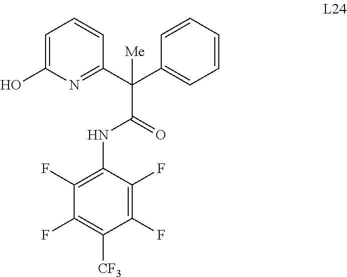 [SMILES: CC(C(=O)Nc1c(F)c(F)c(C(F)(F)F)c(F)c1F)(c1ccccc1)c1cccc(O)n1]

¹H NMR (600 MHz, Acetone-d₆) δ 7.68 (q, J=8.1 Hz, 1H), 7.47-7.35 (m, 2H), 7.36-7.28 (m, 1H), 7.23 (ddd, J=7.8, 3.9, 1.7 Hz, 2H), 6.86-6.68 (m, 1H), 6.58 (t, J=9.8 Hz, 1H), 2.00 (d, J=3.4 Hz, 3H). ¹³C NMR (151 MHz, Acetone-d₆) δ 172.20, 163.88, 156.28, 145.30, 145.29 (dm, J=258.2 Hz), 143.56 (dm, J=237.2 Hz), 141.91, 129.53, 128.34, 128.22, 123.24 (m), 122.19 (q, J=272.5 Hz), 113.43, 112.51, 106.74 (m), 58.51, 25.99. ¹⁹F NMR (376 MHz, Acetone-d₆) δ −59.41, −146.40, −146.78.

HRMS (ESI-TOF) Calculated for C₂₁H₁₄F₇N₂O₂[M+H]⁺: 459.0944, Found: 459.0942.

### Preparation of L25

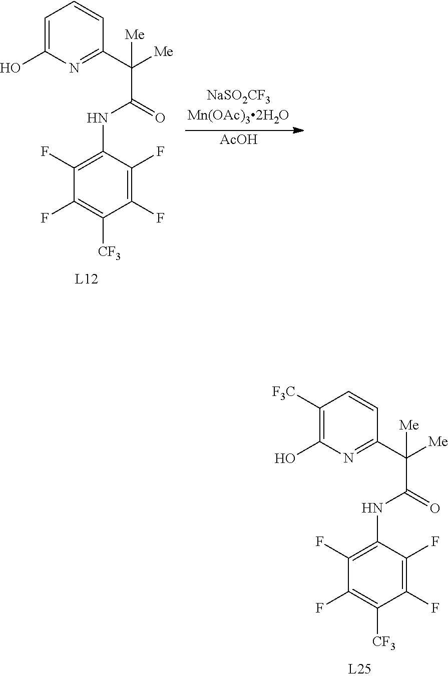 [SMILES: "AcOH" ; CC(C)(C(=O)Nc1c(F)c(F)c(C(F)(F)F)c(F)c1F)c1cccc(O)n1 ; CC(C)(C(=O)Nc1c(F)c(F)c(C(F)(F)F)c(F)c1F)c1ccc(C(F)(F)F)c(O)n1]

To a 12 mL reaction tube was added L12 (0.1 mmol) and sodium trifluoromethanesulfinate (0.3 mmol), followed by addition of 0.1 mL AcOH. Manganese(II) acetate dihydrate (0.3 mmol) was added in batches while the reaction mixture was stirred at room temperature, followed by addition of 0.1 mL AcOH. The reaction mixture was stirred at room temperature for 16 h. After completion, 0.2 mL H₂O was added to the reaction mixture, and the solution was extracted by EtOAc (0.2 mL×3). Then the mixture was purified by pTLC (2% formic acid with 20% EtOAc/hexane), to afford L25 (60%) as a white solid.

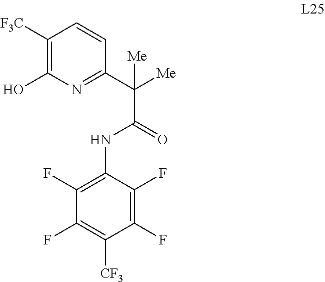 [SMILES: CC(C)(C(=O)Nc1c(F)c(F)c(C(F)(F)F)c(F)c1F)c1ccc(C(F)(F)F)c(O)n1]

¹H NMR (600 MHz, CDCl₃) δ 8.94 (s, 1H), 7.89 (d, J=7.6 Hz, 1H), 6.59 (d, J=7.6 Hz, 1H), 1.87 (s, 6H). ¹³C NMR (151 MHz, CDCl₃) δ 170.56, 161.81, 154.91, 144.35 (dm, J=263.8 Hz), 143.02 (dm, J=250.8 Hz), 141.55, 139.78, 138.29, 122.49 (q, J=271.0 Hz), 120.49 (m), 118.78, 105.03, 47.53, 24.20. (Some multiplicity was not assigned due to low signal from poor solubility) ¹⁹F NMR (376 MHz, CD₃OD) δ −60.32, −69.27, −146.96, −147.43.

HRMS (ESI-TOF) Calculated for C₁₇H₁₁F₁₀N₂O₂[M+H]⁺: 465.0661, Found: 465.0657.

### Preparation of L26 and L27

Preparation of L26 and L27 followed the procedure of preparation of L6 to L8 with L12, and the reaction mixture was purified by pTLC (2% formic acid with 20% EtOAc/hexane) to give L26 and L27 as white solids.

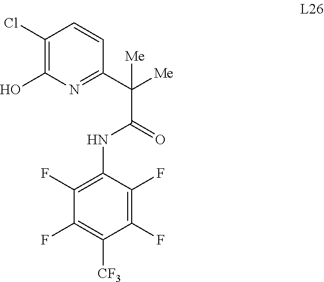 [SMILES: CC(C)(C(=O)Nc1c(F)c(F)c(C(F)(F)F)c(F)c1F)c1ccc(Cl)c(O)n1]

¹H NMR (600 MHz, CDCl₃) δ 9.25 (s, 1H), 7.65 (d, J=7.7 Hz, 1H), 6.47 (d, J=7.8 Hz, 1H), 1.85 (s, 6H). ¹³C NMR (151 MHz, CDCl₃) δ 171.00, 161.27, 148.81, 140.53, 124.43, 106.24, 46.52, 24.55. (Signal of seven carbons on the aniline is too low to read due to poor solubility) ¹⁹F NMR (376 MHz, CD₃OD) δ −60.32, −147.00, −147.44.

HRMS (ESI-TOF) Calculated for C₁₆H₁₁ClF₇N₂O₂ [M+H]⁺: 431.0397, Found: 431.0398.

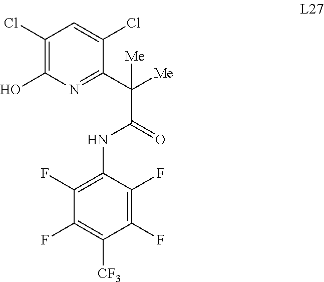 [SMILES: CC(C)(C(=O)Nc1c(F)c(F)c(C(F)(F)F)c(F)c1F)c1nc(O)c(Cl)cc1Cl]

¹H NMR (600 MHz, Acetone-d₆) δ 7.84 (s, 1H), 1.66 (s, 6H). ¹³C NMR (151 MHz, Acetone-d₆) δ 173.86, 158.93, 154.23, 145.25 (dm, J=259.2 Hz), 144.04 (dm, J=254.2 Hz), 141.37, 123.27 (m), 121.28, 122.19 (q, J=273.6 Hz), 117.32, 106.88 (m), 50.55, 25.52. ¹⁹F NMR (376 MHz, CD₃OD) δ −60.30, −147.19, −147.22.

HRMS (ESI-TOF) Calculated for C₁₆H₁₀Cl₂F₇N₂O₂[M+H]⁺: 465.0008, Found: 465.0005.

### Condition Screenings for the [2+2] Annulation Reaction

TABLE S1
Ligands screening for [2 + 2] annulation of cyclic aliphatic acid*

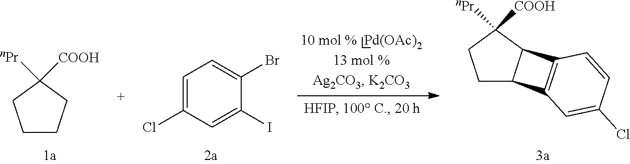

| 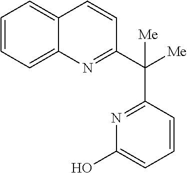 [SMILES: CC(C)(c1cccc(O)n1)c1ccc2ccccc2n1] | L28 |
|---|---|
| 4% | |
| 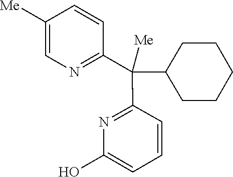 [SMILES: Cc1ccc(C(C)(c2cccc(O)n2)C2CCCCC2)nc1] | L29 |
| 20% | |
| 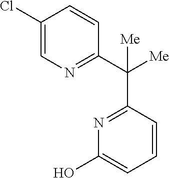 [SMILES: CC(C)(c1ccc(Cl)cn1)c1cccc(O)n1] | L30 |
| 7% | |
| 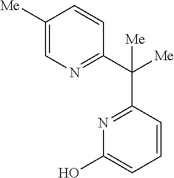 [SMILES: Cc1ccc(C(C)(C)c2cccc(O)n2)nc1] | L31 |
| 7% | |
| 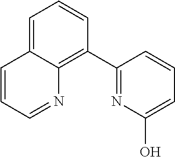 [SMILES: Oc1cccc(-c2cccc3cccnc23)n1] | L32 |
| 8% | |
| 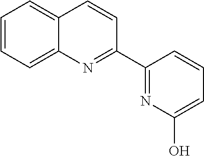 [SMILES: Oc1cccc(-c2ccc3ccccc3n2)n1] | L33 |
| 12% | |
| 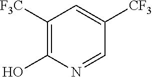 [SMILES: Oc1ncc(C(F)(F)F)cc1C(F)(F)F] | L34 |
| 10% | |
| 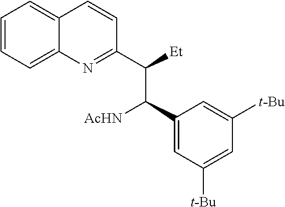 [SMILES: CC[C@H](c1ccc2ccccc2n1)[C@H](NC(C)=O)c1cc(C(C)(C)C)cc(C(C)(C)C)c1] | L35 |
| 17% | |
| 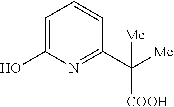 [SMILES: CC(C)(C(=O)O)c1cccc(O)n1] | L36 |
| 17% | |
| 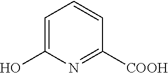 | L37 |
| 22% | |
| 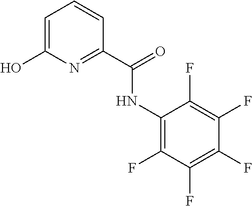 [SMILES: O=C(Nc1c(F)c(F)c(F)c(F)c1F)c1cccc(O)n1] | L1 |
| 38% | |
| 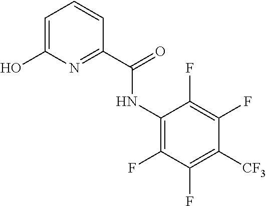 [SMILES: O=C(Nc1c(F)c(F)c(C(F)(F)F)c(F)c1F)c1cccc(O)n1] | L2 |
| 44% | |
| 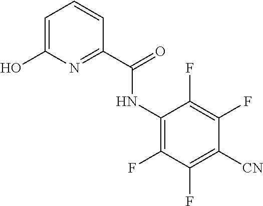 [SMILES: N#Cc1c(F)c(F)c(NC(=O)c2cccc(O)n2)c(F)c1F] | L3 |
| 57% | |
| 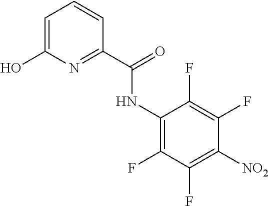 [SMILES: O=C(Nc1c(F)c(F)c([N+](=O)[O-])c(F)c1F)c1cccc(O)n1] | L4 |
| 67% | |
| 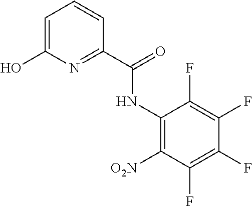 [SMILES: O=C(Nc1c(F)c(F)c(F)c(F)c1[N+](=O)[O-])c1cccc(O)n1] | L5 |
| 60% | |
| 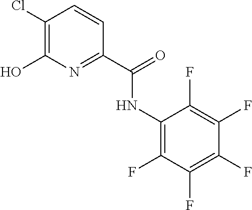 [SMILES: O=C(Nc1c(F)c(F)c(F)c(F)c1F)c1ccc(Cl)c(O)n1] | L6 |
| 14% | |
| 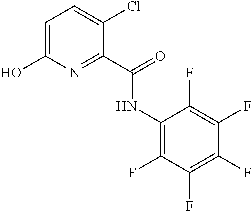 [SMILES: O=C(Nc1c(F)c(F)c(F)c(F)c1F)c1nc(O)ccc1Cl] | L7 |
| 62% | |
| 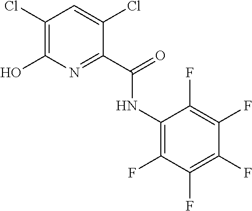 [SMILES: O=C(Nc1c(F)c(F)c(F)c(F)c1F)c1nc(O)c(Cl)cc1Cl] | L8 |
| 67% | |
| 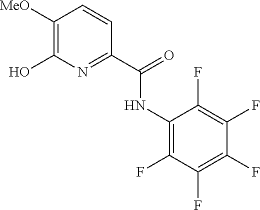 [SMILES: COc1ccc(C(=O)Nc2c(F)c(F)c(F)c(F)c2F)nc1O] | L9 |
| 50% | |
| 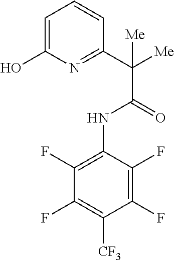 [SMILES: CC(C)(C(=O)Nc1c(F)c(F)c(C(F)(F)F)c(F)c1F)c1cccc(O)n1] | L12 |
| 22% | |
| No Ligand | |
| 7% | |

*Reaction conditions: 1a (0.1 mmol), 2a (0.2 mmol), Pd(OAc) 2 (10 mol %), L (13 mol %), K₂CO₃ (2.5 equiv), Ag₂CO₃ (2.0 equiv), HFIP (1.0 mL), at 100° C. for 20 hours. The yields were determined by ¹H NMR analysis of the crude product using CH₂Br₂ as the internal standard.

TABLE S2
Palladium sources and loading screening for cyclic aliphatic acid*

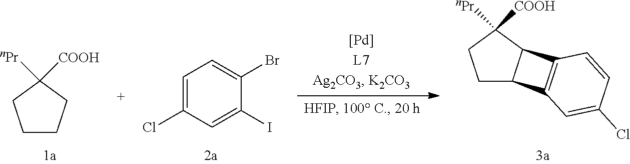

| entry | [Pd] | loading | Time (h) | yield |
|---|---|---|---|---|
| 1 | Pd(OAc)₂ | 10 mol % | 20 | 82% |
| 2 | Pd(CH₃CN)₂Cl₂ | 10 mol % | 20 | 84% |
| 3 | Pd(TFA)₂ | 10 mol % | 20 | 86% |
| 4 | Pda(dba)₃ | 5 mol % | 20 | 60% |
| 5 | Pd(OPiv)₂ | 10 mol % | 20 | 86% |
| 6 | Pd(CH₃CN)₄(BF₄)₂ | 10 mol % | 20 | 90% (90%*) |
| 7 | Pd(CH₃CN)₄(BF₄)₂ | 5 mol % | 40 | 84% |
| 8 | Pd(CH₃CN)₄(BF₄)₂ | 1 mol % | 56 | 61% |

*Reaction conditions: 1a (0.1 mmol), 2a (0.2 mmol), L7 (1.3 equiv to [Pd] loading), K₂CO₃ (2.5 equiv), Ag₂CO₃ (2.0 equiv), HFIP (1.0 mL), at 100° C. The yields were determined by ¹H NMR analysis of the crude product using CH₂Br₂ as the internal standard.
ªIsolated yield.

TABLE S3
Silver salts screening for cyclic aliphatic acid*

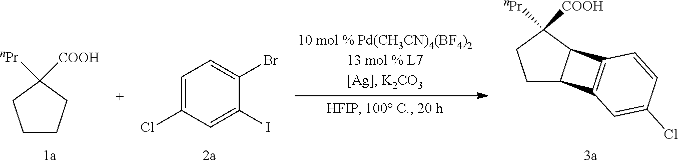

| entry | [Ag] | yield |
|---|---|---|
| 1 | Ag₂CO₃ | 90% |
| 2 | AgOAc | 59% |
| 3 | Ag₃PO₄ | 65% |
| 4 | Ag₂O | 63% |
| 5 | AgF | 77% |

*Reaction conditions: 1a (0.1 mmol), 2a (0.2 mmol), Pd(CH₃CN)₄(BF₄)₂ (10 mol %), L7 (13 mol %), K₂CO₃ (2.5 equiv), HFIP (1.0 mL), at 100° C. for 20 hours. The yields were determined by ¹H NMR analysis of the crude product using CH₂Br₂ as the internal standard.

TABLE S4
Bases screening for cyclic aliphatic acid*

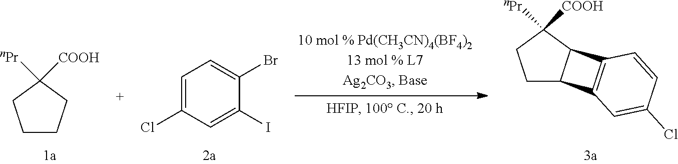

| entry | Base | yield |
|---|---|---|
| 1 | K₂CO₃ | 90% |
| 2 | KHCO₃ | 81% |
| 3 | KH₂PO₄ | 32% |
| 4 | K₃PO₄ | 65% |
| 5 | Na₃CO₃ | 67% |
| 6 | NaHCO₃ | 55% |
| 7 | NaOAc | 39% |
| 8 | Cs₂CO₃ | 80% |
| 9 | Li₂CO₃ | 23% |

*Reaction conditions: 1a (0.1 mmol), 2a (0.2 mmol), Pd(CH₃CN)₄(BF₄)₂ (10 mol %), L7 (13 mol %), Ag₂CO₃ (2.0 equiv), Base (2.5 equiv), HFIP (1.0 mL), at 100° C. for 20 hours. The yields were determined by ¹H NMR analysis of the crude product using CH₂Br₂ as the internal standard.

TABLE S5
Temperature screening for cyclic aliphatic acid*

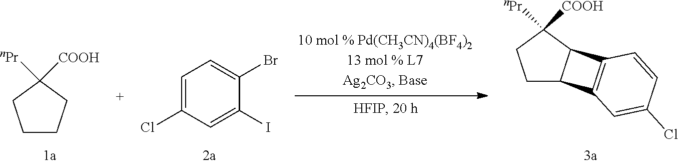

| entry | Temperature | yield |
|---|---|---|
| 1 | 100° C. | 90% |
| 2 | 90° C. | 84% |
| 3 | 80° C. | 77% |

*Reaction conditions: 1a (0.1 mmol), 2a (0.2 mmol), Pd(CH₃CN)₄(BF₄)₂ (10 mol %), L7 (13 mol %), K₂CO₃ (2.5 equiv), Ag₂CO₃ (2.0 equiv), HFIP (1.0 mL), for 20 hours. The yields were determined by ¹H NMR analysis of the crude product using CH₂Br₂ as the internal standard.

TABLE S6
Ligands screening for [2 + 2] annulation of acyclic aliphatic acid*

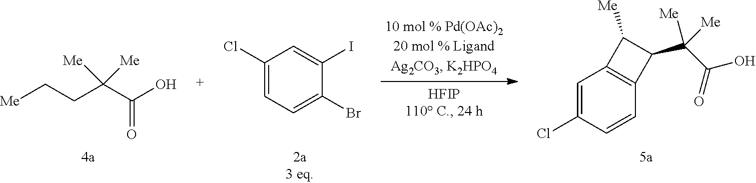 [SMILES: CCCC(C)(C)C(=O)O ; Clc1ccc(Br)c(I)c1 ; C[C@H]1c2cc(Cl)ccc2[C@@H]1C(C)(C)C(=O)O]

| | |
|---|---|
| 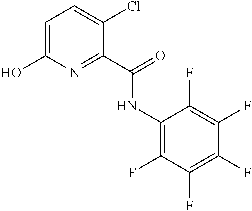 [SMILES: O=C(Nc1c(F)c(F)c(F)c(F)c1F)c1nc(O)ccc1Cl] | L7 |
| 2% | |
| 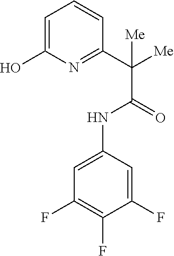 [SMILES: CC(C)(C(=O)Nc1cc(F)c(F)c(F)c1)c1cccc(O)n1] | L10 |
| 18% | |
| 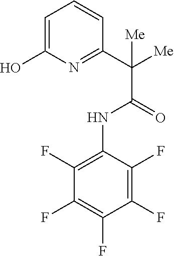 [SMILES: CC(C)(C(=O)Nc1c(F)c(F)c(F)c(F)c1F)c1cccc(O)n1] | L11 |
| 50% | |
| 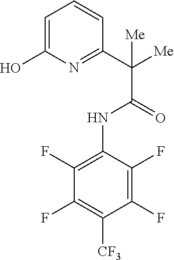 [SMILES: CC(C)(C(=O)Nc1c(F)c(F)c(C(F)(F)F)c(F)c1F)c1cccc(O)n1] | L12 |
| 68% | |
| 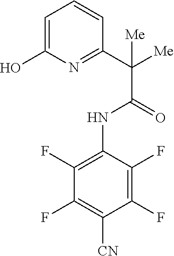 [SMILES: CC(C)(C(=O)Nc1c(F)c(F)c(C#N)c(F)c1F)c1cccc(O)n1] | L13 |
| 43% | |
| 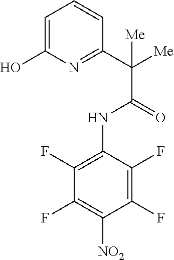 [SMILES: CC(C)(C(=O)Nc1c(F)c(F)c([N+](=O)[O-])c(F)c1F)c1cccc(O)n1] | L14 |
| 47% | |
| 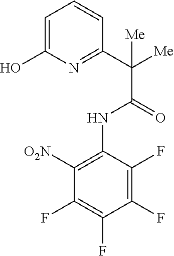 [SMILES: CC(C)(C(=O)Nc1c(F)c(F)c(F)c(F)c1[N+](=O)[O-])c1cccc(O)n1] | L15 |
| 34% | |
| 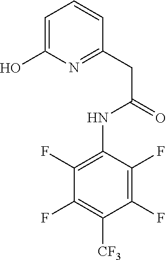 [SMILES: O=C(Cc1cccc(O)n1)Nc1c(F)c(F)c(C(F)(F)F)c(F)c1F] | L16 |
| 14% | |
| 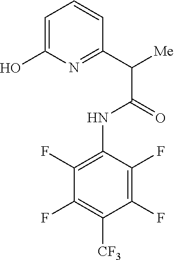 [SMILES: CC(C(=O)Nc1c(F)c(F)c(C(F)(F)F)c(F)c1F)c1cccc(O)n1] | L17 |
| 19% | |
| 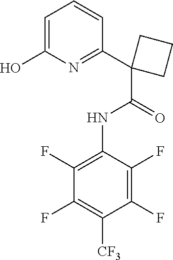 [SMILES: O=C(Nc1c(F)c(F)c(C(F)(F)F)c(F)c1F)C1(c2cccc(O)n2)CCC1] | L18 |
| 42% | |
| 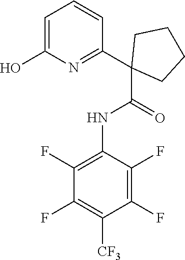 [SMILES: O=C(Nc1c(F)c(F)c(C(F)(F)F)c(F)c1F)C1(c2cccc(O)n2)CCCC1] | L19 |
| 49% | |
| 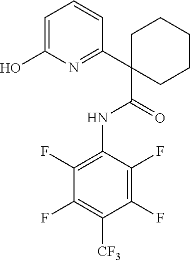 [SMILES: O=C(Nc1c(F)c(F)c(C(F)(F)F)c(F)c1F)C1(c2cccc(O)n2)CCCCC1] | L20 |
| 50% | |
| 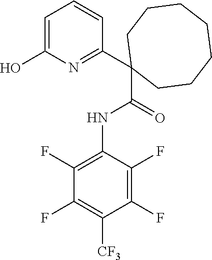 [SMILES: O=C(Nc1c(F)c(F)c(C(F)(F)F)c(F)c1F)C1(c2cccc(O)n2)CCCCCCC1] | L21 |
| 47% | |
| 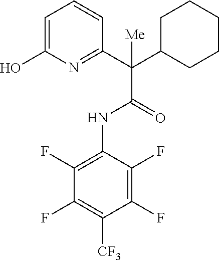 [SMILES: CC(C(=O)Nc1c(F)c(F)c(C(F)(F)F)c(F)c1F)(c1cccc(O)n1)C1CCCCC1] | L22 |
| 43% | |
| 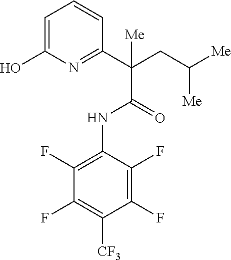 [SMILES: CC(C)CC(C)(C(=O)Nc1c(F)c(F)c(C(F)(F)F)c(F)c1F)c1cccc(O)n1] | L23 |
| 53% | |
| 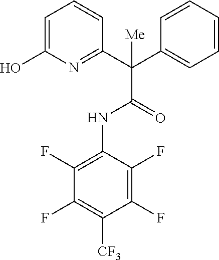 [SMILES: CC(C(=O)Nc1c(F)c(F)c(C(F)(F)F)c(F)c1F)(c1ccccc1)c1cccc(O)n1] | L24 |
| 37% | |
| 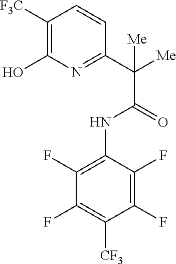 [SMILES: CC(C)(C(=O)Nc1c(F)c(F)c(C(F)(F)F)c(F)c1F)c1ccc(C(F)(F)F)c(O)n1] | L25 |
| 18% | |
| 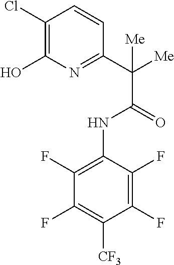 [SMILES: CC(C)(C(=O)Nc1c(F)c(F)c(C(F)(F)F)c(F)c1F)c1ccc(Cl)c(O)n1] | L26 |
| 39% | |
| 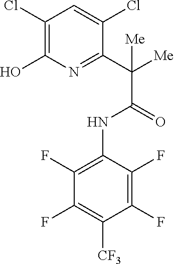 [SMILES: CC(C)(C(=O)Nc1c(F)c(F)c(C(F)(F)F)c(F)c1F)c1nc(O)c(Cl)cc1Cl] | L27 |
| 5% | |
| 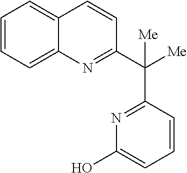 [SMILES: CC(C)(c1cccc(O)n1)c1ccc2ccccc2n1] | L28 |
| 36% | |
| 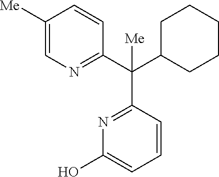 [SMILES: Cc1ccc(C(C)(c2cccc(O)n2)C2CCCCC2)nc1] | L29 |
| 2% | |
| 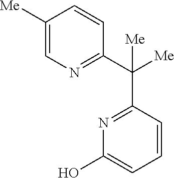 [SMILES: Cc1ccc(C(C)(C)c2cccc(O)n2)nc1] | L31 |
| 2% | |
| 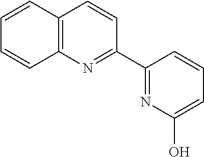 [SMILES: Oc1cccc(-c2ccc3ccccc3n2)n1] | L33 |
| 14% | |
| 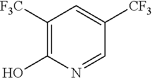 [SMILES: Oc1ncc(C(F)(F)F)cc1C(F)(F)F] | L34 |
| N.D. | |
| 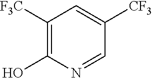 [SMILES: Oc1ncc(C(F)(F)F)cc1C(F)(F)F] | L36 |
| 2% | |
| 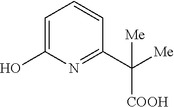 [SMILES: CC(C)(C(=O)O)c1cccc(O)n1] | L38 |
| 10% | |
| 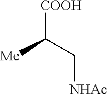 | L39 |
| 4% | |
| 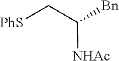 | L40 |
| 4% | |
| No Ligand | |
| N.D. | |

*Reaction conditions: aliphatic acid (0.1 mmol), 2a (0.3 mmol), Pd(OAc)₂ (10 mol %), L (20 mol %), K₂HPO₄ (4.0 equiv), Ag₂CO₃ (2.0 equiv), HFIP (1.2 ml), at 110° C. for 24 hours. The yields were determined by ¹H NMR analysis of the crude product using CH₂Br₂ as the internal standard.

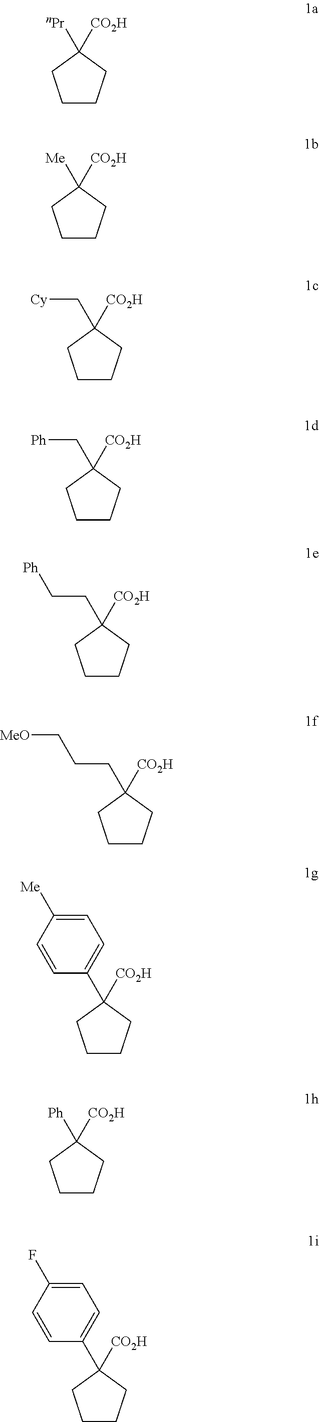

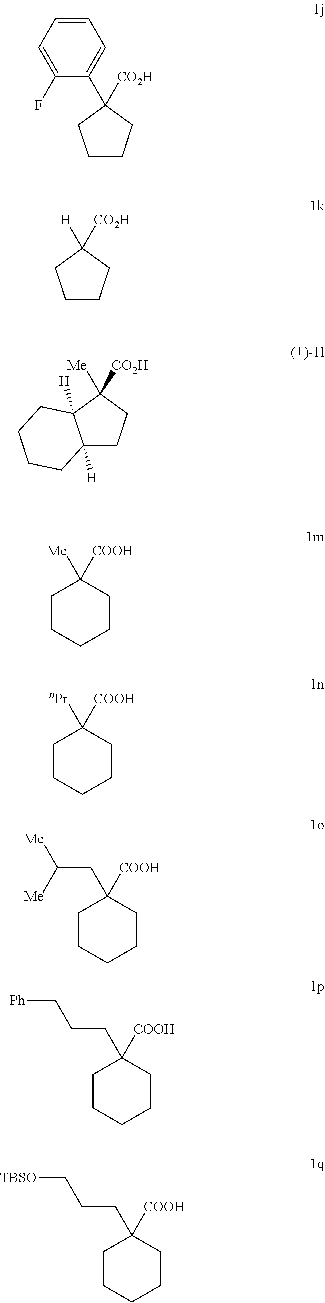

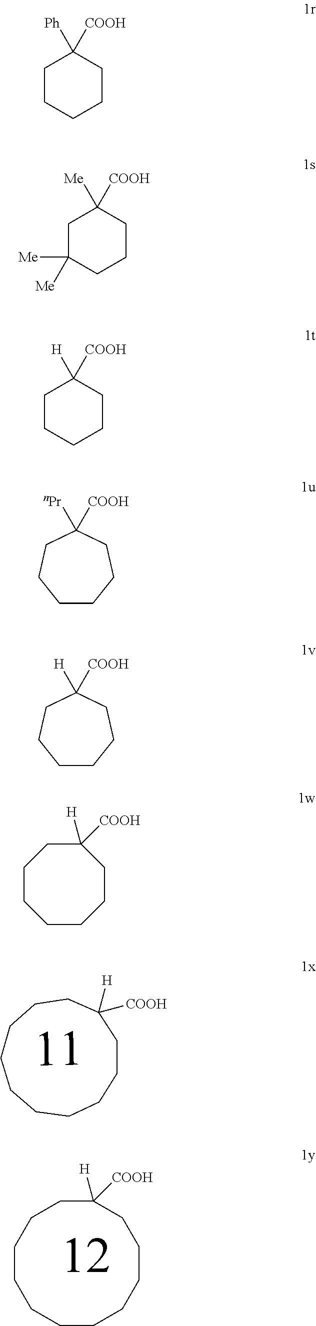

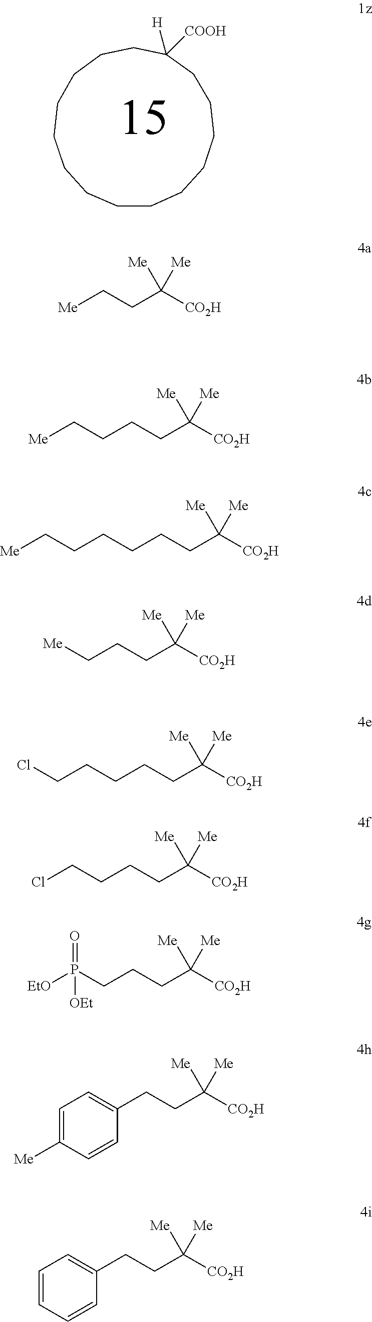

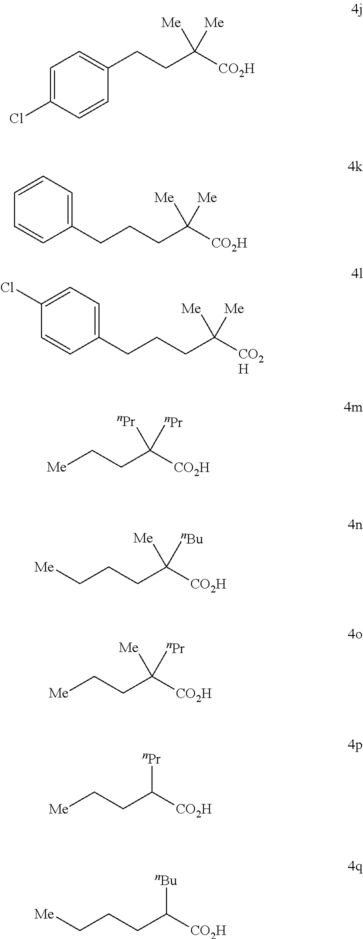

### Preparation for the Substrates

1b, 1g-k, 1m, 1r-t, 1v, 1x-z, 4a-c, 4m-q are commercially available. 1z was prepared according to reported procedure (2). Other aliphatic acids were prepared by the following procedure.

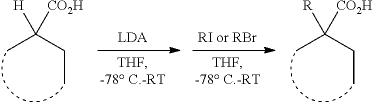

LDA (55 mmol) was added dropwise to a mixture of α-H-aliphatic acid (22 mmol) and THE (50 mL) at −78° C., and the mixture was allowed to warm up to room temperature and stirred for 1 h. Then the reaction mixture was recooled to −78° C. and RI or RBr (22 mmol) was added dropwise. The resulting solution was allowed to warm up to room temperature and stirred overnight. After the reaction finished, the reaction mixture was quenched with 10% HCl solution and extracted with EtOAc. The combined organic extract was washed with brine, dried over anhydrous MgSO₄, and concentrated under reduced pressure to give the crude product. Then purified by chromatography on silica gel to afford the desired aliphatic acid.

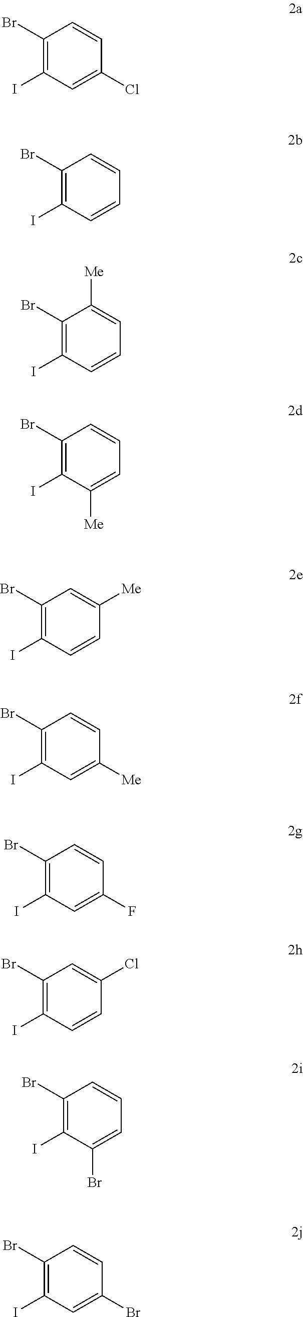

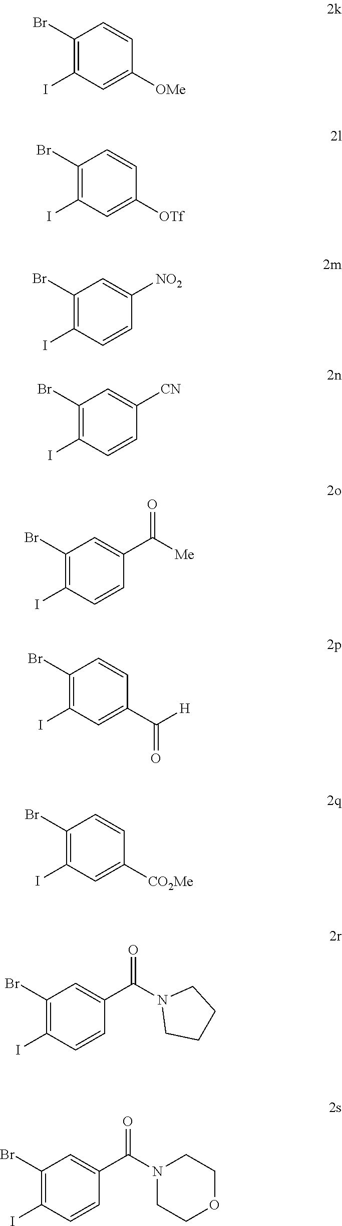

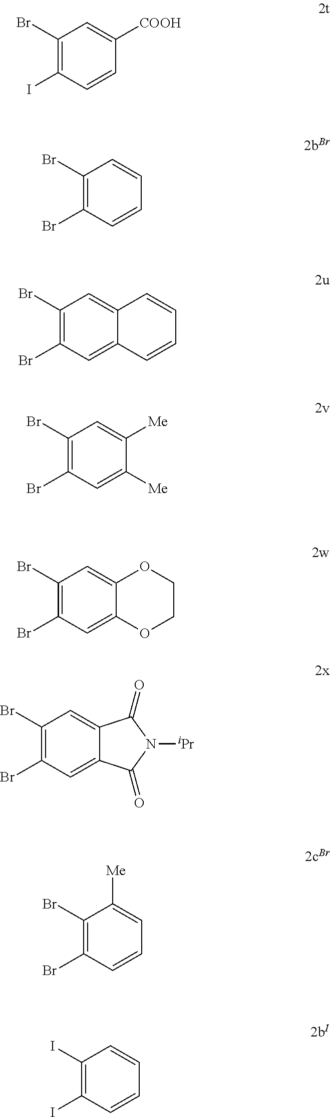

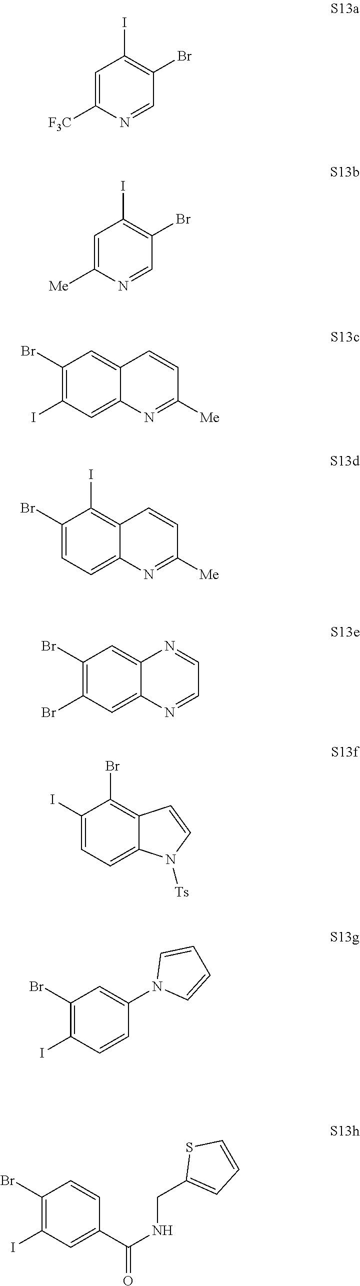

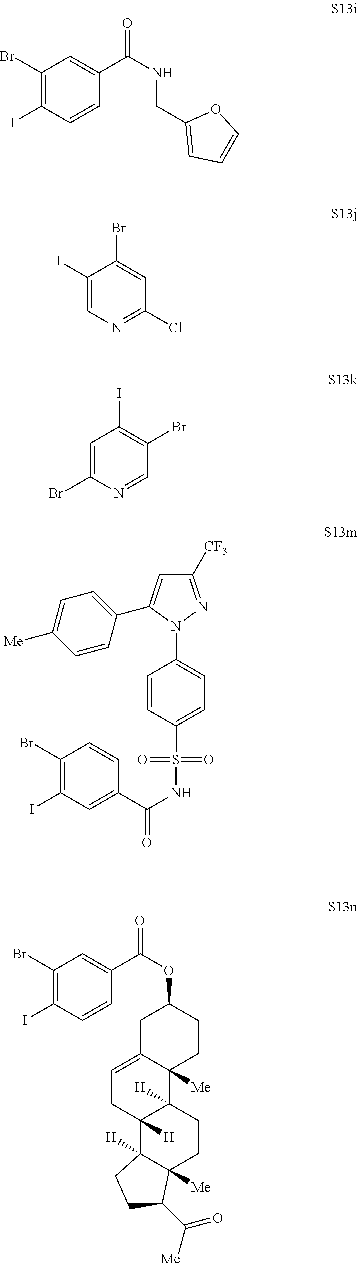

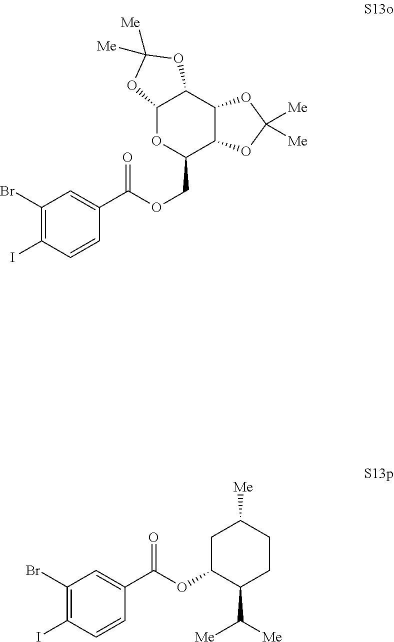 [SMILES: CC1(C)O[C@H]2O[C@H](COC(=O)c3ccc(I)c(Br)c3)[C@H]3OC(C)(C)O[C@H]3[C@H]2O1 ; CC(C)[C@@H]1CC[C@@H](C)C[C@H]1OC(=O)c1ccc(I)c(Br)c1]

2a-q, 2t-w, 2c^{Br}, 2b^{I}, S3b, S13j-k are commercially available. 2x (3), S13a (4), S13c (5), S13d (5), S13e (6) were prepared according to reported procedure.

### Preparation of 2r

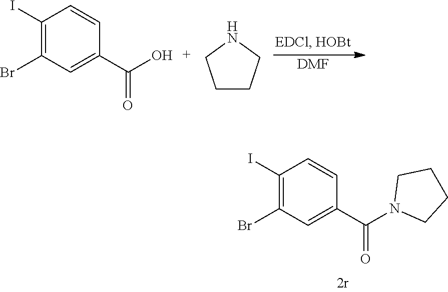

To a solution of 3-bromo-4-iodobenzoic acid (3 mmol, 981 mg) in DMF (10 mL) was added N-(3-(((ethylimino)methylene)amino)propylidene)-N-methylmethanaminium chloride (EDCI, 4.5 mmol, 862 mg) and HOBt (4.5 mmol, 608 mg), then the reaction stirred at room temperature for 10 mins. Thereafter, pyrrolidine (4.5 mmol, 320 mg) was added, and the mixture stirred at room temperature for 18 h. The mixture was quenched with saturated NaHCO₃ (10 mL), extracted with EtOAc (3×10 mL), washed with brine, and dried over Na₂SO₄. After filtration, the filtrate was concentrated under reduced pressure. The residue was purified by flash chromatography on silica gel using EtOAc/hexane to afford the expected product 2r (85% yield, 2.55 mmol, 969 mg) as white solid. ¹H NMR (600 MHz, CDCl₃) δ 7.89 (d, J=8.1 Hz, 1H), 7.77 (d, J=1.9 Hz, 1H), 7.14 (dd, J=8.1, 2.0 Hz, 1H), 3.62 (t, J=7.0 Hz, 2H), 3.41 (t, J=6.7 Hz, 2H), 2.00-1.86 (m, 4H). ¹³C NMR (151 MHz, CDCl₃) δ 167.36, 140.36, 138.68, 131.39, 130.00, 127.06, 103.18, 49.71, 46.52, 26.54, 24.53.

HRMS (ESI-TOF) Calculated for C₁₁H₁₂BrClNO [M+H]⁺: 379.9147, Found: 379.9148.

2s (white solid, 82% yield, 2.46 mmol, 974 mg) is synthesized with the same preparation procedure of 2r by using morpholine instead of pyrrolidine. ¹H NMR (600 MHz, CDCl₃) δ 7.92 (d, J=8.1 Hz, 1H), 7.67 (d, J=1.9 Hz, 1H), 7.03 (dd, J=8.1, 1.9 Hz, 1H), 3.91-3.32 (brm, 8H). ¹³C NMR (151 MHz, CDCl₃) δ 168.02, 140.57, 136.78, 131.26, 130.37, 126.72, 103.31, 66.85, 48.26, 42.73.

HRMS (ESI-TOF) Calculated for C₁₁H₁₂BrClNO₂ [M+H]⁺: 395.9096, Found: 395.9095.

### Preparation of S13f

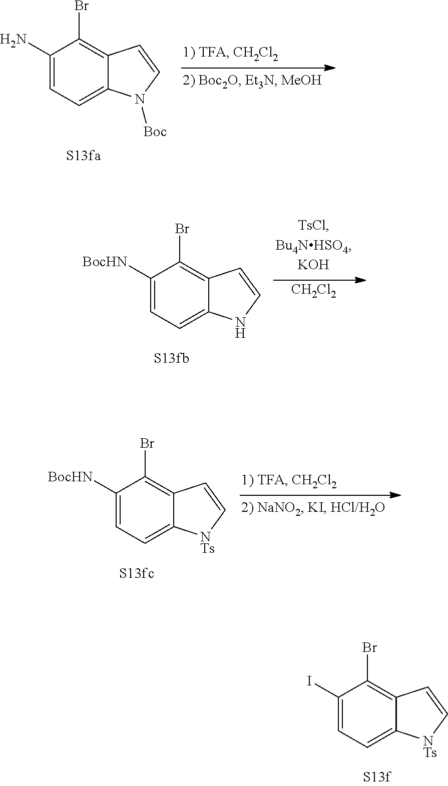

To a stirred solution of S13fa (3.11 g, 10 mmol) in CH₂Cl₂ (50 mL) was added TFA (1.46 g, 15 mmol) and the reaction was stirred at room temperature for 2 h. The reaction mixture was diluted with CH₂Cl₂ and washed with saturated NaHCO₃ solution, water and brine solution.

The separated CH₂Cl₂ layer was dried over anhydrous Na₂SO₄ and concentrated under reduced pressure to afford the crude product for next step without further purification.

After above crude product dissolved in MeOH (50 mL), triethylamine (1.52 g, 15 mmol) and Boc₂O (2.62 g, 12 mmol) were added and the mixture solution was stirred at room temperature for 6 h. The reaction solution was concentrated under reduced pressure, and the residue was partitioned with ethyl acetate (100 mL) and water (50 mL), and the organic layer was washed with a saturated sodium chloride solution. The organic layer was dried and then concentrated under reduced pressure. The residue was purified by a silica gel column to obtain S13fb (93% yield for two steps, 2.89 g, 9.3 mmol) as a white solid. ¹H NMR (600 MHz, CDCl₃) δ 8.30 (s, 1H), 7.86 (s, 1H), 7.30 (d, J=8.8 Hz, 1H), 7.22 (t, J=2.9 Hz, 1H), 6.85 (s, 1H), 6.54 (td, J=2.2, 1.1 Hz, 1H), 1.54 (s, 9H). ¹³C NMR (151 MHz, CDCl₃) δ 153.54, 132.58, 129.18, 128.83, 125.45, 110.61, 103.30, 80.67, 28.53. HRMS (ESI-TOF) Calculated for C₁₃H₁₅BrN₂O₂Na [M+Na]⁺: 333.0215, Found: 333.0223.

Tetra butyl ammonium hydrogen sulfate (68 mg, 0.2 mmol) was added to the solution of S13fb (622 mg, 2 mmol) in CH₂Cl₂ (20 mL) and the reaction mass was stirred for 5 mins at room temperature. Then, potassium hydroxide powder (336 mg, 6 mmol) added to the clear solution and stirred 0° C. for 10 mins. After that, TsCl (572 mg, 3 mmol) was added, and the reaction mass stirred for reaction completion at room temperature overnight. After the completion of the reaction, the mass was filtered out and washed with 20 mL of CH₂Cl₂. Further, the filtered content dried over anhydrous MgSO₄ and concentrated under reduced pressure. Then the residue was purified by flash chromatography on silica gel using EtOAc/hexane to obtain S13fc (90% yield, 838 mg, 1.8 mmol) as a white solid. ¹H NMR (600 MHz, CDCl₃) δ 8.05 (d, J=8.8 Hz, 1H), 7.89 (d, J=9.1 Hz, 1H), 7.72 (d, J=8.5 Hz, 2H), 7.57 (d, J=3.7 Hz, 1H), 7.21 (d, J=8.7 Hz, 2H), 6.89 (s, 1H), 6.65 (d, J=4.4 Hz, 1H), 2.34 (s, 3H), 1.53 (s, 9H). ¹³C NMR (151 MHz, CDCl₃) δ 152.99, 145.41, 135.02, 132.37, 131.70, 130.95, 127.57, 126.96, 126.95, 118.14, 113.16, 109.20, 104.97, 81.20, 28.43, 21.70. HRMS (ESI-TOF) Calculated for C₁₅H₁₄BrN₂O₂S [M-Boc+2H]⁺: 364.9959, Found: 364.9960.

To a stirred solution of S13fc (465 mg, 1 mmol) in CH₂Cl₂ (5 mL) was added TFA (146 mg, 1.5 mmol) and the reaction was stirred at room temperature for 2 h. The reaction mixture was diluted with CH₂Cl₂ and washed with saturated NaHCO₃ solution, water and brine solution.

The separated CH₂Cl₂ layer was dried over anhydrous Na₂SO₄ and concentrated under reduced pressure to afford the crude product for next step without further purification.

After above crude product dissolved in in conc. HCl (2 mL) at −5° C., NaNO₂ (86 mg, 1.25 mmol) in H₂O (2 mL) was added dropwise. The resulting mixture was stirred at same temperature for 30 mins. Then a solution of KI (332 mg, 2 mmol) in H₂O (2 mL) was added dropwise. The temperature was raised to room temperature slowly and the reaction mixture was stirred for 2 h. Upon completion of the reaction (monitored by TLC), 5% Na₂S₂O₃ solution was added. The reaction mixture was then extracted with CH₂Cl₂. The organic phase was dried over Na₂SO₄, the crude product was purified by silica gel column chromatography using EtOAc/hexane to provide S13f (60% yield for two steps, 286 mg, 0.6 mmol) as a white solid. ¹H NMR (600 MHz, CDCl₃) δ 7.76-7.67 (m, 4H), 7.56 (d, J=3.8 Hz, 1H), 7.24 (d, J=8.7 Hz, 2H), 6.71 (d, J=3.7 Hz, 1H), 2.36 (s, 3H). ¹³C NMR (151 MHz, CDCl₃) δ 145.73, 135.32, 134.94, 134.30, 130.25, 127.63, 126.99, 122.49, 114.23, 110.47, 95.13, 21.76. HRMS (ESI-TOF) Calculated for C₁₅H₁₂BrClNO₂S [M+H]⁺: 475.8817, Found: 475.8811.

### Preparation of S13g

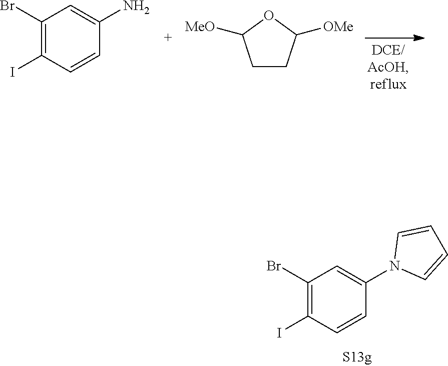 [SMILES: Nc1ccc(I)c(Br)c1 ; COC1CCC(OC)O1 ; Brc1cc(-n2cccc2)ccc1I]

A mixture of the 3-bromo-4-iodoaniline (1.49 g, 5 mmol), 2,5-dimethoxytetrahydrofuran (661 mg, 5 mmol), acetic acid (0.35 mL), 1,2-dichloroethane (10 mL), and water (5 mL) was heated under reflux for 24 h. After removal of the solvent, the crude residue was dissolved in a mixture of water (10 mL) and CH₂Cl₂ (10 mL). The organic extract was washed with a saturated sodium carbonate solution (10 mL) and brine (10 mL). After dried over Mg₂SO₄, the solvent removed in vacuo. The resulting residue was purified by silica gel column chromatography using EtOAc/hexane to afford S13g (96% yield, 4.8 mmol, 1.66 g) as a yellow solid. ¹H NMR (600 MHz, CDCl₃) δ 7.86 (d, J=8.5 Hz, 1H), 7.68 (d, J=2.6 Hz, 1H), 7.08-7.02 (m, 3H), 6.36 (q, J=2.1 Hz, 2H). ¹³C NMR (151 MHz, CDCl₃) δ 141.69, 140.97, 130.71, 124.39, 120.41, 119.18, 111.58, 96.30. HRMS (ESI-TOF) Calculated for C₁₀H₈BrIN [M+H]⁺: 347.8885, Found: 347.8882.

### Preparation of S13p

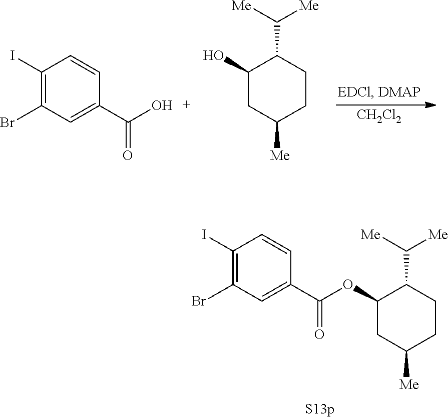

A mixture of L-menthol (781 mg, 5 mmol), 3-bromo-4-iodobenzoic acid (1.96 g, 6 mmol), N-(3-(((ethylimino)methylene)amino)propylidene)-N-methylmethanaminium chloride (EDCI, 1.25 g, 6.5 mmol), and DMAP (122 mg, 1 mmol) in 25 mL of CH₂Cl₂ was stirred at room temperature overnight. The mixture was quenched with saturated NH₄Cl (20 mL), extracted with CH₂Cl₂ (3×20 mL), washed with brine, and dried over Na₂SO₄. After filtration, the filtrate was concentrated under reduced pressure. The residue was purified by flash chromatography on silica gel to afford the expected product S13p (87% yield, 2.02 g, 4.35 mmol) as a colorless oil. ¹H NMR (600 MHz, CDCl₃) δ 8.23 (d, J=1.9 Hz, 1H), 7.94 (d, J=8.2 Hz, 1H), 7.62 (dd, J=8.2, 1.9 Hz, 1H), 4.92 (td, J=10.9, 4.5 Hz, 1H), 2.11-2.05 (m, 1H), 1.88 (pd, J=7.0, 2.9 Hz, 1H), 1.77-1.69 (m, 2H), 1.57-1.50 (m, 2H), 1.17-1.04 (m, 2H), 0.92 (dd, J=10.2, 6.8 Hz, 7H), 0.78 (d, J=6.9 Hz, 3H). ¹³C NMR (151 MHz, CDCl₃) δ 164.59, 140.53, 133.47, 132.45, 130.14, 129.13, 107.45, 75.82, 47.30, 41.00, 34.37, 31.59, 26.66, 23.71, 22.15, 20.88, 16.61.

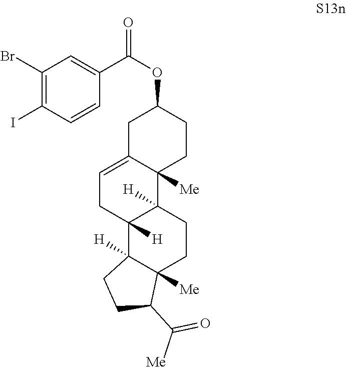

S13n (white solid, 80% yield, 2.5 g, 4.0 mmol) is synthesized with the same preparation procedure of S13p by using dehydroepiandrosterone instead of menthol.

¹H NMR (600 MHz, CDCl₃) δ 8.23 (t, J=2.2 Hz, 1H), 7.94 (dd, J=8.3, 2.8 Hz, 1H), 7.62 (dt, J=8.2, 2.1 Hz, 1H), 5.42 (s, 1H), 4.89-4.74 (m, 1H), 2.54 (td, J=9.0, 2.8 Hz, 1H), 2.48-2.43 (m, 2H), 2.18 (dddd, J=14.2, 9.5, 7.1, 2.5 Hz, 1H), 2.13 (d, J=2.2 Hz, 3H), 2.09-1.96 (m, 3H), 1.93 (dq, J=13.4, 3.5 Hz, 1H), 1.78-1.58 (m, 5H), 1.55-1.43 (m, 3H), 1.28-1.14 (m, 3H), 1.08-1.00 (m, 4H), 0.64 (d, J=2.4 Hz, 3H). ¹³C NMR (151 MHz, CDCl₃) δ 209.65, 164.47, 140.52, 139.53, 133.47, 132.39, 130.12, 129.12, 122.87, 107.52, 75.37, 63.82, 56.98, 50.04, 44.13, 38.94, 38.20, 37.13, 36.79, 31.97, 31.94, 31.71, 27.91, 24.64, 22.99, 21.20, 19.50, 13.38.

HRMS (ESI-TOF) Calculated for C₂₈H₃₅BrIO₃ [M+H]⁺: 625.0814, Found: 625.0798.

### Preparation of S13h, S13i, S13m, S13o

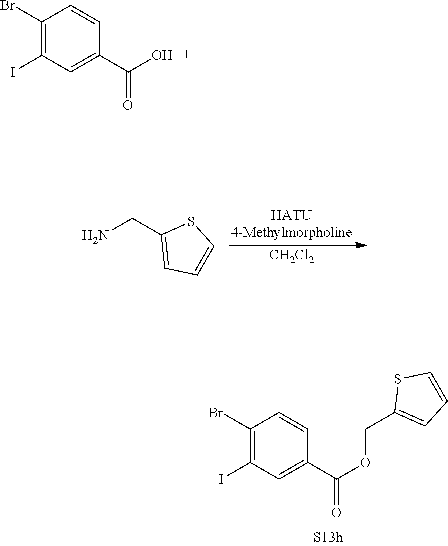

To a 40 mL vial, dihalogenbenzoic acid (2 mmol) and amine (2.5 mmol) were added, followed by 10 mL CH₂Cl₂, and stirred at room temperature for 1 min. HATU (3 mmol) and 4-methylmorpholine (4 mmol) were added sequentially. The vial was capped, and the mixture was stirred at room temperature overnight. After the reaction was finished (monitored by TLC or UPLC), the reaction mixture was passed through a pad of silica, washed by 10 mL EtOAc. The solvent in the filtrate was removed, and the mixture was purified by flash chromatography on silica gel (0% to 20% EtOAc/hexane) to afford the expected product S13h as a white solid (80%, 1.6 mmol, 675 mg).

¹H NMR (600 MHz, CDCl₃) δ 8.24 (d, J=2.1 Hz, 1H), 7.67 (d, J=8.2 Hz, 1H), 7.59 (dd, J=8.3, 2.1 Hz, 1H), 7.26 (dd, J=5.1, 1.2 Hz, 1H), 7.04 (dq, J=3.5, 0.9 Hz, 1H), 6.98 (dd, J=5.1, 3.4 Hz, 1H), 6.38 (s, 1H), 4.79 (dd, J=5.6, 0.8 Hz, 2H). ¹³C NMR (151 MHz, CDCl₃) δ 164.87, 140.21, 138.90, 134.45, 133.81, 132.92, 127.97, 127.24, 126.74, 125.80, 101.61, 39.11.

HRMS (ESI-TOF) Calculated for C₁₂H₁₀BrINOS [M+H]⁺: 421.8711, Found: 421.8705.

### Preparation of S13i

Preparation of S13i followed the procedure of the preparation of S13h with corresponding dihalogenbenzoic acid and amine, the product S13i was purified by flash chromatography on silica gel (20% to 50% EtOAc in hexane) as a white solid (90%, 1.8 mmol, 730 mg).

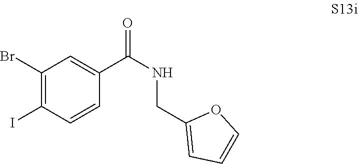 [SMILES: O=C(NCc1ccco1)c1ccc(I)c(Br)c1]

¹H NMR (600 MHz, Acetone-d₆) δ 8.31 (brs, 1H), 8.20 (d, J=2.0 Hz, 1H), 8.06 (d, J=8.2 Hz, 1H), 7.64 (dd, J=8.2, 2.0 Hz, 1H), 7.46 (dd, J=1.9, 0.9 Hz, 1H), 6.36 (dd, J=3.2, 1.9 Hz, 1H), 6.30 (dt, J=3.2, 0.8 Hz, 1H), 4.57 (dd, J=5.7, 0.8 Hz, 2H). ¹³C NMR (151 MHz, CDCl₃) δ 165.26, 150.71, 142.67, 140.66, 135.70, 131.23, 130.47, 126.72, 110.71, 108.22, 105.70, 37.28.

HRMS (ESI-TOF) Calculated for C₁₂H₁₀BrINO₂ [M+H]⁺: 405.8940, Found: 405.8935.

### Preparation of S13m

Preparation of S13m followed the procedure of the preparation of S13h with corresponding dihalogenbenzoic acid and Celecoxib, the product S13m was purified by chromatography on silica gel (0% to 3% MeOH in EtOAc) as a yellow solid (50%, 1.0 mmol, 690 mg).

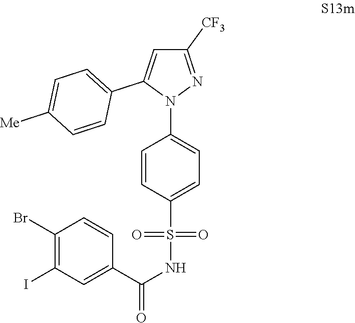 [SMILES: Cc1ccc(-c2cc(C(F)(F)F)nn2-c2ccc(S(=O)(=O)NC(=O)c3ccc(Br)c(I)c3)cc2)cc1]

¹H NMR (600 MHz, CDCl₃) δ 8.22 (d, J=2.2 Hz, 1H), 8.11-8.05 (m, 2H), 7.69 (d, J=8.4 Hz, 1H), 7.58 (dd, J=8.4, 2.2 Hz, 1H), 7.49-7.42 (m, 2H), 7.18 (d, J=7.9 Hz, 2H), 7.15-7.07 (m, 2H), 6.75 (s, 1H), 2.39 (s, 3H). ¹³C NMR (151 MHz, CDCl₃) δ 162.35, 145.58 (q, J=38.7 Hz), 144.38, 143.77, 140.17, 139.56, 137.42, 136.31, 133.26, 131.29, 130.02, 129.99, 128.87, 128.59, 125.65, 125.28, 120.21 (q, J=269.3 Hz), 106.80, 101.93, 21.51. ¹⁹F NMR (376 MHz, CDCl₃) δ −65.11.

HRMS (ESI-TOF) Calculated for C₂₄H₁₇BrF₃IN₃O₃S [M+H]⁺: 689.9171, Found: 689.9177.

### Preparation of S13o

Preparation of S13o followed the procedure of the preparation of S13h with corresponding dihalogenbenzoic acid and 1,2:3,4-Di-O-isopropylidene-α-D-galactopyranose, the product S13o was purified by flash chromatography on silica gel (0% to 20% EtOAc in hexane) as sticky liquid (82%, 1.64 mmol, 933 mg, tends to become white solid over time).

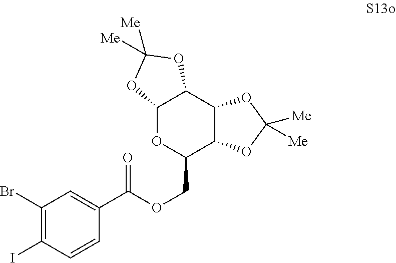 [SMILES: CC1(C)O[C@H]2O[C@H](COC(=O)c3ccc(I)c(Br)c3)[C@H]3OC(C)(C)O[C@H]3[C@H]2O1]

¹H NMR (400 MHz, CDCl₃) δ 8.23 (d, J=1.9 Hz, 1H), 7.93 (d, J=8.2 Hz, 1H), 7.61 (dd, J=8.2, 2.0 Hz, 1H), 5.54 (d, J=4.9 Hz, 1H), 4.64 (dd, J=7.9, 2.5 Hz, 1H), 4.50 (dd, J=11.6, 4.6 Hz, 1H), 4.42 (dd, J=11.5, 7.6 Hz, 1H), 4.33 (dd, J=5.0, 2.5 Hz, 1H), 4.29 (dd, J=7.9, 1.9 Hz, 1H), 4.19-4.11 (m, 1H), 1.50 (s, 3H), 1.46 (s, 3H), 1.34 (s, 3H), 1.32 (s, 3H). ¹³C NMR (151 MHz, CDCl₃) δ 164.82, 140.50, 133.49, 131.54, 130.12, 129.09, 109.81, 108.86, 107.88, 96.34, 71.14, 70.79, 70.54, 66.08, 64.57, 26.11, 26.06, 25.05, 24.58.

HRMS (ESI-TOF) Calculated for C₁₉H₂₃BrIO₇ [M+H]⁺: 568.9672, Found: 568.9675.

### General Procedure for the [2+2] Annulation

### General Procedure for the [2+2] Annulation of Cyclic Aliphatic Acids and Haloarenes

In a sealed 8 mL vial equipped with a magnetic stir bar was charged with the appropriate aliphatic acid substrate (0.10 mmol), dihaloarene (0.20 mmol), Ag₂CO₃ (55.0 mg, 0.20 mmol), and K₂CO₃ (34.5 mg, 0.25 mmol). A solution of Pd(CH₃CN)₄(BF₄)₂(4.4 mg, 10 mol %) and L7 (4.4 mg, 13 mol %) in HFIP (1.0 mL) was premixed and added before the vial was briefly flushed with nitrogen. Subsequently the vial was capped and closed tightly. The reaction mixture was then stirred at the rate of 300 rpm at 100° C. for 20 h. After being allowed to cool to room temperature, the mixture was acidified with 80 μL of formic acid and stirred for 30 seconds. The mixture was passed through a pad of celite with EtOAc as the eluent to remove any insoluble precipitate. The resulting solutions was concentrated, and the residual mixture was purified using pTLC or reverse phase chromatography to afford BCB products.

### General Procedure for the [2+2] Annulation of Acyclic Aliphatic Acids and Haloarenes

In a sealed 12 mL reaction tube equipped with a magnetic stir bar was charged with the appropriate dihaloarene (0.30 mmol), Ag₂CO₃ (55.0 mg, 0.20 mmol), K₂HPO₄ (61.0 mg, 0.35 mmol), Pd(OAc)₂ (2.2 mg, 10 mol %) and L12 (7.9 mg, 20 mol %), followed by aliphatic acid substrate (0.10 mmol) and HFIP (1.2 mL). Subsequently the tube was capped and closed tightly. The reaction mixture was then stirred at the rate of 200 rpm at 110° C. for 24 h. After being allowed to cool to room temperature, the mixture was acidified with 80 μL of formic acid and stirred for 30 seconds. Spherical solid was crushed, and the mixture was passed through a pad of celite with EtOAc as the eluent to remove any insoluble precipitate. The resulting solutions was concentrated, and the residual mixture was purified using pTLC to afford BCB products.

### Procedure for the Gram Scale [2+2] Annulation of Cyclic Aliphatic Acids and Haloarenes

To a 150 mL heavy wall pressure vessel with an internal thread was added Pd(CH₃CN)₄(BF₄)₂(200 mg, 10 mol %), L7 (200 mg, 13 mol %), HFIP (45 mL) and a stir bar. The reaction mixture stirred for 5 mins before aliphatic acid (4.5 mmol), dihaloarene (9.0 mmol), Ag₂CO₃ (2.48 g, 9.0 mmol), and K₂CO₃ (1.55 g, 11.25 mmol) were added. Then the reaction mixture stirred at 300 rpm and 100° C. (oil bath temperature) for 20 h. The reaction mixture was then cooled to room temperature and formic acid (4.0 mL) was added, followed by filtration through a short plug of Celite®. The filtrate was then concentrated under reduced pressure. The product was purified by column chromatography (20% EtOAc/hexane+1% formic acid). The desired product 3a was isolated as a white solid (1.05 g, 3.96 mmol, 88% yield).

### 6. Characterization Data of Products Obtained from the [2+2] Annulation

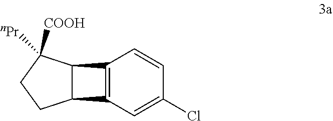

Following the general procedure for the [2+2] annulation of cyclic aliphatic acids and dihaloarenes, the compound was purified by pTLC (20% EtOAc/hexane+1% formic acid) to afford 3a in 90% yield (white solid, 23.8 mg, 0.09 mmol).

¹H NMR (600 MHz, CDCl₃) δ 7.15 (d, J=7.8 Hz, 1H), 7.03 (s, 1H), 6.94 (d, J=8.0 Hz, 1H), 3.87 (dd, J=5.3, 4.4 Hz, 1H), 3.67 (d, J=3.7 Hz, 1H), 1.85-1.76 (m, 4H), 1.74-1.69 (m, 1H), 1.55-1.49 (m, 1H), 1.44-1.32 (m, 2H), 0.97 (t, J=7.3 Hz, 3H). ¹³C NMR (151 MHz, CDCl₃) δ 181.55, 147.91, 142.59, 134.00, 128.17, 124.64, 122.67, 55.35, 54.35, 47.33, 37.60, 29.15, 26.02, 18.40, 14.59.

HRMS (ESI-TOF) Calculated for C₁₅H₁₈ClO₂ [M+H]⁺: 265.0995, Found: 265.0999.

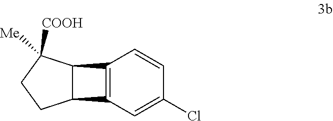 [SMILES: C[C@@]1(C(=O)O)CC[C@@H]2c3cc(Cl)ccc3[C@@H]21]

Following the general procedure for the [2+2] annulation of cyclic aliphatic acids and dihaloarenes, the compound was purified by pTLC (20% EtOAc/hexane+1% formic acid) to afford 3b in 86% yield (white solid, 20.4 mg, 0.086 mmol).

¹H NMR (600 MHz, CDCl₃) δ 7.17 (dd, J=7.7, 2.1 Hz, 1H), 7.04 (s, 1H), 6.97 (d, J=8.0 Hz, 1H), 3.90 (dd, J=3.9, 3.9 Hz, 1H), 3.64 (d, J=3.9 Hz, 1H), 1.92-1.83 (m, 3H), 1.69-1.61 (m, 1H), 1.34 (s, 3H). ¹³C NMR (151 MHz, CDCl₃) δ 182.67, 147.67, 142.63, 134.03, 128.19, 124.88, 122.67, 55.68, 49.92, 47.60, 32.41, 26.03, 22.86.

HRMS (ESI-TOF) Calculated for C₁₃H₁₄ClO₂ [M+H]⁺: 237.0682, Found: 237.0679.

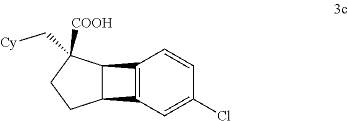

Following the general procedure for the [2+2] annulation of cyclic aliphatic acids and dihaloarenes, the compound was purified by pTLC (20% EtOAc/hexane+1% formic acid) to afford 3c in 75% yield (white solid, 23.9 mg, 0.075 mmol).

¹H NMR (600 MHz, CDCl₃) δ 7.15 (dd, J=7.8, 1.8 Hz, 1H), 7.07-7.00 (m, 1H), 6.90 (d, J=7.8 Hz, 1H), 3.89-3.84 (m, 1H), 3.62 (d, J=3.9 Hz, 1H), 1.85-1.72 (m, 6H), 1.72-1.61 (m, 4H), 1.48-1.40 (m, 2H), 1.32-1.11 (m, 3H), 1.08-0.93 (m, 2H). ¹³C NMR (151 MHz, CDCl₃) δ 182.52, 147.87, 142.28, 134.07, 128.16, 124.24, 122.75, 56.60, 53.18, 47.32, 42.18, 35.26, 34.53, 33.28, 28.42, 26.54, 26.42, 26.35, 26.15.

HRMS (ESI-TOF) Calculated for C₁₉H₂₄ClO₂ [M+H]⁺: 319.1465, Found: 319.1462.

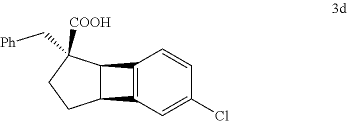 [SMILES: O=C(O)[C@]1(Cc2ccccc2)CC[C@@H]2c3cc(Cl)ccc3[C@@H]21]

Following the general procedure for the [2+2] annulation of cyclic aliphatic acids and dihaloarenes, the compound was purified by pTLC (20% EtOAc/hexane+1% formic acid) to afford 3d in 60% yield (colorless oil, 18.7 mg, 0.06 mmol).

¹H NMR (600 MHz, CDCl₃) δ 7.34-7.26 (m, 3H), 7.22-7.16 (m, 3H), 7.06 (dt, J=1.8, 0.7 Hz, 1H), 6.94 (dt, J=7.8, 0.8 Hz, 1H), 3.99 (dd, J=7.6, 3.7 Hz, 1H), 3.83 (d, J=3.7 Hz, 1H), 3.19 (d, J=13.8 Hz, 1H), 2.79 (d, J=13.8 Hz, 1H), 2.01-1.92 (m, 1H), 1.92-1.86 (m, 1H), 1.79-1.72 (m, 2H). ¹³C NMR (151 MHz, CDCl₃) δ 180.70, 147.84, 142.22, 137.29, 134.22, 129.83, 128.57, 128.31, 127.11, 124.42, 122.81, 55.82, 55.55, 47.58, 40.94, 28.31, 26.13.

HRMS (ESI-TOF) Calculated for C₁₉H₁₆ClO₂ [M−H]⁻: 311.0839, Found: 311.0831.

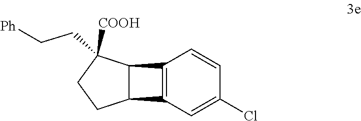 [SMILES: O=C(O)[C@]1(CCc2ccccc2)CC[C@@H]2c3cc(Cl)ccc3[C@@H]21]

Following the general procedure for the [2+2] annulation of cyclic aliphatic acids and dihaloarenes, the compound was purified by pTLC (20% EtOAc/hexane+1% formic acid) to afford 3e in 54% yield (colorless oil, 17.6 mg, 0.054 mmol).

¹H NMR (600 MHz, CDCl₃) δ 7.33-7.27 (m, 2H), 7.24-7.18 (m, 3H), 7.16 (ddd, J=7.7, 1.8, 0.6 Hz, 1H), 7.05 (dt, J=1.6, 0.7 Hz, 1H), 6.98 (dt, J=8.0, 0.8 Hz, 1H), 3.89 (dd, J=6.6, 3.7 Hz, 1H), 3.73 (d, J=3.7 Hz, 1H), 2.75-2.62 (m, 2H), 2.09 (ddd, J=13.9, 12.1, 5.2 Hz, 1H), 1.94-1.81 (m, 5H). ¹³C NMR (151 MHz, CDCl₃) δ 180.82, 147.78, 142.33, 141.59, 134.16, 128.68, 128.47, 128.30, 126.27, 124.65, 122.76, 55.37, 54.34, 47.34, 37.16, 31.56, 29.22, 26.02.

HRMS (ESI-TOF) Calculated for C₂₀H₁₈ClO₂ [M−H]⁻: 327.1152, Found: 327.1151.

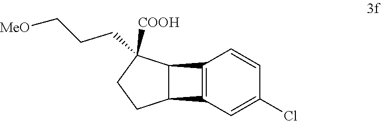 [SMILES: COCCC[C@@]1(C(=O)O)CC[C@@H]2c3cc(Cl)ccc3[C@@H]21]

Following the general procedure for the [2+2] annulation of cyclic aliphatic acids and dihaloarenes, the compound was purified by pTLC (20% EtOAc/hexane+1% formic acid) to afford 3f in 90% yield (white solid, 26.5 mg, 0.09 mmol).

¹H NMR (600 MHz, CDCl₃) δ 7.16 (d, J=7.8 Hz, 1H), 7.03 (s, 1H), 6.94 (d, J=7.8 Hz, 1H), 3.89-3.84 (m, 1H), 3.68 (d, J=3.9 Hz, 1H), 3.43 (s, 2H), 3.35 (s, 3H), 1.85-1.75 (m, 5H), 1.71-1.59 (m, 3H). ¹³C NMR (151 MHz, CDCl₃) δ 180.57, 147.84, 142.48, 134.05, 128.23, 124.67, 122.69, 72.76, 58.71, 55.31, 54.01, 47.35, 31.87, 29.20, 25.97, 25.32.

HRMS (ESI-TOF) Calculated for C₁₆H₁₈ClO₃ [M−H]⁻: 293.0944, Found: 293.0942.

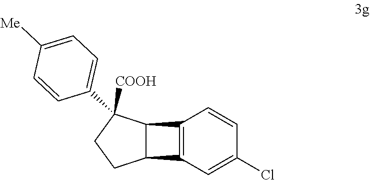 [SMILES: Cc1ccc([C@@]2(C(=O)O)CC[C@@H]3c4cc(Cl)ccc4[C@@H]32)cc1]

Following the general procedure for the [2+2] annulation of cyclic aliphatic acids and dihaloarenes, the compound was purified by pTLC (20% EtOAc/hexane+1% formic acid) to afford 3g in 72% yield (white solid, 22.5 mg, 0.072 mmol).

¹H NMR (600 MHz, CDCl₃) δ 7.35 (d, J=7.9 Hz, 2H), 7.21 (d, J=7.8 Hz, 2H), 7.17 (d, J=7.6 Hz, 1H), 7.06 (s, 1H), 6.98 (d, J=7.8 Hz, 1H), 4.42 (d, J=3.6 Hz, 1H), 3.96 (dd, J=7.6, 3.7 Hz, 1H), 2.37 (s, 3H), 2.12-2.06 (m, 2H), 1.79-1.73 (m, 1H), 1.67-1.57 (m, 1H). ¹³C NMR (151 MHz, CDCl₃) δ 179.78, 147.61, 142.13, 137.58, 137.33, 134.33, 129.62, 128.44, 126.57, 124.67, 122.73, 57.76, 53.41, 48.01, 33.33, 25.96, 21.14.

HRMS (ESI-TOF) Calculated for C₁₉H₁₆ClO₂ [M−H]⁻: 311.0839, Found: 311.0835.

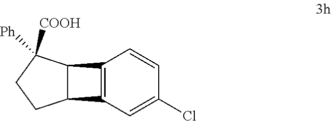 [SMILES: O=C(O)[C@]1(c2ccccc2)CC[C@@H]2c3cc(Cl)ccc3[C@@H]21]

Following the general procedure for the [2+2] annulation of cyclic aliphatic acids and dihaloarenes, the compound was purified by pTLC (20% EtOAc/hexane+1% formic acid) to afford 3h in 68% yield (white solid, 20.3 mg, 0.068 mmol).

¹H NMR (600 MHz, CDCl₃) δ 7.48-7.43 (m, 2H), 7.40 (dd, J=8.5, 6.9 Hz, 2H), 7.36-7.30 (m, 1H), 7.18 (dd, J=7.8, 1.8 Hz, 1H), 7.06-7.08 (m, 1H), 6.99 (d, J=7.8 Hz, 1H), 4.44 (d, J=3.7 Hz, 1H), 3.99 (dd, J=7.6, 3.7 Hz, 1H), 2.17-2.06 (m, 2H), 1.80-1.74 (m, 1H), 1.67-1.58 (m, 1H). ¹³C NMR (151 MHz, CDCl₃) δ 179.49, 147.56, 142.00, 140.66, 134.41, 128.91, 128.48, 127.58, 126.66, 124.64, 122.78, 58.10, 53.35, 48.08, 33.52, 25.93.

HRMS (ESI-TOF) Calculated for C₁₈H₁₄ClO₂ [M−H]⁻: 297.0682, Found: 297.0677.

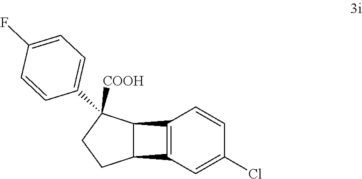 [SMILES: O=C(O)[C@]1(c2ccc(F)cc2)CC[C@@H]2c3cc(Cl)ccc3[C@@H]21]

Following the general procedure for the [2+2] annulation of cyclic aliphatic acids and dihaloarenes, the compound was purified by pTLC (20% EtOAc/hexane+1% formic acid) to afford 3i in 81% yield (white solid, 25.7 mg, 0.081 mmol).

¹H NMR (600 MHz, CDCl₃) δ 7.46-7.40 (m, 2H), 7.19 (ddd, J=7.8, 1.8, 0.6 Hz, 1H), 7.12-7.05 (m, 3H), 6.98 (dt, J=7.9, 0.8 Hz, 1H), 4.39 (d, J=3.7 Hz, 1H), 3.97 (dd, J=7.6, 3.7 Hz, 1H), 2.16-2.03 (m, 2H), 1.81-1.74 (m, 1H), 1.59 (tdd, J=13.3, 7.8, 6.4 Hz, 1H). ¹³C NMR (151 MHz, CDCl₃) δ 179.52, 162.17 (d, J=246.5 Hz), 147.43, 141.78, 136.25 (d, J=3.3 Hz), 134.54, 128.48 (d, J=14.6 Hz), 128.38, 124.62, 122.84, 115.78 (d, J=21.5 Hz), 57.58, 53.48, 47.93, 33.47, 25.88. ¹⁹F NMR (376 MHz, CDCl₃) δ −117.72.

HRMS (ESI-TOF) Calculated for C₁₈H₁₃ClFO₂ [M−H]⁻: 315.0588, Found: 315.0579.

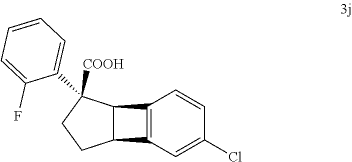 [SMILES: O=C(O)[C@]1(c2ccccc2F)CC[C@@H]2c3cc(Cl)ccc3[C@@H]21]

Following the general procedure for the [2+2] annulation of cyclic aliphatic acids and dihaloarenes, the compound was purified by pTLC (20% EtOAc/hexane+1% formic acid) to afford 3j in 43% yield (white solid, 13.6 mg, 0.043 mmol).

¹H NMR (600 MHz, CDCl₃) δ 7.37-7.27 (m, 2H), 7.23 (ddd, J=7.8, 1.8, 0.6 Hz, 1H), 7.19 (td, J=7.5, 1.3 Hz, 1H), 7.12 (ddd, J=11.3, 8.2, 1.3 Hz, 1H), 7.09-7.07 (m, 1H), 6.97 (dt, J=7.8, 0.8 Hz, 1H), 4.38 (d, J=3.5 Hz, 1H), 4.08 (dd, J=7.8, 3.8 Hz, 1H), 2.31 (td, J=13.6, 6.5 Hz, 1H), 1.89 (dd, J=12.8, 6.0 Hz, 1H), 1.85-1.80 (m, 1H), 1.62-1.52 (m, 1H). ¹³C NMR (151 MHz, CDCl₃) δ 178.74, 160.47 (d, J=246.7 Hz), 146.90, 134.61, 129.61 (d, J=13.6 Hz), 129.17 (d, J=8.3 Hz), 128.58, 127.83 (d, J=4.0 Hz), 124.61, 124.04 (d, J=3.4 Hz), 122.78, 116.26, 116.12, 54.21, 53.83, 48.66, 33.12, 26.20. ¹⁹F NMR (376 MHz, CDCl₃) δ −114.20.

HRMS (ESI-TOF) Calculated for C₁₈H₁₅ClFO₂ [M+H]⁺: 317.0745, Found: 317.0741.

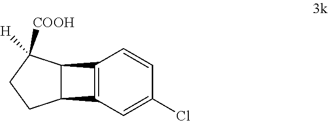 [SMILES: O=C(O)[C@@H]1CC[C@@H]2c3cc(Cl)ccc3[C@@H]21]

Following the general procedure for the [2+2] annulation of cyclic aliphatic acids and dihaloarenes using aliphatic acid (0.2 mmol), 2a (0.1 mmol), K₂CO₃ (3.5 equiv). the compound was purified by pTLC (20% EtOAc/hexane+1% formic acid) to afford 3k in 36% yield (colorless oil, 8.0 mg, 0.036 mmol).

¹H NMR (600 MHz, CDCl₃) δ 7.19 (dd, J=8.5, 1.8 Hz, 1H), 7.03-7.01 (m, 1H), 6.98 (d, J=7.8 Hz, 1H), 4.03 (dd, J=7.4, 3.9 Hz, 1H), 3.90 (dd, J=7.3, 3.8 Hz, 1H), 2.90-2.83 (m, 1H), 1.99-1.89 (m, 2H), 1.74-1.58 (m, 2H). ¹³C NMR (151 MHz, CDCl₃) δ 178.90, 147.65, 141.49, 134.07, 128.31, 125.22, 122.42, 48.56, 47.97, 45.68, 28.07, 26.56.

HRMS (ESI-TOF) Calculated for C₁₂H₁₂ClO₂ [M+H]⁺: 223.0526, Found: 223.0522.

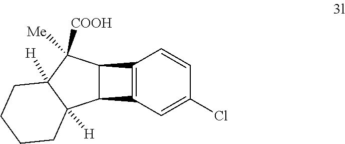 [SMILES: C[C@@]1(C(=O)O)[C@@H]2CCCC[C@@H]2[C@@H]2c3cc(Cl)ccc3[C@@H]21]

Following the general procedure for the [2+2] annulation of cyclic aliphatic acids and dihaloarenes with modifications: aliphatic acid (0.2 mmol), 2a (0.1 mmol), K₂CO₃ (3.5 equiv), the compound was purified by pTLC (20% EtOAc/hexane+1% formic acid) to afford 3l in 32% yield (colorless oil, 9.3 mg, 0.032 mmol).

¹H NMR (600 MHz, CDCl₃) δ 7.13 (dd, J=7.8, 1.9 Hz, 1H), 7.03 (s, 1H), 6.93 (d, J=7.8 Hz, 1H), 3.74 (d, J=4.0 Hz, 1H), 3.61 (d, J=4.0 Hz, 1H), 2.52 (td, J=7.3, 2.1 Hz, 1H), 2.19-2.03 (m, 2H), 1.93-1.85 (m, 1H), 1.66-1.54 (m, 2H), 1.53 (s, 3H), 1.42-1.30 (m, 2H), 1.27-1.18 (m, 2H). ¹³C NMR (151 MHz, CDCl₃) δ 181.89, 148.62, 143.02, 133.95, 127.95, 124.75, 122.49, 57.84, 53.86, 53.26, 42.01, 30.45, 25.07, 24.39, 24.05, 23.43.

HRMS (ESI-TOF) Calculated for C₁₇H₂₀ClO₂ [M+H]⁺: 291.1152, Found: 291.1143.

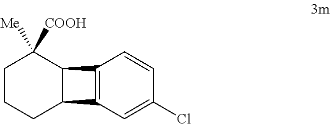 [SMILES: C[C@@]1(C(=O)O)CCC[C@@H]2c3cc(Cl)ccc3[C@@H]21]

Following the general procedure for the [2+2] annulation of cyclic aliphatic acids and dihaloarenes with modifications: aliphatic acid (0.2 mmol), 2a (0.1 mmol), K₂CO₃ (3.5 equiv), the compound was purified by pTLC (20% EtOAc/hexane+1% formic acid) to afford 3m in 50% yield (white solid, 12.5 mg, 0.05 mmol).

¹H NMR (600 MHz, CDCl₃) δ 7.17 (ddd, J=7.8, 1.9, 0.8 Hz, 1H), 7.07 (ddd, J=1.8, 0.8, 0.8 Hz, 1H), 7.01 (d, J=7.8 Hz, 1H), 3.75 (td, J=6.1, 3.1 Hz, 1H), 3.66 (d, J=5.5 Hz, 1H), 2.11-2.01 (m, 1H), 1.89-1.82 (m, 1H), 1.71-1.64 (m, 1H), 1.50-1.60 (m, 2H), 1.48 (s, 4H). ¹³C NMR (151 MHz, CDCl₃) δ 184.17, 149.93, 144.10, 133.58, 127.76, 125.05, 122.40, 47.34, 43.69, 40.83, 26.05, 25.49, 23.12, 16.13.

HRMS (ESI-TOF) Calculated for C₁₄H₁₆ClO₂ [M+H]⁺: 251.0839, Found: 251.0836.

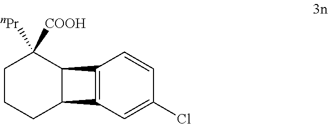

Following the general procedure for the [2+2] annulation of cyclic aliphatic acids and dihaloarenes with modifications: aliphatic acid (0.2 mmol), 2a (0.1 mmol), K₂CO₃ (3.5 equiv), the compound was purified by pTLC (20% EtOAc/hexane+1% formic acid) to afford 3n in 43% yield (colorless oil, 12.0 mg, 0.043 mmol).

¹H NMR (600 MHz, CDCl₃) δ 7.15 (d, J=7.8 Hz, 1H), 7.06 (s, 1H), 6.95 (d, J=7.8 Hz, 1H), δ 3.72 (td, J=6.2, 2.9 Hz, 1H), 3.69 (d, J=5.5 Hz, 1H), 2.08-1.98 (m, 1H), 1.89-1.80 (m, 2H), 1.80-1.72 (m, 1H), 1.60 (t, J=7.1 Hz, 2H), 1.55-1.45 (m, 2H), 1.45-1.30 (m, 2H), 0.98 (t, J=6.8 Hz, 3H). ¹³C NMR (151 MHz, CDCl₃) δ 182.33, 150.31, 143.87, 133.54, 127.76, 124.65, 122.32, 47.63, 47.18, 40.87, 40.24, 23.62, 23.34, 18.41, 16.29, 14.70.

HRMS (ESI-TOF) Calculated for C₁₆H₂₀ClO₂ [M+H]⁺: 279.1152, Found: 279.1152.

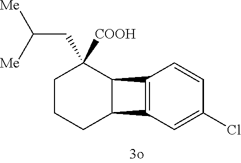 [SMILES: CC(C)C[C@@]1(C(=O)O)CCC[C@@H]2c3cc(Cl)ccc3[C@@H]21]

Following the general procedure for the [2+2] annulation of cyclic aliphatic acids and dihaloarenes with modifications: aliphatic acid (0.2 mmol), 2a (0.1 mmol), K₂CO₃ (3.5 equiv), the compound was purified by pTLC (20% EtOAc/hexane+1% formic acid) to afford 3o in 46% yield (white solid, 13.5 mg, 0.046 mmol).

¹H NMR (600 MHz, CDCl₃) δ 7.16-7.11 (m, 1H), 7.06 (s, 1H), 6.92 (d, J=7.8 Hz, 1H), 3.74-3.68 (m, 1H), 3.66 (d, J=5.5 Hz, 1H), 2.10-2.01 (m, 1H), 1.90-1.76 (m, 4H), 1.66-1.48 (m, 4H), 0.99 (d, J=6.4 Hz, 3H), 0.96 (d, J=6.4 Hz, 3H). ¹³C NMR (151 MHz, CDCl₃) δ 184.18, 150.52, 143.39, 133.60, 127.73, 124.43, 122.28, 47.97, 46.82, 45.96, 40.78, 25.15, 24.64, 23.48, 23.20, 22.71, 16.51.

HRMS (ESI-TOF) Calculated for C₁₇H₂₂ClO₂ [M+H]⁺: 293.1308, Found: 293.1304.

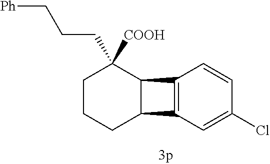 [SMILES: O=C(O)[C@]1(CCCc2ccccc2)CCC[C@@H]2c3cc(Cl)ccc3[C@@H]21]

Following the general procedure for the [2+2] annulation of cyclic aliphatic acids and dihaloarenes with modifications: aliphatic acid (0.2 mmol), 2a (0.1 mmol), K₂CO₃ (3.5 equiv), the compound was purified by pTLC (20% EtOAc/hexane+1% formic acid) to afford 3p in 35% yield (colorless oil, 12.4 mg, 0.035 mmol).

¹H NMR (600 MHz, CDCl₃) δ 7.32-7.27 (m, 2H), 7.21-7.18 (m, 3H), 7.11 (dd, J=7.9, 1.7 Hz, 1H), 7.05 (d, J=2.2 Hz, 1H), 6.93 (d, J=7.8 Hz, 1H), 3.70-3.63 (m, 2H), 2.73-2.61 (m, 2H), 1.95-1.86 (m, 2H), 1.84-1.77 (m, 2H), 1.74-1.65 (m, 2H), 1.59-1.56 (m, 2H), 1.48-1.43 (m, 2H). ¹³C NMR (151 MHz, CDCl₃) δ 181.33, 150.22, 143.70, 141.97, 133.56, 128.53, 128.49, 127.79, 126.09, 124.63, 122.32, 47.45, 47.12, 40.85, 37.25, 36.22, 26.72, 23.49, 23.15, 16.17.

HRMS (ESI-TOF) Calculated for C₂₂H₂₄ClO₂ [M+H]⁺: 355.1465, Found: 355.1464.

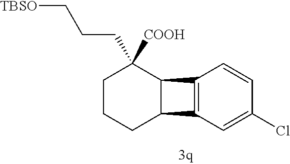 [SMILES: CC(C)(C)[Si](C)(C)OCCC[C@@]1(C(=O)O)CCC[C@@H]2c3cc(Cl)ccc3[C@@H]21]

Following the general procedure for the [2+2] annulation of cyclic aliphatic acids and dihaloarenes with modifications: aliphatic acid (0.2 mmol), 2a (0.1 mmol), K₂CO₃ (3.5 equiv), the compound was purified by pTLC (20% EtOAc/hexane+1% formic acid) to afford 3q in 49% yield (colorless oil, 20.0 mg, 0.049 mmol).

¹H NMR (600 MHz, CDCl₃) δ 7.15 (dd, J=7.8, 1.9 Hz, 1H), 7.12-7.02 (m, 1H), 6.95 (d, J=7.8 Hz, 1H), 3.74-3.59 (m, 4H), 2.09-1.97 (m, 1H), 1.93-1.81 (m, 3H), 1.63-1.46 (m, 6H), 0.91 (s, 9H), 0.07 (s, 6H). ¹³C NMR (151 MHz, CDCl₃) δ 181.22, 150.27, 143.80, 133.54, 127.80, 124.70, 122.33, 63.15, 47.25, 47.12, 40.88, 34.05, 28.44, 26.08, 23.50, 23.10, 18.45, 16.14, −5.11.

HRMS (ESI-TOF) Calculated for C₂₂H₃₂ClO₃Si [M−H]⁻: 407.1809, Found: 407.1802.

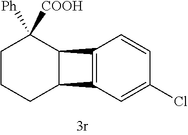 [SMILES: O=C(O)[C@]1(c2ccccc2)CCC[C@@H]2c3cc(Cl)ccc3[C@@H]21]

Following the general procedure for the [2+2] annulation of cyclic aliphatic acids and dihaloarenes with modifications: aliphatic acid (0.2 mmol), 2a (0.1 mmol), K₂CO₃ (3.5 equiv), the compound was purified by pTLC (20% EtOAc/hexane+1% formic acid) to afford 3r in 20% yield (white solid, 6.2 mg, 0.02 mmol).

¹H NMR (600 MHz, CDCl₃) δ 7.60-7.55 (m, 2H), 7.41 (t, J=7.8 Hz, 2H), 7.35-7.30 (m, 1H), 7.19 (d, J=7.8 Hz, 1H), 7.10 (s, 1H), 6.99 (d, J=7.8 Hz, 1H), 4.59 (d, J=4.1 Hz, 1H), 3.81-3.75 (m, 1H), 2.36-2.23 (m, 1H), 1.78-1.70 (m, 1H), 1.70-1.62 (m, 1H), 1.57-1.49 (m, 1H), 1.49-1.41 (m, 2H). ¹³C NMR (151 MHz, CDCl₃) δ 180.34, 149.97, 143.60, 141.09, 133.69, 128.95, 128.09, 127.50, 127.15, 124.89, 122.87, 51.55, 45.83, 41.71, 24.58, 21.86, 16.09.

HRMS (ESI-TOF) Calculated for C₁₉H₁₈ClO₂ [M+H]⁺: 313.0995, Found: 313.0992.

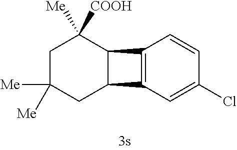 [SMILES: CC1(C)C[C@@H]2c3cc(Cl)ccc3[C@@H]2[C@](C)(C(=O)O)C1]

Following the general procedure for the [2+2] annulation of cyclic aliphatic acids and dihaloarenes with modifications: aliphatic acid (0.2 mmol), 2a (0.1 mmol), K₂CO₃ (3.5 equiv), the compound was purified by pTLC (20% EtOAc/hexane+1% formic acid) to afford 3s in 50% yield (colorless oil, 13.9 mg, 0.05 mmol).

¹H NMR (600 MHz, CDCl₃) δ 7.17 (ddd, J=7.8, 1.8, 0.9 Hz, 1H), 7.11 (dt, J=1.8, 1.0 Hz, 1H), 6.99 (d, J=7.8 Hz, 1H), 3.79 (dt, J=5.8, 2.9 Hz, 1H), 3.65 (dd, J=5.5, 1.0 Hz, 1H), 1.91 (dd, J=15.1, 5.8 Hz, 1H), 1.84 (dd, J=15.0, 2.8 Hz, 1H), 1.51 (dd, J=14.5, 1.7 Hz, 1H), 1.49 (s, 3H), 1.41 (d, J=14.6 Hz, 1H), 1.03 (s, 3H), 0.74 (s, 3H). ¹³C NMR (151 MHz, CDCl₃) δ 182.58, 150.24, 143.75, 133.53, 127.89, 125.33, 122.89, 47.33, 45.58, 41.60, 39.60, 36.82, 35.28, 35.05, 29.45, 24.59.

HRMS (ESI-TOF) Calculated for C₁₆H₂₀ClO₂ [M+H]⁺: 279.1152, Found: 279.1145.

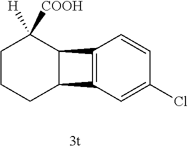 [SMILES: O=C(O)[C@@H]1CCC[C@@H]2c3cc(Cl)ccc3[C@@H]21]

Following the general procedure for the [2+2] annulation of cyclic aliphatic acids and dihaloarenes with modifications: aliphatic acid (0.2 mmol), 2a (0.1 mmol), K₂CO₃ (3.5 equiv), the compound was purified by pTLC (20% EtOAc/hexane+1% formic acid) to afford 3t in 20% yield (colorless oil, 4.7 mg, 0.02 mmol).

¹H NMR (600 MHz, CDCl₃) δ 7.22 (dd, J=7.8, 1.8 Hz, 1H), 7.20 (s, 1H), 6.99 (d, J=7.6 Hz, 1H), 3.92 (t, J=4.8 Hz, 1H), 3.71 (q, J=4.5 Hz, 1H), 2.99 (dt, J=12.9, 5.0 Hz, 1H), 1.90-1.76 (m, 3H), 1.56 (s, 1H), 1.46-1.39 (m, 1H), 1.36-1.29 (m, 1H). ¹³C NMR (151 MHz, CDCl₃) δ 180.36, 146.84, 146.15, 133.09, 128.32, 125.02, 123.80, 42.23, 41.20, 29.85, 23.48, 20.11, 16.69.

HRMS (ESI-TOF) Calculated for C₁₃H₁₄ClO₂ [M+H]⁺: 237.0682, Found: 237.0676.

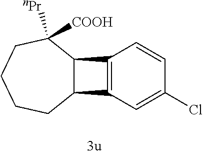

Following the general procedure for the [2+2] annulation of cyclic aliphatic acids and dihaloarenes, the compound was purified by pTLC (20% EtOAc/hexane+1% formic acid) to afford 3u in 54% yield (white solid, 15.8 mg, 0.054 mmol).

¹H NMR (600 MHz, CDCl₃) δ 7.15 (ddd, J=7.7, 1.8, 0.8 Hz, 1H), 7.03 (dt, J=1.8, 0.9 Hz, 1H), 6.93 (dt, J=7.8, 1.0 Hz, 1H), 4.01 (d, J=5.5 Hz, 1H), 3.97-3.94 (m, 1H), 2.24-2.19 (m, 1H), 2.00-1.89 (m, 2H), 1.87-1.76 (m, 2H), 1.65-1.57 (m, 2H), 1.52-1.45 (m, 1H), 1.40-1.23 (m, 3H), 1.14-1.04 (m, 1H), 0.98 (t, J=7.3 Hz, 3H). ¹³C NMR (151 MHz, CDCl₃) δ 182.54, 148.32, 143.55, 133.38, 127.72, 124.41, 122.38, 53.57, 52.05, 47.92, 30.97, 25.93, 24.99, 18.02, 14.81.

HRMS (ESI-TOF) Calculated for C₁₇H₂₂ClO₂ [M+H]⁺: 293.1308, Found: 293.1308.

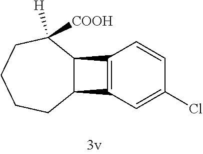 [SMILES: O=C(O)[C@@H]1CCCC[C@@H]2c3cc(Cl)ccc3[C@@H]21]

Following the general procedure for the [2+2] annulation of cyclic aliphatic acids and dihaloarenes, the compound was purified by pTLC (20% EtOAc/hexane+1% formic acid) to afford 3v in 38% yield (white solid, 9.5 mg, 0.038 mmol).

¹H NMR (600 MHz, CDCl₃) δ 7.20 (dd, J=7.4, 1.4 Hz, 1H), 7.14 (d, J=7.8 Hz, 1H), 7.05 (s, 1H), 4.07 (dd, J=5.9, 2.8 Hz, 1H), 3.98-3.92 (m, 1H), 2.82-2.77 (m, 1H), 2.31-2.24 (m, 1H), 1.93 (dd, J=14.3, 5.2 Hz, 1H), 1.79-1.69 (m, 2H), 1.68-1.62 (m, 1H), 1.39-1.30 (m, 1H), 1.19-1.06 (m, 2H). ¹³C NMR (151 MHz, CDCl₃) δ 181.89, 148.64, 143.25, 133.61, 127.83, 125.84, 122.88, 48.05, 47.20, 46.64, 30.71, 29.95, 28.78, 25.45.

HRMS (ESI-TOF) Calculated for C₁₄H₁₆ClO₂ [M+H]⁺: 251.0839, Found: 251.0836.

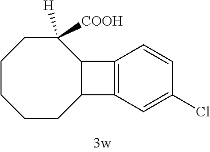 [SMILES: O=C(O)[C@@H]1CCCCCC2c3cc(Cl)ccc3C21]

Following the general procedure for the [2+2] annulation of cyclic aliphatic acids and dihaloarenes, the compound was purified by pTLC (20% EtOAc/hexane+1% formic acid) to afford 3w in 48% yield (white solid, 12.7 mg, 0.048 mmol).

¹H NMR (600 MHz, CDCl₃) δ 7.15 (dd, J=7.8, 2.1 Hz, 1H), 7.05 (s, 1H), 6.97 (d, J=7.8 Hz, 1H), 3.62 (dd, J=11.0, 5.1 Hz, 1H), 3.57 (ddd, J=12.2, 5.2, 1.9 Hz, 1H), 2.82 (td, J=11.4, 3.6 Hz, 1H), 2.15-2.08 (m, 1H), 2.00-1.91 (m, 1H), 1.87-1.62 (m, 5H), 1.54-1.39 (m, 2H), 1.38-1.29 (m, 1H). ¹³C NMR (151 MHz, CDCl₃) δ 182.53, 149.59, 143.03, 133.73, 128.02, 124.71, 122.07, 48.44, 47.46, 43.45, 34.62, 30.44, 29.35, 26.44, 24.88. HRMS (ESI-TOF) Calculated for C₁₅H₁₈ClO₂ [M+H]⁺: 265.0995, Found: 265.0992.

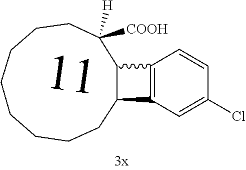

Following the general procedure for the [2+2] annulation of cyclic aliphatic acids and dihaloarenes using L28 instead of L7, KHCO₃ instead of K₂CO₃, the compound was purified by pTLC (20% EtOAc/hexane+1% formic acid) to afford 3x in 50% yield (white solid, 15.3 mg, 0.05 mmol, d.r.=4:1). Trans or cis stereochemistry determined based on the small ³J_{HH} coupling (trans for 2.4 Hz, cis for 5.0 Hz), consistent with the relationship of hydrogens in benzocyclobutenes (7).

trans-3x ¹H NMR (600 MHz, CDCl₃) δ 7.15 (dd, J=7.8, 1.8 Hz, 1H), 7.06-7.03 (m, 1H), 7.00 (d, J=7.9 Hz, 1H), 3.41 (dd, J=11.4, 2.4 Hz, 1H), 3.29 (dt, J=11.4, 2.9 Hz, 1H), 2.65-2.59 (m, 1H), 2.05-1.99 (m, 1H), 1.95-1.90 (m, 2H), 1.82-1.35 (m, 13H). ¹³C NMR (151 MHz, CDCl₃) δ 180.74, 149.22, 143.76, 133.53, 127.86, 124.74, 122.19, 50.40, 49.68, 48.21, 32.25, 29.54, 28.31, 28.07, 27.10, 25.71, 24.99, 21.48.

cis-3x ¹H NMR (600 MHz, CDCl₃) δ 7.13 (dd, J=7.8, 2.7 Hz, 1H), 7.06-7.05 (m, 1H), 6.93 (d, J=7.8 Hz, 1H), 3.73 (dd, J=11.2, 5.0 Hz, 1H), 3.60 (dd, J=10.8, 4.9 Hz, 1H), 2.59-2.53 (m, 1H), 1.82-1.35 (m, 16H). ¹³C NMR (151 MHz, CDCl₃) δ 181.35, 149.46, 143.36, 133.67, 124.28, 122.28, 48.89, 47.86, 46.32, 29.39, 28.07, 27.22, 26.61, 25.20, 25.07, 23.01, 21.39, 21.01.

HRMS (ESI-TOF) Calculated for C₁₈H₂₄ClO₂ [M+H]⁺: 307.1465, Found: 307.1460.

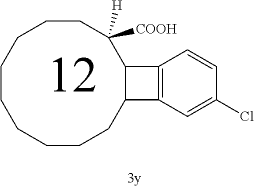 [SMILES: O=C(O)[C@@H]1CCCCCCCCCC2c3cc(Cl)ccc3C21]

Following the general procedure for the [2+2] annulation of cyclic aliphatic acids and dihaloarenes using L28 instead of L7, KHCO₃ instead of K₂CO₃, the compound was purified by pTLC (20% EtOAc/hexane+1% formic acid) to afford 3y in 50% yield (white solid, 16.0 mg, 0.05 mmol, d.r.=4:1, pure cis-3y could not be separable).

trans-3y ¹H NMR (600 MHz, CDCl₃) δ 7.14 (dd, J=7.8, 2.1 Hz, 1H), 7.05 (s, 1H), 7.01 (d, J=7.8 Hz, 1H), 3.34 (dd, J=11.7, 2.1 Hz, 1H), 3.24 (dd, J=12.1, 4.2 Hz, 1H), 2.62 (dt, J=11.9, 4.8 Hz, 1H), 1.94 (tt, J=13.8, 4.7 Hz, 2H), 1.82 (tt, J=13.5, 4.1 Hz, 1H), 1.75-1.60 (m, 4H), 1.59-1.17 (m, 11H). ¹³C NMR (151 MHz, CDCl₃) δ 180.35, 149.88, 144.77, 133.34, 127.68, 124.84, 122.37, 49.29, 47.96, 46.89, 33.60, 29.59, 27.51, 27.24, 25.89, 24.31, 23.47, 23.05, 22.89.

cis-3y ¹H NMR (600 MHz, CDCl₃) δ 7.16-7.13 (m, 1H), 7.05 (dt, J=1.9, 1.0 Hz, 1H), 6.91 (d, J=7.8 Hz, 1H), 3.70-3.64 (m, 1H), 3.59 (ddd, J=10.4, 5.0, 2.2 Hz, 1H), 2.82-2.75 (m, 1H), 1.82-1.17 (m, 18H). ¹³C NMR (151 MHz, CDCl₃) δ 180.98, 149.45, 142.87, 133.77, 127.88, 124.11, 122.24, 48.61, 47.38, 44.26, 31.13, 28.33, 28.28, 28.12, 27.72, 27.61, 26.15, 23.58, 23.32.

HRMS (ESI-TOF) Calculated for C₁₉H₂₆ClO₂ [M+H]⁺: 321.1621, Found: 321.1621.

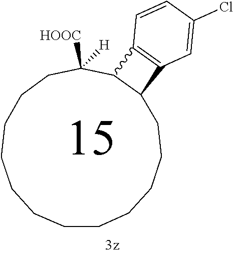

Following the general procedure for the [2+2] annulation of cyclic aliphatic acids and dihaloarenes using L28 instead of L7, KHCO₃ instead of K₂CO₃, the compound was purified by pTLC (20% EtOAc/hexane+1% formic acid) to afford 3z in 52% yield (white solid, 18.9 mg, 0.052 mmol, d.r.>10:1, cis-3z was too little to be collected).

trans-3z ¹H NMR (600 MHz, CDCl₃) δ 7.16 (dd, J=7.4, 1.4 Hz, 1H), 7.08 (s, 1H), 7.00 (d, J=7.8 Hz, 1H), 3.22 (dd, J=11.1, 2.6 Hz, 1H), 3.17 (td, J=7.6, 2.3 Hz, 1H), 2.58 (ddd, J=11.3, 6.7, 5.0 Hz, 1H), 1.88-1.74 (m, 3H), 1.72-1.66 (m, 1H), 1.61-1.50 (m, 4H), 1.46-1.29 (m, 16H). ¹³C NMR (151 MHz, CDCl₃) δ 180.58, 148.89, 143.50, 133.49, 127.84, 124.70, 122.83, 49.88, 49.22, 48.89, 32.23, 31.09, 27.10, 26.94, 26.79, 26.70, 26.48, 26.16, 26.12, 26.08, 25.65, 25.10.

HRMS (ESI-TOF) Calculated for C₂₂H₃₂ClO₂ [M+H]⁺: 363.2091, Found: 363.2089.

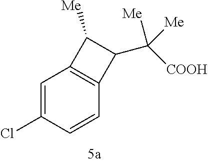 [SMILES: C[C@H]1c2cc(Cl)ccc2C1C(C)(C)C(=O)O]

Following the general procedure for the [2+2] annulation of acyclic aliphatic acids and dihaloarenes, the compound was purified by pTLC (20% EtOAc/hexane+1% formic acid) to afford 5a in 68% yield (yellow oil, 16.2 mg, 0.068 mmol). Trans stereochemistry determined based on the small ³J_{HH} coupling (2.7 Hz), consistent with the trans relationship of hydrogens in benzocyclobutenes.⁷.

¹H NMR (600 MHz, CDCl₃) δ 7.18 (dd, J=7.8, 2.6 Hz, 1H), 7.06 (s, 1H), 7.04 (d, J=7.7 Hz, 1H), 3.36 (qd, J=7.1, 2.8 Hz, 1H), 3.24 (d, J=2.7 Hz, 1H), 1.39 (d, J=7.1 Hz, 3H), 1.27 (s, 3H), 1.22 (s, 3H). ¹³C NMR (151 MHz, CDCl₃) δ 183.59, 150.11, 141.84, 133.46, 127.82, 125.02, 122.35, 58.08, 43.58, 40.50, 22.92, 21.87, 18.72.

HRMS (ESI-TOF) Calculated for C₁₃H₁₆ClO₂ [M+H]⁺: 239.0839, Found: 239.0838.

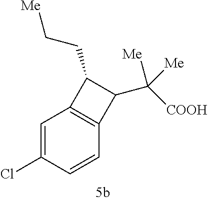 [SMILES: CCC[C@H]1c2cc(Cl)ccc2C1C(C)(C)C(=O)O]

Following the general procedure for the [2+2] annulation of acyclic aliphatic acids and dihaloarenes, the compound was purified by pTLC (20% EtOAc/hexane+1% formic acid) to afford 5b in 65% yield (yellow oil, 17.3 mg, 0.065 mmol).

¹H NMR (600 MHz, CDCl₃) δ 7.18 (dd, J=7.8, 1.8 Hz, 1H), 7.09 (s, 1H), 7.04 (d, J=7.8 Hz, 1H), 3.30 (d, J=2.5 Hz, 1H), 3.28 (ddd, J=8.8, 6.1, 2.5 Hz, 1H), 1.79-1.71 (m, 1H), 1.65-1.55 (m, 1H), 1.55-1.43 (m, 2H), 1.24 (s, 3H), 1.22 (s, 3H), 0.96 (t, J=7.3 Hz, 3H). ¹³C NMR (151 MHz, CDCl₃) δ 183.96, 149.46, 142.20, 133.29, 127.73, 124.92, 122.93, 56.47, 45.58, 43.61, 36.26, 22.69, 22.14, 21.55, 14.35.

HRMS (ESI-TOF) Calculated for C₁₅H₂₀ClO₂ [M+H]⁺: 267.1152, Found: 267.1152.

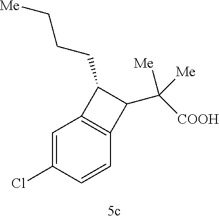 [SMILES: CCCC[C@H]1c2cc(Cl)ccc2C1C(C)(C)C(=O)O]

Following the general procedure for the [2+2] annulation of acyclic aliphatic acids and dihaloarenes, the compound was purified by pTLC (20% EtOAc/hexane+1% formic acid) to afford 5c in 59% yield (colorless oil, 17.4 mg, 0.059 mmol).

¹H NMR (600 MHz, CDCl₃) δ 7.18 (dd, J=8.2, 1.5 Hz, 1H), 7.09 (s, 1H), 7.04 (d, J=7.8 Hz, 1H), 3.30 (d, J=2.6 Hz, 1H), 3.26 (ddd, J=8.9, 6.2, 2.5 Hz, 1H), 1.80-1.71 (m, 1H), 1.65-1.56 (m, 1H), 1.51-1.38 (m, 2H), 1.36-1.29 (m, 4H), 1.24 (s, 3H), 1.21 (s, 3H), 0.93-0.88 (m, 3H). ¹³C NMR (151 MHz, CDCl₃) δ 183.75, 149.49, 142.19, 133.30, 127.74, 124.93, 122.92, 56.46, 45.80, 43.61, 34.03, 32.07, 28.05, 22.75, 22.70, 22.14, 14.23.

HRMS (ESI-TOF) Calculated for C₁₇H₂₄ClO₂ [M+H]⁺: 295.1465, Found: 295.1463.

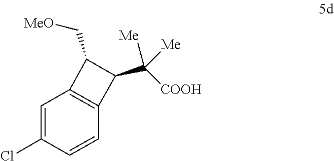 [SMILES: COC[C@H]1c2cc(Cl)ccc2[C@@H]1C(C)(C)C(=O)O]

Following the general procedure for the [2+2] annulation of acyclic aliphatic acids and dihaloarenes, the compound was purified by pTLC (20% EtOAc/hexane+1% formic acid) to afford 5d in 50% yield (colorless oil, 13.4 mg, 0.05 mmol).

¹H NMR (600 MHz, CDCl₃) δ 7.20 (dd, J=7.8, 1.9 Hz, 1H), 7.14 (s, 1H), 7.05 (d, J=7.8 Hz, 1H), 3.70 (dd, J=8.9, 5.5 Hz, 1H), 3.58-3.49 (m, 2H), 3.44 (d, J=3.1 Hz, 1H), 3.38 (s, 3H), 1.26 (s, 3H), 1.23 (s, 3H). ¹³C NMR (151 MHz, CDCl₃) δ 183.16, 146.67, 142.48, 133.56, 128.23, 124.85, 123.27, 74.45, 59.24, 53.63, 45.20, 43.41, 22.80, 22.14.

HRMS (ESI-TOF) Calculated for C₁₄H₁₈ClO₃ [M+H]⁺: 269.0944, Found: 269.0938.

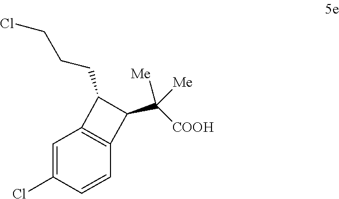 [SMILES: CC(C)(C(=O)O)[C@H]1c2ccc(Cl)cc2[C@@H]1CCCCl]

Following the general procedure for the [2+2] annulation of acyclic aliphatic acids and dihaloarenes, the compound was purified by pTLC (20% EtOAc/hexane+1% formic acid) to afford 5e in 55% yield (colorless oil, 16.6 mg, 0.055 mmol).

¹H NMR (600 MHz, CDCl₃) δ 7.20 (dd, J=8.2, 2.2 Hz, 1H), 7.09 (s, 1H), 7.05 (d, J=7.7 Hz, 1H), 3.57 (t, J=6.4 Hz, 2H), 3.33 (d, J=3.5 Hz, 1H), 3.30 (ddd, J=5.5, 5.5, 2.7 Hz, 1H), 2.00-1.86 (m, 3H), 1.83-1.68 (m, 1H), 1.26 (s, 3H), 1.21 (s, 3H). ¹³C NMR (151 MHz, CDCl₃) δ 182.87, 148.53, 142.02, 133.52, 128.10, 125.05, 122.89, 56.37, 44.96, 44.93, 43.56, 31.25, 31.21, 22.97, 22.01.

HRMS (ESI-TOF) Calculated for C₁₅H₁₉Cl₂O₂[M+H]⁺: 301.0762, Found: 301.0766.

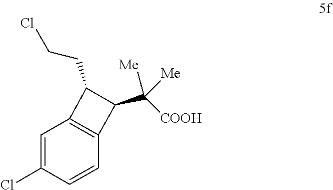 [SMILES: CC(C)(C(=O)O)[C@H]1c2ccc(Cl)cc2[C@@H]1CCCl]

Following the general procedure for the [2+2] annulation of acyclic aliphatic acids and haloarenes, the compound was purified by pTLC (10% EtOAc/hexane+1% formic acid) to afford 5f in 42% yield (colorless oil, 12.1 mg, 0.042 mmol).

¹H NMR (600 MHz, CDCl₃) δ 7.22 (ddd, J=7.8, 1.8, 0.7 Hz, 1H), 7.13 (dt, J=1.7, 0.8 Hz, 1H), 7.06 (dt, J=7.8, 0.9 Hz, 1H), 3.72-3.64 (m, 2H), 3.50 (ddd, J=8.8, 6.3, 2.4 Hz, 1H), 3.38 (dd, J=2.5, 0.9 Hz, 1H), 2.32-2.22 (m, 1H), 2.10 (ddt, J=14.1, 8.6, 6.3 Hz, 1H), 1.28 (s, 3H), 1.22 (s, 3H). ¹³C NMR (151 MHz, CDCl₃) δ 177.09, 144.32, 138.84, 130.98, 125.86, 122.76, 120.90, 57.32, 45.14, 45.05, 44.60, 38.57, 25.63, 24.50.

HRMS (ESI-TOF) Calculated for C₁₄H₁₇Cl₂O₂[M+H]⁺: 287.0606, Found: 287.0606.

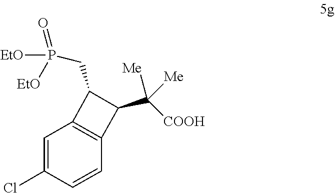 [SMILES: CCOP(=O)(C[C@H]1c2cc(Cl)ccc2[C@@H]1C(C)(C)C(=O)O)OCC]

Following the general procedure for the [2+2] annulation of acyclic aliphatic acids and haloarenes, the compound was purified by pTLC (50% EtOAc/hexane+1% formic acid) to afford 5g in 45% yield (colorless oil, 16.9 mg, 0.045 mmol).

¹H NMR (600 MHz, CDCl₃) δ 7.26 (s, 1H), 7.20 (dd, J=7.8, 1.8 Hz, 1H), 7.02 (d, J=7.8 Hz, 1H), 4.21-4.09 (m, 4H), 3.57 (tt, J=10.8, 3.2 Hz, 1H), 3.42 (d, J=2.5 Hz, 1H), 2.49 (ddd, J=19.1, 15.3, 3.8 Hz, 1H), 2.07 (ddd, J=17.3, 15.3, 11.1 Hz, 1H), 1.35 (dt, J=12.7, 7.0 Hz, 6H), 1.31 (s, 3H), 1.12 (s, 3H). ¹³C NMR (151 MHz, CDCl₃) δ 180.76, 147.51 (d, J=5.2 Hz), 141.88, 133.46, 128.39, 124.67, 124.29, 62.22 (d, J=6.6 Hz), 62.03 (d, J=6.9 Hz), 57.85 (d, J=18.9 Hz), 43.48, 39.27 (d, J=6.3 Hz), 29.51 (d, J=139.3 Hz), 24.14, 20.98, 16.58, 16.54. ³¹P NMR (162 MHz, CDCl₃) δ 30.15.

HRMS (ESI-TOF) Calculated for C₁₇H₂₅ClO₅P [M+H]⁺: 375.1128, Found: 375.1123.

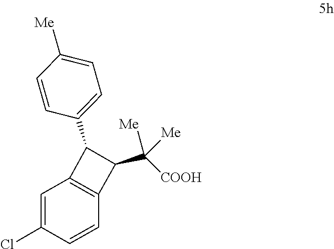 [SMILES: Cc1ccc([C@H]2c3cc(Cl)ccc3[C@@H]2C(C)(C)C(=O)O)cc1]

Following the general procedure for the [2+2] annulation of acyclic aliphatic acids and dihaloarenes, the compound was purified by pTLC (20% EtOAc/hexane+1% formic acid) to afford 5h in 45% yield (colorless oil, 14.2 mg, 0.045 mmol).

¹H NMR (600 MHz, CDCl₃) δ 7.26-7.24 (m, 1H), 7.15-7.05 (m, 6H), 4.42 (d, J=2.6 Hz, 1H), 3.55 (d, J=2.6 Hz, 1H), 2.32 (s, 3H), 1.30 (s, 6H). ¹³C NMR (151 MHz, CDCl₃) δ 182.03, 147.59, 142.95, 138.38, 136.65, 133.88, 129.40, 128.37, 127.27, 125.25, 123.40, 60.50, 49.59, 43.95, 22.51, 22.48, 21.21.

HRMS (ESI-TOF) Calculated for C₁₉H₂₀ClO₂ [M+H]⁺: 315.1152, Found: 315.1154.

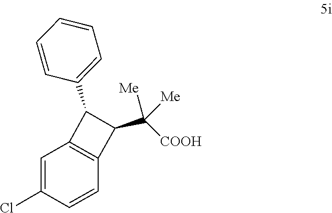 [SMILES: CC(C)(C(=O)O)[C@H]1c2ccc(Cl)cc2[C@@H]1c1ccccc1]

Following the general procedure for the [2+2] annulation of acyclic aliphatic acids and dihaloarenes, the compound was purified by pTLC (20% EtOAc/hexane+1% formic acid) to afford 5i in 52% yield (colorless oil, 15.6 mg, 0.052 mmol).

¹H NMR (400 MHz, CDCl₃) δ 7.30-7.23 (m, 4H), 7.20-7.16 (m, 2H), 7.16-7.09 (m, 2H), 4.46 (d, J=2.9 Hz, 1H), 3.58 (d, J=2.9 Hz, 1H), 1.31 (s, 6H). ¹³C NMR (151 MHz, CDCl₃) δ 182.73, 147.33, 142.92, 141.37, 133.95, 128.72, 128.47, 127.38, 127.04, 125.29, 123.45, 60.42, 49.90, 44.01, 22.50, 22.47.

HRMS (ESI-TOF) Calculated for C₁₈H₁₈ClO₂ [M+H]⁺: 301.0995, Found: 301.0995.

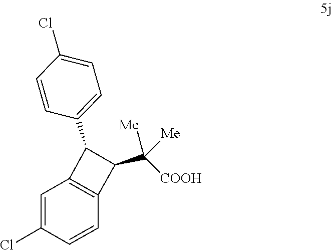 [SMILES: CC(C)(C(=O)O)[C@H]1c2ccc(Cl)cc2[C@@H]1c1ccc(Cl)cc1]

Following the general procedure for the [2+2] annulation of acyclic aliphatic acids and dihaloarenes, the compound was purified by pTLC (20% EtOAc/hexane+1% formic acid) to afford 5j in 60% yield (colorless oil, 20.1 mg, 0.06 mmol).

¹H NMR (600 MHz, CDCl₃) δ 7.28 (dd, J=8.7, 1.9 Hz, 1H), 7.26-7.23 (m, 2H), 7.14 (d, J=8.0 Hz, 1H), 7.12-7.08 (m, 3H), 4.42 (d, J=2.7 Hz, 1H), 3.53 (d, J=2.7 Hz, 1H), 1.31 (s, 3H), 1.29 (s, 3H). ¹³C NMR (151 MHz, CDCl₃) δ 182.14, 146.87, 142.72, 139.89, 134.14, 132.79, 129.84, 128.76, 128.72, 125.34, 123.39, 60.60, 49.25, 43.96, 22.75, 22.21.

HRMS (ESI-TOF) Calculated for C₁₈H₁₅Cl₂O₂[M−H]⁻: 333.0449, Found: 333.0435.

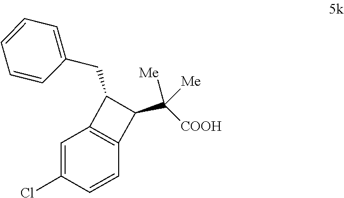 [SMILES: CC(C)(C(=O)O)[C@H]1c2ccc(Cl)cc2[C@@H]1Cc1ccccc1]

Following the general procedure for the [2+2] annulation of acyclic aliphatic acids and dihaloarenes, the compound was purified by pTLC (20% EtOAc/hexane+1% formic acid) to afford 5k in 40% yield (colorless oil, 12.6 mg, 0.04 mmol).

¹H NMR (600 MHz, CDCl₃) δ 7.32 (t, J=7.4 Hz, 2H), 7.26-7.21 (m, 3H), 7.18 (dd, J=7.7, 2.0 Hz, 1H), 7.04 (d, J=7.8 Hz, 1H), 6.78 (s, 1H), 3.54 (td, J=6.3, 3.0 Hz, 1H), 3.43 (d, J=2.6 Hz, 1H), 3.12 (dd, J=13.6, 6.4 Hz, 1H), 2.88 (dd, J=13.6, 9.5 Hz, 1H), 1.21 (s, 3H), 1.11 (s, 3H).

¹³C NMR (151 MHz, CDCl₃) δ 182.95, 148.54, 141.97, 140.25, 133.28, 129.14, 128.63, 128.12, 126.54, 124.99, 123.13, 56.18, 47.07, 43.53, 40.11, 22.72, 22.13.

HRMS (ESI-TOF) Calculated for C₁₉H₂₀ClO₂ [M+H]⁺: 315.1152, Found: 315.1151.

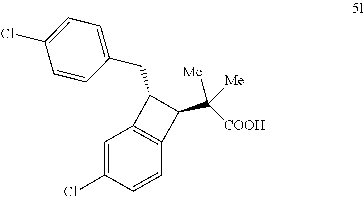 [SMILES: CC(C)(C(=O)O)[C@H]1c2ccc(Cl)cc2[C@@H]1Cc1ccc(Cl)cc1]

Following the general procedure for the [2+2] annulation of acyclic aliphatic acids and haloarenes, the compound was purified by pTLC (20% EtOAc/hexane+1% formic acid) to afford 5l in 30% yield (colorless oil, 10.5 mg, 0.030 mmol).

¹H NMR (600 MHz, CDCl₃) δ 7.31-7.28 (m, 2H), 7.18 (ddd, J=7.9, 1.9, 0.7 Hz, 1H), 7.17-7.13 (m, 2H), 7.06-7.02 (m, 1H), 6.79-6.75 (m, 1H), 3.51 (ddd, J=9.1, 6.2, 2.4 Hz, 1H), 3.44-3.36 (m, 1H), 3.09 (dd, J=13.6, 6.2 Hz, 1H), 2.83 (dd, J=13.6, 9.4 Hz, 1H), 1.22 (s, 3H), 1.12 (s, 3H). ¹³C NMR (151 MHz, CDCl₃) δ 182.75, 148.14, 141.87, 138.64, 133.39, 132.33, 130.48, 128.74, 128.28, 125.05, 123.04, 56.11, 46.79, 43.52, 39.37, 22.95, 22.03.

HRMS (ESI-TOF) Calculated for C₁₉H₁₉Cl₂O₂[M+H]⁺: 349.0762, Found: 349.0751.

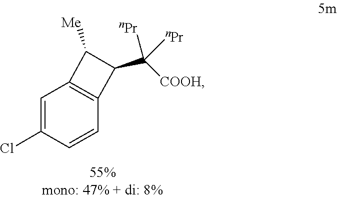

Following the general procedure for the [2+2] annulation of acyclic aliphatic acids and haloarenes, the compound was purified by pTLC (20% EtOAc/hexane+1% formic acid) to afford 5m in 47% yield (colorless oil, 13.9 mg, 0.047 mmol). Di-BCB products were identified by ¹H NMR of a mixture of diastereomers.

¹H NMR (600 MHz, CDCl₃) δ 7.15 (ddd, J=8.0, 1.7, 0.8 Hz, 1H), 7.07-7.01 (m, 2H), 3.48 (qd, J=7.0, 2.6 Hz, 1H), 3.28 (dd, J=2.7, 0.9 Hz, 1H), 1.70-1.58 (m, 4H), 1.36 (d, J=7.0 Hz, 3H), 1.35-1.26 (m, 4H), 0.90 (td, J=7.3, 3.2 Hz, 6H). ¹³C NMR (151 MHz, CDCl₃) δ 181.44, 150.01, 142.47, 133.20, 127.67, 125.40, 122.12, 55.85, 50.87, 40.47, 36.56, 18.88, 18.11, 17.93, 17.33, 14.92, 14.77.

HRMS (ESI-TOF) Calculated for C₁₇H₂₅ClO₂ [M+H]⁺: 295.1465, Found: 295.1460.

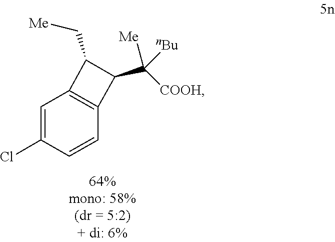

Following the general procedure for the [2+2] annulation of acyclic aliphatic acids and haloarenes, the compound was purified by pTLC (20% EA/hexane+1% formic acid) to afford 5n in 58% yield (white solid, 17.1 mg, 0.058 mmol, dr=5:2). Diastereomers were not separable by column chromatography or pTLC, ¹H and ¹³C NMR data is reported as a mixture of diastereomers. Diastereomeric ratio was identified by ¹H NMR of the mixture of the diastereomers.

Di-BCB products were identified by ¹H NMR of a mixture of diastereomers, which were not separable, and the yield was identified by crude ¹H NMR using CH₂Br₂ as internal standard.

¹H NMR (600 MHz, CDCl₃) δ 7.20-7.14 (m, 1.37H), 7.09 (ddt, J=6.3, 1.7, 0.8 Hz, 1.37H), 7.04 (dt, J=7.8, 0.9 Hz, 0.37H), 7.00-6.96 (m, 1H), 3.35 (d, J=2.2 Hz, 1H), 3.32 (d, J=2.1 Hz, 0.37H), 3.24 (m, 1.37H), 1.88-1.73 (m, 2.74H), 1.66-1.45 (m, 3.11H), 1.36-1.26 (m, 5.11H), 1.11 (s, 3H), 1.07 (s, 1.11H), 1.06 (t, J=7.4 Hz, 3H), 1.03 (t, J=7.4 Hz, 1.11H), 0.96-0.87 (m, 4.11H). ¹³C NMR (151 MHz, CDCl₃) δ 182.52, 182.45, 149.45, 149.11, 142.38, 142.01, 133.32, 133.17, 127.78, 127.71, 124.87, 124.95, 122.97, 122.84, 56.07, 55.55, 47.61, 47.45, 47.31, 47.10, 37.86, 37.04, 27.27, 27.19, 27.12, 26.76, 23.35, 23.33, 18.58, 18.16, 14.15, 14.14, 12.64, 12.51.

HRMS (ESI-TOF) Calculated for C₁₇H₂₄ClO₂ [M−H]⁻: 293.1308, Found: 293.1307.

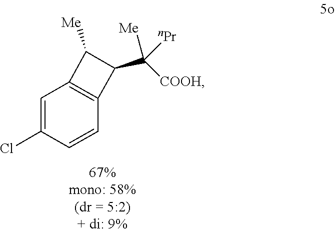

Following the general procedure for the [2+2] annulation of acyclic aliphatic acids and haloarenes, the compound was purified by pTLC (20% EtOAc/hexane+1% formic acid) to afford 5o in 58% yield (colorless oil, 15.5 mg, 0.058 mmol, dr=5:2). Diastereomers were not separable by column chromatography or pTLC, ¹H and ¹³C NMR data is reported as a mixture of diastereomers. Diastereomeric ratio was identified by ¹H NMR of the mixture of the diastereomers.

Di-BCB products were identified by ¹H NMR of a mixture of diastereomers, which were not separable, and the yield was identified by crude ¹H NMR using CH₂Br₂ as internal standard.

¹H NMR (600 MHz, CDCl₃) δ 7.17 (tdd, J=7.9, 1.8, 0.7 Hz, 1.38H), 7.07-7.01 (m, 1.76H), 6.98 (dd, J=7.8, 0.9 Hz, 1H), 3.38 (dtd, J=9.6, 7.0, 4.3 Hz, 1.38H), 3.30 (dd, J=2.7, 0.9 Hz, 1H), 3.26 (dd, J=2.6, 0.9 Hz, 0.38H), 1.77 (m, 1.38H), 1.61-1.47 (m, 1.38H), 1.44-1.29 (m, 6.9H), 1.12 (s, 3H), 1.09 (s, 1.14H), 0.94 (td, J=7.3, 5.1 Hz, 4.14H). ¹³C NMR (151 MHz, CDCl₃) δ 182.74, 182.62, 150.25, 149.87, 142.08, 141.69, 133.46, 133.32, 127.83, 127.75, 125.04, 125.02, 122.38, 122.23, 57.77, 57.46, 47.71, 47.42, 40.45, 40.33, 40.12, 39.46, 18.94, 18.72, 18.59, 18.44, 18.38, 18.26, 14.74, 14.71.

HRMS (ESI-TOF) Calculated for C₁₅H₂₀ClO₂ [M+H]⁺: 267.1152, Found: 267.1149.

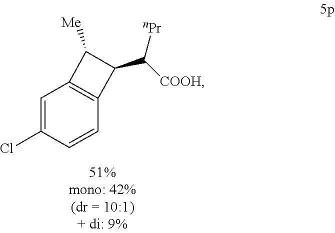

Following the general procedure for the [2+2] annulation of acyclic aliphatic acids and haloarenes with modifications: 2a (0.35 mmol), K₂HPO₄ (0.3 mmol), HFIP (1.8 mL), the compound was purified by pTLC (20% EtOAc/hexane+1% formic acid) to afford 5p in 42% yield (colorless oil, 10.6 mg, 0.042 mmol, dr=10:1). Diastereomers were not separable by column chromatography or pTLC, ¹H and ¹³C NMR data is reported as a mixture of diastereomers. Diastereomeric ratio was identified by ¹H NMR of the mixture of the diastereomers.

Di-BCB products were identified by ¹H NMR of a mixture of diastereomers, which were not separable, and the yield was identified by crude ¹H NMR using CH₂Br₂ as internal standard.

¹H NMR (600 MHz, CDCl₃) δ 7.18 (ddd, J=7.8, 1.9, 0.7 Hz, 0.1H), 7.15 (ddd, J=7.8, 1.9, 0.7 Hz, 1H), 7.09-7.04 (m, 1.1H), 6.97 (d, J=7.8 Hz, 1H), 6.92 (d, J=7.8 Hz, 0.1H), 3.29 (qd, J=7.0, 2.3 Hz, 0.1H), 3.24 (qd, J=7.0, 2.3 Hz, 1H), 3.14 (dd, J=10.0, 2.09 Hz, 0.1H), 3.11 (dd, J=10.0, 2.09 Hz, 1H), 2.62 (td, J=9.8, 4.0 Hz, 1.1H), 1.79 (m, 1.1H), 1.65-1.57 (m, 1.1H), 1.54-1.45 (m, 1.1H), 1.41 (d, J=7.1 Hz, 3H), 1.39-1.33 (m, 1.4H), 0.95 (t, J=7.3 Hz, 3.3H). ¹³C NMR (151 MHz, CDCl₃) δ 180.69, 180.61, 149.94, 149.56, 143.23, 142.73, 133.70, 133.60, 127.92, 127.86, 124.78, 124.50, 122.70, 122.51, 52.23, 52.19, 49.07, 47.43, 43.86, 43.63, 33.05, 32.94, 20.74, 20.59, 18.83, 18.32, 14.54, 14.18.

HRMS (ESI-TOF) Calculated for C₁₄H₁₈ClO₂ [M+H]⁺: 253.0995, Found: 253.0996.

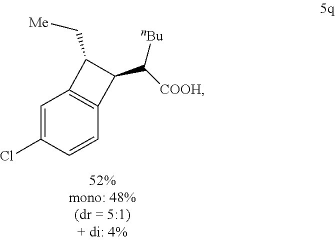

Following the general procedure for the [2+2] annulation of acyclic aliphatic acids and haloarenes with modifications: 2a (0.35 mmol), K₂HPO₄ (0.3 mmol), HFIP (1.8 mL), the compound was purified by pTLC (20% EtOAc/hexane+1% formic acid) to afford 5q in 48% yield (colorless oil, 10.7 mg, 0.048 mmol, dr=5:1). Diastereomers were not separable by column chromatography or pTLC, ¹H and ¹³C NMR data is reported as a mixture of diastereomers. Diastereomeric ratio was identified by ¹H NMR of the mixture of the diastereomers.

Di-BCB products were identified by ¹H NMR of a mixture of diastereomers, which were not separable, and the yield was identified by crude ¹H NMR using CH₂Br₂ as internal standard.

¹H NMR (600 MHz, CDCl₃) δ 7.18 (ddd, J=7.8, 1.9, 0.7 Hz, 0.2H), 7.16 (ddd, J=7.8, 1.9, 0.7 Hz, 1H), 7.11 (m, 0.2H), 7.10 (m, 1H), 7.06 (dt, J=7.8, 0.9 Hz, 0.2H), 6.98 (dt, J=7.8, 0.9 Hz, 1H), 3.22-3.16 (m, 1.4H), 3.11 (ddd, J=8.5, 6.5, 2.2 Hz, 1H), 2.59 (td, J=9.9, 3.9 Hz, 1.2H), 1.85-1.62 (m, 4.8H), 1.47-1.30 (m, 4.8H), 1.07 (t, J=7.4 Hz, 3H), 1.03 (t, J=7.4 Hz, 0.6H), 0.91 (t, J=7.0 Hz, 3.6H). ¹³C NMR (151 MHz, CDCl₃) δ 180.15, 179.91, 149.09, 148.75, 143.11, 143.10, 133.56, 133.46, 127.88, 127.75, 124.72, 124.38, 123.20, 123.08, 50.72, 50.54, 50.45, 50.16, 49.12, 49.02, 30.70, 30.46, 29.85, 29.63, 26.93, 26.55, 22.76, 22.75, 14.06, 14.05, 12.65, 12.46.

HRMS (ESI-TOF) Calculated for C₁₆H₂₀ClO₂ [M−H]⁻: 279.1152, Found: 279.1145.

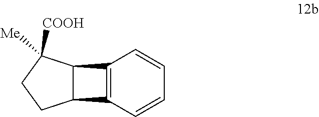 [SMILES: C[C@@]1(C(=O)O)CC[C@@H]2c3ccccc3[C@@H]21]

Following the general procedure for the [2+2] annulation of cyclic aliphatic acids and dihaloarenes, the compound was purified by pTLC (20% EtOAc/hexane+1% formic acid) to afford 12b in 72% yield (white solid, 14.6 mg, 0.072 mmol).

¹H NMR (600 MHz, CDCl₃) δ 7.26-7.17 (m, 2H), 7.05 (dd, J=13.0, 7.2 Hz, 2H), 3.94 (dd, J=3.6, 3.5 Hz, 1H), 3.71 (d, J=3.9 Hz, 1H), 1.97-1.83 (m, 3H), 1.66-1.60 (m, 1H), 1.35 (s, 3H). ¹³C NMR (151 MHz, CDCl₃) δ 183.17, 146.51, 144.63, 128.39, 127.65, 123.25, 122.01, 56.43, 49.94, 47.82, 32.40, 26.17, 22.91.

HRMS (ESI-TOF) Calculated for C₁₃H₁₅O₂[M+H]⁺: 203.1072, Found: 203.1069.

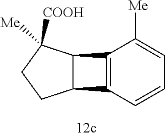 [SMILES: Cc1cccc2c1[C@H]1[C@@H]2CC[C@@]1(C)C(=O)O]

Following the general procedure for the [2+2] annulation of cyclic aliphatic acids and dihaloarenes, the compound was purified by pTLC (20% EtOAc/hexane+1% formic acid) to afford 12c in 62% yield (white solid, 13.4 mg, 0.062 mmol).

¹H NMR (600 MHz, CDCl₃) δ 7.14 (t, J=7.6 Hz, 1H), 6.97 (d, J=7.1 Hz, 1H), 6.85 (d, J=8.1 Hz, 1H), 3.84 (dd, J=7.4, 4.0 Hz, 1H), 3.75 (d, J=3.7 Hz, 1H), 2.13 (s, 3H), 2.12-2.07 (m, 1H), 1.91-1.76 (m, 2H), 1.60-1.53 (m, 1H), 1.31 (s, 3H). ¹³C NMR (151 MHz, CDCl₃) δ 182.82, 146.30, 142.01, 133.70, 128.95, 128.57, 118.99, 57.65, 49.17, 47.13, 32.65, 26.05, 22.41, 17.36.

HRMS (ESI-TOF) Calculated for C₁₄H₁₇O₂[M+H]⁺: 217.1229, Found: 217.1228.

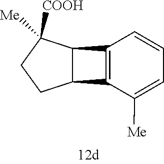 [SMILES: Cc1cccc2c1[C@H]1CC[C@@](C)(C(=O)O)[C@@H]21]

Following the general procedure for the [2+2] annulation of cyclic aliphatic acids and dihaloarenes, the compound was purified by pTLC (20% EtOAc/hexane+1% formic acid) to afford 12d in 66% yield (white solid, 14.3 mg, 0.066 mmol).

¹H NMR (600 MHz, CDCl₃) δ 7.11 (t, J=7.6 Hz, 1H), 7.02 (dq, J=7.8, 0.8 Hz, 1H), 6.87 (d, J=6.6 Hz, 1H), 3.91 (dd, J=7.4, 3.9 Hz, 1H), 3.65 (d, J=3.9 Hz, 1H), 2.20 (s, 3H), 1.97-1.78 (m, 3H), 1.65-1.59 (m, 1H), 1.34 (s, 3H). ¹³C NMR (151 MHz, CDCl₃) δ 182.64, 144.41, 144.15, 132.30, 129.13, 128.00, 120.39, 55.85, 49.71, 46.85, 32.50, 25.07, 22.90, 16.30.

HRMS (ESI-TOF) Calculated for C₁₄H₁₇O₂[M+H]⁺: 217.1229, Found: 217.1225.

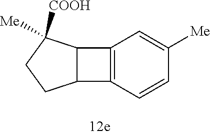 [SMILES: Cc1ccc2c(c1)C1C2CC[C@@]1(C)C(=O)O]

Following the general procedure for the [2+2] annulation of cyclic aliphatic acids and dihaloarenes, the compound was purified by pTLC (20% EtOAc/hexane+1% formic acid) to afford 12e in 65% yield (white solid, 14.1 mg, 0.065 mmol).

¹H NMR (600 MHz, CDCl₃) δ 7.04 (d, J=7.4 Hz, 1H), 6.92 (d, J=7.4 Hz, 1H), 6.87 (s, 1H), 3.88 (dd, J=3.9, 3.9 Hz, 1H), 3.66 (d, J=3.9 Hz, 1H), 2.28 (s, 3H), 1.97-1.89 (m, 1H), 1.87-1.81 (m, 2H), 1.65-1.60 (m, 1H), 1.34 (s, 3H). ¹³C NMR (151 MHz, CDCl₃) δ 182.68, 144.49, 143.17, 137.28, 129.16, 123.82, 121.71, 56.07, 49.87, 47.22, 32.40, 26.23, 22.97, 22.23.

HRMS (ESI-TOF) Calculated for C₁₄H₁₇O₂[M+H]⁺: 217.1229, Found: 217.1226.

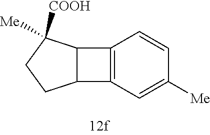 [SMILES: Cc1ccc2c(c1)C1CC[C@@](C)(C(=O)O)C21]

Following the general procedure for the [2+2] annulation of cyclic aliphatic acids and dihaloarenes, the compound was purified by pTLC (20% EtOAc/hexane+1% formic acid) to afford 12f in 72% yield (white solid, 15.6 mg, 0.072 mmol).

¹H NMR (600 MHz, CDCl₃) δ 7.01 (d, J=7.5 Hz, 1H), 6.94 (d, J=7.5 Hz, 1H), 6.86 (s, 1H), 3.89 (dd, J=3.9, 3.9 Hz, 1H), 3.65 (d, J=3.7 Hz, 1H), 2.33 (s, 3H), 1.96-1.88 (m, 1H), 1.87-1.81 (m, 2H), 1.64-1.58 (m, 1H), 1.33 (s, 3H). ¹³C NMR (151 MHz, CDCl₃) δ 182.73, 146.39, 141.33, 138.12, 128.41, 122.92, 122.63, 55.94, 49.86, 47.49, 32.43, 26.17, 22.85, 22.30.

HRMS (ESI-TOF) Calculated for C₁₄H₁₅O₂[M−H]⁻: 215.1072, Found: 215.1073.

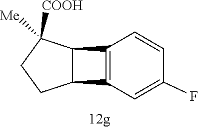 [SMILES: C[C@@]1(C(=O)O)CC[C@@H]2c3cc(F)ccc3[C@@H]21]

Following the general procedure for the [2+2] annulation of cyclic aliphatic acids and dihaloarenes, the compound was purified by pTLC (20% EtOAc/hexane+1% formic acid) to afford 12g in 84% yield (white solid, 18.5 mg, 0.084 mmol).

¹H NMR (600 MHz, CDCl₃) δ 6.99 (dd, J=8.1, 4.8 Hz, 1H), 6.91-6.84 (m, 1H), 6.77 (dd, J=7.8, 2.3 Hz, 1H), 3.88 (dd, J=3.5, 3.1 Hz, 1H), 3.63 (d, J=3.7 Hz, 1H), 1.93-1.83 (m, 3H), 1.68-1.61 (m, 1H), 1.34 (s, 3H). ¹³C NMR (151 MHz, CDCl₃) δ 182.65, 163.54 (d, J=244.8 Hz), 147.59 (d, J=6.6 Hz), 139.57 (d, J=2.8 Hz), 125.00 (d, J=8.8 Hz), 115.06 (d, J=23.2 Hz), 109.82 (d, J=22.0 Hz), 55.40, 49.90, 47.13, 32.37, 25.98, 22.83. ¹⁹F NMR (376 MHz, CDCl₃) δ −114.53.

HRMS (ESI-TOF) Calculated for C₁₃H₁₂FO₂ [M−H]⁻: 219.0821, Found: 219.0820.

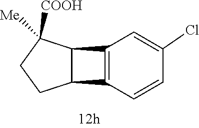 [SMILES: C[C@@]1(C(=O)O)CC[C@@H]2c3ccc(Cl)cc3[C@@H]21]

Following the general procedure for the [2+2] annulation of cyclic aliphatic acids and dihaloarenes, the compound was purified by pTLC (20% EtOAc/hexane+1% formic acid) to afford 12h in 92% yield (white solid, 21.8 mg, 0.092 mmol).

¹H NMR (600 MHz, CDCl₃) δ 7.21 (ddd, J=7.8, 1.9, 0.8 Hz, 1H), 7.06 (dt, J=1.8, 0.9 Hz, 1H), 6.95 (dt, J=7.8, 0.8 Hz, 1H), 3.90-3.85 (m, 1H), 3.67 (d, J=3.9 Hz, 1H), 1.94-1.82 (m, 3H), 1.70-1.62 (m, 1H), 1.35 (s, 3H).

¹³C NMR (151 MHz, CDCl₃) δ 182.29, 145.81, 144.48, 133.37, 128.90, 124.00, 123.51, 55.83, 49.91, 47.18, 32.42, 26.08, 22.97.

HRMS (ESI-TOF) Calculated for C₁₃H₁₄ClO₂ [M+H]⁺: 237.0682, Found: 237.0679.

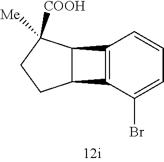 [SMILES: C[C@@]1(C(=O)O)CC[C@@H]2c3c(Br)cccc3[C@@H]21]

Following the general procedure for the [2+2] annulation of cyclic aliphatic acids and dihaloarenes, the compound was purified by pTLC (20% EtOAc/hexane+1% formic acid) to afford 12i in 79% yield (white solid, 22.2 mg, 0.079 mmol).

¹H NMR (600 MHz, CDCl₃) δ 7.33 (d, J=8.1 Hz, 1H), 7.07 (t, J=8.0 Hz, 1H), 6.99 (d, J=7.2 Hz, 1H), 3.91 (dd, J=6.6, 3.8 Hz, 1H), 3.67 (d, J=3.7 Hz, 1H), 2.07-2.01 (m, 1H), 1.93-1.78 (m, 2H), 1.70-1.63 (m, 1H), 1.35 (s, 3H). ¹³C NMR (151 MHz, CDCl₃) δ 182.58, 146.44, 145.41, 131.00, 129.67, 122.25, 115.45, 55.50, 49.74, 47.96, 32.47, 24.21, 22.92.

HRMS (ESI-TOF) Calculated for C₁₃H₁₄BrO₂ [M+H]⁺: 281.0177, Found: 281.0174.

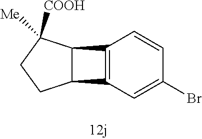 [SMILES: C[C@@]1(C(=O)O)CC[C@@H]2c3cc(Br)ccc3[C@@H]21]

Following the general procedure for the [2+2] annulation of cyclic aliphatic acids and dihaloarenes, the compound was purified by pTLC (20% EtOAc/hexane+1% formic acid) to afford 12j in 87% yield (white solid, 24.5 mg, 0.087 mmol).

¹H NMR (600 MHz, CDCl₃) δ 7.34 (dd, J=7.8, 1.7 Hz, 1H), 7.19 (s, 1H), 6.92 (d, J=7.8 Hz, 1H), 3.94-3.88 (m, 1H), 3.62 (d, J=3.7 Hz, 1H), 1.94-1.83 (m, 3H), 1.68-1.61 (m, 1H), 1.34 (s, 3H). ¹³C NMR (151 MHz, CDCl₃) δ 182.46, 148.04, 143.12, 130.99, 125.55, 125.29, 122.15, 55.80, 49.86, 47.80, 32.43, 26.06, 22.87.

HRMS (ESI-TOF) Calculated for C₁₃H₁₄BrO₂ [M+H]⁺: 281.0177, Found: 281.0172.

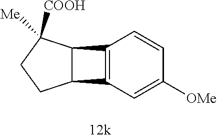 [SMILES: COc1ccc2c(c1)[C@H]1CC[C@@](C)(C(=O)O)[C@@H]21]

Following the general procedure for the [2+2] annulation of cyclic aliphatic acids and dihaloarenes, the compound was purified by pTLC (20% EtOAc/hexane+1% formic acid) to afford 12k in 79% yield (colorless oil, 18.4 mg, 0.079 mmol).

¹H NMR (600 MHz, CDCl₃) δ 6.95 (d, J=8.1 Hz, 1H), 6.74 (dd, J=8.1, 2.3 Hz, 1H), 6.63 (d, J=2.4 Hz, 1H), 3.86 (dd, J=3.9, 3.9 Hz, 1H), 3.77 (s, 3H), 3.62 (d, J=3.7 Hz, 1H), 1.96-1.87 (m, 1H), 1.86-1.82 (m, 2H), 1.64-1.58 (m, 1H), 1.32 (s, 3H). ¹³C NMR (151 MHz, CDCl₃) δ 182.38, 160.50, 147.20, 136.14, 124.35, 114.19, 107.73, 55.54, 49.84, 47.17, 32.44, 26.04, 22.79.

HRMS (ESI-TOF) Calculated for C₁₄H₁₇O₃[M+H]⁺: 233.1178, Found: 233.1179.

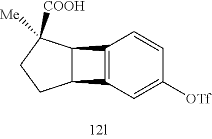 [SMILES: C[C@@]1(C(=O)O)CC[C@@H]2c3cc(OS(=O)(=O)C(F)(F)F)ccc3[C@@H]21]

Following the general procedure for the [2+2] annulation of cyclic aliphatic acids and dihaloarenes, the compound was purified by pTLC (20% EtOAc/hexane+1% formic acid) to afford 12l in 90% yield (colorless oil, 31.5 mg, 0.09 mmol).

¹H NMR (600 MHz, CDCl₃) δ 7.12 (d, J=8.1 Hz, 1H), 7.07 (dd, J=8.1, 2.2 Hz, 1H), 6.99 (d, J=2.3 Hz, 1H), 3.94 (dd, J=6.9, 3.8 Hz, 1H), 3.68 (d, J=3.7 Hz, 1H), 1.96-1.82 (m, 3H), 1.72-1.66 (m, 1H), 1.36 (s, 3H). ¹³C NMR (151 MHz, CDCl₃) δ 182.35, 149.96, 148.12, 144.77, 125.63, 121.20, 115.86, 118.88 (q, J=320.8 Hz), 55.58, 49.95, 47.42, 32.46, 25.92, 22.91. ¹⁹F NMR (376 MHz, CDCl₃) δ −75.59.

HRMS (ESI-TOF) Calculated for C₁₄H₁₂F₃O₅S [M−H]⁻: 349.0358, Found: 349.0349.

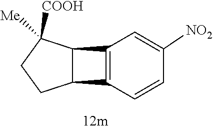 [SMILES: C[C@@]1(C(=O)O)CC[C@@H]2c3ccc([N+](=O)[O-])cc3[C@@H]21]

Following the general procedure for the [2+2] annulation of cyclic aliphatic acids and dihaloarenes, the compound was purified by pTLC (30% EtOAc/hexane+1% formic acid) to afford 12m in 85% yield (white solid, 21.0 mg, 0.085 mmol).

¹H NMR (600 MHz, CDCl₃) δ 8.18 (d, J=8.1 Hz, 1H), 7.89 (s, 1H), 7.18 (d, J=8.1 Hz, 1H), 3.99 (dd, J=8.0, 3.7 Hz, 1H), 3.76 (d, J=4.5 Hz, 1H), 2.02-1.83 (m, 3H), 1.72 (dd, J=12.8, 6.5 Hz, 1H), 1.39 (s, 3H). ¹³C NMR (151 MHz, CDCl₃) δ 181.13, 154.39, 148.41, 145.60, 124.77, 122.93, 119.20, 55.67, 50.01, 47.97, 32.55, 26.05, 22.82.

HRMS (ESI-TOF) Calculated for C₁₃H₁₂NO₄ [M−H]⁻: 246.0766, Found: 246.0765.

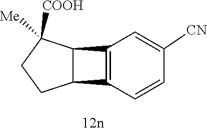 [SMILES: C[C@@]1(C(=O)O)CC[C@@H]2c3ccc(C#N)cc3[C@@H]21]

Following the general procedure for the [2+2] annulation of cyclic aliphatic acids and dihaloarenes, the compound was purified by pTLC (40% EtOAc/hexane+1% formic acid) to afford 12n in 78% yield (white solid, 17.7 mg, 0.078 mmol).

¹H NMR (600 MHz, CDCl₃) δ 7.54 (d, J=7.6 Hz, 1H), 7.34 (s, 1H), 7.14 (d, J=7.5 Hz, 1H), 3.98 (dd, J=7.5, 3.8 Hz, 1H), 3.74 (d, J=3.8 Hz, 1H), 2.00-1.82 (m, 3H), 1.75-1.69 (m, 1H), 1.38 (s, 3H). ¹³C NMR (151 MHz, CDCl₃) δ 181.60, 152.23, 145.63, 132.98, 127.24, 123.02, 119.74, 111.38, 56.07, 49.99, 48.39, 32.62, 25.97, 22.87.

HRMS (ESI-TOF) Calculated for C₁₄H₁₄NO₂ [M+H]⁺: 228.1025, Found: 228.1023.

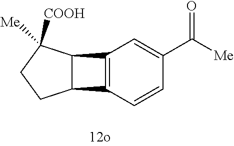 [SMILES: CC(=O)c1ccc2c(c1)[C@H]1[C@@H]2CC[C@@]1(C)C(=O)O]

Following the general procedure for the [2+2] annulation of cyclic aliphatic acids and dihaloarenes, the compound was purified by pTLC (20% EtOAc/hexane+1% formic acid) to afford 12o in 85% yield (colorless oil, 20.8 mg, 0.085 mmol).

¹H NMR (600 MHz, CDCl₃) δ 7.90 (ddd, J=7.7, 1.5, 0.6 Hz, 1H), 7.62 (s, 1H), 7.12 (d, J=7.7 Hz, 1H), 3.95 (dd, J=6.9, 3.7 Hz, 1H), 3.73 (d, J=4.1 Hz, 1H), 2.50 (s, 3H), 1.96-1.84 (m, 3H), 1.70-1.64 (m, 1H), 1.37 (s, 3H). ¹³C NMR (151 MHz, CDCl₃) δ 198.52, 181.86, 152.66, 144.98, 137.14, 129.31, 123.46, 122.15, 56.09, 49.99, 47.96, 32.52, 26.78, 26.08, 22.86.

HRMS (ESI-TOF) Calculated for C₁₅H₁₇O₃[M+H]⁺: 245.1178, Found: 245.1181.

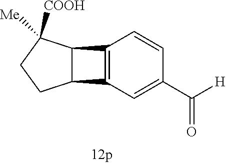 [SMILES: C[C@@]1(C(=O)O)CC[C@@H]2c3cc(C=O)ccc3[C@@H]21]

Following the general procedure for the [2+2] annulation of cyclic aliphatic acids and dihaloarenes, the compound was purified by pTLC (20% EtOAc/hexane+1% formic acid) to afford 12p in 78% yield (colorless oil, 18.0 mg, 0.078 mmol).

¹H NMR (600 MHz, CDCl₃) δ 9.94 (s, 1H), 7.73 (dd, J=7.5, 1.9 Hz, 1H), 7.59 (s, 1H), 7.22 (d, J=7.6 Hz, 1H), 3.99 (dd, J=8.0, 3.9 Hz, 1H), 3.75 (d, J=3.9 Hz, 1H), 1.98-1.83 (m, 3H), 1.71-1.65 (m, 1H), 1.38 (s, 3H). ¹³C NMR (151 MHz, CDCl₃) δ 192.69, 182.26, 152.49, 147.54, 137.20, 131.38, 123.97, 122.55, 56.51, 50.21, 47.52, 32.55, 26.17, 23.01.

HRMS (ESI-TOF) Calculated for C₁₄H₁₅O₃[M+H]⁺: 231.1021, Found: 231.1022.

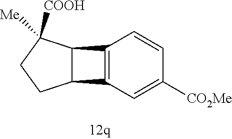 [SMILES: COC(=O)c1ccc2c(c1)[C@H]1CC[C@@](C)(C(=O)O)[C@@H]21]

Following the general procedure for the [2+2] annulation of cyclic aliphatic acids and dihaloarenes, the compound was purified by pTLC (20% EtOAc/hexane+1% formic acid) to afford 12q in 91% yield (white solid, 23.7 mg, 0.091 mmol).

¹H NMR (600 MHz, CDCl₃) δ 7.95 (dd, J=8.0, 1.3 Hz, 1H), 7.72 (s, 1H), 7.12 (d, J=7.7 Hz, 1H), 3.95 (dd, J=3.9, 3.7 Hz, 1H), 3.90 (s, 3H), 3.72 (d, J=3.9 Hz, 1H), 1.95-1.82 (m, 3H), 1.69-1.62 (m, 1H), 1.36 (s, 3H). ¹³C NMR (151 MHz, CDCl₃) δ 182.25, 167.69, 150.32, 146.58, 130.42, 129.69, 123.34, 123.31, 56.37, 52.21, 50.06, 47.55, 32.48, 26.14, 22.97.

HRMS (ESI-TOF) Calculated for C₁₅H₁₇O₄[M+H]⁺: 261.1127, Found: 261.1128.

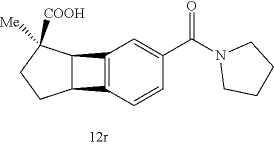 [SMILES: C[C@@]1(C(=O)O)CC[C@@H]2c3ccc(C(=O)N4CCCC4)cc3[C@@H]21]

Following the general procedure for the [2+2] annulation of cyclic aliphatic acids and dihaloarenes, the compound was purified by pTLC (60% EtOAc/hexane+1% formic acid) to afford 12r in 89% yield (white solid, 26.6 mg, 0.089 mmol).

¹H NMR (600 MHz, CDCl₃) δ 7.38 (d, J=7.6 Hz, 1H), 7.22 (s, 1H), 7.04 (d, J=7.4 Hz, 1H), 3.93-3.89 (m, 1H), 3.68 (d, J=3.9 Hz, 1H), 3.62 (t, J=7.0 Hz, 2H), 3.38 (t, J=6.7 Hz, 2H), 1.95-1.79 (m, 7H), 1.64-1.59 (m, 1H), 1.31 (s, 3H). ¹³C NMR (151 MHz, CDCl₃) δ 180.38, 170.79, 148.68, 144.65, 136.15, 127.51, 122.44, 121.74, 56.26, 49.99, 49.86, 47.81, 46.47, 32.59, 26.47, 26.16, 24.59, 22.84.

HRMS (ESI-TOF) Calculated for C₁₈H₂₂NO₃ [M+H]⁺: 300.1600, Found: 300.1602.

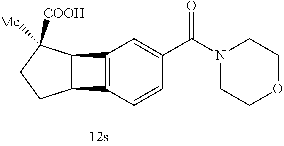 [SMILES: C[C@@]1(C(=O)O)CC[C@@H]2c3ccc(C(=O)N4CCOCC4)cc3[C@@H]21]

Following the general procedure for the [2+2] annulation of cyclic aliphatic acids and dihaloarenes, the compound was purified by pTLC (60% EtOAc/hexane+1% formic acid) to afford 12s in 89% yield (colorless oil, 28.1 mg, 0.089 mmol).

¹H NMR (600 MHz, CDCl₃) δ 7.29 (dd, J=7.5, 2.1 Hz, 1H), 7.08 (s, 1H), 7.07 (d, J=7.6 Hz, 1H), 3.95-3.90 (m, 1H), 3.89-3.21 (m, 9H), 1.91-1.83 (m, 3H), 1.69-1.60 (m, 1H), 1.33 (s, 3H). ¹³C NMR (151 MHz, CDCl₃) δ 180.87, 171.41, 148.78, 144.84, 134.25, 127.64, 122.32, 122.25, 67.00, 56.24, 49.87, 47.90, 32.62, 26.11, 22.81.

HRMS (ESI-TOF) Calculated for C₁₈H₂₂NO₄ [M+H]⁺: 316.1549, Found: 316.1552.

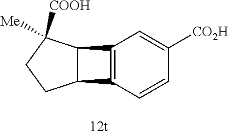 [SMILES: C[C@@]1(C(=O)O)CC[C@@H]2c3ccc(C(=O)O)cc3[C@@H]21]

Following the general procedure for the [2+2] annulation of cyclic aliphatic acids and dihaloarenes, the compound was purified by pTLC (50% EtOAc/hexane+1% formic acid) to afford 12t in 64% yield (white solid, 15.8 mg, 0.064 mmol).

¹H NMR (600 MHz, CD₃CN) δ 7.96-7.91 (m, 1H), 7.60 (s, 1H), 7.16 (d, J=7.6 Hz, 1H), 3.94 (dd, J=7.9, 3.8 Hz, 1H), 3.67 (d, J=3.6 Hz, 1H), 1.93-1.88 (m, 1H), 1.83 (dd, J=13.3, 6.8 Hz, 1H), 1.71 (td, J=13.1, 6.9 Hz, 1H), 1.56 (dd, J=12.9, 6.6 Hz, 1H), 1.29 (s, 3H). ¹³C NMR (151 MHz, CD₃CN) δ 177.83, 168.39, 153.87, 146.15, 131.25, 130.21, 125.08, 123.12, 56.86, 50.48, 48.79, 33.25, 26.55, 22.98.

HRMS (ESI-TOF) Calculated for C₁₄H₁₅O₄[M+H]⁺: 247.0970, Found: 247.0973.

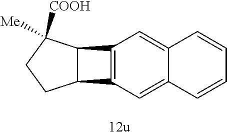 [SMILES: C[C@@]1(C(=O)O)CC[C@@H]2c3cc4ccccc4cc3[C@@H]21]

Following the general procedure for the [2+2] annulation of cyclic aliphatic acids and dihaloarenes, the compound was purified by pTLC (20% EtOAc/hexane+1% formic acid) to afford 12u in 67% yield (white solid, 16.9 mg, 0.067 mmol).

¹H NMR (600 MHz, CDCl₃) δ 7.79 (d, J=8.1 Hz, 1H), 7.72 (d, J=8.0 Hz, 1H), 7.49 (s, 1H), 7.45 (s, 1H), 7.42-7.32 (m, 2H), 4.10 (dd, J=3.9, 3.9 Hz, 1H), 3.89 (d, J=4.4 Hz, 1H), 2.04-1.91 (m, 3H), 1.71-1.65 (m, 1H), 1.43 (s, 3H). ¹³C NMR (151 MHz, CDCl₃) δ 182.22, 145.42, 143.32, 134.88, 134.38, 128.61, 128.17, 125.00, 124.80, 121.54, 119.99, 56.09, 51.06, 47.63, 32.64, 27.41, 23.34.

HRMS (ESI-TOF) Calculated for C₁₇H₁₇O₂[M+H]⁺: 253.1229, Found: 253.1225.

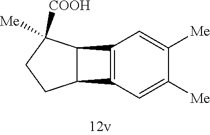 [SMILES: Cc1cc2c(cc1C)[C@H]1[C@@H]2CC[C@@]1(C)C(=O)O]

Following the general procedure for the [2+2] annulation of cyclic aliphatic acids and dihaloarenes, the compound was purified by pTLC (20% EtOAc/hexane+1% formic acid) to afford 12v in 54% yield (white solid, 12.4 mg, 0.054 mmol).

¹H NMR (600 MHz, CDCl₃) δ 6.82 (s, 1H), 6.81 (s, 1H), 3.87 (dd, J=3.7, 3.7 Hz, 1H), 3.65 (d, J=3.7 Hz, 1H), 2.23 (s, 3H), 2.19 (s, 3H), 1.98-1.91 (m, 1H), 1.84-1.79 (m, 2H), 1.65-1.58 (m, 1H), 1.32 (s, 3H). ¹³C NMR (151 MHz, CDCl₃) δ 182.24, 143.84, 142.00, 136.80, 135.94, 124.20, 123.03, 56.13, 49.75, 47.44, 32.46, 26.20, 22.89, 20.58, 20.51.

HRMS (ESI-TOF) Calculated for C₁₅H₁₉O₂[M+H]⁺: 231.1385, Found: 231.1387.

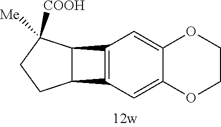 [SMILES: C[C@@]1(C(=O)O)CC[C@@H]2c3cc4c(cc3[C@@H]21)OCCO4]

Following the general procedure for the [2+2] annulation of cyclic aliphatic acids and dihaloarenes, the compound was purified by pTLC (30% EtOAc/hexane+1% formic acid) to afford 12w in 39% yield (white solid, 10.1 mg, 0.039 mmol).

¹H NMR (600 MHz, CDCl₃) δ 6.59 (s, 1H), 6.55 (s, 1H), 4.24-4.15 (m, 4H), 3.81 (dd, J=3.8, 3.8 Hz, 1H), 3.58 (d, J=3.8 Hz, 1H), 1.97-1.89 (m, 1H), 1.83-1.76 (m, 2H), 1.65-1.59 (m, 1H), 1.30 (s, 3H). ¹³C NMR (151 MHz, CDCl₃) δ 181.62, 144.68, 144.02, 138.73, 136.84, 112.96, 111.38, 64.39, 64.29, 55.53, 49.79, 47.07, 32.49, 26.30, 22.89.

HRMS (ESI-TOF) Calculated for C₁₅H₁₇O₄[M+H]⁺: 261.1127, Found: 261.1126.

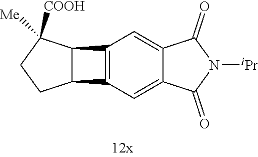 [SMILES: CC(C)N1C(=O)c2cc3c(cc2C1=O)[C@H]1[C@@H]3CC[C@@]1(C)C(=O)O]

Following the general procedure for the [2+2] annulation of cyclic aliphatic acids and dihaloarenes, the compound was purified by pTLC (50% EtOAc/hexane+1% formic acid) to afford 12x in 47% yield (white solid, 14.7 mg, 0.047 mmol).

¹H NMR (600 MHz, CDCl₃) δ 7.52 (s, 1H), 7.47 (s, 1H), 4.53-4.44 (m, 1H), 3.97 (dd, J=7.7, 3.9 Hz, 1H), 3.75 (d, J=4.1 Hz, 1H), 2.02-1.80 (m, 3H), 1.71 (dd, J=13.0, 6.4 Hz, 1H), 1.46 (s, 3H), 1.45 (s, 3H), 1.39 (s, 3H). ¹³C NMR (151 MHz, CDCl₃) δ 181.47, 169.09, 169.02, 153.42, 151.41, 132.64, 131.89, 118.94, 117.18, 55.87, 50.13, 47.53, 43.00, 32.54, 25.99, 22.93, 20.29.

HRMS (ESI-TOF) Calculated for C₁₈H₂₀NO₄ [M+H]⁺: 314.1392, Found: 314.1395.

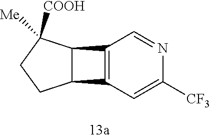 [SMILES: C[C@@]1(C(=O)O)CC[C@@H]2c3cc(C(F)(F)F)ncc3[C@@H]21]

Following the general procedure for the [2+2] annulation of cyclic aliphatic acids and dihaloarenes with modifications: aliphatic acid (0.2 mmol), dihaloarene (0.1 mmol), K₂CO₃ (3.5 equiv), the compound was purified by reverse phase chromatography (0%-50% MeCN/H₂O) using Biotage Isolera™ one with Biotage SNAP C18 to afford 13a in 59% yield (white solid, 16.0 mg, 0.059 mmol).

¹H NMR (600 MHz, CD₃OD) δ 8.26 (d, J=1.4 Hz, 1H), 7.58 (d, J=1.4 Hz, 1H), 4.08 (dd, J=8.1, 3.7 Hz, 1H), 3.88 (d, J=3.7 Hz, 1H), 2.03 (tdd, J=13.4, 8.2, 6.9 Hz, 1H), 1.96-1.90 (m, 1H), 1.77 (td, J=13.2, 6.8 Hz, 1H), 1.68 (dd, J=12.8, 6.5 Hz, 1H), 1.35 (s, 3H). ¹³C NMR (151 MHz, CD₃OD) δ 179.68, 160.09, 148.09 (q, J=34.0 Hz), 147.03 (q, J=1.7 Hz), 144.65, 123.08 (q, J=273.5 Hz), 116.25 (q, J=2.8 Hz). 57.81, 51.05, 50.10, 33.76, 26.21, 22.99. ¹⁹F NMR (376 MHz, CDCl₃) δ −67.69.

HRMS (ESI-TOF) Calculated for C₁₃H₁₃F₃NO₂ [M+H]⁺: 272.0898, Found: 272.0901.

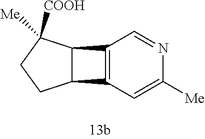 [SMILES: Cc1cc2c(cn1)[C@H]1[C@@H]2CC[C@@]1(C)C(=O)O]

Following the general procedure for the [2+2] annulation of cyclic aliphatic acids and dihaloarenes with modifications: aliphatic acid (0.2 mmol), dihaloarene (0.1 mmol), K₂CO₃ (3.5 equiv), the compound was purified by reverse phase chromatography (0%-50% MeCN/H₂O) using Biotage Isolera™ one with Biotage SNAP C18 to afford 13b in 32% yield (white solid, 6.9 mg, 0.032 mmol).

¹H NMR (600 MHz, CDCl₃) δ 8.12 (s, 1H), 6.95 (s, 1H), 4.00-3.95 (m, 1H), 3.81 (d, J=3.7 Hz, 1H), 2.59 (s, 3H), 2.00-1.86 (m, 3H), 1.66 (dd, J=13.5, 4.8 Hz, 1H), 1.36 (s, 3H). ¹³C NMR (151 MHz, CDCl₃) δ 179.52, 159.16, 156.45, 140.61, 140.05, 118.25, 56.74, 50.70, 49.26, 32.96, 26.19, 23.90, 22.75.

HRMS (ESI-TOF) Calculated for C₁₃H₁₆NO₂ [M+H]⁺: 218.1181, Found: 218.1185.

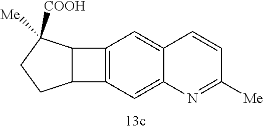 [SMILES: Cc1ccc2cc3c(cc2n1)C1CC[C@@](C)(C(=O)O)C31]

Following the general procedure for the [2+2] annulation of cyclic aliphatic acids and dihaloarenes with modifications: aliphatic acid (0.2 mmol), dihaloarene (0.1 mmol), K₂CO₃ (3.5 equiv), the compound was purified by reverse phase chromatography (0%-50% MeCN/H₂O) using Biotage Isolera™ one with Biotage SNAP C18 to afford 13c in 40% yield (white solid, 10.7 mg, 0.04 mmol).

¹H NMR (600 MHz, CDCl₃) δ 7.86 (d, J=8.4 Hz, 1H), 7.70 (s, 1H), 7.41 (s, 1H), 7.15 (d, J=8.4 Hz, 1H), 4.14-4.09 (m, 1H), 3.89 (d, J=4.2 Hz, 1H), 2.72 (s, 3H), 2.07-1.97 (m, 2H), 1.96-1.88 (m, 1H), 1.74-1.67 (m, 1H), 1.43 (s, 3H). ¹³C NMR (151 MHz, CDCl₃) δ 180.89, 157.52, 149.89, 148.92, 143.57, 137.17, 127.61, 121.51, 120.92, 120.56, 55.97, 51.16, 47.76, 32.76, 27.35, 24.91, 23.33.

HRMS (ESI-TOF) Calculated for C₁₇H₁₈NO₂ [M+H]⁺: 268.1338, Found: 268.1339.

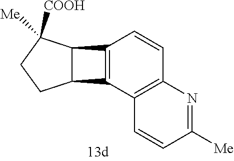 [SMILES: Cc1ccc2c3c(ccc2n1)[C@H]1[C@@H]3CC[C@@]1(C)C(=O)O]

Following the general procedure for the [2+2] annulation of cyclic aliphatic acids and dihaloarenes with modifications: aliphatic acid (0.2 mmol), dihaloarene (0.1 mmol), K₂CO₃ (3.5 equiv), the compound was purified by reverse phase chromatography (0%-50% MeCN/H₂O) using Biotage Isolera™ one with Biotage SNAP C18 to afford 13d in 43% yield (white solid, 11.5 mg, 0.043 mmol).

¹H NMR (600 MHz, CDCl₃) δ 8.01 (d, J=9.2 Hz, 1H), 7.70 (d, J=8.5 Hz, 1H), 7.30 (d, J=8.4 Hz, 1H), 7.24 (d, J=8.4 Hz, 1H), 4.20-4.15 (m, 1H), 3.81 (d, J=3.4 Hz, 1H), 2.80 (s, 3H), 2.03-1.92 (m, 3H), 1.75-1.67 (m, 1H), 1.41 (s, 3H). ¹³C NMR (151 MHz, CDCl₃) δ 179.59, 158.04, 147.65, 142.05, 141.43, 131.30, 128.44, 125.85, 122.31, 122.26, 56.64, 48.82, 46.66, 32.94, 25.38, 24.95, 22.79.

HRMS (ESI-TOF) Calculated for C₁₇H₁₈NO₂ [M+H]⁺: 268.1338, Found: 268.1339.

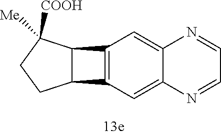 [SMILES: C[C@@]1(C(=O)O)CC[C@@H]2c3cc4nccnc4cc3[C@@H]21]

Following the general procedure for the [2+2] annulation of cyclic aliphatic acids and dihaloarenes with modifications: aliphatic acid (0.2 mmol), dihaloarene (0.1 mmol), K₂CO₃ (3.5 equiv), the compound was purified by pTLC (50% EA/hexane+1% formic acid) to afford 13e in 49% yield (white solid, 12.5 mg, 0.049 mmol).

¹H NMR (600 MHz, CDCl₃) δ 8.86 (d, J=1.9 Hz, 1H), 8.77 (d, J=2.1 Hz, 1H), 8.10 (t, J=1.1 Hz, 1H), 7.78 (t, J=1.0 Hz, 1H), 4.22-4.15 (m, 1H), 4.01 (d, J=4.3 Hz, 1H), 2.15-2.04 (m, 2H), 1.94 (td, J=13.1, 7.2 Hz, 1H), 1.79 (dd, J=13.8, 6.0 Hz, 1H), 1.47 (s, 3H). ¹³C NMR (151 MHz, CDCl₃) δ 179.19, 151.54, 150.02, 145.34, 143.76, 142.61, 141.61, 122.58, 122.05, 56.17, 51.53, 48.04, 33.19, 27.47, 23.71.

HRMS (ESI-TOF) Calculated for C₁₅H₁₅N₂O₂ [M+H]⁺: 255.1134, Found: 255.1134.

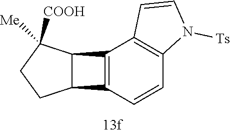 [SMILES: Cc1ccc(S(=O)(=O)n2ccc3c4c(ccc32)[C@H]2CC[C@@](C)(C(=O)O)[C@@H]42)cc1]

Following the general procedure for the [2+2] annulation of cyclic aliphatic acids and dihaloarenes, the compound was purified by pTLC (50% EtOAc/hexane+1% formic acid) to afford 13f in 48% yield (white solid, 18.9 mg, 0.048 mmol).

¹H NMR (600 MHz, CDCl₃) δ 7.91 (d, J=8.2 Hz, 1H), 7.73-7.67 (m, 2H), 7.32 (d, J=3.7 Hz, 1H), 7.16 (d, J=8.0 Hz, 2H), 6.97 (d, J=8.2 Hz, 1H), 6.39 (dd, J=3.7, 0.9 Hz, 1H), 3.95-3.91 (m, 1H), 3.80 (d, J=3.6 Hz, 1H), 2.30 (s, 3H), 1.88-1.80 (m, 3H), 1.62-1.55 (m, 1H), 1.35 (s, 3H). ¹³C NMR (151 MHz, CDCl₃) δ 182.58, 144.96, 140.91, 135.88, 135.41, 135.27, 129.95, 127.19, 126.96, 126.53, 117.79, 114.04, 106.64, 56.47, 49.22, 47.76, 32.41, 25.77, 22.85, 21.68.

HRMS (ESI-TOF) Calculated for C₂₂H₂₂NO₄S [M+H]⁺: 396.1270, Found: 396.1266.

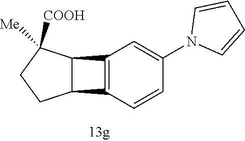 [SMILES: C[C@@]1(C(=O)O)CC[C@@H]2c3ccc(-n4cccc4)cc3[C@@H]21]

Following the general procedure for the [2+2] annulation of cyclic aliphatic acids and dihaloarenes, the compound was purified by pTLC (20% EtOAc/hexane+1% formic acid) to afford 13g in 41% yield (white solid, 11.0 mg, 0.041 mmol).

¹H NMR (600 MHz, CDCl₃) δ 7.25 (dddd, J=7.8, 2.0, 0.7 Hz, 1H), 7.09-7.04 (m, 2H), 6.98 (t, J=2.2 Hz, 2H), 6.28 (t, J=2.1 Hz, 2H), 3.93 (dd, J=3.7, 3.7 Hz, 1H), 3.70 (d, J=3.7 Hz, 1H), 1.99-1.85 (m, 3H), 1.73-1.60 (m, 1H), 1.35 (s, 3H). ¹³C NMR (151 MHz, CDCl₃) δ 180.05, 145.64, 143.85, 140.81, 123.09, 121.85, 119.91, 116.66, 110.05, 55.79, 49.73, 47.33, 32.56, 26.23, 22.96.

HRMS (ESI-TOF) Calculated for C₁₇H₁₈NO₂ [M+H]⁺: 268.1338, Found: 268.1338.

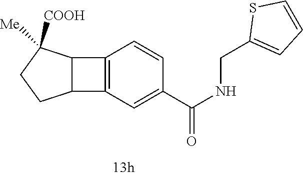 [SMILES: C[C@@]1(C(=O)O)CCC2c3cc(C(=O)NCc4cccs4)ccc3C21]

Following the general procedure for the [2+2] annulation of cyclic aliphatic acids and dihaloarenes, the compound was purified by pTLC (70% EtOAc/hexane+1% formic acid) to afford 13h in 81% yield (white solid, 27.7 mg, 0.081 mmol).

¹H NMR (600 MHz, CDCl₃) δ 7.69 (dd, J=7.8, 1.6 Hz, 1H), 7.48 (s, 1H), 7.22 (dd, J=5.1, 1.3 Hz, 1H), 7.06-7.00 (m, 2H), 6.95 (dd, J=5.1, 3.4 Hz, 1H), 6.82 (t, J=5.7 Hz, 1H), 4.80 (d, J=5.7 Hz, 2H), 3.96-3.91 (m, 1H), 3.70 (d, J=3.8 Hz, 1H), 1.91-1.82 (m, 3H), 1.68-1.60 (m, 1H), 1.35 (s, 3H). ¹³C NMR (151 MHz, CDCl₃) δ 182.07, 167.98, 148.93, 146.89, 141.17, 134.42, 127.14, 127.05, 126.26, 125.35, 123.36, 120.94, 56.32, 50.07, 47.69, 39.04, 32.44, 26.12, 22.96.

HRMS (ESI-TOF) Calculated for C₁₉H₂₀NO₃S [M+H]⁺: 342.1164, Found: 342.1161.

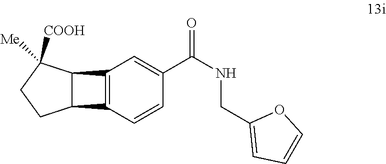 [SMILES: C[C@@]1(C(=O)O)CC[C@@H]2c3ccc(C(=O)NCc4ccco4)cc3[C@@H]21]

Following the general procedure for the [2+2] annulation of cyclic aliphatic acids and dihaloarenes, the compound was purified by pTLC (70% EtOAc/hexane+1% formic acid) to afford 13i in 62% yield (white solid, 20.2 mg, 0.062 mmol).

¹H NMR (600 MHz, CDCl₃) δ 7.71 (dd, J=7.6, 1.4 Hz, 1H), 7.51 (s, 1H), 7.31 (dd, J=1.9, 0.9 Hz, 1H), 7.09 (d, J=7.6 Hz, 1H), 6.66 (t, J=6.0 Hz, 1H), 6.29 (dd, J=3.2, 1.9 Hz, 1H), 6.23 (d, J=3.2 Hz, 1H), 4.54 (qd, J=15.5, 5.5 Hz, 2H), 3.96-3.87 (m, 1H), 3.71 (d, J=3.7 Hz, 1H), 1.94-1.82 (m, 3H), 1.63 (q, J=4.7 Hz, 1H), 1.34 (s, 3H). ¹³C NMR (151 MHz, CDCl₃) δ 180.45, 168.19, 151.48, 150.72, 144.99, 133.83, 127.86, 122.15, 122.09, 110.60, 107.69, 56.27, 49.83, 47.86, 37.18, 32.57, 26.16, 22.72.

HRMS (ESI-TOF) Calculated for C₁₉H₂₀NO₄ [M+H]⁺: 326.1392, Found: 326.1396.

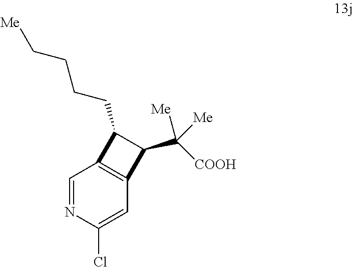

Following the general procedure for the [2+2] annulation of cyclic aliphatic acids and dihaloarenes with modifications: L28 instead of L7, KHCO₃ instead of K₂CO₃, the compound was purified by reverse phase chromatography (0%-50% MeCN/H₂O) using Biotage Isolera™ one with Biotage SNAP C₁₈ to afford 13j in 34% yield (white solid, 10.1 mg, 0.034 mmol).

¹H NMR (600 MHz, CDCl₃) δ 8.11 (q, J=1.2 Hz, 1H), 7.10 (s, 1H), 3.44 (dd, J=2.7, 0.9 Hz, 1H), 3.35 (ddd, J=8.9, 6.3, 2.7 Hz, 1H), 1.78 (ddt, J=13.1, 9.9, 6.3 Hz, 1H), 1.62 (dtd, J=14.1, 9.4, 5.3 Hz, 1H), 1.47-1.40 (m, 2H), 1.36-1.28 (m, 4H), 1.26 (s, 3H), 1.24 (s, 3H), 0.93-0.86 (m, 3H). ¹³C NMR (151 MHz, CDCl₃) δ 182.24, 160.77, 149.20, 143.39, 139.86, 118.95, 56.40, 46.64, 43.37, 33.20, 31.88, 27.95, 22.90, 22.66, 22.32, 14.15.

HRMS (ESI-TOF) Calculated for C₁₆H₂₃ClNO₂ [M+H]⁺: 296.1417, Found: 296.1417.

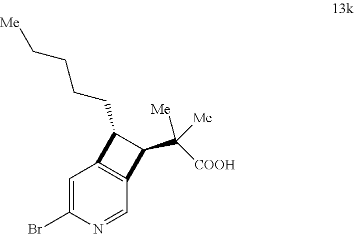

Following the general procedure for the [2+2] annulation of cyclic aliphatic acids and dihaloarenes with modifications: L28 instead of L7, KHCO₃ instead of K₂CO₃, the compound was purified by reverse phase chromatography (0%-50% MeCN/H₂O) using Biotage Isolera™ one with Biotage SNAP C₁₈ to afford 13k in 25% yield (white solid, 8.5 mg, 0.025 mmol).

¹H NMR (600 MHz, CDCl₃) δ 8.10 (s, 1H), 7.26 (s, 1H), 3.42 (d, J=2.8 Hz, 1H), 3.36 (td, J=6.3, 3.1 Hz, 1H), 1.83-1.74 (m, 1H), 1.62 (qd, J=9.0, 4.8 Hz, 1H), 1.48-1.41 (m, 2H), 1.35-1.30 (m, 4H), 1.26 (s, 3H), 1.25 (s, 3H), 0.93-0.88 (m, 3H). ¹³C NMR (151 MHz, CDCl₃) δ 181.81, 160.39, 144.06, 140.23, 139.72, 122.66, 56.50, 46.75, 43.32, 33.24, 31.90, 27.97, 22.95, 22.67, 22.35, 14.18.

HRMS (ESI-TOF) Calculated for C₁₆H₂₃BrNO₂ [M+H]⁺: 340.0912, Found: 342.0893.

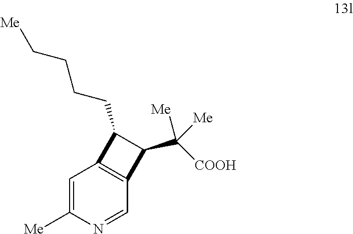

Following the general procedure for the [2+2] annulation of cyclic aliphatic acids and dihaloarenes with modifications: L28 instead of L7, KHCO₃ instead of K₂CO₃, the compound was purified by reverse phase chromatography (0%-50% MeCN/H₂O) using Biotage Isolera™ one with Biotage SNAP C₁₈ to afford 13l in 47% yield (white solid, 12.9 mg, 0.047 mmol).

¹H NMR (600 MHz, CDCl₃) δ 8.23 (s, 1H), 6.92 (s, 1H), 3.43 (d, J=2.6 Hz, 1H), 3.30 (ddd, J=9.1, 6.1, 2.5 Hz, 1H), 2.51 (s, 3H), 1.78-1.70 (m, 1H), 1.60-1.51 (m, 1H), 1.47-1.37 (m, 2H), 1.32-1.24 (m, 4H), 1.19 (s, 3H), 1.12 (s, 3H), 0.90-0.84 (m, 3H). ¹³C NMR (151 MHz, CDCl₃) δ 182.02, 159.86, 155.80, 141.48, 139.56, 117.90, 57.47, 46.76, 43.68, 33.51, 32.02, 28.15, 24.05, 23.30, 22.70, 22.57, 14.19.

HRMS (ESI-TOF) Calculated for C₁₇H₂₆NO₂ [M+H]⁺: 276.1964, Found: 276.1964.

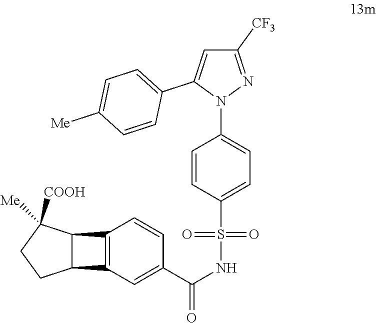 [SMILES: Cc1ccc(-c2cc(C(F)(F)F)nn2-c2ccc(S(=O)(=O)NC(=O)c3ccc4c(c3)[C@H]3CC[C@@](C)(C(=O)O)[C@@H]43)cc2)cc1]

Following the general procedure for the [2+2] annulation of cyclic aliphatic acids and dihaloarenes with modifications: aliphatic acid (0.2 mmol), 2a (0.1 mmol), K₂CO₃ (3.5 equiv), the compound was purified by pTLC (3% MeOH in EtOAc+1% formic acid) to afford 13m in 41% yield (white solid, 25.0 mg, 0.041 mmol).

¹H NMR (600 MHz, CDCl₃) δ 8.12-8.07 (m, 2H), 7.58 (dd, J=7.7, 1.7 Hz, 1H), 7.48-7.40 (m, 3H), 7.15 (d, J=8.5 Hz, 2H), 7.12-7.07 (m, 2H), 7.02 (d, J=7.8 Hz, 1H), 6.73 (s, 1H), 3.89 (dd, J=7.4, 3.9 Hz, 1H), 3.67 (d, J=3.8 Hz, 1H), 2.35 (s, 3H), 1.89-1.70 (m, 3H), 1.60 (dd, J=12.8, 6.4 Hz, 1H), 1.32 (s, 3H). ¹³C NMR (151 MHz, CDCl₃) δ 181.58, 165.22, 151.18, 147.25, 145.53, 144.30 (q, J=38.7 Hz), 143.51, 140.01, 137.97, 131.36, 129.94, 129.88, 128.84, 127.92, 125.68, 125.27, 123.65, 121.90, 121.12 (d, J=269.3 Hz), 106.58, 56.48, 50.02, 47.70, 32.53, 26.17, 26.04, 21.44. ¹⁹F NMR (376 MHz, CDCl₃) δ −65.09.

HRMS (ESI-TOF) Calculated for C₃₁H₂₇F₃N₃O₅S [M+H]⁺: 610.1624, Found: 610.1627.

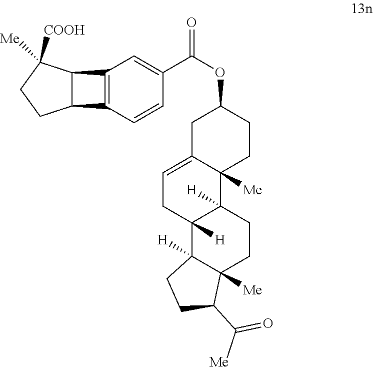

Following the general procedure for the [2+2] annulation of cyclic aliphatic acids and dihaloarenes, the compound was purified by pTLC (20% EtOAc/hexane+1% formic acid) to afford 13n in 86% yield (white solid, 46.8 mg, 0.086 mmol). A 1:1 diastereomers were obtained as the product. Some ¹³C peaks overlap.

¹H NMR (600 MHz, CDCl₃) δ 7.98 (dd, J=7.8, 1.6 Hz, 1H), 7.71 (s, 1H), 7.09 (d, J=7.8 Hz, 1H), 5.41-5.36 (m, 1H), 4.85-4.72 (m, 1H), 3.94 (q, J=3.7 Hz, 1H), 3.71 (d, J=3.8 Hz, 1H), 2.55 (t, J=9.0 Hz, 1H), 2.43 (s, 2H), 2.23-2.16 (m, 1H), 2.14 (s, 3H), 2.07-1.86 (m, 7H), 1.74-1.57 (m, 6H), 1.53-1.44 (m, 3H), 1.35 (d, J=0.9 Hz, 3H), 1.28-1.15 (m, 3H), 1.07-1.00 (m, 4H), 0.64 (s, 3H). ¹³C NMR (151 MHz, CDCl₃) δ 209.65, 164.45, 140.52, 139.53, 133.47, 132.39, 130.12, 129.12, 122.87, 107.52, 75.37, 63.82, 56.98, 50.04, 44.13, 38.94, 38.20, 37.13, 36.79, 31.97, 31.94, 31.71, 27.91, 24.64, 22.99, 21.20, 19.50, 13.38.

HRMS (ESI-TOF) Calculated for C₃₅H₄₅O₅ [M+H]⁺: 545.3267, Found: 545.3262.

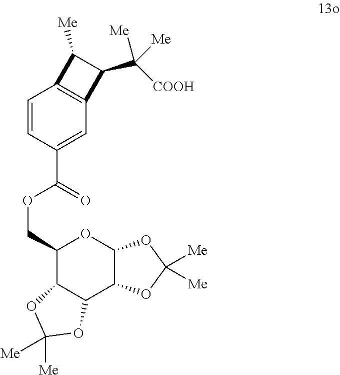

Following the general procedure for the [2+2] annulation of acyclic aliphatic acids and haloarenes, the compound was purified by pTLC (30% EtOAc/hexane+1% formic acid) to afford 13o in 47% yield (colorless oil, 23.1 mg, 0.047 mmol). A 1:1 diastereomers were obtained as the product. ¹H NMR and ¹³C NMR were reported as a mixture of diastereomers. Some peaks overlap.

¹H NMR (600 MHz, CDCl₃) δ 7.97 (dt, J=7.7, 1.0 Hz, 1H), 7.80 (dt, J=2.3, 1.2 Hz, 1H), 7.12 (d, J=7.6 Hz, 1H), 5.56 (d, J=4.9 Hz, 1H), 4.65 (dd, J=7.9, 2.4 Hz, 1H), 4.51 (ddd, J=16.3, 11.5, 4.8 Hz, 1H), 4.41 (ddd, J=14.1, 11.5, 7.6 Hz, 1H), 4.33 (dd, J=4.9, 2.5 Hz, 1H), 4.31 (dd, J=7.9, 1.9 Hz, 1H), 4.19 (ddt, J=8.0, 4.7, 1.6 Hz, 1H), 3.41 (qd, J=7.0, 2.5 Hz, 1H), 3.33 (dd, J=2.6, 1.1 Hz, 1H), 1.51 (s, 3H), 1.47 (s, 3H), 1.40 (d, J=7.0 Hz, 3H), 1.35 (s, 3H), 1.33 (s, 3H), 1.32 (d, J=3.0 Hz, 3H), 1.20 (s, 3H). ¹³C NMR (151 MHz, CDCl₃) δ 182.90, 182.83, 167.11, 167.08, 154.87, 143.95, 143.93, 130.05, 129.35, 129.32, 124.82, 121.58, 109.84, 109.00, 96.45, 71.30, 71.29, 70.87, 70.71, 66.38, 66.37, 63.98, 58.37, 58.35, 43.55, 40.88, 26.16, 26.15, 26.11, 25.14, 24.62, 23.59, 23.51, 21.58, 21.55, 18.53.

HRMS (ESI-TOF) Calculated for C₂₆H₃₅O₉[M+H]⁺: 491.2281, Found: 491.2285.

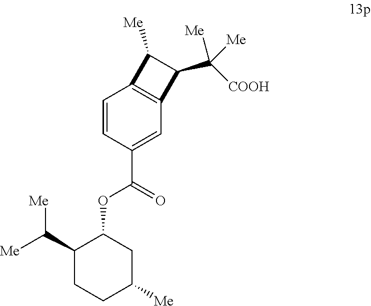

Following the general procedure for the [2+2] annulation of acyclic aliphatic acids and haloarenes, the compound was purified by pTLC (30% EtOAc/hexane+1% formic acid) to afford 13p in 42% yield (colorless oil, 16.2 mg, 0.042 mmol). A 1:1 diastereomers were obtained as the product. ¹H NMR and ¹³C NMR were reported as a mixture of diastereomers. Some peaks overlap.

¹H NMR (600 MHz, CDCl₃) δ 7.97 (ddt, J=7.7, 4.2, 1.0 Hz, 1H), 7.78 (dq, J=12.3, 1.1 Hz, 1H), 7.12 (dd, J=7.7, 1.0 Hz, 1H), 4.92 (tdd, J=10.9, 4.4, 1.5 Hz, 1H), 3.41 (qt, J=7.0, 2.5 Hz, 1H), 3.34 (dd, J=4.3, 2.7 Hz, 1H), 2.11 (dtd, J=12.1, 4.0, 1.7 Hz, 1H), 1.95 (dqd, J=16.6, 6.9, 3.5 Hz, 1H), 1.80-1.68 (m, 2H), 1.61-1.48 (m, 2H), 1.40 (d, J=7.0 Hz, 3H), 1.34 (d, J=7.2 Hz, 3H), 1.21 (d, J=2.2 Hz, 3H), 1.17-1.05 (m, 2H), 0.92 (ddd, J=7.1, 4.5, 2.3 Hz, 7H), 0.79 (dd, J=6.9, 3.1 Hz, 3H). ¹³C NMR (151 MHz, CDCl₃) δ 183.09, 166.76, 154.53, 143.90, 143.88, 130.18, 130.15, 129.91, 129.82, 124.73, 124.65, 121.50, 74.89, 74.84, 58.36, 58.35, 47.41, 47.39, 43.58, 43.56, 41.15, 41.12, 40.84, 40.80, 34.49, 31.60, 31.58, 26.76, 26.56, 23.92, 23.72, 23.59, 23.53, 22.20, 21.58, 21.57, 20.96, 20.86, 18.58, 16.80, 16.62.

HRMS (ESI-TOF) Calculated for C₂₄H₃₃O₄ [M−H]⁻: 385.2379, Found: 385.2372.

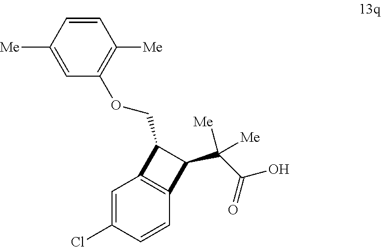

Following the general procedure for the [2+2] annulation of acyclic aliphatic acids and dihaloarenes, the compound was purified by pTLC (20% EtOAc/hexane+1% formic acid) to afford 13q in 45% yield (white solid, 16.2 mg, 0.045 mmol).

¹H NMR (600 MHz, CDCl₃) δ 7.23 (d, J=7.7 Hz, 1H), 7.17 (d, J=2.1 Hz, 1H), 7.08 (d, J=8.0 Hz, 1H), 7.01 (d, J=7.5 Hz, 1H), 6.68 (d, J=7.5 Hz, 1H), 6.62 (s, 1H), 4.25 (ddd, J=8.1, 6.2, 1.6 Hz, 1H), 4.15-4.09 (m, 1H), 3.77 (t, J=7.1 Hz, 1H), 3.59-3.55 (m, 1H), 2.30 (s, 3H), 2.18 (s, 3H), 1.31 (s, 3H), 1.31 (s, 3H). ¹³C NMR (151 MHz, CDCl₃) δ 182.49, 156.84, 146.16, 142.48, 136.74, 133.65, 130.60, 128.49, 124.98, 123.60, 123.43, 121.16, 111.89, 69.46, 53.94, 44.93, 43.45, 22.75, 22.57, 21.53, 16.10.

HRMS (ESI-TOF) Calculated for C₂₁H₂₂ClO₃ [M−H]⁻: 357.1257, Found: 357.1251.

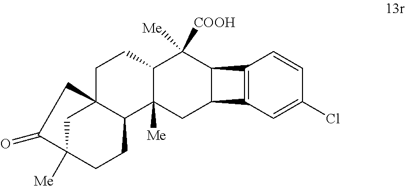 [SMILES: C[C@]12CC[C@H]3[C@](CC[C@H]4[C@]3(C)C[C@@H]3c5cc(Cl)ccc5[C@@H]3[C@]4(C)C(=O)O)(CC1=O)C2]

Following the general procedure for the [2+2] annulation of cyclic aliphatic acids and dihaloarenes using aliphatic acid (0.2 mmol), 2a (0.1 mmol), K₂CO₃ (3.5 equiv), the compound was purified by pTLC (20% EtOAc/hexane+1% formic acid) to afford 13r in 35% yield (white solid, 14.9 mg, 0.035 mmol).

¹H NMR (600 MHz, CDCl₃) δ 7.09 (dd, J=8.0, 2.0 Hz, 1H), 7.03 (s, 1H), 6.90 (d, J=7.9 Hz, 1H), 3.85-3.81 (m, 1H), 3.56 (d, J=5.7 Hz, 1H), 2.69-2.63 (m, 1H), 2.25 (d, J=13.9 Hz, 1H), 1.84-1.75 (m, 3H), 1.66-1.62 (m, 3H), 1.58 (s, 3H), 1.54-1.46 (m, 3H), 1.42-1.37 (m, 3H), 1.21-1.12 (m, 2H), 0.98 (s, 3H), 0.45 (s, 3H). ¹³C NMR (151 MHz, CDCl₃) δ 222.37, 177.74, 150.44, 144.65, 133.73, 127.65, 123.93, 121.78, 55.37, 55.15, 54.52, 48.87, 48.63, 48.14, 47.01, 41.65, 41.27, 40.62, 39.85, 38.11, 37.30, 31.88, 23.74, 20.84, 19.95, 17.45.

HRMS (ESI-TOF) Calculated for C₂₆H₃₂ClO₃ [M+H]⁺: 427.2040, Found: 427.2039.

### Synthetic Transformations

### Synthesis of 6

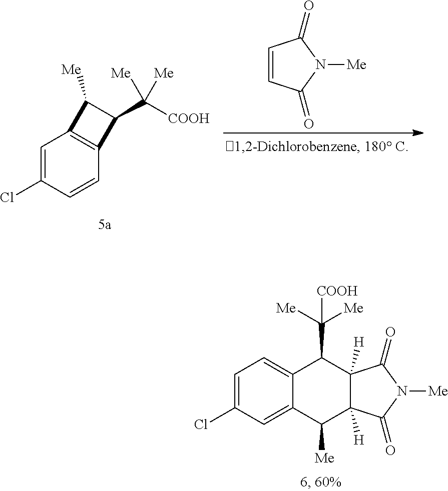

The mixture of 5a (0.1 mmol, 23.8 mg) and N-methylmaleimide (0.2 mmol, 22.2 mg) in o-dichlorobenzene (0.3 mL) was stirred at 180° C. for 72 h. Solvent was evaporated and the residue was purified by chromatography on silica gel using EtOAc/hexane as eluent to give the cycloaddition product 6 (60% yield, 0.06 mmol, 21.0 mg) as colorless oil.

¹H NMR (600 MHz, CDCl₃) δ 7.17-7.08 (m, 3H), 3.84 (s, 1H), 3.29-3.19 (m, 2H), 2.98 (dd, J=9.1, 3.6 Hz, 1H), 2.91 (s, 3H), 1.61 (d, J=7.4 Hz, 3H), 1.29 (s, 3H), 1.20 (s, 3H). ¹³C NMR (151 MHz, CDCl₃) δ 181.79, 180.09, 178.91, 141.40, 133.84, 133.24, 129.64, 129.34, 126.37, 47.06, 44.93, 43.84, 41.82, 34.12, 25.60, 25.57, 25.41, 23.22.

HRMS (ESI-TOF) Calculated for C₁₈H₂₁ClNO₄ [M+H]⁺: 350.1159, Found: 350.1155.

### Synthesis of 7

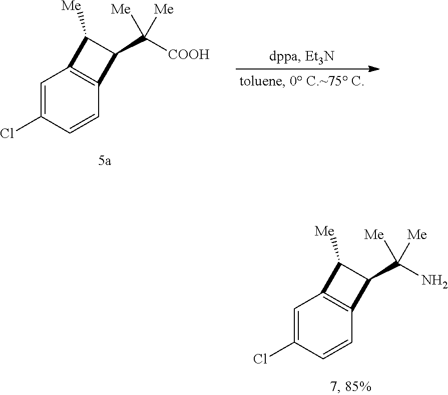

To a solution of 5a (0.1 mmol, 23.8 mg) in toluene (1.0 mL), Et₃N (0.11 mmol, 11.1 mg) was added and cooled to 0° C. To this cooled mixture was added diphenylphosphonicazide (dppa, 0.11 mmol, 30.3 mg) and the mixture was stirred at room temperature for 15 mins and then heated to 75° C. for 2.5 h. Then the reaction was cooled to room temperature, quenched with water, and extracted with EtOAc. The organic phase was washed with brine, dried over Na₂SO₄, and concentrated in vacuo. The residue was purified by chromatography on silica gel using EtOAc/hexane to afford the expected product 7 (85% yield, 0.085 mmol, 17.8 mg) as colorless oil.

¹H NMR (600 MHz, CDCl₃) δ 7.21 (ddd, J=7.8, 1.9, 0.9 Hz, 1H), 7.12-7.08 (m, 1H), 7.06 (dt, J=7.8, 1.0 Hz, 1H), 3.38 (qd, J=7.1, 2.5 Hz, 1H), 3.01 (d, J=2.5 Hz, 1H), 1.45 (s, 3H), 1.41 (d, J=7.0 Hz, 3H), 1.40 (s, 3H). ¹³C NMR (151 MHz, CDCl₃) δ 149.98, 140.81, 133.99, 128.07, 124.65, 122.67, 61.67, 59.45, 40.78, 28.65, 28.30, 18.55.

HRMS (ESI-TOF) Calculated for C₁₂H₁₇ClN [M+H]⁺: 210.1050, Found: 210.1048.

### Synthesis of 10

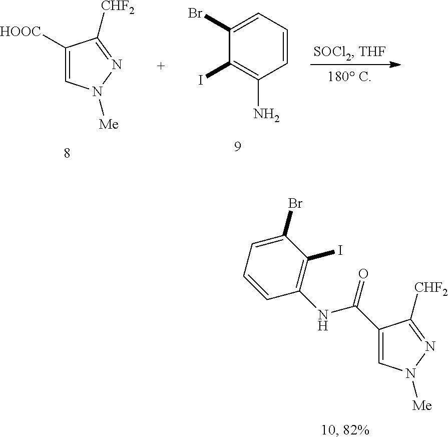

To a dry 12 mL reaction tube charged a stir bar added 8 (0.5 mmol, 88.1 mg) and 0.5 mL thionyl chloride (SOCl₂). The reaction tube was capped, and put into a heating bath of 80° C., stirred under this temperature for 1 h. After cooling down to room temperature, the cap was removed (inner pressure can be high. When scaling up, condenser with balloon is recommended). SOCl₂ in the reaction tube was removed by a rotavapor. Then 9 (0.6 mmol, 0.179 g) in 0.3 mL dry THE was added dropwisely into the reaction tube under room temperature. The reaction tube was capped and heated under 100° C. for 1 h. After cooling down to room temperature, water (1 mL) was added into the reaction mixture. The mixture was extracted by EtOAc (3×1 mL) and washed by brine (2×1 mL). The organic phase was combined, dried by anhydrous Na₂SO₄, and the solvent was removed by a rotavapor. The mixture was purified by a flash chromatography on silica gel (0~20% EtOAc/hexane), to afford 10 (82% yield, 0.41 mmol, 187.8 mg) as a white solid.

¹H NMR (600 MHz, Acetone-d₆) δ 8.79 (s, 1H), 8.45 (s, 1H), 7.81 (ddd, J=8.3, 6.3, 1.5 Hz, 1H), 7.59 (dd, J=8.0, 1.4 Hz, 1H), 7.38 (t, J=8.0 Hz, 1H), 7.30 (t, J=54.1 Hz, 1H), 4.04 (s, 3H). ¹³C NMR (151 MHz, Acetone-d₆) δ 160.85, 146.54 (t, J=24.3 Hz), 142.57, 133.75, 131.01, 130.96, 130.57, 125.04, 117.03, 110.99 (t, J=234.3 Hz), 102.89, 40.07. ¹⁹F NMR (376 MHz, CDCl₃) δ −113.02.

HRMS (ESI-TOF) Calculated for C₁₂H₁₀BrF₂IN₃O [M+H]⁺: 455.9020, Found: 455.9018.

### Synthesis of 11

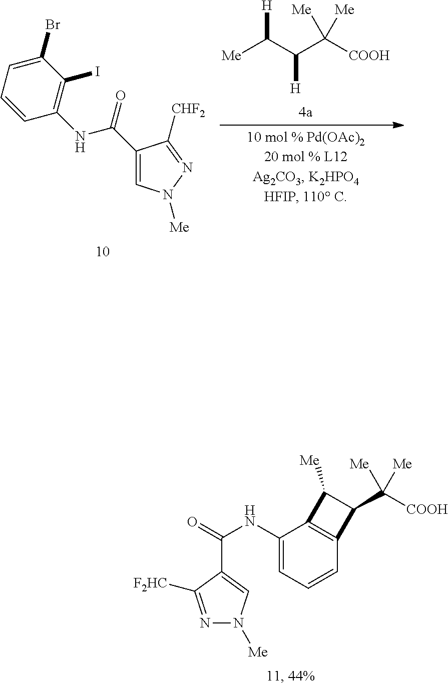

Synthesis of 11 followed general procedure for the [2+2] annulation of acyclic aliphatic acids and haloarenes with a modification: all components excepted from 10 and Ag₂CO₃ were mixed and stirred for 3 mins before adding 10 and Ag₂CO₃. 11 (44% yield, 0.044 mmol, 16.6 mg) was isolated by pTLC (50% EtOAc/hexane) as a white solid.

¹H NMR (600 MHz, Acetone-d₆) δ 9.07 (s, 1H), 8.32 (s, 1H), 7.35 (t, J=54.3 Hz, 1H), 7.27 (d, J=8.2 Hz, 1H), 7.15 (t, J=7.8 Hz, 1H), 6.91 (d, J=7.2 Hz, 1H), 3.99 (s, 3H), 3.61 (q, J=6.9 Hz, 1H), 3.21 (d, J=2.4 Hz, 1H), 1.31 (d, J=6.9 Hz, 3H), 1.27 (s, 3H), 1.19 (s, 3H). ¹³C NMR (151 MHz, Acetone-d₆) δ 178.90, 160.10, 146.49, 139.87, 133.57, 133.13, 128.46, 120.32, 120.15, 117.45, 111.07 (t, J=234.3 Hz), 108.10, 59.06, 44.01, 42.72, 39.93, 23.71, 22.38, 19.01. ¹⁹F NMR (376 MHz, CDCl₃) δ −110.26 (d, J=102.3 Hz).

HRMS (ESI-TOF) Calculated for C₁₉H₂₂F₂N₃O₃[M+H]⁺: 378.1629, Found: 378.1632.

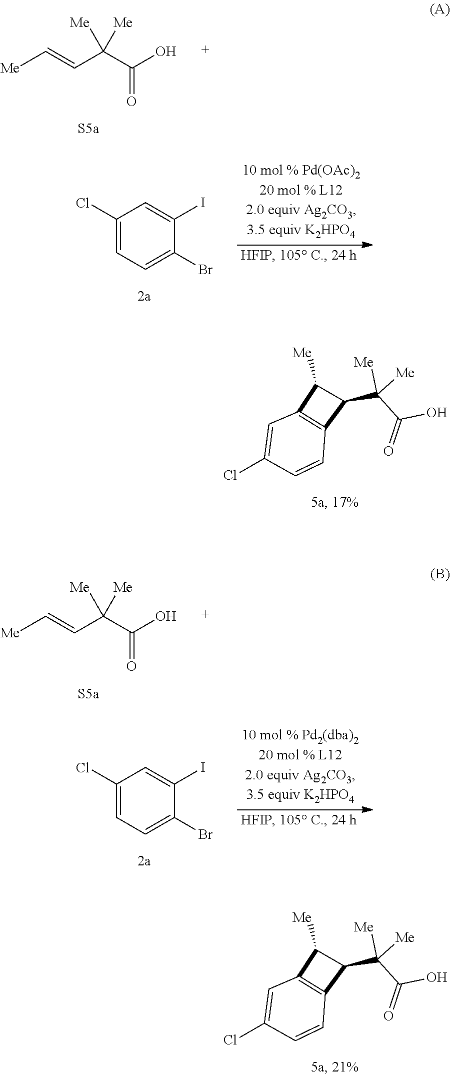

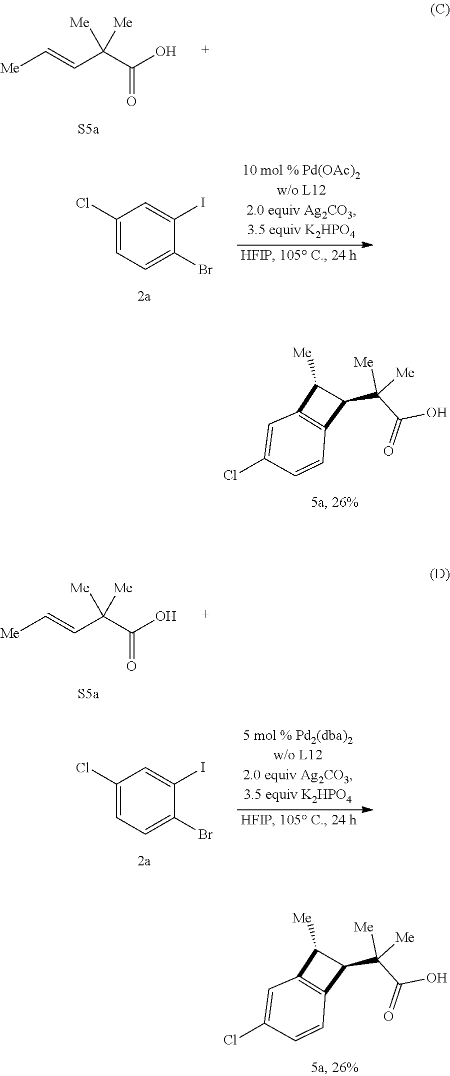

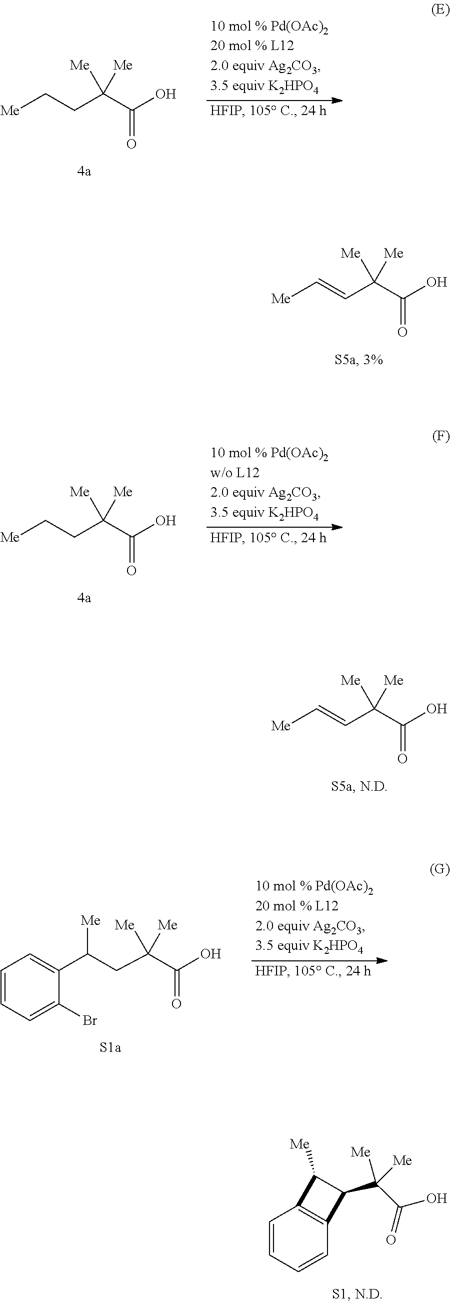

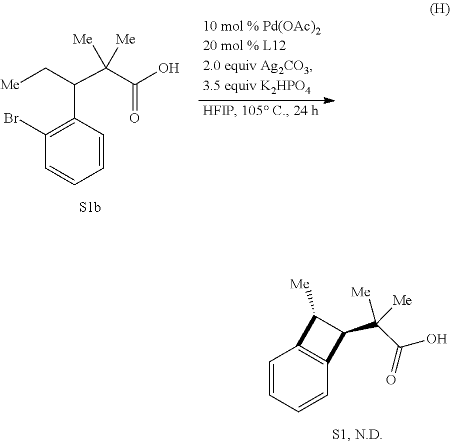

To gain mechanistic insights of this Pd-catalyzed [2+2] annulation, the reaction of 4a and 2a was monitored by ¹H NMR. A trace of β,γ-dehydrogenation product S5a (<3% yield) was observed since the beginning of the reaction (FIG. S1A). Next, using dehydrogenation product S5a as the substrate, the desired BCB product 5a could be obtained in 17% or 21% yield with Pd(OAc)₂ or Pd₂(dba)₃ as the catalyst (FIG. S2A, B). In the absence of L12, a slightly higher yield of 5a was observed when using Pd(OAc)₂ or Pd₂(dba)₃ as the catalyst (FIG. S2C, D), indicating ligand L12 is unessential for this process. Pd₂(dba)₃ gave higher yield than Pd(OAc)₂ with S5a as the substrate, showing that Pd⁰ probably be the active species with S5a. Under the standard conditions without dihaloarene coupling partner, the formation of S5a in 3% yield (FIG. S2E) was observed, and S5a was not formed under the ligandless conditions (FIG. S2F), indicating ligand L12 is crucial for the dehydrogenation process. No desired BCB product could be observed when using R or γ-C—H monoarylation intermediates S1a and S1b as the substrates under the standard conditions without dihaloarene (FIG. S2G, H). Taken together, these results suggest that dehydrogenation product S5a is the reasonable intermediate in this reaction.

Based on the above control experiments, that this transformation proceeds via a Pd^{II}/Pd⁰/Pd^{II}/Pd^{IV} catalytic cycle outlined in FIG. S3 was proposed. Firstly, Pd^{II} coordinates with a substrate and an amide-pyridone ligand to form int-I for the cleavage of γ-C—H bond, promoted by the bidentate ligand. Then, it goes through a β-hydride elimination to form int-II as a Pd⁰ species. That dehydrogenated acid S5a is an ideal ligand for Pd⁰ was proposed. Int-IT can be a resting state if there is no dihaloarene pushing it forward since the yield is very low in the absence of dihaloarene (FIG. S2E). A 3-C—H activation followed by β-hydride elimination with the γ-C—H bond cannot be excluded. After a ligand dissociation and an intermolecular oxidative addition of the Pd⁰ into the aryl-iodo bond, int-III is formed as a Pd^{II} species. Then, it goes through a carbopalladation to form int-IV. Next, proposed was an intramolecular oxidative addition of the Pd^{II} into the aryl-bromo bond to form int-V as a Pd^{IV} species. Then, after a reductive elimination, the product can be obtained with a regenerated Pd^{II} species. Silver salt is proposed to be a halogen scavenger. From which intermediate silver takes over halogens, as well as its roles in other steps is under research. Diastereoselectivity of BCB products is proposed to be from the dehydrogenation step, in which small cyclic acids tend to form cis-alkene leading to cis-BCBs, and the acyclic acids generate trans-alkenes leading to trans-BCBs. Further studies on the reaction mechanism are under way.

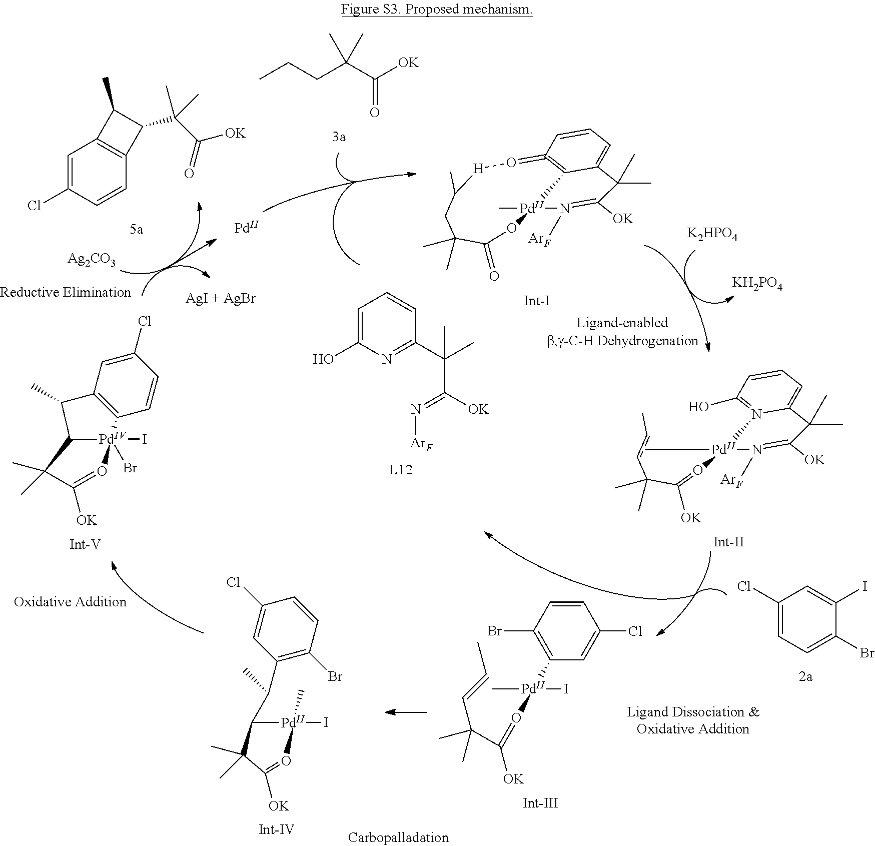

Compound S1a was synthesized according to the procedure below (8).

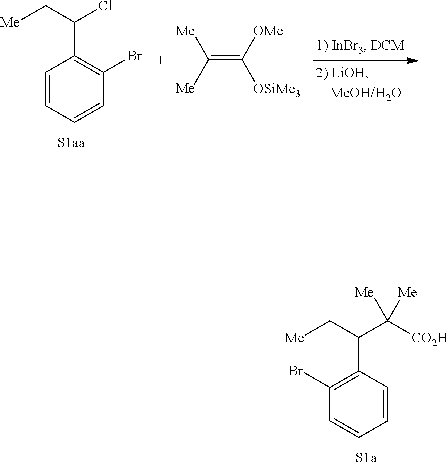

S1aa was synthesized according to the reported procedure (9). To a mixture of InBr₃ (0.05 mmol, 17.7 mg) and dimethylketene methyl trimethylsilyl acetal (1.5 mmol, 261 mg) in CH₂Cl₂ (1.0 mL) was added S1aa (1 mmol, 233 mg) under nitrogen. The reaction mixture was stirred overnight. The resulting mixture was poured into Et₂O (10 mL) and aqueous NaHCO₃ (10 mL). The solution was extracted with Et₂O and the organic layer was dried over MgSO₄. The evaporation of the solvent gave the crude product which could be used for next step without purification.

Redissolved the crude product in MeOH (10 mL) followed by adding water (5 mL) and LiOH (2 mmol, 48 mg). Then the reaction stirred at 50° C. for 18 h. The volatiles were removed in vacuo and the resulting residue was partitioned between a saturated solution of NH₄Cl and CH₂Cl₂. The aqueous phase was further extracted with CH₂Cl₂ and the combined organic layers was dried by anhydrous Na₂SO₄ and concentrated under reduced pressure. The residue was purified by chromatography on silica gel using EtOAc/hexane to afford the expected product S1a (75% yield for two steps, 0.75 mmol, 214 mg, white solid). ¹H NMR (600 MHz, CDCl₃) δ 7.62-7.58 (m, 1H), 7.31-7.24 (m, 2H), 7.09 (ddd, J=8.8, 6.4, 2.4 Hz, 1H), 3.79 (dd, J=11.3, 3.8 Hz, 1H), 1.84-1.69 (m, 2H), 1.18 (s, 3H), 1.17 (s, 3H), 0.71 (t, J=7.3 Hz, 3H). ¹³C NMR (151 MHz, CDCl₃) δ 184.52, 140.07, 133.16, 129.18, 128.59, 128.14, 127.25, 51.35, 47.32, 25.32, 24.82, 19.98, 12.48.

HRMS (ESI-TOF) Calculated for C₁₃H₁₆BrO₂ [M−H]⁻: 283.0334, Found: 283.0326.

Compound S1b was synthesized according to the procedure below.

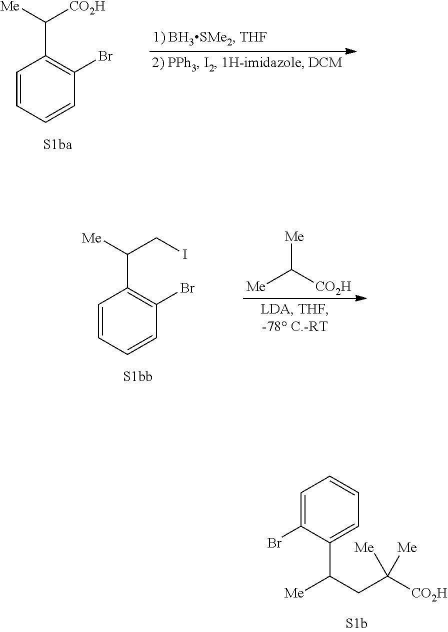 [SMILES: CC(C(=O)O)c1ccccc1Br ; CC(CI)c1ccccc1Br ; CC(C)C(=O)O ; CC(CC(C)(C)C(=O)O)c1ccccc1Br]

To an ice-cold solution of acid S1ba (10 mmol, 2.29 g) in THF (100 mL), a borane dimethyl sulfide complex in THF (10 mL, 20 mmol) was added dropwise. The solution was warmed at room temperature and stirred for overnight. Then the reaction was cooled at 0° C. and quenched with MeOH (20 mL) and stirred for 10 mins. The residue was dissolved in EtOAc, washed with brine, dried over Na₂SO₄ and concentrated in vacuo to obtain the alcohol as colorless oil without further purification.

A solution of triphenylphosphine (15 mmol, 3.93 g) and iodine (15 mmol, 3.81 g) in 50 mL of CH₂Cl₂ was stirred 10 mins at room temperature, then treated with imidazole (25 mmol, 1.7 g), and stirred an additional 10 mins. Then add the alcohol obtained in the last step in 40 mL of CH₂Cl₂ to the solution. After being left to stand overnight at room temperature, the mixture was washed with 10% aqueous sodium bisulfate, the organic layer was separated, and the aqueous layer was extracted three times with Et₂O. The combined organic layers were dried (MgSO₄), filtered, and concentrated in vacuo. The residue was purified by chromatography on silica gel using EtOAc/hexane to afford the expected product S1bb (46% yield for two steps, 4.6 mmol, 1.49 g) as yellow oil. ¹H NMR (600 MHz, CDCl₃) δ 7.57 (d, J=7.9 Hz, 1H), 7.31 (t, J=7.0 Hz, 1H), 7.27-7.21 (m, 1H), 7.14-7.08 (m, 1H), 3.52 (h, J=6.9 Hz, 1H), 3.45 (dd, J=9.7, 5.4 Hz, 1H), 3.30 (dd, J=9.8, 7.8 Hz, 1H), 1.41 (d, J=6.9 Hz, 3H). ¹³C NMR (151 MHz, CDCl₃) δ 143.13, 133.26, 128.51, 127.81, 127.25, 124.76, 40.54, 20.77, 13.29.

LDA solution in THE (10 mL, 5.0 mmol) was added dropwise to a mixture of isobutyric acid (2.0 mmol, 176 mg) and THE (10 mL) at −78° C., and the mixture was allowed to warm up to room temperature and stirred for 1 h. Then the reaction mixture was recooled to −78° C. and S1bb (2.0 mmol, 650 mg) in THE (5 mL) was added dropwise. The resulting solution was allowed to warm up to room temperature and stirred overnight. ¹H NMR (600 MHz, CDCl₃) δ 7.50 (d, J=8.0 Hz, 1H), 7.30-7.21 (m, 2H), 7.01 (td, J=7.6, 1.9 Hz, 1H), 3.44 (h, J=6.9 Hz, 1H), 2.01-1.91 (m, 2H), 1.20 (d, J=7.0 Hz, 3H), 1.18 (s, 3H), 1.18 (s, 3H). ¹³C NMR (151 MHz, CDCl₃) δ 184.30, 146.47, 132.94, 128.07, 127.66, 127.58, 124.25, 47.45, 42.08, 35.30, 26.82, 24.58, 22.79.

HRMS (ESI-TOF) Calculated for C₁₃H₁₆BrO₂ [M−H]⁻: 283.0334, Found: 283.0327.

### REFERENCES

1. O. H. Oldenziel, D. Van Leusen, A. M. Van Leusen, J. Org. Chem. 1977, 42, 3114-3118.
2. A. Muth, V. Pandey, N. Kaur, M. Wason, C. Baker, X. Han, T. Johnson, D. A. Altomare, O. Phanstoel IV, J. Med. Chem. 2014, 57, 4023-4034.
3. X. Huang, M. Hu, X. Zhao, C. Li, Z. Yuan, X. Liu, C. Cai, Y. Zhang, Y. Hu, Y. Chen, Org. Lett. 2019, 21, 3382-3386.
4. E. J. Hennessy, V. Oza, A. Adam, K. Byth, L. Castriotta, G. Grewal, G. A. Hamilton, V. M. 5. Kamhi, P. Lewis, D. Li, P. Lyne, L. Oster, M. T. Rooney, J. C. Saeh, L. Sha, Q. Su, S. Wen, Y. Xue, B. Yang, J. Med. Chem. 2015, 58, 7057-7075.
5. Y. Han, M. Belley, C. I. Bayly, J. Colucci, C. Dufresne, A. Giroux, C. K. Lau, Y. Leblanc, D. McKay, M. Therien, M. C. Wilson, K. Skorey, C. C. Chan, G. Scapin, B. P. Kennedy, Bioorg. Med. Chem. Lett, 2008, 18, 3200-3205.
6. M. Winiewska-Szajewska, A. M. Maciejewska, E. Speina, J. Poznanski, D. Paprocki, Molecules 2021, 26, 3163.
7. I. Fleming, D. H. Williams, Tetrahedron. 1967, 23, 2747-2765.
8. Y. Nishimoto, M. Yasuda, A. Baba, Org. Lett. 2007, 9, 4931-4934.
9. U. Ikuo, U. Suminori, Bulletin of the Chemical Society of Japan, 1975, 48, 2323-2327.

The foregoing disclosure has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the disclosure should, therefore, be determined not with reference to the above description, but should instead be determined with reference to the following appended claims, along with the full scope of equivalents to which such claims are entitled.

This application refers to various issued patents, published patent applications, journal articles, and other publications, each of which are incorporated herein by reference.

What is claimed is:

1. A method of twofold β,γ-methylene C(sp³)-H activation/C—C bond formation, comprising regio-controllable [2+2] annulation between an aliphatic acid and a dihaloarene in the presence of a Pd(II) catalyst bound to an amide-pyridone ligand.

2. The method of claim 1, wherein the regio-controllable [2+2] annulation between an aliphatic acid and a dihaloarene occurs according to the following reaction scheme: 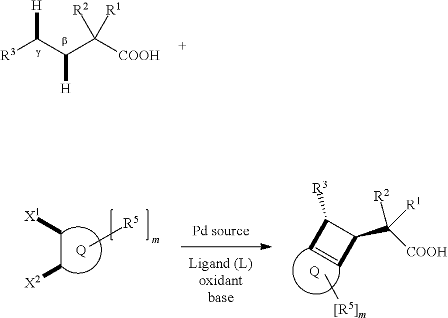
wherein:
Q is Ar or Het;
Ar is (C₆-C₁₀)aryl, optionally substituted with one or more R^{a};
Het is (C₅-C₈)heteroaryl, optionally substituted with one or more R^{a};
X¹ and X² are independently Br or I;
R¹ and R² are independently H or (C₁-C₆)alkyl;
R³ is (C₁-C₆)alkyl, (C₁-C₆)heteroalkyl, halo (C₁-C₆)alkyl, (C₁-C₆)alkyl-OTBS, —C(═O)OH, —C(═O)O(C₁-C₆)alkyl, (C₃-C₇)cycloalkyl, (C₃-C₇)heterocycloalkyl, (C₆-C₁₀)aryl, (C₅-C₆)heteroaryl, (C₁-C₆)alkyl (C₃-C₇)cycloalkyl, (C₁-C₆)alkyl (C₃-C₇)heterocycloalkyl, (C₁-C₆)alkyl (C₆-C₁₀)aryl, —O(C₆-C₁₀)aryl, and (C₁-C₆)alkyl (C₅-C₆)heteroaryl, wherein each is optionally and independently substituted with one or more R^{a};
each R^{a} is independently selected from halo, —OH, —C(═O)O(C₁-C₆)alkyl, CN, NO₂, —P(═O)(O(C₁-C₆)alkyl)₂-C(═O)H, NH₂, (C₁-C₆)alkyl, halo (C₁-C₆)alkyl, (C₁-C₆)heteroalkyl, —C(═O)(C₁-C₆)alkyl, oxo, Ts, —S(═O)₂F, —OS(═O)₂F, or —S(═O)₂(C₁-C₆)alkyl, and —C(═O)NH(C₁-C₆)alkyl;
each R⁵ is independently halo, OH, CN, NO₂,-OTf, (C₁-C₆)alkyl, —C(═O)OH, —C(═O)O(C₁-C₆)alkyl, —C(═O)H, —C(═O)(C₁-C₆)alkyl, (C₁-C₆)alkyl, (C₁-C₆)heteroalkyl, halo (C₁-C₆)alkyl, —C(═O)(C₁-C₆)alkyl, —C(═O)(C₃-C₇)heterocycloalkyl, (C₅-C₆)heteroaryl, or —C(═O)NH(C₁-C₆)alkyl(C₅-C₆)heteroaryl;
or two R⁵ may together form (C₃-C₇)cycloalkyl, (C₃-C₇)heterocycloalkyl, (C₆-C₁₀)aryl, or (C₅-C₆)heteroaryl, wherein each is optionally and independently substituted with one or more R^{a}; and
m is 0, 1, or 2;
including enantiomers, scalemic or racemic mixtures, and pharmaceutically acceptable salts thereof.

3. The method of claim 2, wherein R¹ is H.

4. The method of claim 2, wherein R¹ is (C₁-C₆)alkyl.

5. The method of claim 4, wherein R¹ is Me.

6. The method of claim 4, wherein R¹ is Et.

7. The method of claim 4, wherein R¹ is nPr.

8. The method of claim 4, wherein R¹ is nBu.

9. The method of any one of claims 2-8, wherein R² is H.

10. The method of any one of claims 2-8, wherein R² is (C₁-C₆)alkyl.

11. The method of claim 10, wherein R² is Me.

12. The method of claim 10, wherein R² is Et.

13. The method of claim 10, wherein R² is nPr.

14. The method of claim 10, wherein R² is nBu.

15. The method of any one of claims 2-14, wherein R³ is (C₁-C₆)alkyl.

16. The method of claim 15, wherein R³ is Me.

17. The method of claim 15, wherein R³ is Et.

18. The method of claim 15, wherein R³ is nPr.

19. The method of claim 15, wherein R³ is nBu.

20. The method of claim 15, wherein R³ is pentane.

21. The method of any one of claims 2-14, wherein R³ is (C₁-C₆)heteroalkyl.

22. The method of claim 21, wherein R³ is —O(C₁-C₆)alkyl.

23. The method of any one of claims 2-14, wherein R³ is halo (C₁-C₆)alkyl.

24. The method of any one of claims 2-8, wherein R³ is —(C₁-C₆)alkyl chloride.

25. The method of any one of claims 2-8, wherein R³ is —CH₂P(═O)(EtO)₂.

26. The method of any one of claims 2-8, wherein R³ is Ph, optionally substituted with one or more R^{a}.

27. The method of any one of claims 2-8, wherein R³ is Bz, optionally substituted with one or more R^{a}.

28. The method of either claim 26 or 27, wherein R^{a} is Cl.

29. The method of any one of claims 2-28, wherein n is 1.

30. The method of claim 29, wherein R⁵ is Cl.

31. The method of any one of claims 2-30, wherein X¹ is Br and X² is I.

32. The method of any one of claims 2-30, wherein X¹ is Br and X² is Br.

33. The method of any one of claims 2-30, wherein X¹ is I and X² is I.

34. The method of claim 1, wherein the regio-controllable [2+2] annulation between an aliphatic acid and a dihaloarene occurs according to the following reaction scheme: 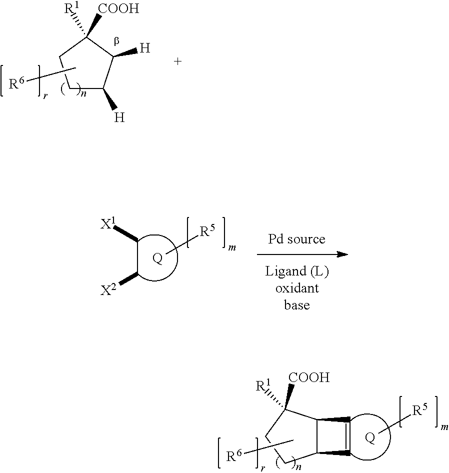
wherein:
Q is Ar or Het;
Ar is (C₆-C₁₀)aryl, optionally substituted with one or more R^{a};
Het is (C₅-C₈)heteroaryl, optionally substituted with one or more R^{a};
X¹ and X² are independently Br or I;
R¹ is R^{1a} or R^{1b};
R^{1a} is H, CN, or halo;
R^{1b} is (C₁-C₆)alkyl, (C₁-C₆)heteroalkyl, halo (C₁-C₆)alkyl, (C₁-C₆)alkyl-OTBS, (C₃-C₇)cycloalkyl, (C₃-C₇)heterocycloalkyl, (C₆-C₁₀)aryl, (C₅-C₆)heteroaryl, (C₁-C₆)alkyl (C₃-C₇)cycloalkyl, (C₁-C₆)alkyl (C₃-C₇)heterocycloalkyl, (C₁-C₆)alkyl (C₆-C₁₀)aryl, and (C₁-C₆)alkyl (C₅-C₆)heteroaryl, wherein each is optionally and independently substituted with one or more R^{a};
each R^{a} is independently selected from halo, —OH, (C₁-C₆)alkyl, —C(═O)O(C₁-C₆)alkyl, CN, NO₂, —P(═O)(O(C₁-C₆)alkyl)₂-C(═O)H, —C(═O)(C₁-C₆)alkyl, —C(═O)OH, —C(═O)O(C₁-C₆)alkyl, NH₂, halo (C₁-C₆)alkyl, (C₁-C₆)heteroalkyl, —C(═O)(C₁-C₆)alkyl, oxo, Ts, —S(═O)₂F, —OS(═O)₂F, or —S(═O)₂(C₁-C₆)alkyl, and —C(═O)NH(C₁-C₆)alkyl;
each R⁵ is independently halo, OH, CN, NO₂,-OTf, (C₁-C₆)alkyl, —C(═O)OH, —C(═O)O(C₁-C₆)alkyl, —C(═O)H, —C(═O)(C₁-C₆)alkyl, (C₁-C₆)alkyl, (C₁-C₆)heteroalkyl, halo (C₁-C₆)alkyl, —C(═O)(C₁-C₆)alkyl, —C(═O)(C₃-C₇)heterocycloalkyl, (C₅-C₆)heteroaryl, —C(═O)NH(C₁-C₆)alkyl(C₅-C₆)heteroaryl;
or two R⁵ may together form (C₃-C₇)cycloalkyl, (C₃-C₇)heterocycloalkyl, (C₆-C₁₀)aryl, or (C₅-C₆)heteroaryl, wherein each is optionally and independently substituted with one or more R^{a}; and
each R⁶ is independently halo, OH, CN, (C₁-C₆)alkyl, (C₁-C₆)heteroalkyl, or halo (C₁-C₆)alkyl;
m is 0, 1, or 2;
n is 1, 2, 3, 4, 7, 8, or 11; and
r is 0, 1, or 2;
including enantiomers, scalemic or racemic mixtures, and pharmaceutically acceptable salts thereof.

35. The method of claim 34, wherein R¹ is H.

36. The method of claim 34, wherein R¹ is (C₁-C₆)alkyl.

37. The method of claim 36, wherein R¹ is Me.

38. The method of claim 36, wherein R¹ is Et.

39. The method of claim 36, wherein R¹ is nPr.

40. The method of claim 36, wherein R¹ is sec-Bu.

41. The method of claim 34, wherein R¹ is Ph, optionally substituted with one or more R^{a}.

42. The method of claim 34, wherein R¹ is (C₁-C₆)alkyl (C₆-C₁₀)aryl, optionally substituted with one or more R^{a}.

43. The method of claim 34, wherein R¹ is (C₁-C₆)heteroalkyl.

44. The method of claim 34, wherein R¹ is (C₁-C₆)alkyl (C₃-C₇)cycloalkyl.

45. The method of any one of claims 34-45, wherein r is 0.

46. The method of any one of claims 34-45, wherein r is 1.

47. The method of any one of claims 34-45, wherein r is 2.

48. The method of either one of claim 46 or 47, wherein one R⁶ is Me.

49. The method of claim 47, wherein both R⁶ are Me.

50. The method of any one of claims 34-49, wherein m is 0.

51. The method of any one of claims 34-49, wherein m is 1.

52. The method of any one of claims 34-49, wherein m is 2.

53. The method of any one of claims 34-52, wherein p is 1.

54. The method of any one of claims 34-52, wherein p is 2.

55. The method of any one of claims 34-52, wherein p is 3.

56. The method of any one of claims 34-52, wherein p is 4.

57. The method of any one of claims 34-52, wherein p is 7.

58. The method of any one of claims 34-52, wherein p is 8.

59. The method of any one of claims 34-52, wherein p is 11.

60. The method of any one of claims 1-59, wherein the Pd source is Pd(OAc)₂.

61. The method of any one of claims 1-59, wherein the Pd source is Pd(CH₃CN)₂Cl₂.

62. The method of any one of claims 1-59, wherein the Pd source is Pd(TFA)₂.

63. The method of any one of claims 1-59, wherein the Pd source is Pd₂(dba)₃.

64. The method of any one of claims 1-59, wherein the Pd source is Pd(OPiv)₂.

65. The method of any one of claims 1-59, wherein the Pd source is Pd(CH₃CN)₄(BF₄)₂.

66. The method of any one of claims 1-59, wherein the Pd source is approximately 10 mol %.

67. The method of any one of claims 1-59, wherein the Pd source is approximately 5 mol %.

68. The method of any one of claims 1-59, wherein the Ligand (L) is selected from the group consisting of:

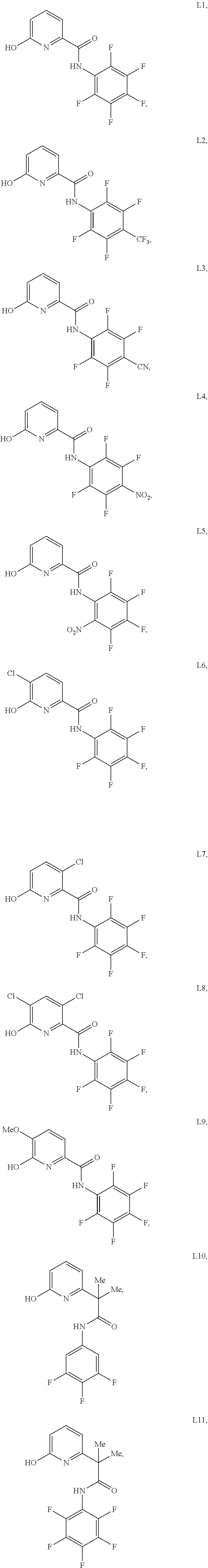

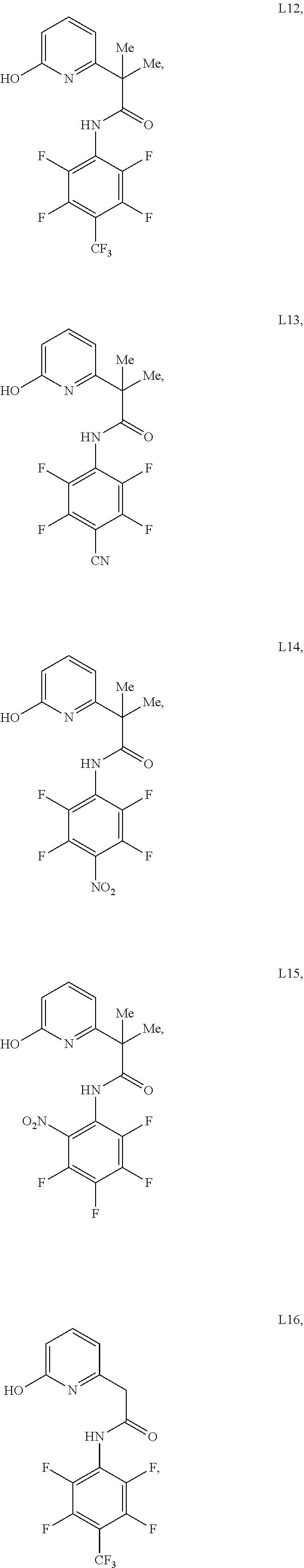 [SMILES: CC(C)(C(=O)Nc1c(F)c(F)c(C(F)(F)F)c(F)c1F)c1cccc(O)n1 ; CC(C)(C(=O)Nc1c(F)c(F)c(C#N)c(F)c1F)c1cccc(O)n1 ; CC(C)(C(=O)Nc1c(F)c(F)c([N+](=O)[O-])c(F)c1F)c1cccc(O)n1 ; CC(C)(C(=O)Nc1c(F)c(F)c(F)c(F)c1[N+](=O)[O-])c1cccc(O)n1 ; O=C(Cc1cccc(O)n1)Nc1c(F)c(F)c(C(F)(F)F)c(F)c1F]

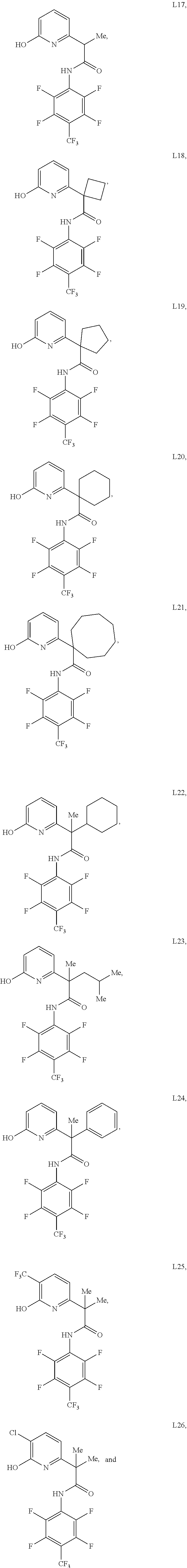

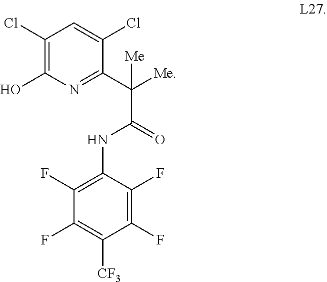 [SMILES: CC(C)(C(=O)Nc1c(F)c(F)c(C(F)(F)F)c(F)c1F)c1nc(O)c(Cl)cc1Cl]

69. The method of any one of claims 1-59, wherein the Ligand (L) is L7.

70. The method of any one of claims 1-59, wherein the Ligand (L) is L12.

71. The method of any one of claims 1-59, wherein the Ligand (L) is selected from the group consisting of:

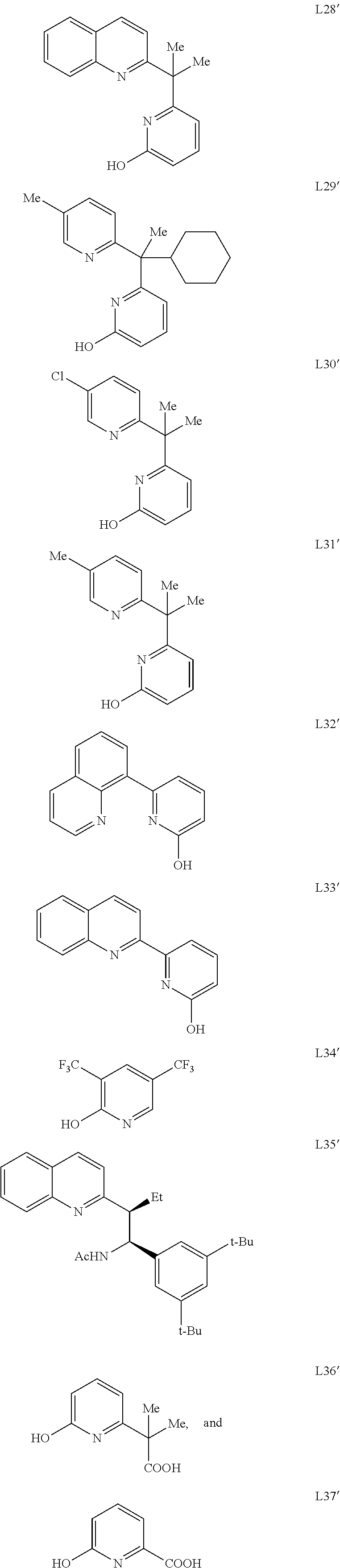

72. The method of any one of claims 2-71, wherein Q is Ph optionally substituted with one or more R^{a}.

73. The method of any one of claims 2-71, wherein Q is Het optionally substituted with one or more R^{a}.

74. The method of claim 73, wherein Het is pyridinyl.

75. The method of claim 73, wherein Het is quinolinyl.

76. The method of claim 73, wherein Het is quinoxalinyl.

77. The method of claim 73, wherein Het is indolyl.

78. The method of any one of claims 2-77, wherein the oxidant is AgOAc.

79. The method of any one of claims 2-77, wherein the oxidant is Ag₂CO₃.

80. The method of any one of claims 2-77, wherein the oxidant is Ag₂O.

81. The method of any one of claims 2-77, wherein the oxidant is AgF.

82. The method of any one of claims 2-77, wherein the oxidant is Ag₃PO₄.

83. The method of any one of claims 2-82, wherein the base is Na₂CO₃.

84. The method of any one of claims 2-82, wherein the base is Li₂CO₃.

85. The method of any one of claims 2-82, wherein the base is K₂CO₃.

86. The method of any one of claims 2-82, wherein the base is Cs₂CO₃.

87. The method of any one of claims 2-82, wherein the base is NaHCO₃.

88. The method of any one of claims 2-82, wherein the base is KHCO₃.

89. The method of any one of claims 2-82, wherein the base is KH₂PO₄.

90. The method of any one of claims 2-82, wherein the base is K₃PO₄.

91. The method of any one of claims 2-82, wherein the base is NaOAc.

92. The method of any one of claims 2-99, wherein the solvent is HFIP.

93. The method of any one of claims 2-92, wherein the reaction temperature is between approximately 60-120° C.

94. The method of any one of claims 2-107, wherein the reaction temperature is between approximately 70-110° C.

95. The method of any one of claims 2-107, wherein the reaction temperature is between approximately 100-110° C.

96. The method of any one of claims 2-95, wherein the mol % of Pd source is approximately between 5-15%.

97. The method of any one of claims 2-95, wherein the mol % of Pd source is approximately 10%.

98. The method of any one of claims 2-97, wherein the mol % of Ligand is approximately between 5-25%.

99. The method of any one of claims 2-97, wherein the mol % of Ligand is approximately between 10-20%.

100. The method of any one of claims 2-97, wherein the mol % of Ligand is approximately 13%.

101. The method of any one of claims 2-100, wherein the equivalents of aliphatic acid compared to dihaloarene is approximately 1:2.

102. The method of any one of claims 2-101, wherein the equivalents of aryl or heteroaryl iodide compared to carboxylic acid substrate is approximately 2.0.

103. The method of any one of claims 2-102, wherein the equivalents of oxidant compared to carboxylic acid substrate is approximately 20:1.

104. The method of any one of claims 2-103, wherein the equivalents of base compared to carboxylic acid substrate is approximately 25:1.

105. A compound having the structure selected from the group consisting of: 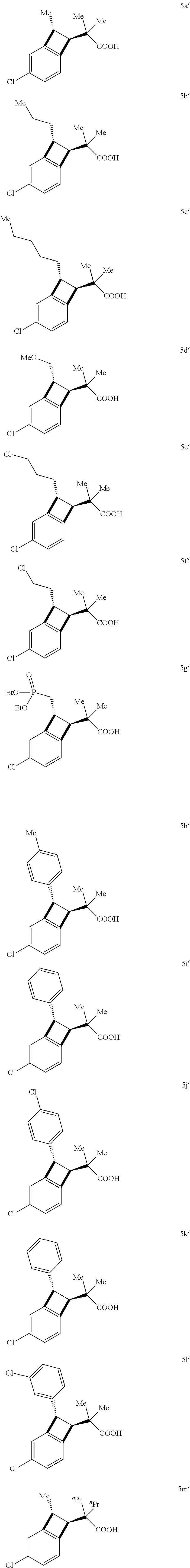 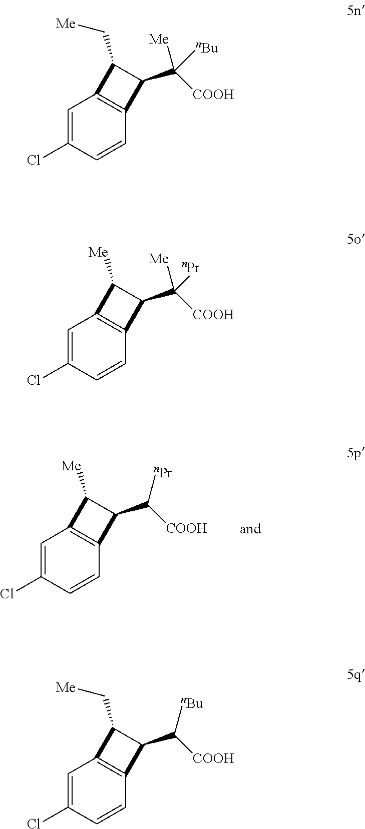
including enantiomers, scalemic or racemic mixtures, and pharmaceutically acceptable salts thereof.

106. A method of preparing the compound of claim 105, comprising the following step: 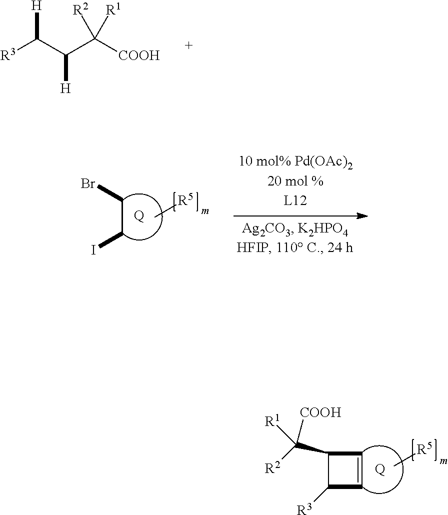
wherein:
Q is Ar or Het;
Ar is (C₆-C₁₀)aryl, optionally substituted with one or more R^{a};
Het is (C₅-C₈)heteroaryl, optionally substituted with one or more R^{a};
X¹ and X² are independently Br or I;
R¹ and R² are independently H or (C₁-C₆)alkyl;
R³ is (C₁-C₆)alkyl, (C₁-C₆)heteroalkyl, halo (C₁-C₆)alkyl, (C₁-C₆)alkyl-OTBS, —C(═O)OH, —C(═O)O(C₁-C₆)alkyl, (C₃-C₇)cycloalkyl, (C₃-C₇)heterocycloalkyl, (C₆-C₁₀)aryl, (C₅-C₆)heteroaryl, (C₁-C₆)alkyl (C₃-C₇)cycloalkyl, (C₁-C₆)alkyl (C₃-C₇)heterocycloalkyl, (C₁-C₆)alkyl (C₆-C₁₀)aryl, —O(C₆-C₁₀)aryl, and (C₁-C₆)alkyl (C₅-C₆)heteroaryl, wherein each is optionally and independently substituted with one or more R^{a};
each R^{a} is independently selected from halo, —OH, —C(═O)O(C₁-C₆)alkyl, CN, NO₂, —P(═O)(O(C₁-C₆)alkyl)₂-C(═O)H, NH₂, (C₁-C₆)alkyl, halo (C₁-C₆)alkyl, (C₁-C₆)heteroalkyl, —C(═O)(C₁-C₆)alkyl, oxo, Ts, —S(═O)₂F, —OS(═O)₂F, or —S(═O)₂(C₁-C₆)alkyl, and —C(═O)NH(C₁-C₆)alkyl;
each R⁵ is independently halo, OH, CN, NO₂,-OTf, (C₁-C₆)alkyl, —C(═O)OH, —C(═O)O(C₁-C₆)alkyl, —C(═O)H, —C(═O)(C₁-C₆)alkyl, (C₁-C₆)alkyl, (C₁-C₆)heteroalkyl, halo (C₁-C₆)alkyl, —C(═O)(C₁-C₆)alkyl, —C(═O)(C₃-C₇)heterocycloalkyl, (C₅-C₆)heteroaryl, or —C(═O)NH(C₁-C₆)alkyl(C₅-C₆)heteroaryl;
or two R⁵ may together form (C₃-C₇)cycloalkyl, (C₃-C₇)heterocycloalkyl, (C₆-C₁₀)aryl, or (C₅-C₆)heteroaryl, wherein each is optionally and independently substituted with one or more R^{a}; and
m is 0, 1, or 2;
including enantiomers, scalemic or racemic mixtures, and pharmaceutically acceptable salts thereof.

107. A compound having the structure selected from the group consisting of: 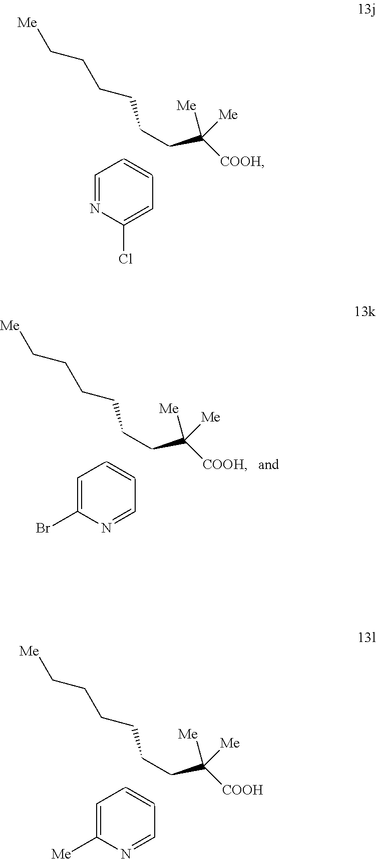
including enantiomers, scalemic or racemic mixtures, and pharmaceutically acceptable salts thereof.

108. A method of preparing the compound of claim 107, comprising the following step: 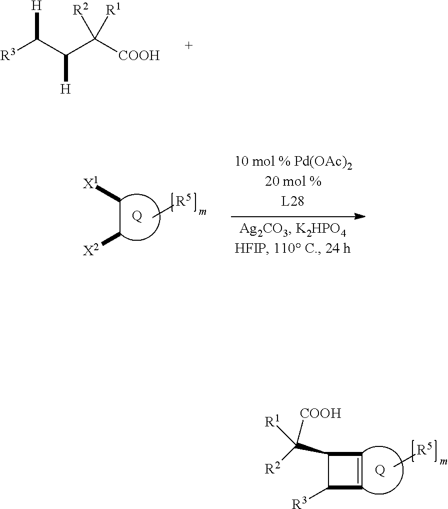
wherein:
X¹ and X² are independently Br or I;
Q is Ar or Het;
Ar is (C₆-C₁₀)aryl, optionally substituted with one or more R^{a};
Het is (C₅-C₈)heteroaryl, optionally substituted with one or more R^{a};
R¹ and R² are independently H or (C₁-C₆)alkyl;
R³ is (C₁-C₆)alkyl, (C₁-C₆)heteroalkyl, halo (C₁-C₆)alkyl, (C₁-C₆)alkyl-OTBS, —C(═O)OH, —C(═O)O(C₁-C₆)alkyl, (C₃-C₇)cycloalkyl, (C₃-C₇)heterocycloalkyl, (C₆-C₁₀)aryl, (C₅-C₆)heteroaryl, (C₁-C₆)alkyl (C₃-C₇)cycloalkyl, (C₁-C₆)alkyl (C₃-C₇)heterocycloalkyl, (C₁-C₆)alkyl (C₆-C₁₀)aryl, —O(C₆-C₁₀)aryl, and (C₁-C₆)alkyl (C₅-C₆)heteroaryl, wherein each is optionally and independently substituted with one or more R^{a};
each R^{a} is independently selected from halo, —OH, —C(═O)O(C₁-C₆)alkyl, CN, NO₂, —P(═O)(O(C₁-C₆)alkyl)₂-C(═O)H, NH₂, (C₁-C₆)alkyl, halo (C₁-C₆)alkyl, (C₁-C₆)heteroalkyl, —C(═O)(C₁-C₆)alkyl, oxo, Ts, —S(═O)₂F, —OS(═O)₂F, or —S(═O)₂(C₁-C₆)alkyl, and —C(═O)NH(C₁-C₆)alkyl;
each R⁵ is independently halo, OH, CN, NO₂,-OTf, (C₁-C₆)alkyl, —C(═O)OH, —C(═O)O(C₁-C₆)alkyl, —C(═O)H, —C(═O)(C₁-C₆)alkyl, (C₁-C₆)alkyl, (C₁-C₆)heteroalkyl, halo (C₁-C₆)alkyl, —C(═O)(C₁-C₆)alkyl, —C(═O)(C₃-C₇)heterocycloalkyl, (C₅-C₆)heteroaryl, or —C(═O)NH(C₁-C₆)alkyl(C₅-C₆)heteroaryl;
or two R⁵ may together form (C₃-C₇)cycloalkyl, (C₃-C₇)heterocycloalkyl, (C₆-C₁₀)aryl, or (C₅-C₆)heteroaryl, wherein each is optionally and independently substituted with one or more R^{a}; and
m is 0, 1, or 2;
including enantiomers, scalemic or racemic mixtures, and pharmaceutically acceptable salts thereof.

109. A compound having the structure selected from the group consisting of: 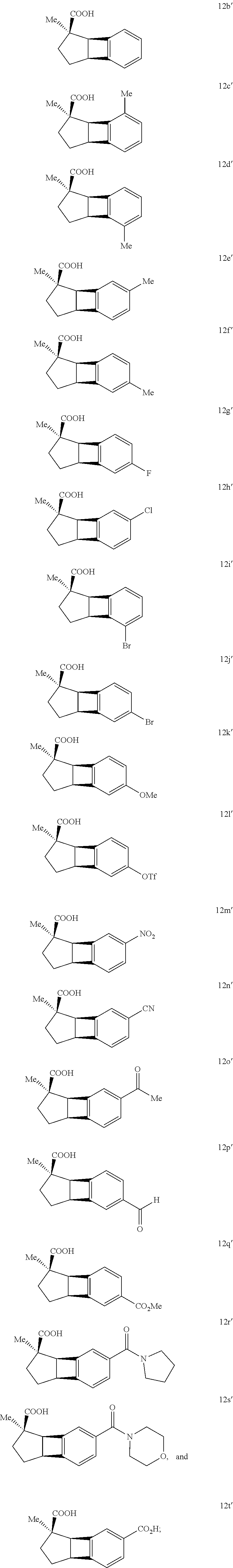
including enantiomers, scalemic or racemic mixtures, and pharmaceutically acceptable salts thereof.

110. A compound having the structure selected from the group consisting of: 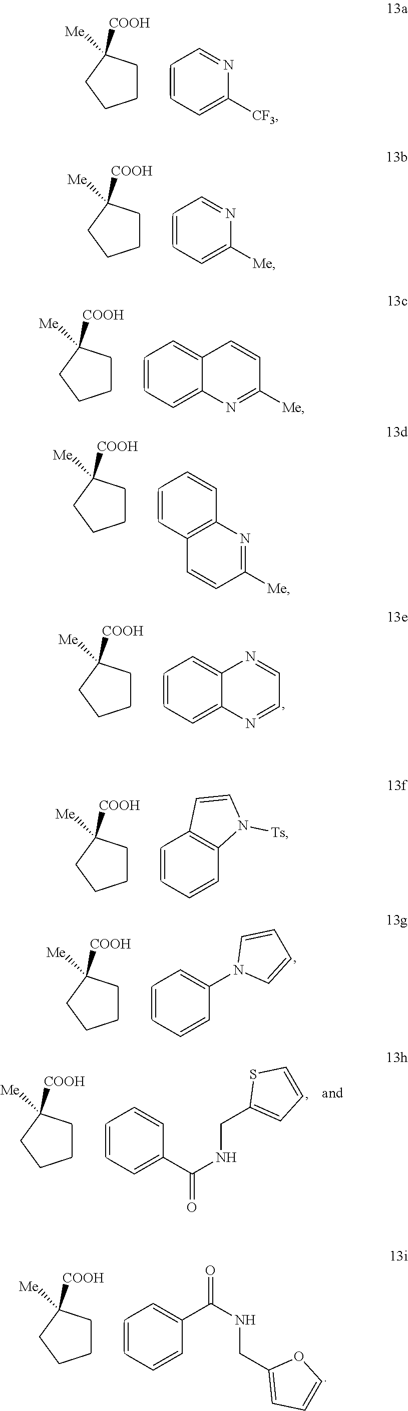
including enantiomers, scalemic or racemic mixtures, and pharmaceutically acceptable salts thereof.

111. A compound having the structure selected from the group consisting of:

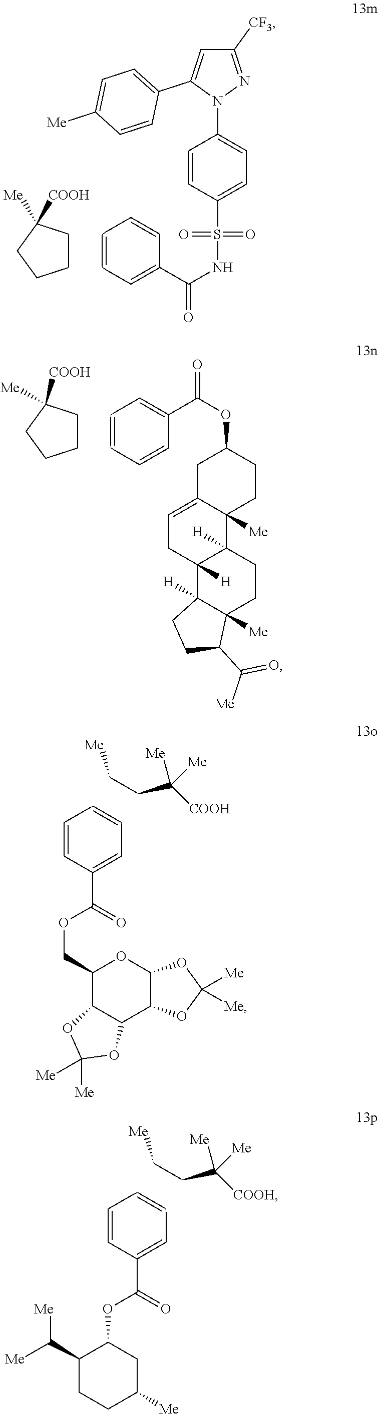

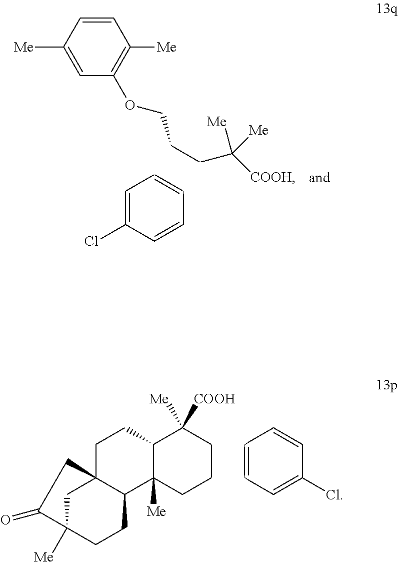

112. A method of preparing the compound of any one of claims 109-111, comprising the following step: 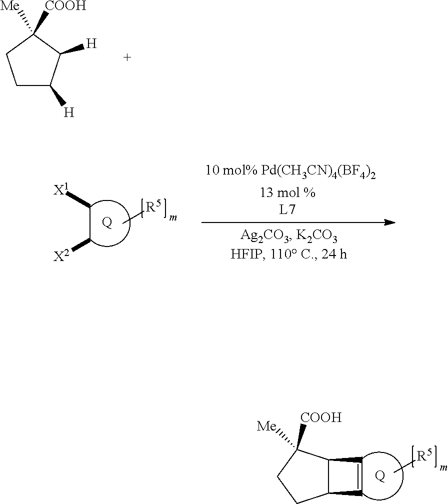
wherein
X¹ and X² are independently Br or I;
Q is Ar or Het;
Ar is (C₆-C₁₀)aryl, optionally substituted with one or more R^{a};
Het is (C₅-C₈)heteroaryl, optionally substituted with one or more R^{a};
each R^{a} is independently selected from halo, —OH, (C₁-C₆)alkyl, —C(═O)O(C₁-C₆)alkyl, CN, NO₂, —P(═O)(O(C₁-C₆)alkyl)₂-C(═O)H, —C(═O)(C₁-C₆)alkyl, —C(═O)OH, —C(═O)O(C₁-C₆)alkyl, NH₂, halo (C₁-C₆)alkyl, (C₁-C₆)heteroalkyl, —C(═O)(C₁-C₆)alkyl, oxo, Ts, —S(═O)₂F, —OS(═O)₂F, or —S(═O)₂(C₁-C₆)alkyl, and —C(═O)NH(C₁-C₆)alkyl;
each R⁵ is independently halo, OH, CN, NO₂,-OTf, (C₁-C₆)alkyl, —C(═O)OH, —C(═O)O(C₁-C₆)alkyl, —C(═O)H, —C(═O)(C₁-C₆)alkyl, (C₁-C₆)alkyl, (C₁-C₆)heteroalkyl, halo (C₁-C₆)alkyl, —C(═O)(C₁-C₆)alkyl, —C(═O)(C₃-C₇)heterocycloalkyl, (C₅-C₆)heteroaryl, —C(═O)NH(C₁-C₆)alkyl(C₅-C₆)heteroaryl;
or two R⁵ may together form (C₃-C₇)cycloalkyl, (C₃-C₇)heterocycloalkyl, (C₆-C₁₀)aryl, or (C₅-C₆)heteroaryl, wherein each is optionally and independently substituted with one or more R^{a}; and
m is 0, 1, or 2;
including enantiomers, scalemic or racemic mixtures, and pharmaceutically acceptable salts thereof.

113. A method of preparing the Compound 6, comprising the following steps:

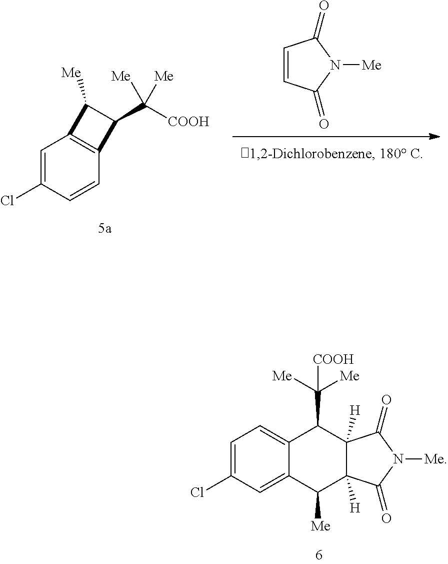

114. A method of preparing the Compound 7, comprising the following steps:

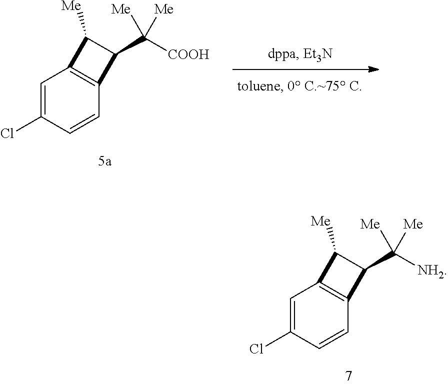

115. A method of preparing the Compound 10, comprising the following steps:

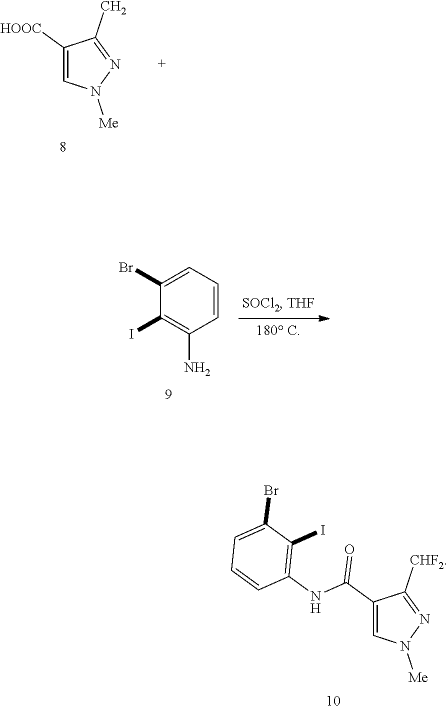

116. A method of preparing the Compound 11, comprising the following steps:

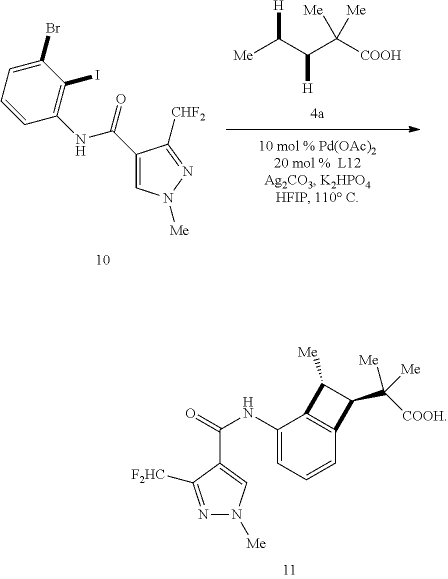

117. A compound having the Formula I 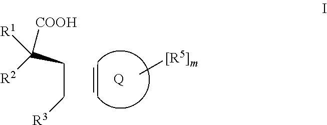
wherein:
Q is Ar or Het;
Ar is (C₆-C₁₀)aryl, optionally substituted with one or more R^{a};
Het is (C₅-C₈)heteroaryl, optionally substituted with one or more R^{a};
R¹ and R² are independently H or (C₁-C₆)alkyl, wherein both R¹ and R² are not H;
R³ is (C₁-C₆)alkyl, (C₁-C₆)heteroalkyl, halo (C₁-C₆)alkyl, (C₁-C₆)alkyl-OTBS, —C(═O)OH, —C(═O)O(C₁-C₆)alkyl, (C₃-C₇)cycloalkyl, (C₃-C₇)heterocycloalkyl, (C₆-C₁₀)aryl, (C₅-C₆)heteroaryl, (C₁-C₆)alkyl (C₃-C₇)cycloalkyl, (C₁-C₆)alkyl (C₃-C₇)heterocycloalkyl, (C₁-C₆)alkyl (C₆-C₁₀)aryl, —O(C₆-C₁₀)aryl, and (C₁-C₆)alkyl (C₅-C₆)heteroaryl, wherein each is optionally and independently substituted with one or more R^{a};
each R^{a} is independently selected from halo, —OH, —C(═O)O(C₁-C₆)alkyl, CN, NO₂, —P(═O)(O(C₁-C₆)alkyl)₂-C(═O)H, NH₂, (C₁-C₆)alkyl, halo (C₁-C₆)alkyl, (C₁-C₆)heteroalkyl, —C(═O)(C₁-C₆)alkyl, oxo, Ts, —S(═O)₂F, —OS(═O)₂F, or —S(═O)₂(C₁-C₆)alkyl, and —C(═O)NH(C₁-C₆)alkyl;
each R⁵ is independently halo, OH, CN, NO₂,-OTf, (C₁-C₆)alkyl, —C(═O)OH, —C(═O)O(C₁-C₆)alkyl, —C(═O)H, —C(═O)(C₁-C₆)alkyl, (C₁-C₆)alkyl, (C₁-C₆)heteroalkyl, halo (C₁-C₆)alkyl, —C(═O)(C₁-C₆)alkyl, —C(═O)(C₃-C₇)heterocycloalkyl, (C₅-C₆)heteroaryl, or —C(═O)NH(C₁-C₆)alkyl(C₅-C₆)heteroaryl;
or two R⁵ may together form (C₃-C₇)cycloalkyl, (C₃-C₇)heterocycloalkyl, (C₆-C₁₀)aryl, or (C₅-C₆)heteroaryl, wherein each is optionally and independently substituted with one or more R^{a}; and
m is 0, 1, or 2;
including enantiomers, scalemic or racemic mixtures, and pharmaceutically acceptable salts thereof.

118. A compound having the Formula II 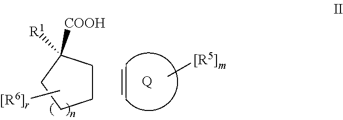
wherein:
Q is Ar or Het;
Ar is (C₆-C₁₀)aryl, optionally substituted with one or more R^{a};
Het is (C₅-C₈)heteroaryl, optionally substituted with one or more R^{a};
R¹ is (C₁-C₆)alkyl, (C₁-C₆)heteroalkyl, halo (C₁-C₆)alkyl, (C₁-C₆)alkyl-OTBS, (C₃-C₇)cycloalkyl, (C₃-C₇)heterocycloalkyl, (C₆-C₁₀)aryl, (C₅-C₆)heteroaryl, (C₁-C₆)alkyl (C₃-C₇)cycloalkyl, (C₁-C₆)alkyl (C₃-C₇)heterocycloalkyl, (C₁-C₆)alkyl (C₆-C₁₀)aryl, and (C₁-C₆)alkyl (C₅-C₆)heteroaryl, wherein each is optionally and independently substituted with one or more R^{a};
each R^{a} is independently selected from halo, —OH, (C₁-C₆)alkyl, —C(═O)O(C₁-C₆)alkyl, CN, NO₂, —P(═O)(O(C₁-C₆)alkyl)₂-C(═O)H, —C(═O)(C₁-C₆)alkyl, —C(═O)OH, —C(═O)O(C₁-C₆)alkyl, NH₂, halo (C₁-C₆)alkyl, (C₁-C₆)heteroalkyl, —C(═O)(C₁-C₆)alkyl, oxo, Ts, —S(═O)₂F, —OS(═O)₂F, or —S(═O)₂(C₁-C₆)alkyl, and —C(═O)NH(C₁-C₆)alkyl;
each R⁵ is independently halo, OH, CN, NO₂,-OTf, (C₁-C₆)alkyl, —C(═O)OH, —C(═O)O(C₁-C₆)alkyl, —C(═O)H, —C(═O)(C₁-C₆)alkyl, (C₁-C₆)alkyl, (C₁-C₆)heteroalkyl, halo (C₁-C₆)alkyl, —C(═O)(C₁-C₆)alkyl, —C(═O)(C₃-C₇)heterocycloalkyl, (C₅-C₆)heteroaryl, —C(═O)NH(C₁-C₆)alkyl(C₅-C₆)heteroaryl;
or two R⁵ may together form (C₃-C₇)cycloalkyl, (C₃-C₇)heterocycloalkyl, (C₆-C₁₀)aryl, or (C₅-C₆)heteroaryl, wherein each is optionally and independently substituted with one or more R^{a}; and
each R⁶ is independently halo, OH, CN, (C₁-C₆)alkyl, (C₁-C₆)heteroalkyl, or halo (C₁-C₆)alkyl;
n is 1, 2, 3, 4, 7, 8, or 11; and
r is 0, 1, or 2;
including enantiomers, scalemic or racemic mixtures, and pharmaceutically acceptable salts thereof.

119. Any method of twofold β,γ-methylene C(sp³)-H activation/C—C bond formation, comprising regio-controllable [2+2] annulation between an aliphatic acid and a dihaloarene in the presence of a Pd(II) catalyst bound to an amide-pyridone ligand, the product thereof, any amide-pyridone ligand, or preparation thereof, as described herein.
